(19) United States
(12) Patent Application Publication
(10) Pub. No.: US 2025/0136579 A1
(43) Pub. Date: May 1, 2025

(54) SALTS AND FREE BASES OF BIFUNCTIONAL COMPOUND AND POLYMORPH FORMS THEREOF

(71) Applicant: NURIX THERAPEUTICS, INC., San Francisco, CA (US)

(72) Inventors: Pasit PHIASIVONGSA, San Francisco, CA (US); Xinnan ZHANG, San Francisco, CA (US); Chih-Tsung LIN, San Francisco, CA (US); Asha SUNKARA, San Francisco, CA (US)

(21) Appl. No.: 18/923,580

(22) Filed: Oct. 22, 2024

Related U.S. Application Data
(60) Provisional application No. 63/670,621, filed on Jul. 12, 2024.
(60) Provisional application No. 63/545,346, filed on Oct. 23, 2023.

(51) Int. Cl. C07D 401/14; A61K 31/497; C07C 57/145; C07C 57/15; C07C 59/235; C07C 59/245; C07C 309/30; C07C 309/35; C07C 309/45

(52) U.S. Cl. CPC C07D 401/14; A61K 31/497; C07C 57/145; C07C 57/15; C07C 59/235; C07C 59/245; C07C 309/30; C07C 309/35; C07C 309/45

(57) ABSTRACT

The present disclosure is directed to salts and free bases of Compounds of Formula (I), including crystalline, semi-crystalline, amorphous, and polymorph forms thereof, and processes for their preparation.

### CROSS-REFERENCE TO RELATED APPLICATIONS

[RESERVED]

### FIELD

The present disclosure is directed to salts of Compounds of Formula (I), including crystalline and polymorph forms thereof, and processes for their preparation. Also provided are free bases of Compounds of Formula (I) and crystalline and polymorph forms thereof, and processes for their preparation.

### BACKGROUND

B cell receptor (BCR) signaling controls B cell development, as well as mature B cell activation, signaling, and survival. Mis-regulation of the BCR signaling pathway is associated with numerous disease indications involving B cell function, and targeting B cells and BCR signaling has clear therapeutic potential (Woyach, et al.; Blood, 120 (6); 1175-1184, 2012). For example, depletion of B cells with monoclonal antibodies targeting CD20 has significant effects in treatment of B cell malignancies and auto-immune and inflammatory diseases (Cang, et al.: J. Hematolo. Oncol. 5:64, 2012.).

BTK is a member of the TEC family of kinases and is a crucial signaling hub in the BCR pathway. Mutations in BTK result in X-linked agammaglobulinaemia (XLA), in which B cell maturation is impaired, resulting in reduced immunoglobulin production (Hendriks, et al.; Expert Opin. Ther. Targets 15; 1002-1021, 2011). The central role of BTK in B cell signaling and function makes BTK an attractive therapeutic target for B cell malignancies as well as autoimmune and inflammatory diseases. Ibrutinib, a covalent inhibitor of BTK, has been approved to treat chronic lymphocytic leukemia (CLL), mantle cell lymphoma (MCL) and other B cell malignancies, as well as graft-versus-host disease (GvHD) (Miklos, et al.; Blood. 120 (21); 2243-2250, 2017). Currently, ibrutinib and second-generation BTK inhibitors are being investigated for oncology and immune-related indications such as rheumatoid arthritis (Akinleye, et al.: J of Hematolo Oncol. 6:59, 2013; Liu, et al.: J. Pharm. and Exper. Ther. 338 (I): 154-163.2011; Di Paolo, et al.: Nat Chem Biol. 7 (1): 41-50, 2011).

As an alternative to stoichiometric inhibition, proteolytic degradation of BTK could have dramatic consequences for B cell function by effectively blocking BCR signaling. Removal of BTK protein would eliminate BTK kinase activity as well as any protein interaction or scaffolding function of BTK. Specific degradation of BTK could be accomplished using heterobifunctional small molecules to recruit BTK to a ubiquitin ligase and thus promoting ubiquitylation and proteasomal degradation of BTK. Thalidomide derivatives, such as lenalidomide or pomalidomide, can be used to recruit potential substrates to cereblon (CRBN), a component of a ubiquitin ligase complex. This unique therapeutic approach could present a mechanism of action for interfering with BTK activity and BCR signaling that is distinct from the mechanism of stoichiometric BTK inhibition. Furthermore, this degradative approach could effectively target the C481S mutated form of BTK, which mutation has been clinically observed and confers resistance to inhibition by ibrutinib (Woyach, et al.; Blood, 120 (6): 1175-1184, 2012).

Given the importance of bifunctional molecules such as the compound of Formula (I), it would be beneficial to develop highly purified and stable forms of compounds of Formula (I) that can be easily manufactured and formulated. There is also a need for salts and polymorph forms of compounds of Formula (I) that can act as and provide these advantageous forms.

### SUMMARY

In one aspect, provided herein is a free base of a compound of Formula (I) (or free base compound of Formula (I)).

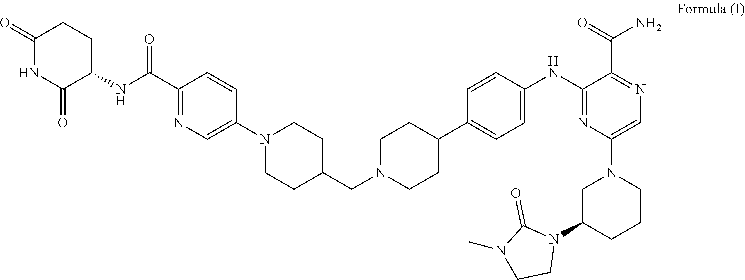 [SMILES: CN1CCN([C@@H]2CCCN(c3cnc(C(N)=O)c(Nc4ccc(C5CCN(CC6CCN(c7ccc(C(=O)N[C@H]8CCC(=O)NC8=O)nc7)CC6)CC5)cc4)n3)C2)C1=O]

In one aspect, provided herein is a free base of a compound of Formula (I) selected from the group consisting of Free Base Form A, Free Base Amorphous Form, Free Base Form B, Free Base Form C, Free Base Form D, Free Base Form E, Free Base Form F, Free Base Form G, Free Base Form H, and Free Base Form J.

In another aspect, provided herein is a solvate of a compound of Formula (I). In one or more embodiments, the free base can be or is a solvate of a compound of Formula (I).

In another aspect, provided herein is a hydrate of a compound of Formula (I). In one or more embodiments, the salt or the free base can be or is a hydrate of a compound of Formula (I).

In another aspect, provided herein is an amorphous form of a compound of Formula (I). In one or more embodiments, the salt or the free base can be or is an amorphous form of a compound of Formula (I).

In one aspect, provided is an amorphous solid dispersion of a free base of a compound of Formula (I). In one aspect, provided is an amorphous solid dispersion that comprises a dispersion agent and a free base. In one aspect, provided is an amorphous solid dispersion of a free base of a compound of Formula (I) selected from the group consisting of Free Base Form A, Free Base Amorphous Form, Free Base Form B, Free Base Form C, Free Base Form D, Free Base Form E, Free Base Form F, Free Base Form G, Free Base Form H, and Free Base Form J.

In another aspect, the present disclosure provides salts of a compound of Formula (I).

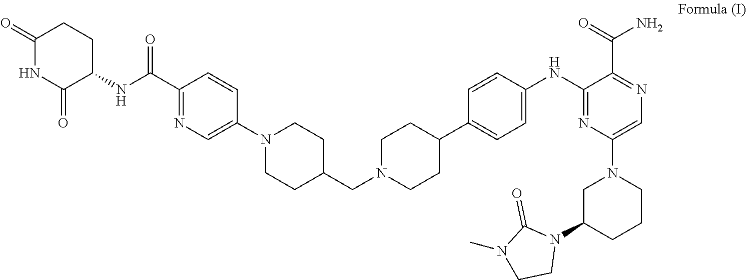 [SMILES: CN1CCN([C@@H]2CCCN(c3cnc(C(N)=O)c(Nc4ccc(C5CCN(CC6CCN(c7ccc(C(=O)N[C@H]8CCC(=O)NC8=O)nc7)CC6)CC5)cc4)n3)C2)C1=O]

In one aspect, provided herein is a salt of a compound of Formula (I) selected from the group consisting of Fumarate Salt Form A, Fumarate Salt Amorphous Form, Fumarate Salt Form B, Fumarate Salt Form C, Fumarate Salt Form D, Fumarate Salt Form E, Maleate Salt Form A, Tosylate Salt Form A, Tosylate Salt Form B, Besylate Salt Form A, Cyclamate Salt Form A, Malate Salt Form A, Malonate Salt Form A, Napsylate Salt Form A, Napsylate Salt Form B, Napsylate Salt Form C, Napsylate Salt Form D, Succinate Salt Form A, Succinate Salt Form B, and Succinate Salt Form C.

In one or more embodiments, the salt of the compound of Formula (I) is provided as a fumarate salt, a maleate salt, or a tosylate salt. In one or more embodiments, the fumarate salt is a salt of fumaric acid, the maleate salt is a salt of maleic acid, and the tosylate salt is a salt of p-toluenesulfonic acid.

In one or more embodiments, the salt of the salt of the compound of Formula (I) is provided as a fumarate salt, a maleate salt, a tosylate salt, a besylate salt, a cyclamate salt, a malate salt, a malonate salt, a napsylate salt, or a succinate salt.

In one or more embodiments, the besylate salt is a salt of benzene sulfonate, the cyclamate salt is a salt of cyclohexylsulfamic acid, the malate salt is a salt of malic acid, a malonate salt is a salt of malonic acid, a napsylate salt is a salt of naphthalene sulfonic acid (including but not limited to naphthalene-1-sulfonic acid, naphthalene-2-sulfonic acid), or a succinate salt is a salt of succinic acid.

In the following schemes, salts of Formula (I-A), (I-B), (I-C), (I-D), (I-E), (I-F), (I-G), (I-H), and (I-J) are depicted without ionic charges. One of ordinary skill in the art would understand structures of salts that form between organic molecules depicted in schemes Formula (I-A), (I-B), (I-C), (I-D), (I-E), (I-F), (I-G), (I-H), and (I-J), and possibilities of ionic charge placement in these schemes. Without wanting to be bound by theory, an ionic charge on a molecule may be a mixture of contributing ionic charges within a probability distribution. As well, ionic charges on the salts of Formula (I-A), (I-B), (I-C), (I-D), (I-E), (I-F), (I-G), (I-H), and (I-J) may include ionic character on a molecule therein, portion(s) of a molecule therein (multiple atoms or substituents), or on an atom or substituent. Thus, salts of Formula (I-A), (I-B), (I-C), (I-D), (I-E), (I-F), (I-G), (I-H), and (I-J) are understood to include ionic charges.

In one or more embodiments, the salt of the compound of Formula (I) is provided as Formula (I-A).

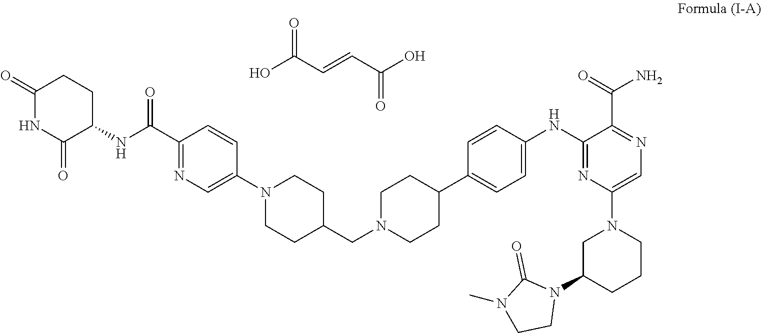 [SMILES: CN1CCN([C@@H]2CCCN(c3cnc(C(N)=O)c(Nc4ccc(C5CCN(CC6CCN(c7ccc(C(=O)N[C@H]8CCC(=O)NC8=O)nc7)CC6)CC5)cc4)n3)C2)C1=O ; O=C(O)/C=C/C(=O)O]

In one or more embodiments, the salt of the compound of Formula (I) is provided as a hemi-fumarate salt of Formula (I-A):

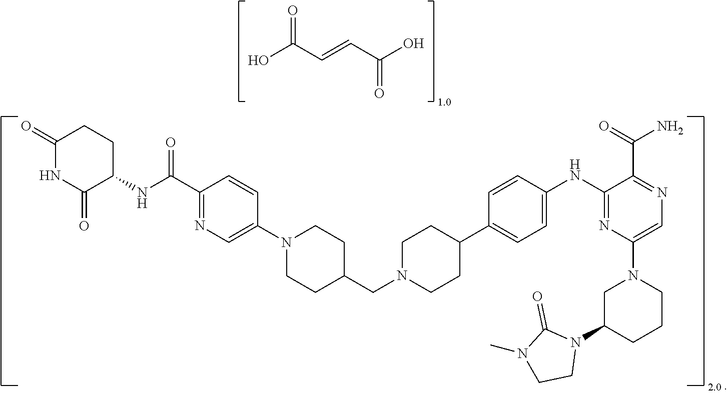

In one or more embodiments, the salt of the compound of Formula (I) is provided as Formula (I-B).

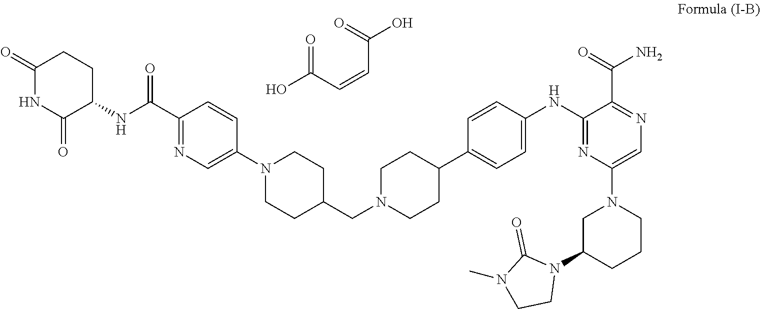 [SMILES: CN1CCN([C@@H]2CCCN(c3cnc(C(N)=O)c(Nc4ccc(C5CCN(CC6CCN(c7ccc(C(=O)N[C@H]8CCC(=O)NC8=O)nc7)CC6)CC5)cc4)n3)C2)C1=O ; O=C(O)/C=C\C(=O)O]

In one or more embodiments, the salt of the compound of Formula (I) is provided as Formula (I-C).

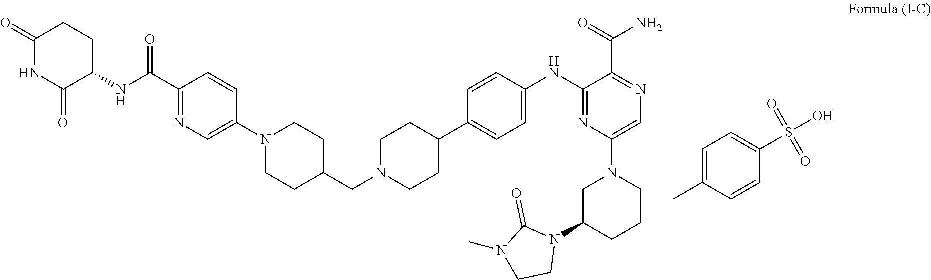

In one or more embodiments, the salt of the compound of Formula (I) is provided as Formula (I-D).

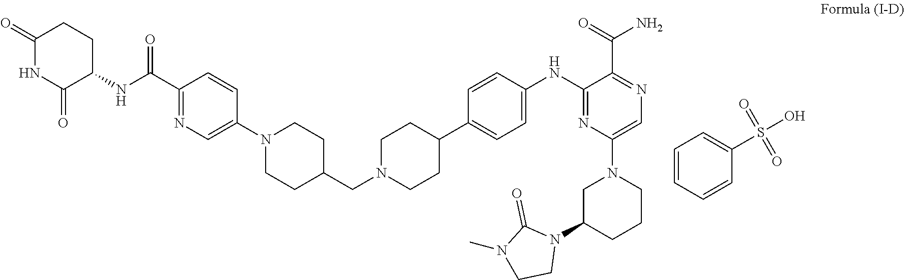

In one or more embodiments, the salt of the compound of Formula (I) is provided as Formula (I-E).

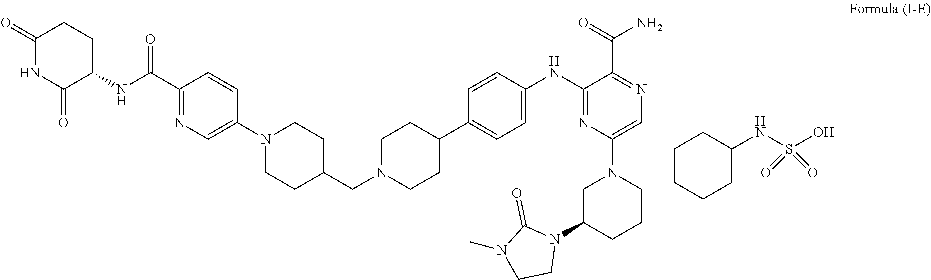

In one or more embodiments, the salt of the compound of Formula (I) is provided as Formula (I-F).

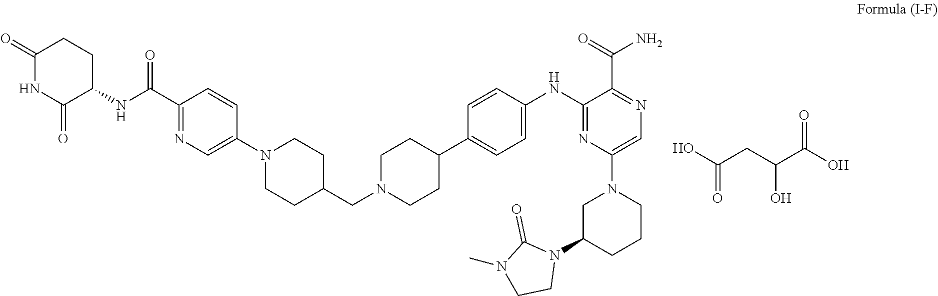 [SMILES: CN1CCN([C@@H]2CCCN(c3cnc(C(N)=O)c(Nc4ccc(C5CCN(CC6CCN(c7ccc(C(=O)N[C@H]8CCC(=O)NC8=O)nc7)CC6)CC5)cc4)n3)C2)C1=O ; O=C(O)CC(O)C(=O)O]

In one or more embodiments, the salt of the compound of Formula (I) is provided as Formula (I-G).

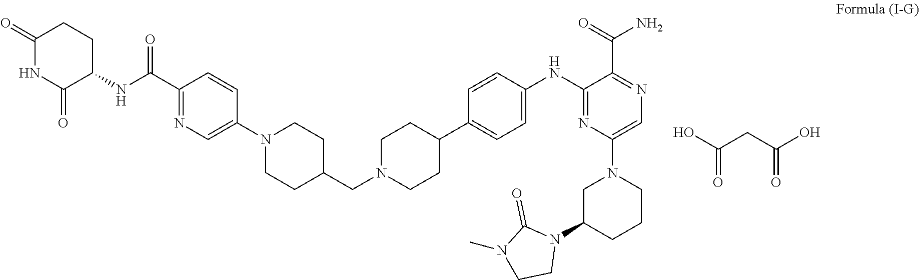 [SMILES: CN1CCN([C@@H]2CCCN(c3cnc(C(N)=O)c(Nc4ccc(C5CCN(CC6CCN(c7ccc(C(=O)N[C@H]8CCC(=O)NC8=O)nc7)CC6)CC5)cc4)n3)C2)C1=O ; O=C(O)CC(=O)O]

In one or more embodiments, the salt of the compound of Formula (I) is provided as Formula (I-H).

US

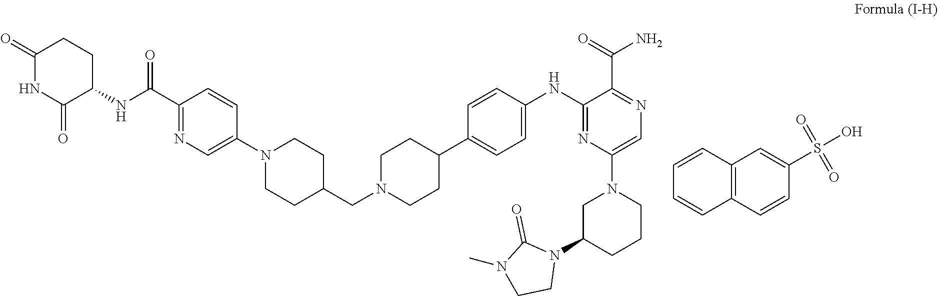

In one or more embodiments, the salt of the compound of Formula (I) is provided as Formula (I-J).

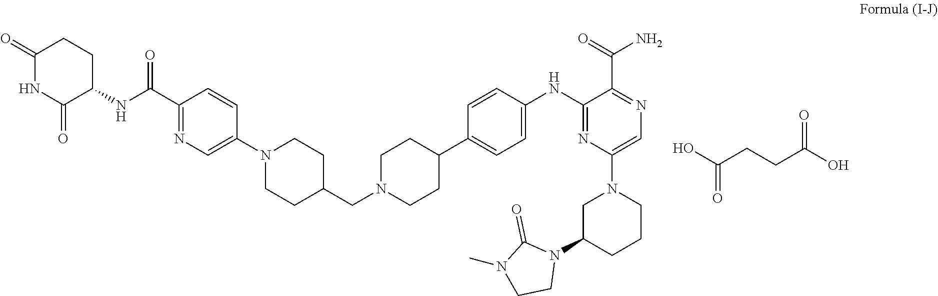 [SMILES: CN1CCN([C@@H]2CCCN(c3cnc(C(N)=O)c(Nc4ccc(C5CCN(CC6CCN(c7ccc(C(=O)N[C@H]8CCC(=O)NC8=O)nc7)CC6)CC5)cc4)n3)C2)C1=O ; O=C(O)CCC(=O)O]

In one aspect, provided is an amorphous solid dispersion of a salt of a compound of Formula (I). In one aspect, provided is an amorphous solid dispersion that comprises a dispersion agent and a salt. In one aspect, provided is an amorphous solid dispersion of a salt of a compound of Formula (I) selected from the group consisting of Fumarate Salt Form A, Fumarate Salt Amorphous Form, Fumarate Salt Form B, Fumarate Salt Form C, Fumarate Salt Form D, Fumarate Salt Form E, Maleate Salt Form A, Tosylate Salt Form A, Tosylate Salt Form B, Besylate Salt Form A, Cyclamate Salt Form A, Malate Salt Form A, Malonate Salt Form A, Napsylate Salt Form A, Napsylate Salt Form B, Napsylate Salt Form C, Napsylate Salt Form D, Succinate Salt Form A, Succinate Salt Form B, and Succinate Salt Form C.

Also provided herein is a pharmaceutical composition comprising a salt or free base of the compound of Formula (I) and a pharmaceutically acceptable carrier, vehicle, or adjuvant.

Provided herein is a method of treating a disease, disorder, or condition mediated by degrading Bruton's tyrosine kinase comprising administering to a patient or biological sample a salt or free base of the compound of Formula (I) or a pharmaceutical composition comprising a salt or free base of the compound of Formula (I) and a pharmaceutically acceptable carrier, vehicle, or adjuvant.

Further provided is a method of degrading splenocyte Bruton's tyrosine kinase in a subject in need thereof comprising the step of orally administering to the subject an amount of the salt or the free base of the compound of Formula (I) or the pharmaceutical composition comprising the compound of Formula (I) and a pharmaceutically acceptable carrier, vehicle, or adjuvant, wherein said salt or free base is capable of inducing proteolytic degradation of Bruton's tyrosine kinase, and wherein said amount is effective to degrade splenocyte Bruton's tyrosine kinase in the subject.

### BRIEF DESCRIPTION OF FIGURES

FIG. 1 shows an X-ray powder diffraction (XRPD) analysis of Free Base Form A.

FIG. 2 shows a thermogravimetric analysis-differential scanning calorimetry (TGA-DSC) thermogram of Free Base Form A.

FIG. 3 shows a dynamic vapor sorption (DVS) analysis of Free Base Form A.

FIG. 4 shows an XRPD pattern of Free Base Amorphous Form.

FIG. 5 shows a proton nuclear magnetic resonance imaging (¹H NMR) spectrum of Free Base Amorphous Form.

FIG. 6 shows a thermogravimetric analysis/differential thermal analysis (TGA/DTA) thermogram of Free Base Amorphous Form.

FIG. 7 shows a DSC thermogram of Free Base Amorphous Form.

FIG. 8 shows an XRPD comparison of Free Base Amorphous Form after one week stress at 40° C./75% relative humidity (RH) (top), and after DVS analysis (bottom).

FIG. 9 shows a DVS analysis of Free Base Amorphous Form (weight change dry-bulb (DB) % vs. relative humidity (RH) %).

FIG. 10 shows an overlay plot of Free Base Forms A, B, C, and D.

FIG. 11 shows an XRPD analysis of Free Base Form B.

FIG. 12 shows a TGA-DSC thermogram of Free Base Form B.

FIG. 13 shows an XRPD analysis of Free Base Form C.

FIG. 14 shows a TGA-DSC thermogram of Free Base Form C.

FIG. 15 shows an XRPD analysis of Free Base Form D.

FIG. 16 shows a TGA-DSC thermogram of Free Base Form D.

FIG. 17 shows a TGA/DTA analysis of Free Base Form D.

FIG. 18 shows an XPRD pattern of Free Base Form E.

FIG. 19 shows a ¹H NMR spectrum of Free Base Form E in DMSO-d₆.

FIG. 20 shows a TGA/DTA thermogram of Free Base Form E.

FIG. 21 shows an XRPD pattern of Free Base Form F.

FIG. 22 shows an XRPD pattern of Free Base Form G.

FIG. 23 shows a ¹H NMR spectrum of Free Base Form G in DMSO-d₆.

FIG. 24 shows a TGA/DTA thermogram of Free Base Form G.

FIG. 25 shows an XRPD pattern of Free Base Form H.

FIG. 26 shows a ¹H NMR spectrum of Free Base Form H in DMSO-d₆.

FIG. 27 shows a TGA/DTA thermogram of Free Base Form H.

FIG. 28 shows an XRPD pattern of Free Base Form J.

FIG. 29 shows a ¹H NMR spectrum of Free Base Form J in DMSO-d₆.

FIG. 30 shows a TGA/DTA thermogram of Free Base Form J.

FIG. 31 shows an overlay plot of XRPD patterns for Fumarate Salt Form A (top: scale up in 90 acetone: 10 water, slurry at 45° C.; upper middle: milling free base and Fumaric acid in 95 methanol: 5 water; lower middle: dioxane, slurry at 60° C.; bottom: 1-pentanol, slurry at 60° C.).

FIG. 32 shows an XRPD analysis of Fumarate Salt Form A.

FIG. 33 shows an overlay plot of XRPD patterns for fumarate salt (of the compound of Formula (I); top: vacuum dried Fumarate Salt Form A; bottom: before drying Fumarate Salt Form A).

FIG. 34 shows an FT-IR spectra of Fumarate Salt Form A.

FIG. 35 shows a Raman spectra of Fumarate Salt Form A.

FIG. 36 shows an overlay plot of XRPD patterns stability samples-fumarate salt (top to bottom: 0%, 20%, 40%, 60%, 75%, 97% relative humidity (RH) Fumarate Salt Form A).

FIG. 37 shows a thermogravimetric analysis-differential scanning calorimetry thermogram (TGA-DSC) of Fumarate Salt Form A.

FIG. 38 shows a dynamic vapor sorption (DVS) analysis of Fumarate Salt Form A.

FIG. 39 shows an XRPD pattern of Fumarate Salt Amorphous Form.

FIG. 40 shows a ¹H NMR spectrum of Fumarate Salt Amorphous Form.

FIG. 41 shows a TGA/DTA thermogram of Fumarate Salt Amorphous Form.

FIG. 42 shows a DSC thermogram of Fumarate Salt Amorphous Form.

FIG. 43 shows an XRPD comparison of Fumarate Salt Amorphous Form after one week stress at 40° C./75% relative humidity (RH) (top), and after DVS analysis (bottom).

FIG. 44 shows a DVS analysis of Fumarate Salt Amorphous Form (weight change DB % vs. RH %).

FIG. 45 shows an XRPD comparison of Fumarate Salt Form A and Fumarate Salt Form B: Fumarate Salt Form A (top); Fumarate Salt Form A+Fumarate Salt Form B (2^{nd} from top); Fumarate Salt Form B (2^{nd} from bottom); and Fumarate Salt Form B after stressing for one week at 40° C. and 75% relative humidity (bottom).

FIG. 46 shows a ¹H NMR spectrum of Fumarate Salt Form B.

FIG. 47 shows an XRPD comparison of Fumarate Salt Form A and Fumarate Salt Form C: Fumarate Salt Form A (top); Fumarate Salt Form A+Fumarate Salt Form C (2^{nd} from top); Fumarate Salt Form C (2^{nd} from bottom); and Fumarate Salt Form C after stressing for one week at 40° C. and 75% relative humidity (bottom).

FIG. 48 shows a ¹H NMR spectrum of Fumarate Salt Form C.

FIG. 49 shows an XRPD pattern of Fumarate Salt Form D.

FIG. 50 shows a thermogravimetric analysis-differential scanning calorimetry thermogram (TGA/DTA) of Fumarate Salt Form D.

FIG. 51 shows an XRPD pattern of Fumarate Salt Form E.

FIG. 52 shows a thermogravimetric analysis-differential scanning calorimetry thermogram (TGA/DTA) of Fumarate Salt Form E.

FIG. 53 shows an overlay plot of a fumarate salt (top) and a maleate salt (bottom).

FIG. 54 shows an overlay plot of tosylate salts (top: Tosylate Salt Form A; bottom: Tosylate Salt Form B).

FIG. 55 shows an XRPD analysis of Maleate Salt Form A.

FIG. 56 shows a TGA-DSC thermogram of Maleate Salt Form A.

FIG. 57 shows an XRPD analysis of Tosylate Salt Form A.

FIG. 58 shows an XRPD analysis of Tosylate Salt Form B.

FIG. 59 shows an XRPD analysis of Besylate Salt Form A.

FIG. 60 shows a TGA/DTA thermogram of Besylate Salt Form A.

FIG. 61 shows an XRPD analysis of Cyclamate Salt Form A.

FIG. 62 shows a TGA/DTA thermogram of Cyclamate Salt Form A.

FIG. 63 shows an XRPD analysis of Malate Salt Form A.

FIG. 64 shows a TGA/DTA thermogram of Malate Salt Form A.

FIG. 65 shows an XRPD analysis of Malonate Salt Form A.

FIG. 66 shows a TGA/DTA thermogram of Malonate Salt Form A.

FIG. 67 shows an XRPD analysis of Napsylate Salt Form A.

FIG. 68 shows a TGA/DTA thermogram of Napsylate Salt Form A.

FIG. 69 shows XRPD patterns of Napsylate Salt Forms A, B, C, and D: Napsylate Salt Form A (top); Napsylate Salt Form B (2^{nd} from top); Napsylate Salt Form C (2^{nd} from bottom); and Napsylate Salt Form D (bottom).

FIG. 70 shows an XRPD analysis of Napsylate Salt Form D.

FIG. 71 shows a TGA/DTA thermogram of Napsylate Salt Form D.

FIG. 72 shows XRPD patterns of Succinate Salt Form A and Succinate Salt Form B, compared to Succinate Salt Form A: Free Base Form A (top); Succinate Salt Form A obtained after stressing Succinate Salt Form B at 40° C./75% RH for 1 week (2^{nd} from top); Succinate Salt Form B (2^{nd} from bottom); and Succinate Salt Form A formed from the reaction in THF (bottom).

FIG. 73 shows an XRPD analysis of Succinate Salt Form A.

FIG. 74 shows a TGA/DTA thermogram of Succinate Salt Form B.

FIG. 75 shows an XRPD analysis of Succinate Salt Form C.

FIG. 76 shows a TGA/DTA thermogram of Succinate Salt Form C.

FIG. 77 shows an XRPD of an amorphous solid dispersion (ASD) prepared from Fumarate Salt Form A. When converted and presented in the amorphous solid dispersion, the form is amorphous.

FIG. 78 shows a DSC of an amorphous solid dispersion (ASD) prepared from Fumarate Salt Form A. When converted and presented in the amorphous solid dispersion, the form is amorphous.

### DETAILED DESCRIPTION

The present disclosure provides free bases of compounds of Formula (I), and crystalline, semi-crystalline, and non-crystalline forms thereof. In one or more embodiments, the free base of compound of Formula (I) is crystalline. In one or more embodiments, the free base of compound of Formula (I) is semi-crystalline or non-crystalline. In one or more embodiments, the semi-crystalline free base has lower crystallinity than crystalline free base without being amorphous. In one or more embodiments, the non-crystalline free base is amorphous. In one or more embodiments, the free base of compound of Formula (I) is amorphous.

The present disclosure provides a crystalline form of free base form of compound of Formula (I). In one or more embodiments, the free base is Free Base Form A.

In one or more embodiments, the amorphous form of free base of compound of Formula (I) is Free Base Amorphous Form.

The present disclosure also provides a semi-crystalline or non-crystalline form of a free base of compound of Formula (I). In one or more embodiments, the semi-crystalline form of free base of compound of Formula (I) is Free Base Form D.

In one or more embodiments, the free base is selected from the group consisting of Free Base Form A, B, C, and D.

In one or more embodiments, the free base is selected from the group consisting of Free Base Form A, B, C, D, E, F, G, H, and J. In one or more embodiments, the free base is selected from the group consisting of Free Base Form A, B, C, D, E, F, G, H, J, and Free Base Amorphous Form.

The present disclosure provides salts of compounds of Formula (I), and crystalline, semi-crystalline, and non-crystalline forms thereof. In one or more embodiments, the salt form of compound of Formula (I) is a fumarate salt, a maleate salt, a tosylate salt, a besylate salt, a cyclamate salt, a malate salt, a malonate salt, a napsylate salt, or a succinate salt. In one or more embodiments, the salt form of a compound of Formula (I) is crystalline. For example, a compound of Formula (I) that is a fumarate salt, a maleate salt, a tosylate salt, a besylate salt, a cyclamate salt, a malate salt, a malonate salt, a napsylate salt, or a succinate salt can be a crystalline salt. In one or more embodiments, the salt form of compound of Formula (I) is semi-crystalline or non-crystalline. In one or more embodiments, the semi-crystalline salt has lower crystallinity than crystalline salt without being amorphous. In one or more embodiments, the non-crystalline salt is amorphous. In one or more embodiments, the salt form of compound of Formula (I) is amorphous. In one or more embodiments, the salt is a hemi-salt including, but not limited to, a hemi-fumarate salt, wherein a hemi-salt has a stoichiometry of 1 equivalent counterion (such as 1 equivalent fumarate) with 2 equivalents of a compound of Formula (I).

The present disclosure provides a crystalline form of compound of Formula (I), fumarate salt. In one or more embodiments, the fumarate salt is Fumarate Salt Form A.

The present disclosure provides an amorphous form of a compound of Formula (I), fumarate salt. In one or more embodiments, the compound of Formula (I), fumarate salt, is Fumarate Salt Amorphous Form.

The present disclosure provides a crystalline form of compound of Formula (I), maleate salt. In one or more embodiments, the maleate salt is Maleate Salt Form A.

The present disclosure also provides a crystalline form of compound of Formula (I), tosylate salt. In one or more embodiments, the tosylate salt is Tosylate Salt Form A or Tosylate Salt Form B.

The present disclosure provides at least the following embodiments:
a) salt or free base of a compound of Formula (I);
b) crystalline, semi-crystalline, or non-crystalline fumarate salt(s) of compound of Formula (I), for example, Fumarate Salt Forms A-E, Maleate Salt Form A, Tosylate Salt Form A, Tosylate Salt Form B, Besylate Salt Form A, Cyclamate Salt Form A, Malate Salt Form A, Malonate Salt Form A, Napsylate Salt Forms A-D, Succinate Salt Forms A-C and Fumarate Salt Amorphous Form;
c) crystalline, semi-crystalline, or non-crystalline free bases of compound of Formula (I), for example, Free Base Forms A, B, C, D, E, F, G, H, J, and Free Base Amorphous Form;
d) amorphous form of compound of Formula (I), for example, Fumarate Salt Amorphous Form and Free Base Amorphous Form;
e) a hydrate or solvate of any one of (a)-(d);
f) a pharmaceutical composition comprising any one of (a)-(e);
g) the pharmaceutical composition of (f) further comprising a pharmaceutically acceptable carrier, vehicle, or adjuvant;
h) a method to treat a disease, disorder, or condition mediated by degrading Bruton's tyrosine kinase comprising administering to a patient in need thereof any one of (a)-(g);
i) a method of degrading splenocyte Bruton's tyrosine kinase in a subject in need thereof, comprising the step of orally administering to the subject an amount of any one of (a)-(g) in a patient in need thereof.

In one or more embodiments, the compound of Formula (I) is a solvate and the solvate is selected from the group consisting of: an acetonitrile solvate, a tetrahydrofuran solvate, a dioxane solvate, an ethanol solvate, an ethyl acetate solvate, a methanol solvate, a 2-methyl-tetrahydrofuran solvate, a methyl ethyl ketone solvate, an isopropanol solvate, a toluene solvate, a dimethylacetamide solvate, a dimethylsulfonamide solvate, a dimethylsulfoxide solvate, a dimethylformamide solvate, a diethyl ether solvate, an anisole solvate, an acetone solvate, a dimethylacetamide solvate, or a combination thereof.

In one or more embodiments, the compound of Formula (I) is a solvate and the solvate is selected from the group consisting of: an acetonitrile solvate, a tetrahydrofuran solvate, a dioxane solvate, an ethanol solvate, an ethyl acetate solvate, a methanol solvate, a 2-methyl-tetrahydrofuran solvate, a methyl ethyl ketone solvate, an isopropanol solvate, a toluene solvate, a dimethylacetamide solvate, a dimethylsulfonamide solvate, a dimethylsulfoxide solvate, a dimethylformamide solvate, a diethyl ether solvate, and an anisole solvate.

In In one or more embodiments, a salt of a compound of Formula (I) exhibits at least one diffraction peak at about 5.0−40° 2θ in its X-ray powder diffraction pattern. For example, the salt of a compound of Formula (I) can exhibit at least one diffraction peak at about 5.0−35.0° 2θ, 5.0−30.0° 2θ, 5.0−25.0° 2θ, 5.0−20.0° 2θ, 10.0−40.0° 2θ, 10.0−35.0° 2θ, 10.0−30.0° 2θ, 10.0−25.0° 2θ, 10.0−20.0° 2θ, 15.0−40.0° 2θ, 15.0−35.0° 2θ, 15.0−30.0° 2θ, 15.0−25.0° 2θ, 15.0−20.0° 2θ, 17.0−25.0° 2θ, 17.0−21.0° 2θ, 19.0−20.0° 20, or 20.0−22.0° 20 in its X-ray powder diffraction pattern.

In one or more embodiments, the salt of a compound of Formula (I) is a solvate, and the solvate is a tetrahydrofuran (THF) solvate, a methyl ethyl ketone (MEK) solvate, a dimethyl sulfoxide (DMSO) solvate, a dimethylformamide (DMF) solvate, or a diethyl ether solvate.

In one or more embodiments, a free base of a compound of Formula (I) exhibits at least one diffraction peak at about 5.0−40° 2θ in its X-ray powder diffraction pattern. For example, the free base of a compound of Formula (I) can exhibit at least one diffraction peak at about 5.0−35.0° 2θ, 5.0−30.0° 2θ, 5.0−25.0° 2θ, 5.0−20.0° 2θ, 10.0−40.0° 2θ, 10.0−35.0° 2θ, 10.0−30.0° 2θ, 10.0−25.0° 2θ, 10.0−20.0° 2θ, 15.0−40.0° 2θ, 15.0−35.0° 2θ, 15.0−30.0° 2θ, 15.0−25.0° 2θ, 15.0−20.0° 2θ, 17.0−25.0° 2θ, 17.0−21.0° 2θ, 19.0−20.0° 2θ, or 20.0−22.0° 2θ in its X-ray powder diffraction pattern.

In one or more embodiments, a free base of a compound of Formula (I) exhibits at least one diffraction peak at about 3.9−40° 2θ in its X-ray powder diffraction pattern.

In one or more embodiments, the free base of a compound of Formula (I) is a solvate, and the solvate is a tetrahydrofuran (THF) solvate, a methyl ethyl ketone (MEK) solvate, a dimethyl sulfoxide (DMSO) solvate, a dimethylformamide (DMF) solvate, or a diethyl ether solvate.

In one or more embodiments, the free base of a compound of Formula (I) is a solvate and the solvate is an acetonitrile solvate, a tetrahydrofuran solvate, a dioxane solvate, an ethanol solvate, an ethyl acetate solvate, a methanol solvate, a 2-methyl-tetrahydrofuran solvate, an isopropanol solvate, a toluene solvate, a dimethylacetamide solvate, a dimethylsulfonamide solvate, or an anisole solvate.

In one or more embodiments, the compound of Formula (I) is a solvate and the solvate is a tetrahydrofuran (THF) solvate, a methyl ethyl ketone (MEK) solvate, a dimethyl sulfoxide (DMSO) solvate, a dimethylformamide (DMF) solvate, a diethyl ether solvate, an acetone solvate, a dimethylacetamide (DMAc) solvate, or a combination thereof.

### Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures known in the art that are described or referenced herein are generally well understood and commonly employed using conventional methodologies by those skilled in the art.

As used herein, the singular forms “a,” “an,” and “the” include the plural referents unless the context clearly indicates otherwise.

As used herein, the term “about” refers to the stated value plus or minus 10%, plus or minus 5%, or plus or minus 1%. For example, a value of “about 10” can encompass a range of 9 to 11. For logarithmic scales, the term “about” refers to the stated value plus or minus 0.3 log units, or plus or minus 0.2 log units, or plus or minus 0.1 log units. For example, a value of “about pH 4.6” can encompass a pH range of 4.5-4.7. For 2 theta (20) values, the term “about” refers to the stated value plus or minus 0.3, 0.2, or 0.1 degrees 2 theta (20).

The term “substantially free of” or “substantially in the absence of” with respect to a composition refers to a composition that includes at least about 85 or 90% by weight, in one or more embodiments at least about 95%, 98%, 99% or 100% by weight, of a designated enantiomer or stereoisomer of a compound. For example, “substantially free of” or “substantially in the absence of” with respect to a composition can refer to a composition that includes about 85%, 86%, 87%, 88%, 89%, 90%, 91%, 92%, 93%, 94%, 95%, 96%, 97%, 98%, 99%, or 100% by weight of a designated enantiomer or stereoisomer of a compound. In one or more embodiments, in the methods and compounds provided herein, the compounds are substantially free of other enantiomers or stereoisomers.

Similarly, the term “isolated” with respect to a composition refers to a composition that includes at least 85%, 90%, 95%, 98%, and 99% to 100% by weight, of a designated compound, enantiomer, or stereoisomer, the remainder comprising other chemical species, enantiomers, or stereoisomers. For example, “isolated” with respect to a composition can refer to a composition that includes about 85%, 86%, 87%, 88%, 89%, 90%, 91%, 92%, 93%, 94%, 95%, 96%, 97%, 98%, 99%, or 100% by weight of a designated compound, enantiomer, or stereoisomer, the remainder comprising other chemical species, enantiomers, or stereoisomers.

As used herein, the terms “subject” and “patient” are used interchangeably herein. The terms “subject” and “subjects” refer to an animal, such as a mammal including a non-primate (e.g., a cow, pig, horse, cat, dog, rat, and mouse) and a primate (e.g., a monkey such as a cynomolgus monkey, a chimpanzee and a human), and for example, a human. In one or more embodiments, the subject is a farm animal (e.g., a horse, a cow, a pig, etc.) or a pet (e.g., a dog or a cat). In one or more embodiments, the subject is a human.

“Therapeutically effective amount” refers to an amount of a compound or composition that, when administered to a subject for treating a disease, disorder, or condition, is sufficient to effect such treatment for the disease, disorder, or condition. A “therapeutically effective amount” can vary depending on, inter alia, the compound, the disease, disorder, or condition and its severity, and the age, weight, etc., of the subject to be treated.

“Treating” or “treatment” of any disease, disorder, or condition refers, in one or more embodiments, to ameliorating a disease, disorder, or condition that exists in a subject. In one or more embodiments, “treating” or “treatment” includes ameliorating at least one physical parameter, which may be indiscernible by the subject. In one or more embodiments, “treating” or “treatment” includes modulating the disease, disorder, or condition, cither physically (e.g., stabilization of a discernible symptom) or physiologically (e.g., stabilization of a physical parameter) or both. In one or more embodiments, “treating” or “treatment” includes delaying the onset of the disease, disorder, or condition.

As used herein, the term “free base” refers to a conjugate base (deprotonated) form of a basic group. In some instances, a free base form or free base may be a standard form of a molecule as it exists at equilibrium, standard temperature and pressure, and/or as it sits on a shelf (not a salt); this may also be called a free form. In other instances, a free base form or free base may be a form that is briefly identified, such as a molecule produced by a reaction under inert gas that is identified and then decomposes, reverts, or otherwise changes to another form. In one or more embodiments, the basic group is an amine.

As used herein, the terms “prophylactic agent” and “prophylactic agents” as used refer to any agent(s) which can be used in the prevention of a disease, disorder, or condition or one or more symptoms thereof. In one or more embodiments, the term “prophylactic agent” includes a compound provided herein. In one or more other embodiments, the term “prophylactic agent” does not refer a compound provided herein. For example, a prophylactic agent is an agent which is known to be useful for, or has been or is currently being used to prevent or impede the onset, development, progression and/or severity of a disease, disorder, or condition.

As used herein, the phrase “prophylactically effective amount” refers to the amount of a therapy (e.g., prophylactic agent) which is sufficient to result in the prevention or reduction of the development, recurrence or onset of one or more symptoms associated with a disease, disorder, or condition (or to enhance or improve the prophylactic effect(s) of another therapy, e.g., another prophylactic agent).

“Substantially” when describing XRPD patterns is meant that the reported peaks can vary by ±0.2°.

“Substantially” when describing differential scanning calorimetry (DSC) thermograms and thermogravimetric analysis (TGA) is meant that the reported temperatures can vary by ±0.5° C.

### Free Base Form A

In one or more embodiments, provided herein is a free base of a compound of Formula (I). In one or more embodiments, the free base of the compound of Formula (I) is Free Base Form A.

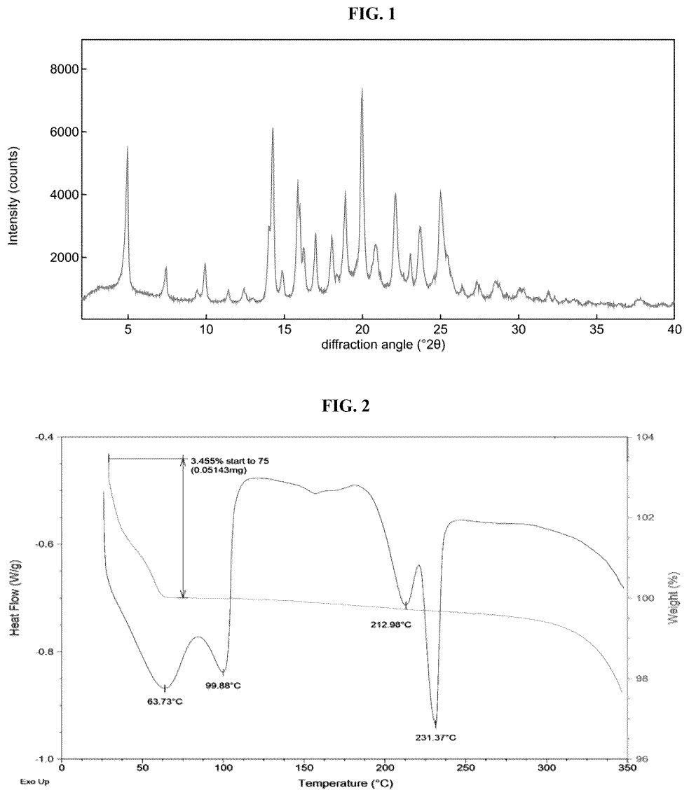

In one or more embodiments, Free Base Form A is characterized by an X-ray powder diffraction (XRPD) pattern substantially similar to that set forth in FIG. 1. In one or more embodiments, Free Base Form A is characterized by an X-ray powder diffraction (XRPD) pattern comprising peaks substantially similar to those set forth in Table 21. In one or more embodiments, Free Base Form A is characterized by an XRPD pattern comprising:
a) 2θ values of about 4.96, 14.26, 15.86, 18.90, 19.98, 22.12, 23.68, and 25.00±0.2° 2θ;
b) 2θ values of about 4.96, 7.44, 9.92, 14.26, 15.86, 17.00, 18.04, 18.90, 19.98, 22.12, 23.68, and 25.00±0.2° 2θ;
c) 2θ values including at least or selected from about 4.96, 7.44, 9.42, 9.92, 11.42, 12.42, 14.00, 14.26, 14.86, 15.86, 16.24, 17.00, 18.04, 18.90, 19.98, 20.82, 22.12, 23.06, 23.68, 25.00, 26.36, 27.32, 28.50, 30.06, 31.88, and 32.32±0.2° 2θ;
d) at least two, three, four, or five 2θ values selected from about 4.96, 14.00, 14.26, 15.86, 16.24, 17.00, 18.04, 18.90, 19.98, 20.82, 20.82, 23.68, and 25.00±0.2° 2θ;
e) at least six, seven, or eight 2θ values selected from about 4.96, 9.92, 14.00, 14.26, 15.86, 16.24, 17.00, 18.04, 18.90, 19.98, 20.82, 20.82, 23.06, 23.68, and 25.00±0.2° 2θ;
f) 2θ values of about 9.92, 14.00, 14.26, 15.86, 16.24, 17.00, 18.04, 18.90, 19.98, 20.82, 20.82, 23.06, 23.68, and 25.00±0.2° 2θ;
g) 2θ values including at least or selected from about 9.92, 14.00, 14.26, 15.86, 16.24, 17.00, 18.04, 18.90, 19.98, 20.82, 20.82, 23.06, 23.68, and 25.00±0.2° 2θ;
h) 2θ values of about 4.96, 14.26, 15.86, and 19.98±0.2° 2θ;
i) 2θ values including at least or selected from about 4.96, 14.26, 15.86, and 19.98±0.2° 2θ;
j) 2θ values of about 4.96, 14.26, and 19.98±0.2° 2θ;
k) 2θ values including at least or selected from about 4.96, 14.26, and 19.98±0.2° 2θ;
l) 2θ values of about 14.26, 15.86, 16.24, 17.00, 18.04, 18.90, 19.98, 20.82, 22.12, 23.06, 23.68, and 25.00±0.2° 2θ;
m) 2θ values including at least or selected from about 14.26, 15.86, 16.24, 17.00, 18.04, 18.90, 19.98, 20.82, 22.12, 23.06, 23.68, and 25.00±0.2° 2θ;
n) 2θ values of about 14.26, 15.86, 17.00, 18.04, 18.90, 19.98, 22.12, 23.68, and 25.00±0.2° 2θ;
o) 2θ values including at least or selected from about 14.26, 15.86, 17.00, 18.04, 18.90, 19.98, 22.12, 23.68, and 25.00±0.2° 2θ;
p) 2θ values of about 4.96, 14.26, 18.90, and 19.98±0.2° 2θ;
q) 2θ values including at least or selected from about 4.96, 14.26, 18.90, and 19.98±0.2° 2θ;
r) 2θ values of about 4.96, 14.26, 9.98, and 22.12±0.2° 2θ;
s) 2θ values including at least or selected from about 4.96, 14.26, 9.98, and 22.12±0.2° 2θ;
t) 2θ values of about 4.96, 14.26, 9.98, and 23.68±0.2° 2θ;
u) 2θ values including at least or selected from about 4.96, 14.26, 9.98, and 23.68±0.2° 2θ;
v) 2θ values of about 4.96, 14.26, 9.98, and 25.00±0.2° 2θ; and
w) 2θ values including at least or selected from about 4.96, 14.26, 9.98, and 25.00±0.2° 2θ.

In one or more embodiments, Free Base Form A is characterized by a differential scanning calorimetry (DSC) thermogram having four endotherms with endotherm maxima between about 60° C. and about 70° C., between about 95° C. and about 105° C., between about 210° C. and about 220° C., and between about 225° C. and about 235° C. In one or more embodiments, Free Base Form A is characterized by a differential scanning calorimetry (DSC) thermogram having four endotherms with endotherm maxima at about 64° C., 100° C., 213° C., and 231° C. In one or more embodiments, Free Base Form A is characterized by DSC thermogram substantially similar to that set forth in FIG. 2.

In one or more embodiments, Free Base Form A is characterized by a weight loss in the range of about 0.1% to about 5% when heated from about 25° C. to about 75° C. in a thermogravimetric analysis (TGA). In one or more embodiments, Free Base Form A is characterized by a weight loss of about 3.5% when heated from about 25° C. to about 75° C. in a TGA. In one or more embodiments, Free Base Form A is characterized by a TGA substantially similar to that set forth in FIG. 2.

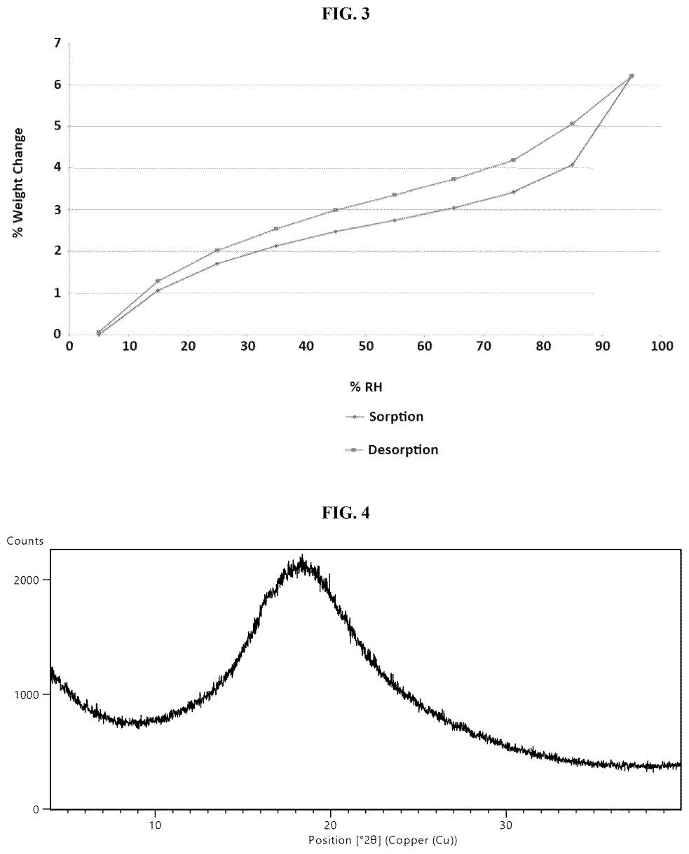

A DVS analysis of Free Base Form A is shown in FIG. 3.

Free Base Form A can be synthesized as described in Synthetic Example 1. In one or more embodiments, Free Base Form A is synthesized by mixing free base compound of Formula (I) in an aprotic solvent. In one or more embodiments, the solvent is selected from the group consisting of tetrahydrofuran, acetone, ethyl acetate, diethyl ether, toluene, hexane, dichloromethane, methyl ethyl ketone, dimethylsulfoxide, dimethylformamide, acetonitrile, and water. In one or more embodiments, the mixture comprises more than one solvent. In one or more embodiments, the mixture comprises tetrahydrofuran and methyl ethyl ketone. After stirring, for instance overnight, additional solvent may be added. The additional solvent is selected from the group in this paragraph, such as methyl ethyl ketone. The mixture is allowed to sit for 1-6 days and then cooled for 1-2 days. In one or more embodiments, evaporating the mixture provides a precipitate. The precipitate is dried to afford Free Base Form A.

In one or more embodiments, Free Base Form A is synthesized by a method comprising:
a) mixing free base compound of Formula (I) in tetrahydrofuran and methyl ethyl ketone;
b) stirring overnight;
c) adding additional methyl ethyl ketone;
d) sitting, cooling, and evaporating to afford a precipitate; and
e) isolating the precipitate to afford Free Base Form A.

In one or more embodiments, the mixture is allowed to cool for at least 12 hours, at least 14 hours, at least 16 hours, at least 18 hours, at least 20 hours, at least 24 hours, or at least 48 hours in step (d).

### Free Base Amorphous Form

In one or more embodiments, provided herein is a free base of a compound of Formula (I). In one or more embodiments, the free base of the compound of Formula (I) is Free Base Amorphous Form. In one or more embodiments, the Free Base Amorphous Form is a hydrate.

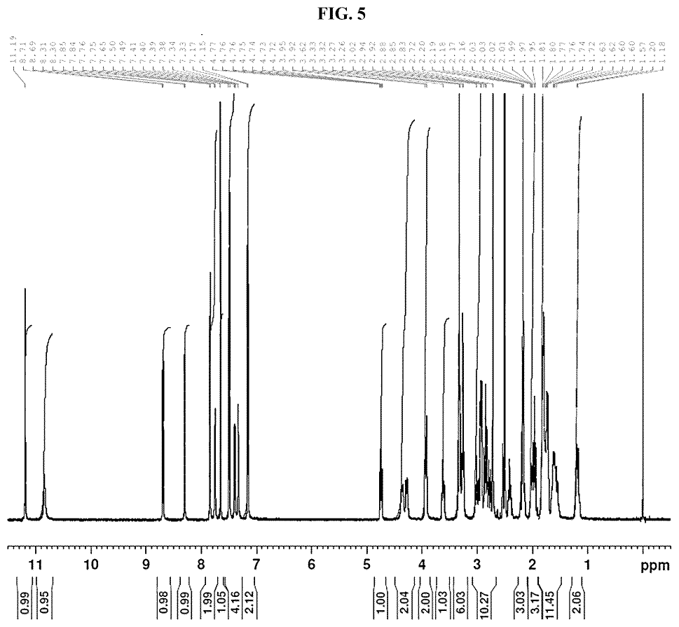

In one or more embodiments, Free Base Amorphous Form is characterized by an X-ray powder diffraction (XRPD) pattern substantially similar to that set forth in FIG. 4. In one or more embodiments, Free Base Amorphous Form is shown to be amorphous by XRPD.

In one or more embodiments, Free Base Amorphous Form is characterized by a differential thermal analysis (DTA) thermogram having an endotherm with an endotherm maxima between about 140° C. and about 180° C. In one or more embodiments, Free Base Amorphous Form is characterized by a differential thermal analysis (DTA) thermogram having an endotherm with endotherm maxima at about 144° C. and about 177° C. In one or more embodiments, Free Base Amorphous Form is characterized by DTA thermogram substantially similar to that set forth in FIG. 6.

In one or more embodiments, Free Base Amorphous Form is characterized by a weight loss in the range of about 0.1% to about 1% when heated from about 35° C. to about 190° C. in a thermogravimetric analysis (TGA). In one or more embodiments, Free Base Amorphous Form is characterized by a weight loss of about 0.5% when heated from about 36° C. to about 184° C. in a TGA. In one or more embodiments, Free Base Amorphous Form is characterized by a TGA substantially similar to that set forth in FIG. 6.

Free Base Amorphous Form can be synthesized as described in Synthetic Example 2. In one or more embodiments, Free Base Amorphous Form is synthesized by melt and quench (melt/quench) of free base compound of Formula (I) (Free Base Form A). In one or more embodiments, the melt is at 260° C. and the quench is in liquid nitrogen.

In one or more embodiments, Free Base Amorphous Form is synthesized by a method comprising:
a) heating Free Base Form A to 260° C.; and
b) removing from heat and quenching in liquid nitrogen.

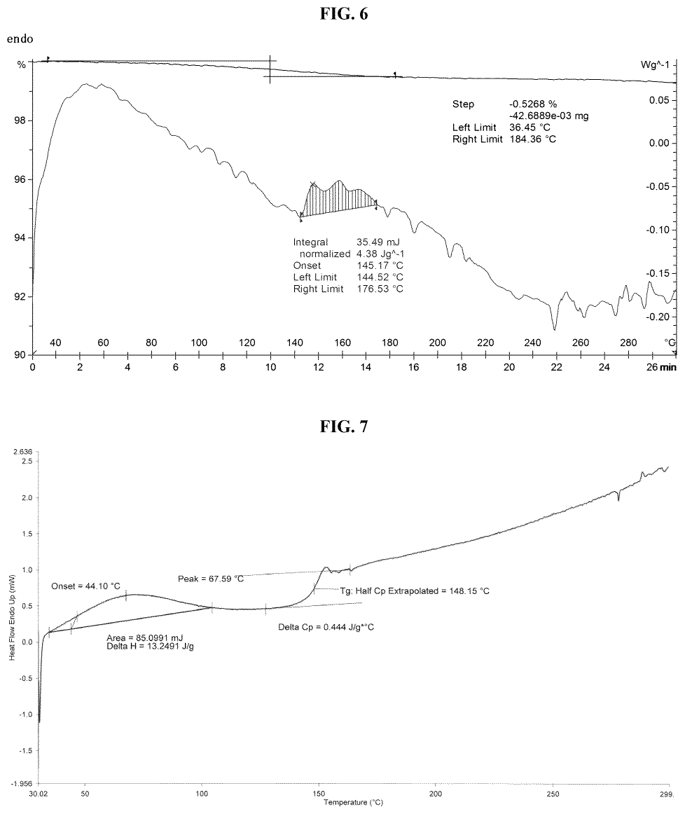

In one or more embodiments, Free Base Amorphous Form is characterized by a differential scanning calorimetry (DSC) thermogram having an endotherm with an endotherm maxima between about 30° C. and about 100° C. In one or more embodiments, Free Base Amorphous Form is characterized by a differential scanning calorimetry (DSC) thermogram having an endotherm with endotherm maxima at about 65° C. In one or more embodiments, Free Base Amorphous Form is characterized by DSC thermogram substantially similar to that set forth in FIG. 7.

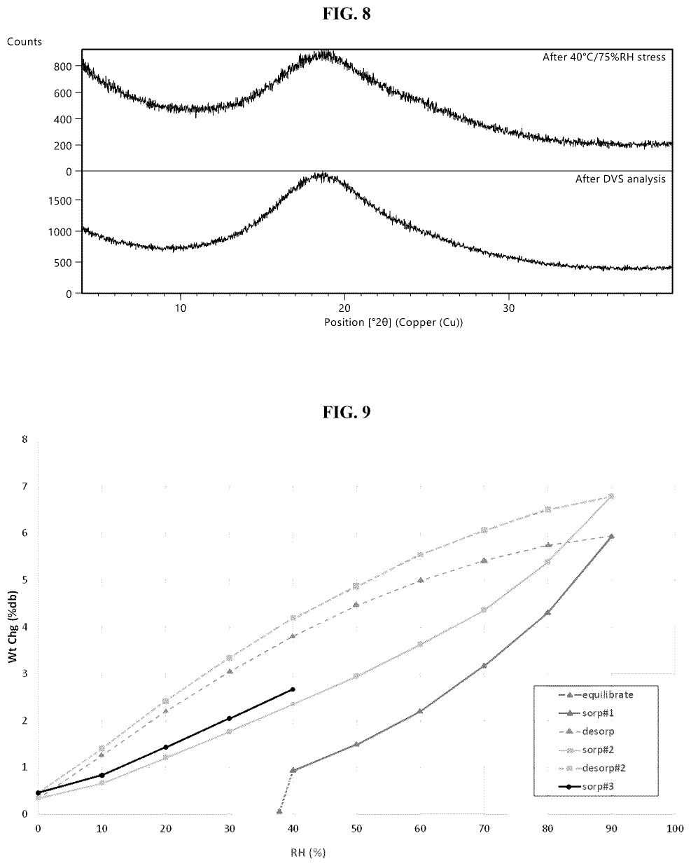

A DVS analysis of Free Base Amorphous Form is shown in FIG. 9.

### Free Base Form B

In one or more embodiments, provided herein is a free base of a compound of Formula (I). In one or more embodiments, the free base of the compound of Formula (I) is Free Base Form B.

In one or more embodiments, Free Base Form B is characterized by an X-ray powder diffraction (XRPD) pattern substantially similar to that set forth in FIG. 11. In one or more embodiments, Free Base Form B is characterized by an X-ray powder diffraction (XRPD) pattern comprising peaks substantially similar to those set forth in Table 22. In one or more embodiments, Free Base Form B is characterized by an XRPD pattern comprising:
a) 2θ values of about 16.46, 17.98, 21.22, 21.70, 22.92, and 24.46±0.2° 2θ;
b) 2θ values of about 3.76, 7.48, 10.20, 15.80, 16.14, 16.46, 17.98, 21.22, 21.70, 21.96, 22.92, 24.46, and 24.70±0.2° 2θ;
c) 2θ values including at least or selected from about 3.76, 7.48, 10.20, 10.70, 11.18, 11.46, 12.50, 13.68, 14.92, 15.80, 16.04, 16.14, 16.46, 16.98, 17.38, 17.70, 17.98, 18.28, 18.56, 19.30, 19.58, 20.10, 20.44, 20.82, 21.22, 21.42, 21.70, 21.96, 22.18, 22.42, 22.92, 23.78, 24.00, 24.46, 24.70, 25.08, 25.36, 25.78, 25.94, 26.38, 26.94, 27.50, 27.68, 28.16, 29.06, 29.48, 30.00, 30.48, 30.86, 31.24, 31.72, 32.36, 33.24, 33.60, 34.28, 34.82, 35.78, 36.38, 36.90, 38.52, and 39.40±0.2° 2θ;
d) at least two, three, four, or five 2θ values selected from about 15.80, 16.04, 16.14, 16.46, 17.98, 18.28, 21.22, 21.70, 21.96, 22.92, 24.46, 24.70±0.2° 2θ;
e) at least six, seven, or eight 2θ values selected from about 15.80, 16.04, 16.14, 16.46, 16.98, 17.70, 17.98, 18.28, 18.56, 19.58, 20.10, 20.44, 21.22, 21.42, 21.70, 21.96, 22.18, 22.92, 24.46, 24.70±0.2° 2θ;
f) 2θ values of about 15.80, 16.14, 16.46, 17.98, 21.22, 21.70, 22.92, 24.46, and 24.70±0.2° 2θ;
g) 2θ values including at least or selected from about 15.80, 16.14, 16.46, 17.98, 21.22, 21.70, 22.92, 24.46, and 24.70±0.2° 2θ;
h) 2θ values of about 16.46, 17.98, 21.70, and 22.92±0.2° 2θ;
i) 2θ values including at least or selected from about 16.46, 17.98, 21.70, and 22.92±0.2° 2θ;
j) 2θ values of about 16.46, 21.70, and 22.92±0.2° 2θ;
k) 2θ values including at least or selected from about 16.46, 21.70, and 22.92±0.2° 2θ;
l) 2θ values of about 10.20, 14.92, 15.80, 16.14, 16.46, 17.98, 21.22, 21.70, 22.92, 24.46, and 24.70±0.2° 2θ;
m) 2θ values including at least or selected from about 10.20, 14.92, 15.80, 16.14, 16.46, 17.98, 21.22, 21.70, 22.92, 24.46, and 24.70±0.2° 2θ;
n) 2θ values of about 15.80, 16.14, 16.46, 17.98, 21.22, 21.70, 22.92, 24.46, 24.70, and 25.94±0.2° 2θ;
o) 2θ values including at least or selected from about 15.80, 16.14, 16.46, 17.98, 21.22, 21.70, 22.92, 24.46, 24.70, and 25.94±0.2° 2θ;
p) 2θ values of about 16.46, 21.22, 21.70, and 22.92±0.2° 2θ;
q) 2θ values including at least or selected from about 16.46, 21.22, 21.70, and 22.92±0.2° 2θ;
r) 2θ values of about 16.46, 21.70, 21.96, and 22.92±0.2° 2θ;
s) 2θ values including at least or selected from about 16.46, 21.70, 21.96, and 22.92±0.2° 2θ;
t) 2θ values of about 16.46, 21.70, 22.92, and 24.46±0.2° 2θ;
u) 2θ values including at least or selected from about 16.46, 21.70, 22.92, and 24.46±0.2° 2θ;
v) 2θ values of about 16.46, 21.70, 22.92, and 24.70±0.2° 2θ; and
w) 2θ values including at least or selected from about 16.46, 21.70, 22.92, and 24.70±0.2° 2θ.

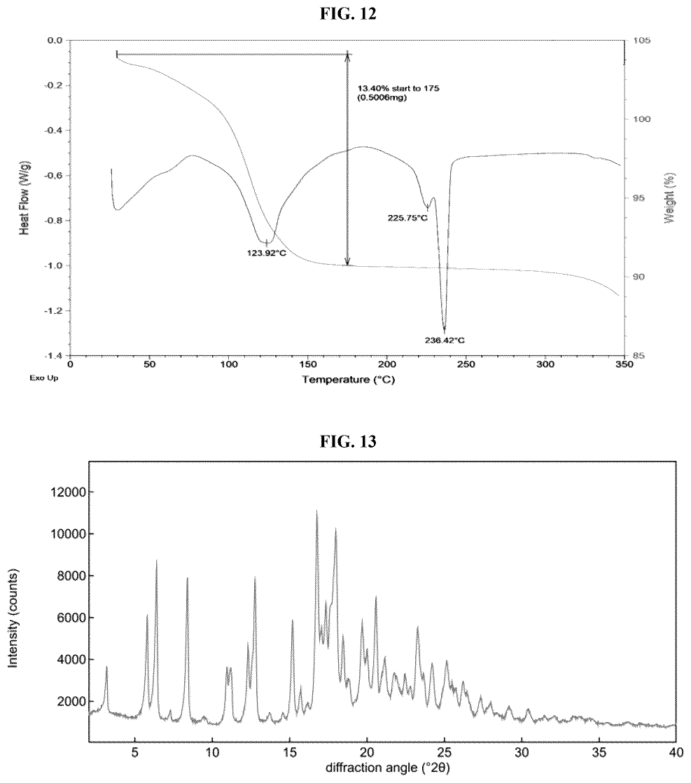

In one or more embodiments, Free Base Form B is characterized by a differential scanning calorimetry (DSC) thermogram having three endotherms with endotherm maxima between about 120° C. and about 130° C., between about 220° C. and about 230° C., and between about 230° C. and about 240° C. In one or more embodiments, Free Base Form B is characterized by a differential scanning calorimetry (DSC) thermogram having three endotherms with endotherm maxima at about 124° C., 226° C., and 236° C. In one or more embodiments, Free Base Form B is characterized by DSC thermogram substantially similar to that set forth in FIG. 12.

In one or more embodiments, Free Base Form B is characterized by a weight loss in the range of about 10% to about 15% when heated from about 25° C. to about 175° C. (single ramp) in a thermogravimetric analysis (TGA). In one or more embodiments, Free Base Form B is characterized by a weight loss of about 13.4% when heated from about 25° C. to about 175° C. in a TGA. In one or more embodiments, Free Base Form B is characterized by a TGA substantially similar to that set forth in FIG. 12.

Free Base Form B can be synthesized as described in Synthetic Example 3. In one or more embodiments, Free Base Form B is synthesized by mixing free base compound of Formula (I) in an aprotic solvent. In one or more embodiments, the solvent is selected from the group consisting of tetrahydrofuran, acetone, ethyl acetate, diethyl ether, toluene, hexane, dichloromethane, methyl ethyl ketone, dimethylsulfoxide, and dimethylformamide. After stirring, for instance overnight to 1 week, a precipitate is formed. The precipitate is centrifuged, decanted, and dried to afford Free Base Form B.

In one or more embodiments, Free Base Form B is synthesized by a method comprising:
a) mixing free base compound of Formula (I) in DMSO;
b) stirring; and
c) isolating the precipitate to afford Free Base Form B.

In one or more embodiments, the mixture is allowed to stir for at least 12 hours, at least 14 hours, at least 16 hours, at least 18 hours, at least 20 hours, at least 24 hours, at least 48 hours, at least 3 days, at least 4 days, at least 5 days, at least 6 days, or at least 7 days in step (b).

### Free Base Form C

In one or more embodiments, provided herein is a free base of a compound of Formula (I). In one or more embodiments, the free base of the compound of Formula (I) is Free Base Form C.

In one or more embodiments, Free Base Form C is characterized by an X-ray powder diffraction (XRPD) pattern substantially similar to that set forth in FIG. 13. In one or more embodiments, Free Base Form C is characterized by an X-ray powder diffraction (XRPD) pattern comprising peaks substantially similar to those set forth in Table 23. In one or more embodiments, Free Base Form C is characterized by an XRPD pattern comprising:
a) 2θ values of about 6.40, 8.40, 12.76, 16.76, and 17.98±0.2° 2θ;
b) 2θ values of about 3.18, 5.80, 6.40, 8.40, 12.76, 15.18, 16.76, 17.34, 17.98, 19.68, 20.58, and 23.28±0.2° 2θ;
c) 2θ values including at least or selected from about 3.18, 5.80, 6.40, 7.30, 8.40, 9.44, 10.96, 11.18, 12.30, 12.76, 13.68, 14.54, 15.18, 15.72, 16.16, 16.76, 17.06, 17.34, 17.98, 18.44, 18.82, 19.68, 20.02, 20.58, 21.16, 21.76, 22.46, 22.80, 23.28, 23.64, 24.20, 25.16, 25.48, 26.20, 26.46, 27.34, 27.98, 29.18, 30.40, 31.48, 32.06, 33.30, 33.70, 34.56, and 36.84±0.2° 2θ;
d) at least two, three, four, or five 2θ values selected from about 6.40, 8.40, 12.76, 16.76, 17.34, 17.98, 19.68, 20.58, and 23.28±0.2° 2θ;
e) at least six, seven, or eight 2θ values selected from about 5.80, 6.40, 8.40, 12.76, 15.18, 16.76, 17.06, 17.34, 17.98, 19.68, 20.58, and 23.28±0.2° 2θ;
f) 2θ values of about 5.80, 6.40, 8.40, 12.76, 15.18, 16.76, 17.06, 17.34, 17.98, 18.44, 19.68, 20.58, and 23.28±0.2° 2θ;
g) 2θ values including at least or selected from about 5.80, 6.40, 8.40, 12.76, 15.18, 16.76, 17.06, 17.34, 17.98, 18.44, 19.68, 20.58, and 23.28±0.2° 2θ;
h) 2θ values of about 6.40, 12.76, 16.76, and 17.98±0.2° 2θ;
i) 2θ values including at least or selected from about 6.40, 12.76, 16.76, and 17.98±0.2° 2θ;
j) 2θ values of about 12.76, 16.76, and 17.98±0.2° 2θ;
k) 2θ values including at least or selected from about 12.76, 16.76, and 17.98±0.2° 2θ;
l) 2θ values of about 5.80, 6.40, 8.40, 12.76, 15.18, 16.76, 17.06, 17.34, 17.98, 18.44, 19.68, and 20.58±0.2° 2θ;
m) 2θ values including at least or selected from about 5.80, 6.40, 8.40, 12.76, 15.18, 16.76, 17.06, 17.34, 17.98, 18.44, 19.68, and 20.58±0.2° 2θ;
n) 2θ values of about 8.40, 12.76, 15.18, 16.76, 17.06, 17.34, 17.98, 18.44, 19.68, 20.58, and 23.28±0.2° 2θ;
o) 2θ values including at least or selected from about 8.40, 12.76, 15.18, 16.76, 17.06, 17.34, 17.98, 18.44, 19.68, 20.58, and 23.28±0.2° 2θ;
p) 2θ values of about 8.40, 12.76, 16.76, and 17.98±0.2° 2θ;
q) 2θ values including at least or selected from about 8.40, 12.76, 16.76, and 17.98±0.2° 2θ;
r) 2θ values of about 12.76, 16.76, 17.34, and 17.98±0.2° 2θ;
s) 2θ values including at least or selected from about 12.76, 16.76, 17.34, and 17.98±0.2° 2θ;
t) 2θ values of about 12.76, 16.76, 17.98, and 20.58±0.2° 2θ;
u) 2θ values including at least or selected from about 12.76, 16.76, 17.98, and 20.58±0.2° 2θ;
v) 2θ values of about 12.76, 16.76, 17.98, and 23.28±0.2° 2θ; and
w) 2θ values including at least or selected from about 12.76, 16.76, 17.98, and 23.28±0.2° 2θ.

In one or more embodiments, Free Base Form C is characterized by a differential scanning calorimetry (DSC) thermogram having two endotherms with endotherm maxima between about 60° C. and about 70° C. and between about 195° C. and about 205° C. In one or more embodiments, Free Base Form C is characterized by a differential scanning calorimetry (DSC) thermogram having two endotherms with endotherm maxima at about 64° C. and about 202° C. In one or more embodiments, Free Base Form C is characterized by DSC thermogram substantially similar to that set forth in FIG. 14.

In one or more embodiments, Free Base Form C is characterized by a weight loss in the range of about 10% to about 15% when heated from about 25° C. to about 205° C. (such as to about 200° C.) in a thermogravimetric analysis (TGA). In one or more embodiments, Free Base Form C is characterized by a weight loss of about 11.4% when heated from about 25° C. to about 205° C. (such as to about 200° C.) in a TGA. In one or more embodiments, Free Base Form C is characterized by a TGA substantially similar to that set forth in FIG. 14.

Free Base Form C can be synthesized as described in Synthetic Example 4. In one or more embodiments, Free Base Form C is synthesized by mixing free base compound of Formula (I) in an aprotic solvent. In one or more embodiments, the solvent is selected from the group consisting of tetrahydrofuran, acetone, ethyl acetate, diethyl ether, toluene, hexane, dichloromethane, methyl ethyl ketone, dimethylsulfoxide, and dimethylformamide. After stirring, for instance overnight to 1 week, a precipitate is formed. The precipitate is centrifuged, decanted, and dried to afford Free Base Form C.

In one or more embodiments, Free Base Form C is synthesized by a method comprising:
a) mixing free base compound of Formula (I) in DMF;
b) stirring; and
c) isolating the precipitate to afford Free Base Form C.

In one or more embodiments, the mixture is allowed to stir for at least 12 hours, at least 14 hours, at least 16 hours, at least 18 hours, at least 20 hours, at least 24 hours, at least 48 hours, at least 3 days, at least 4 days, at least 5 days, at least 6 days, or at least 7 days in step (b).

### Free Base Form D

In one or more embodiments, provided herein is a free base of a compound of Formula (I). In one or more embodiments, the free base of the compound of Formula (I) is Free Base Form D.

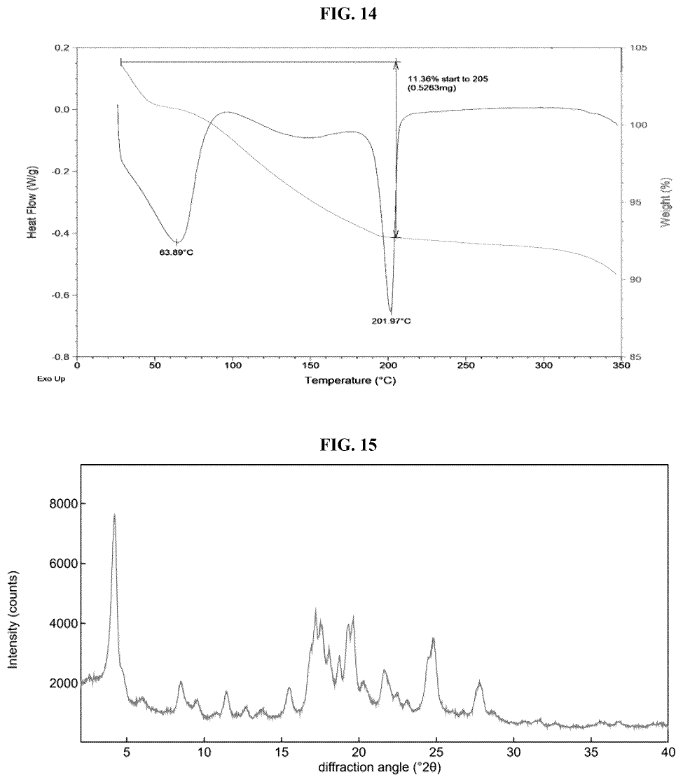

In one or more embodiments, Free Base Form D is characterized by an X-ray powder diffraction (XRPD) pattern substantially similar to that set forth in FIG. 15. In one or more embodiments, Free Base Form D is characterized by an X-ray powder diffraction (XRPD) pattern comprising peaks substantially similar to those set forth in Table 24. In one or more embodiments, Free Base Form D is characterized by an XRPD pattern comprising:
a) 2θ values of about 4.18, 17.20, 17.52, 19.32, and 19.64±0.2° 2θ;
b) 2θ values of about 4.18, 17.20, 17.52, 18.06, 19.32, 19.64, and 24.84±0.2° 2θ;
c) 2θ values including at least or selected from about 4.18, 5.98, 8.48, 9.54, 11.42, 12.68, 13.78, 15.48, 17.20, 17.52, 18.06, 18.74, 19.32, 19.64, 20.26, 21.62, 22.44, 23.12, 24.84, 27.78, 30.74, 31.68, 32.68, 35.84, and 36.84±0.2° 2θ;
d) at least two, three, four, or five 2θ values selected from about 4.18, 17.20, 17.52, 19.32, 19.64, and 24.84±0.2° 2θ;
e) at least six, seven, or eight 2θ values selected from about 4.18, 8.48, 17.20, 17.52, 18.06, 18.74, 19.32, 19.64, 20.26, 21.62, 24.84, and 27.78±0.2° 2θ;
f) 2θ values of about 4.18, 8.48, 17.20, 17.52, 18.74, 19.32, 19.64, 20.26, 21.62, and 24.84±0.2° 2θ;
g) 2θ values including at least or selected from about 4.18, 8.48, 17.20, 17.52, 18.74, 19.32, 19.64, 20.26, 21.62, and 24.84±0.2° 2θ;
h) 2θ values of about 4.18, 17.20, 17.52, and 19.64±0.2° 2θ;
i) 2θ values including at least or selected from about 4.18, 17.20, 17.52, and 19.64±0.2° 2θ;
j) 2θ values of about 4.18, 17.20, and 19.64±0.2° 2θ;
k) 2θ values including at least or selected from about 4.18, 17.20, and 19.64±0.2° 2θ;
l) 2θ values of about 4.18, 8.48, 17.20, 17.52, 18.06, 18.74, 19.32, 19.64, 20.26, and 21.62±0.2° 2θ;
m) 2θ values including at least or selected from about 4.18, 8.48, 17.20, 17.52, 18.06, 18.74, 19.32, 19.64, 20.26, and 21.62±0.2° 2θ;
n) 2θ values of about 17.20, 17.52, 18.06, 18.74, 19.32, 19.64, 20.26, 21.62, 24.84, and 27.78±0.2° 2θ;
o) 2θ values including at least or selected from about 17.20, 17.52, 18.06, 18.74, 19.32, 19.64, 20.26, 21.62, 24.84, and 27.78±0.2° 2θ;
p) 2θ values of about 4.18, 17.20, 18.06, and 19.64±0.2° 2θ;
q) 2θ values including at least or selected from about 4.18, 17.20, 18.06, and 19.64±0.2° 2θ;
r) 2θ values of about 4.18, 17.20, 19.32, and 19.64±0.2° 2θ;
s) 2θ values including at least or selected from about 4.18, 17.20, 19.32, and 19.64±0.2° 2θ;
t) 2θ values of about 4.18, 17.20, 19.64, and 24.84±0.2° 2θ;
u) 2θ values including at least or selected from about 4.18, 17.20, 19.64, and 24.84±0.2° 2θ;
v) 2θ values of about 4.18, 17.20, 18.74, and 19.64±0.2° 2θ; and
w) 2θ values including at least or selected from about 4.18, 17.20, 18.74, and 19.64±0.2° 2θ.

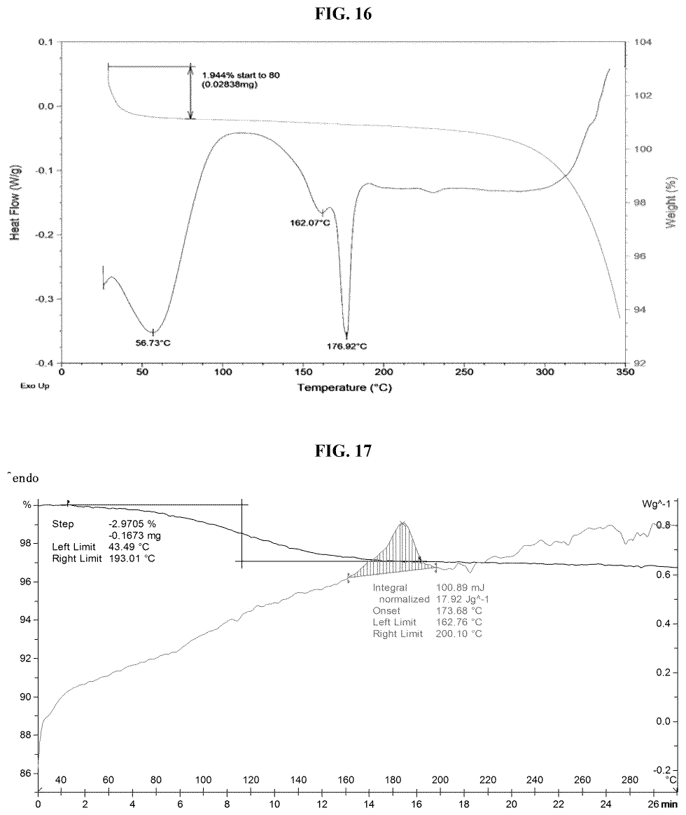

In one or more embodiments, Free Base Form D is characterized by a differential scanning calorimetry (DSC) thermogram having three endotherms with an endotherm maxima between about 50° C. and about 60° C., between about 155° C. and about 165° C., and between about 170° C. and about 180° C. In one or more embodiments, Free Base Form D is characterized by a differential scanning calorimetry (DSC) thermogram having three endotherms with endotherm maxima at about 57° C., 162° C., and 177° C. In one or more embodiments, Free Base Form D is characterized by DSC thermogram substantially similar to that set forth in FIG. 16.

In one or more embodiments, Free Base Form D is characterized by a weight loss in the range of about 0.1% to about 5% when heated from about 25° C. to about 80° C. (such as to about 75° C.) in a thermogravimetric analysis (TGA). In one or more embodiments, Free Base Form D is characterized by a weight loss of about 1.94% when heated from about 25° C. to about 80° C. (such as to about 75° C.) in a TGA. In one or more embodiments, Free Base Form D is characterized by a weight loss of about 2.97% when heated from about 43° C. to about 193° C. in a TGA. In one or more embodiments, Free Base Form D is characterized by a TGA substantially similar to that set forth in FIG. 16. In one or more embodiments, Free Base Form D is characterized by a TGA substantially similar to that set forth in FIG. 16 and/or FIG. 17.

Free Base Form D can be synthesized as described in Synthetic Example 5. In one or more embodiments, Free Base Form D is synthesized by mixing free base compound of Formula (I) in an aprotic solvent. In one or more embodiments, the solvent is selected from the group consisting of tetrahydrofuran, acetone, ethyl acetate, diethyl ether, toluene, hexane, dichloromethane, methyl ethyl ketone, dimethylsulfoxide, and dimethylformamide. After stirring and heating, for instance overnight to 1 week, a precipitate is formed. The precipitate is centrifuged, decanted, and dried to afford Free Base Form D.

In one or more embodiments, Free Base Form D is synthesized by a method comprising:
a) mixing free base compound of Formula (I) in diethyl ether;
b) stirring and heating; and
c) isolating the precipitate to afford Free Base Form D.

In one or more embodiments, the mixture is allowed to stir and heat for at least 12 hours, at least 14 hours, at least 16 hours, at least 18 hours, at least 20 hours, at least 24 hours, at least 48 hours, at least 3 days, at least 4 days, at least 5 days, at least 6 days, or at least 7 days in step (b).

### Free Base Form E

In one or more embodiments, provided herein is a free base of a compound of Formula (I). In one or more embodiments, the free base of the compound of Formula (I) is Free Base Form E.

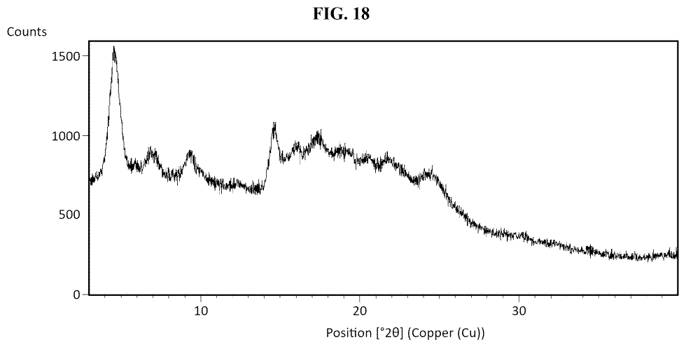

In one or more embodiments, Free Base Form E is characterized by an X-ray powder diffraction (XRPD) pattern substantially similar to that set forth in FIG. 18. In one or more embodiments, Free Base Form E is characterized by an X-ray powder diffraction (XRPD) pattern comprising peaks substantially similar to those set forth in Table 25. In one or more embodiments, Free Base Form E is characterized by an XRPD pattern comprising:
a) 2θ values of about 4.6, 14.6, 17.4, 20.0, and 24.5±0.2° 2θ;
b) 2θ values of about 4.6, 14.6, 15.7, 17.4, 20.0, 22.1, and 24.5±0.2° 2θ;
c) 2θ values including at least or selected from about 4.6, 6.9, 9.4, 12.3, 14.6, 15.7, 17.4, 18.6, 19.2, 20.0, 22.1, and 24.5±0.2° 2θ;
d) at least two, three, four, or five 2θ values selected from about 4.6, 14.6, 15.7, 17.4, 20.0, and 24.5±0.2° 2θ;
e) at least six, seven, or eight 2θ values selected from about 4.6, 6.9, 9.4, 14.6, 15.7, 17.4, 18.6, 19.2, 20.0, 22.1, and 24.5±0.2° 2θ;
f) 2θ values of about 4.6, 6.9, 9.4, 14.6, 15.7, 17.4, 18.6, 20.0, 22.1, and 24.5±0.2° 2θ;
g) 2θ values including at least or selected from about 4.6, 6.9, 9.4, 14.6, 15.7, 17.4, 18.6, 20.0, 22.1, and 24.5±0.2° 2θ;
h) 2θ values of about 4.6, 14.6, 17.4, and 24.5±0.2° 2θ;
i) 2θ values including at least or selected from about 4.6, 14.6, 17.4, and 24.5±0.2° 2θ;
j) 2θ values of about 4.6, 14.6, and 17.4±0.2° 2θ;
k) 2θ values including at least or selected from about 4.6, 14.6, and 17.4±0.2° 2θ;
l) 2θ values of about 4.6, 6.9, 9.4, 14.6, 15.7, 17.4, 19.2, 20.0, 22.1, and 24.5±0.2° 2θ;
m) 2θ values including at least or selected from about 4.6, 6.9, 9.4, 14.6, 15.7, 17.4, 19.2, 20.0, 22.1, and 24.5±0.2° 2θ;
n) 2θ values of about 9.4, 12.3, 14.6, 15.7, 17.4, 18.6, 19.2, 20.0, 22.1, and 24.5±0.2° 2θ;
o) 2θ values including at least or selected from about 9.4, 12.3, 14.6, 15.7, 17.4, 18.6, 19.2, 20.0, 22.1, and 24.5±0.2° 2θ;
p) 2θ values of about 4.6, 14.6, 17.4, and 20.0±0.2° 2θ;
q) 2θ values including at least or selected from about 4.6, 14.6, 17.4, and 20.0±0.2° 2θ;
r) 2θ values of about 4.6, 14.6, 15.7, and 17.4±0.2° 2θ;
s) 2θ values including at least or selected from about 4.6, 14.6, 15.7, and 17.4±0.2° 2θ;
t) 2θ values of about 4.6, 14.6, 17.4, and 22.1±0.2° 2θ;
u) 2θ values including at least or selected from about 4.6, 14.6, 17.4, and 22.1±0.2° 2θ;
v) 2θ values of about 14.6, 17.4, 20.0, and 24.5±0.2° 2θ; and
w) 2θ values including at least or selected from about 14.6, 17.4, 20.0, and 24.5±0.2° 2θ.

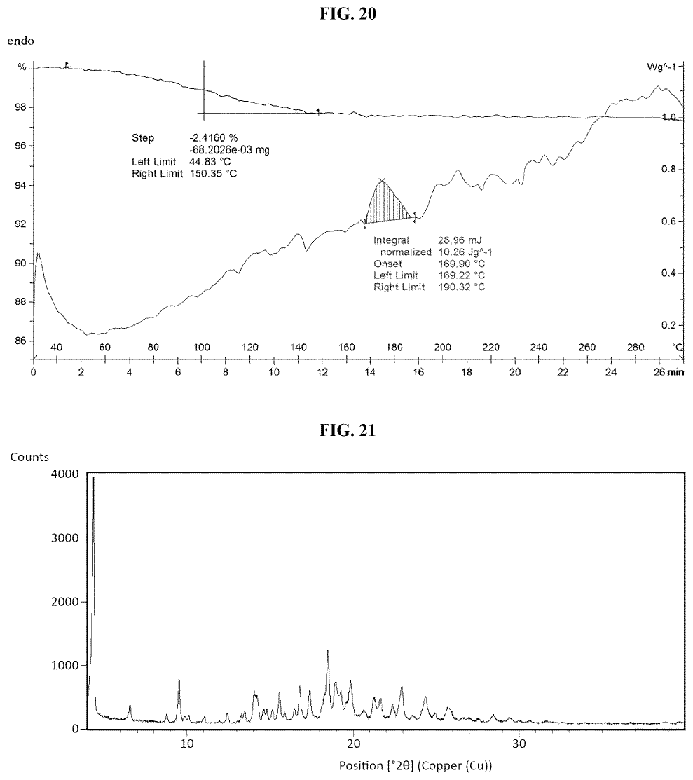

In one or more embodiments, Free Base Form E is characterized by a differential thermal analysis (DTA) thermogram having an endotherm with an endotherm maxima between about 169° C. and about 190° C., or between about 170° C. and about 180° C. In one or more embodiments, Free Base Form E is characterized by a differential thermal analysis (DTA) thermogram having an endotherm with endotherm maxima at about 175° C. In one or more embodiments, Free Base Form E is characterized by DTA thermogram substantially similar to that set forth in FIG. 20.

In one or more embodiments, Free Base Form E is characterized by a weight loss in the range of about 0.1% to about 5% when heated from about 40° C. to about 150° C. in a thermogravimetric analysis (TGA). In one or more embodiments, Free Base Form E is characterized by a weight loss of about 2.42% when heated from about 44° C. to about 150° C. in a TGA. In one or more embodiments, Free Base Form E is characterized by a TGA substantially similar to that set forth in FIG. 20.

Free Base Form E can be synthesized as described in Synthetic Example 6. In one or more embodiments, Free Base Form E is synthesized by mixing free base compound of Formula (I) in an aprotic solvent and temperature cycling or evaporating. In one or more embodiments, when temperature cycling, the solvent is selected from ethanol, ethyl acetate, methanol, 2-methyl-THF, or isopropanol. In one or more embodiments, when evaporating, the solvent is dioxane.

When temperature cycling, after mixing, two temperature cycles (heating followed by cooling) were performed. One temperature cycle (heat cycle) is as follows: heated at a rate of 0.5° C./min (heating rate) from 20° C. to 75° C., and cooled at a rate of 0.2° C./min (cooling rate) from 75° C. to 20° C. This temperature cycle was repeated twice, and a solid was formed. The solid was dried to afford Free Base Form E.

When evaporating, after mixing, dioxane was evaporated and a solid was formed. The solid was dried to afford Free Base Form E.

In one or more embodiments, Free Base Form E is synthesized by a method comprising:
a1) mixing free base compound of Formula (I) in ethanol, ethyl acetate, methanol, 2-methyl-tetrahydrofuran, or isopropanol;
b1) heating at a rate of about 0.5° C./min (heating rate) from about 20° C. to about 75° C., then cooling at a rate of about 0.2° C./min (cooling rate) from about 75° C. to about 20° C.;
c1) heating at a rate of about 0.5° C./min (heating rate) from about 20° C. to about 75° C., then cooling at a rate of about 0.2° C./min (cooling rate) from about 75° C. to about 20° C.; and
d1) isolating the solid to afford Free Base Form E;
or
a2) mixing free base compound of Formula (I) in dioxane;
b2) evaporating dioxane; and
c2) isolating the solid to afford Free Base Form E.

### Free Base Form F

In one or more embodiments, provided herein is a free base of a compound of Formula (I). In one or more embodiments, the free base of the compound of Formula (I) is Free Base Form F.

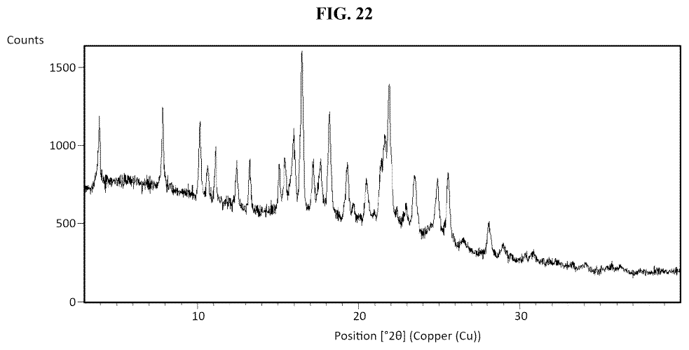

In one or more embodiments, Free Base Form F is characterized by an X-ray powder diffraction (XRPD) pattern substantially similar to that set forth in FIG. 21. In one or more embodiments, Free Base Form F is characterized by an X-ray powder diffraction (XRPD) pattern comprising peaks substantially similar to those set forth in Table 26. In one or more embodiments, Free Base Form F is characterized by an XRPD pattern comprising:
a) 2θ values of about 4.3, 9.5, 16.8, 18.5, and 19.8±0.2° 2θ;
b) 2θ values of about 4.3, 9.5, 14.0, 15.6, 16.8, 17.4, 18.5, 18.9, 19.2, 19.8, 21.3, 21.6, 22.9, 24.4±0.2° 2θ;
c) 2θ values including at least or selected from about 4.3, 6.6, 8.8, 9.5, 9.9, 10.1, 11.0, 11.9, 12.4, 13.2, 13.5, 14.0, 14.1, 14.2, 14.6, 14.8, 15.1, 15.6, 15.9, 16.4, 16.8, 17.4, 18.5, 18.9, 19.2, 19.6, 19.8, 20.6, 21.3, 21.6, 22.3, 22.9, 23.6, 24.2, 24.4, 24.9, 25.7, 25.9, 26.6, 27.0, 27.5, 28.4, 29.4, 30.0, 30.6, 31.7±0.2° 2θ;
d) at least two, three, four, or five 2θ values selected from about 4.3, 9.5, 16.8, 18.5, 19.8, and 22.9±0.2° 2θ;
e) at least six, seven, or eight 2θ values selected from about 4.3, 9.5, 14.0, 15.6, 16.8, 17.4, 18.5, 18.9, 19.2, 19.8, 21.6, 22.9, and 24.4±0.2° 2θ;
f) 2θ values of about 4.3, 9.5, 14.0, 15.6, 16.8, 17.4, 18.5, 18.9, 19.8, and 22.9±0.2° 2θ;
g) 2θ values including at least or selected from about 4.3, 9.5, 14.0, 15.6, 16.8, 17.4, 18.5, 18.9, 19.8, and 22.9±0.2° 2θ;
h) 2θ values of about 4.3, 9.5, 18.5, and 19.8±0.2° 2θ;
i) 2θ values including at least or selected from about 4.3, 9.5, 18.5, and 19.8±0.2° 2θ;
j) 2θ values of about 4.3, 9.5, and 18.5±0.2° 2θ;
k) 2θ values including at least or selected from about 4.3, 9.5, and 18.5±0.2° 2θ;
l) 2θ values of about 4.3, 9.5, 14.0, 15.6, 16.8, 17.4, 18.5, 18.9, 19.2, and 19.8±0.2° 2θ;
m) 2θ values including at least or selected from about 4.3, 9.5, 14.0, 15.6, 16.8, 17.4, 18.5, 18.9, 19.2, and 19.8±0.2° 2θ;
n) 2θ values of about 15.6, 16.8, 17.4, 18.5, 18.9, 19.2, 19.8, 21.6, 22.9, and 24.4±0.2° 2θ;
o) 2θ values including at least or selected from about 15.6, 16.8, 17.4, 18.5, 18.9, 19.2, 19.8, 21.6, 22.9, and 24.4±0.2° 2θ;
p) 2θ values of about 4.3, 9.5, 16.8, and 18.5±0.2° 2θ;
q) 2θ values including at least or selected from about 4.3, 9.5, 16.8, and 18.5±0.2° 2θ;
r) 2θ values of about 4.3, 9.5, 18.5, and 22.9±0.2° 2θ;
s) 2θ values including at least or selected from about 4.3, 9.5, 18.5, and 22.9±0.2° 2θ;
t) 2θ values of about 4.3, 9.5, 17.4, and 18.5±0.2° 2θ;
u) 2θ values including at least or selected from about 4.3, 9.5, 17.4, and 18.5±0.2° 2θ;
v) 2θ values of about 9.5, 16.8, 18.5, and 19.8±0.2° 2θ; and
w) 2θ values including at least or selected from about 9.5, 16.8, 18.5, and 19.8±0.2° 2θ.

Free Base Form F can be synthesized as described in Synthetic Example 7. In one or more embodiments, Free Base Form F is synthesized by mixing free base compound of Formula (I) in an aprotic solvent and temperature cycling. In one or more embodiments, the solvent is toluene. After mixing, two temperature cycles (heating followed by cooling) were performed. One temperature cycle (heat cycle) is as follows: heated at a rate of 0.5° C./min (heating rate) from 20° C. to 100° C., and cooled at a rate of 0.2° C./min (cooling rate) from 100° C. to 20° C. This temperature cycle was repeated twice, and a solid was formed. The solid was dried to afford Free Base Form F.

In one or more embodiments, Free Base Form F is synthesized by a method comprising:
a) mixing free base compound of Formula (I) in toluene;
b) heating at a rate of about 0.5° C./min (heating rate) from about 20° C. to about 100° C., then cooling at a rate of about 0.2° C./min (cooling rate) from about 100° C. to about 20° C.;
c) heating at a rate of about 0.5° C./min (heating rate) from about 20° C. to about 100° C., then cooling at a rate of about 0.2° C./min (cooling rate) from about 100° C. to about 20° C.; and
d) isolating the solid to afford Free Base Form F.

### Free Base Form G

In one or more embodiments, provided herein is a free base of a compound of Formula (I). In one or more embodiments, the free base of the compound of Formula (I) is Free Base Form G.

In one or more embodiments, Free Base Form G is characterized by an X-ray powder diffraction (XRPD) pattern substantially similar to that set forth in FIG. 22. In one or more embodiments, Free Base Form G is characterized by an X-ray powder diffraction (XRPD) pattern comprising peaks substantially similar to those set forth in Table 27. In one or more embodiments, Free Base Form G is characterized by an XRPD pattern comprising:
a) 2θ values of about 7.8, 16.0, 16.5, 18.2, and 21.9±0.2° 2θ;
b) 2θ values of about 7.8, 10.1, 16.0, 16.5, 18.2, 21.6, 21.9, and 25.5±0.2° 2θ;
c) 2θ values including at least or selected from about 3.9, 7.8, 10.1, 10.6, 11.1, 12.4, 13.2, 15.1, 15.4, 15.7, 16.0, 16.4, 16.5, 17.2, 17.6, 18.2, 19.1, 19.3, 19.7, 20.5, 21.4, 21.6, 21.9, 22.9, 23.5, 24.9, 25.5, 26.5, 28.1, 29.0, 30.3, and 30.8±0.2° 2θ;
d) at least two, three, four, or five 2θ values selected from about 7.8, 10.1, 16.0, 16.5, 18.2, and 21.9±0.2° 2θ;
e) at least six, seven, or eight 2θ values selected from about 3.9, 7.8, 10.1, 16.0, 16.5, 17.2, 18.2, 19.3, 21.6, 21.9, 23.5, 24.9, and 25.5±0.2° 2θ;
f) 2θ values of about 3.9, 7.8, 10.1, 16.0, 16.5, 18.2, 21.6, 21.9, 23.5, and 25.5±0.2° 2θ;
g) 2θ values including at least or selected from about 3.9, 7.8, 10.1, 16.0, 16.5, 18.2, 21.6, 21.9, 23.5, and 25.5±0.2° 2θ;
h) 2θ values of about 7.8, 16.5, 18.2, and 21.9±0.2° 2θ;
i) 2θ values including at least or selected from about 7.8, 16.5, 18.2, and 21.9±0.2° 2θ;
j) 2θ values of about 16.5, 18.2, and 21.9±0.2° 2θ;
k) 2θ values including at least or selected from about 16.5, 18.2, and 21.9±0.2° 2θ;
l) 2θ values of about 7.8, 10.1, 16.0, 16.5, 18.2, 19.3, 21.6, 21.9, 23.5, and 25.5±0.2° 2θ;
m) 2θ values including at least or selected from about 7.8, 10.1, 16.0, 16.5, 18.2, 19.3, 21.6, 21.9, 23.5, and 25.5±0.2° 2θ;
n) 2θ values of about 16.0, 16.5, 17.2, 18.2, 19.3, 21.6, 21.9, 23.5, 24.9, and 25.5±0.2° 2θ;
o) 2θ values including at least or selected from about 16.0, 16.5, 17.2, 18.2, 19.3, 21.6, 21.9, 23.5, 24.9, and 25.5±0.2° 2θ;
p) 2θ values of about 16.0, 16.5, 18.2, and 21.9±0.2° 2θ;
q) 2θ values including at least or selected from about 16.0, 16.5, 18.2, and 21.9±0.2° 2θ;
r) 2θ values of about 10.1, 16.5, 18.2, and 21.9±0.2° 2θ;
s) 2θ values including at least or selected from about 10.1, 16.5, 18.2, and 21.9±0.2° 2θ;
t) 2θ values of about 16.5, 18.2, 21.9, and 25.5±0.2° 2θ;
u) 2θ values including at least or selected from about 16.5, 18.2, 21.9, and 25.5±0.2° 2θ;
v) 2θ values of about 16.5, 18.2, 19.3, and 21.9±0.2° 2θ; and
w) 2θ values including at least or selected from about 16.5, 18.2, 19.3, and 21.9±0.2° 2θ.

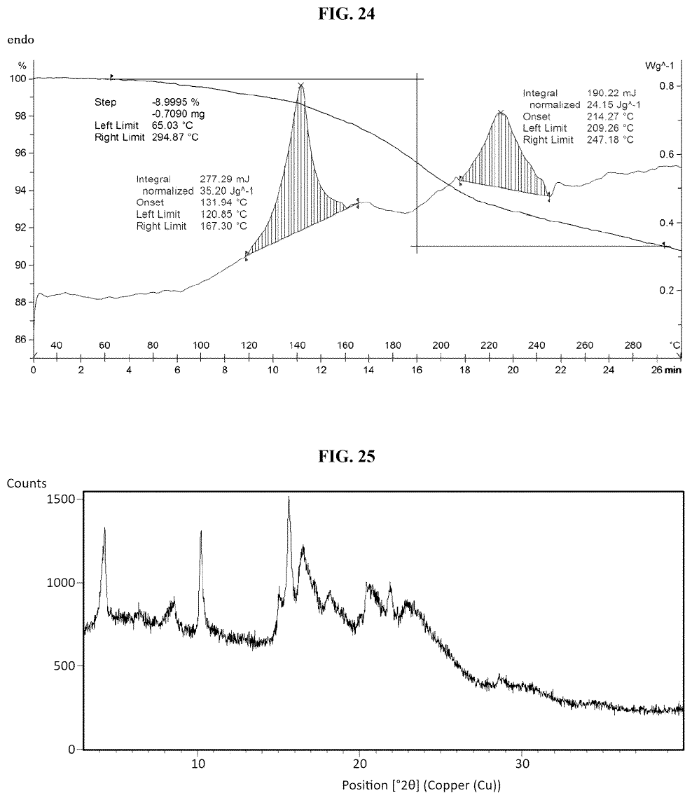

In one or more embodiments, Free Base Form G is characterized by a differential thermal analysis (DTA) thermogram having an endotherm with two endotherm maxima between about 120° C. and about 170° C. and between about 205° C. and about 250° C. In one or more embodiments, Free Base Form G is characterized by a differential thermal analysis (DTA) thermogram having two endotherms with endotherm maxima at about 145° C. and about 225° C. In one or more embodiments, Free Base Form G is characterized by DTA thermogram substantially similar to that set forth in FIG. 24.

In one or more embodiments, Free Base Form G is characterized by a weight loss in the range of about 5% to about 10% when heated from about 65° C. to about 295° C. in a thermogravimetric analysis (TGA). In one or more embodiments, Free Base Form G is characterized by a weight loss of about 8.99% when heated from about 65° C. to about 295° C. in a TGA. In one or more embodiments, Free Base Form G is characterized by a TGA substantially similar to that set forth in FIG. 24.

Free Base Form G can be synthesized as described in Synthetic Example 8. In one or more embodiments, Free Base Form G is synthesized by mixing free base compound of Formula (I) in an aprotic solvent and evaporating. In one or more embodiments, the solvent is dimethylacetamide.

After mixing, dimethylacetamide was evaporated under vacuum at about 50° C. and a solid was formed. The solid was dried to afford Free Base Form G.

In one or more embodiments, Free Base Form G is synthesized by a method comprising:
a) mixing free base compound of Formula (I) in dimethylacetamide;
b) evaporating dimethylacetamide under vacuum at about 50° C.; and
c) isolating the solid to afford Free Base Form G.

### Free Base Form H

In one or more embodiments, provided herein is a free base of a compound of Formula (I). In one or more embodiments, the free base of the compound of Formula (I) is Free Base Form H.

In one or more embodiments, Free Base Form H is characterized by an X-ray powder diffraction (XRPD) pattern substantially similar to that set forth in FIG. 25. In one or more embodiments, Free Base Form H is characterized by an X-ray powder diffraction (XRPD) pattern comprising peaks substantially similar to those set forth in Table 28. In one or more embodiments, Free Base Form H is characterized by an XRPD pattern comprising:
a) 2θ values of about 4.2, 10.2, 15.7, 16.5, and 20.9±0.2° 2θ;
b) 2θ values of about 4.2, 10.2, 15.7, 16.5, 18.1, 20.9, and 23.4±0.2° 2θ;
c) 2θ values including at least or selected from about 4.2, 6.4, 8.4, 10.2, 15.1, 15.7, 16.5, 16.9, 17.2, 18.1, 19.0, 20.6, 20.9, 21.9, 23.4, 25.6, 26.4, 27.0, 28.6, 28.8, 30.0, and 39.7±0.2° 2θ;
d) at least two, three, four, or five 2θ values selected from about 4.2, 10.2, 15.7, 16.5, 20.9, and 23.4±0.2° 2θ;
e) at least six, seven, or eight 2θ values selected from about 4.2, 8.4, 10.2, 15.1, 15.7, 16.5, 17.2, 18.1, 20.6, 20.9, 21.9, and 23.4±0.2° 2θ;
f) 2θ values of about 4.2, 10.2, 15.1, 15.7, 16.5, 17.2, 18.1, 20.6, 20.9, and 23.4±0.2° 2θ;
g) 2θ values including at least or selected from about 4.2, 10.2, 15.1, 15.7, 16.5, 17.2, 18.1, 20.6, 20.9, and 23.4±0.2° 2θ;
h) 2θ values of about 4.2, 10.2, 15.7, and 16.5±0.2° 2θ;
i) 2θ values including at least or selected from about 4.2, 10.2, 15.7, and 16.5±0.2° 2θ;
j) 2θ values of about 10.2, 15.7, and 16.5±0.2° 2θ;
k) 2θ values including at least or selected from about 10.2, 15.7, and 16.5±0.2° 2θ;
l) 2θ values of about 4.2, 10.2, 15.1, 15.7, 16.5, 17.2, 18.1, 20.9, 21.9, and 23.4±0.2° 2θ;
m) 2θ values including at least or selected from about 4.2, 10.2, 15.1, 15.7, 16.5, 17.2, 18.1, 20.9, 21.9, and 23.4±0.2° 2θ;
n) 2θ values of about 10.2, 15.1, 15.7, 16.5, 17.2, 18.1, 20.6, 20.9, 21.9, and 23.4±0.2° 2θ;
o) 2θ values including at least or selected from about 10.2, 15.1, 15.7, 16.5, 17.2, 18.1, 20.6, 20.9, 21.9, and 23.4±0.2° 2θ;
p) 2θ values of about 10.2, 15.7, 16.5, and 20.9±0.2° 2θ;
q) 2θ values including at least or selected from about 10.2, 15.7, 16.5, and 20.9±0.2° 2θ;
r) 2θ values of about 10.2, 15.7, 16.5, and 23.4±0.2° 2θ;
s) 2θ values including at least or selected from about 10.2, 15.7, 16.5, and 23.4±0.2° 2θ;
t) 2θ values of about 10.2, 15.7, 16.5, and 18.1±0.2° 2θ;
u) 2θ values including at least or selected from about 10.2, 15.7, 16.5, and 18.1±0.2° 2θ;
v) 2θ values of about 10.2, 15.7, 16.5, and 17.2±0.2° 2θ; and
w) 2θ values including at least or selected from about 10.2, 15.7, 16.5, and 17.2±0.2° 2θ.

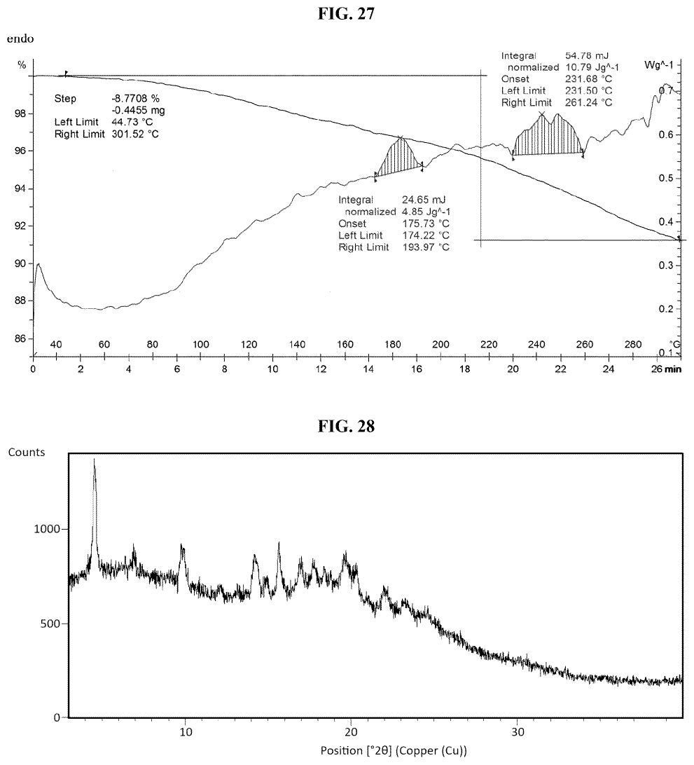

In one or more embodiments, Free Base Form H is characterized by a differential thermal analysis (DTA) thermogram having an endotherm with two endotherm maxima between about 170° C. and about 195° C. and between about 230° C. and about 265° C. or with two endotherm maxima between about 174° C. and about 194° C. and between about 231° C. and about 261° C. In one or more embodiments, Free Base Form H is characterized by a differential thermal analysis (DTA) thermogram having two endotherms with endotherm maxima at about 185° C. and about 245° C. In one or more embodiments, Free Base Form H is characterized by DTA thermogram substantially similar to that set forth in FIG. 27.

In one or more embodiments, Free Base Form H is characterized by a weight loss in the range of about 5% to about 10% when heated from about 40° C. to about 305° C. in a thermogravimetric analysis (TGA). In one or more embodiments, Free Base Form H is characterized by a weight loss of about 8.77% when heated from about 44° C. to about 302° C. in a TGA. In one or more embodiments, Free Base Form H is characterized by a TGA substantially similar to that set forth in FIG. 27.

Free Base Form H can be synthesized as described in Synthetic Example 9. In one or more embodiments, Free Base Form H is synthesized by mixing free base compound of Formula (I) in an aprotic solvent and evaporating. In one or more embodiments, the solvent is dimethylsulfoxide.

After mixing, dimethylsulfoxide was evaporated under vacuum at about 50° C. and a solid was formed. The solid was dried to afford Free Base Form H.

In one or more embodiments, Free Base Form H is synthesized by a method comprising:
a) mixing free base compound of Formula (I) in dimethylsulfoxide;
b) evaporating dimethylsulfoxide under vacuum at about 50° C.; and
c) isolating the solid to afford Free Base Form H.

### Free Base Form J

In one or more embodiments, provided herein is a free base of a compound of Formula (I). In one or more embodiments, the free base of the compound of Formula (I) is Free Base Form J.

In one or more embodiments, Free Base Form J is characterized by an X-ray powder diffraction (XRPD) pattern substantially similar to that set forth in FIG. 28. In one or more embodiments, Free Base Form J is characterized by an X-ray powder diffraction (XRPD) pattern comprising peaks substantially similar to those set forth in Table 29. In one or more embodiments, Free Base Form J is characterized by an XRPD pattern comprising:
a) 2θ values of about 4.6, 9.9, 14.2, 15.7, and 17.0±0.2° 2θ;
b) 2θ values of about 4.6, 9.9, 14.2, 15.7, 17.0, 19.6, and 20.3±0.2° 2θ;
c) 2θ values including at least or selected from about 4.6, 6.9, 9.9, 12.1, 14.2, 14.9, 15.7, 17.0, 17.8, 18.4, 18.8, 19.6, 20.3, 22.0, 22.9, 24.7±0.2° 2θ;
d) at least two, three, four, or five 2θ values selected from about 4.6, 9.9, 14.2, 15.7, 17.0, and 19.6±0.2° 2θ;
e) at least six, seven, or eight 2θ values selected from about 4.6, 9.9, 14.2, 15.7, 17.0, 17.8, 18.4, 19.6, 20.3, 22.0, and 22.9±0.2° 2θ;
f) 2θ values of about 4.6, 9.9, 14.2, 15.7, 17.0, 17.8, 18.4, 19.6, and 20.3±0.2° 2θ;
g) 2θ values including at least or selected from about 4.6, 9.9, 14.2, 15.7, 17.0, 17.8, 18.4, 19.6, and 20.3±0.2° 2θ;
h) 2θ values of about 4.6, 14.2, 15.7, and 17.0±0.2° 2θ;
i) 2θ values including at least or selected from about 4.6, 14.2, 15.7, and 17.0±0.2° 2θ;
j) 2θ values of about 4.6, 14.2, and 15.7±0.2° 2θ;
k) 2θ values including at least or selected from about 4.6, 14.2, and 15.7±0.2° 2θ;
l) 2θ values of about 4.6, 9.9, 14.2, 15.7, 17.0, 18.4, 19.6, 20.3, and 22.9±0.2° 2θ;
m) 2θ values including at least or selected from about 4.6, 9.9, 14.2, 15.7, 17.0, 18.4, 19.6, 20.3, and 22.9±0.2° 2θ;
n) 2θ values of about 14.2, 15.7, 17.0, 17.8, 18.4, 19.6, 20.3, 22.0, and 22.9±0.2° 2θ;
o) 2θ values including at least or selected from about 14.2, 15.7, 17.0, 17.8, 18.4, 19.6, 20.3, 22.0, and 22.9±0.2° 2θ;
p) 2θ values of about 4.6, 9.9, 14.2, and 15.7±0.2° 2θ;
q) 2θ values including at least or selected from about 4.6, 9.9, 14.2, and 15.7±0.2° 2θ;
r) 2θ values of about 4.6, 14.2, 15.7, and 19.6±0.2° 2θ;
s) 2θ values including at least or selected from about 4.6, 14.2, 15.7, and 19.6±0.2° 2θ;
t) 2θ values of about 4.6, 14.2, 15.7, and 20.3±0.2° 2θ;
u) 2θ values including at least or selected from about 4.6, 14.2, 15.7, and 20.3±0.2° 2θ;
v) 2θ values of about 4.6, 14.2, 15.7, and 18.4±0.2° 2θ; and
w) 2θ values including at least or selected from about 4.6, 14.2, 15.7, and 18.4±0.2° 2θ.

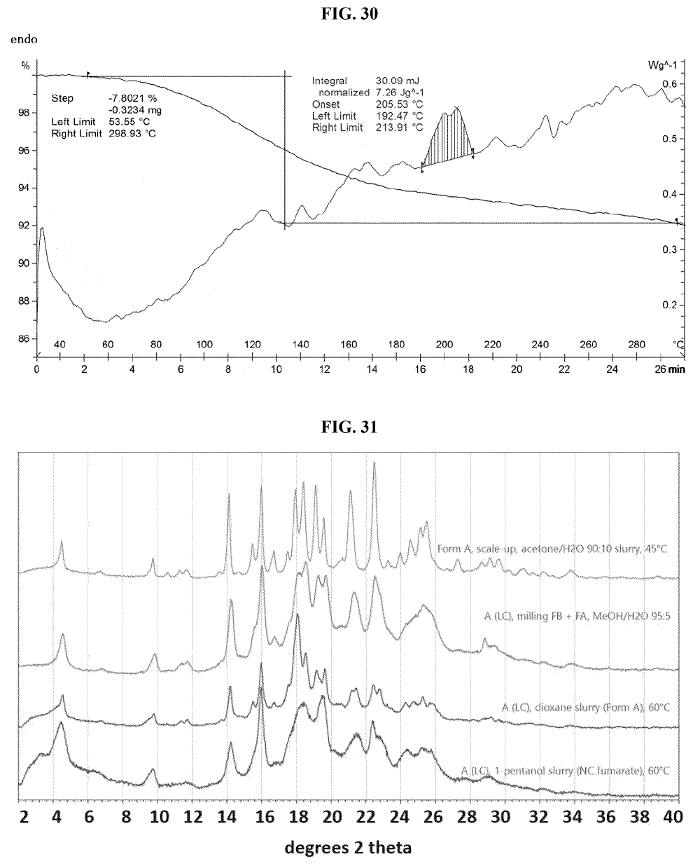

In one or more embodiments, Free Base Form J is characterized by a differential thermal analysis (DTA) thermogram having an endotherm with an endotherm maxima between about 190° C. and about 215° C. In one or more embodiments, Free Base Form J is characterized by a differential thermal analysis (DTA) thermogram having an endotherm with endotherm maxima at about 192° C. and about 214° C. In one or more embodiments, Free Base Form J is characterized by DTA thermogram substantially similar to that set forth in FIG. 30.

In one or more embodiments, Free Base Form J is characterized by a weight loss in the range of about 5% to about 10% when heated from about 50° C. to about 300° C. in a thermogravimetric analysis (TGA). In one or more embodiments, Free Base Form J is characterized by a weight loss of about 7.80% when heated from about 54° C. to about 299° C. in a TGA. In one or more embodiments, Free Base Form J is characterized by a TGA substantially similar to that set forth in FIG. 30.

Free Base Form J can be synthesized as described in Synthetic Example 10. In one or more embodiments, Free Base Form J is synthesized by mixing free base compound of Formula (I) in an aprotic solvent and evaporating. In one or more embodiments, the solvent is anisole. In one or more embodiments, Free Base Form J is synthesized by conversion of Free Base Form F stored under ambient conditions (including a temperature of from 15° C. to 30° C.).

When evaporating, after mixing, anisole was evaporated at ambient temperature (of from 15° C. to 30° C.) and a solid was formed. The solid was dried to afford Free Base Form J.

When converting, Free Base Form F was stored under ambient conditions (including a temperature of from 15° C. to 30° C.) for 4-7 days and a solid was formed. The solid was dried to afford Free Base Form J.

In one or more embodiments, Free Base Form J is synthesized by a method comprising:
a1) mixing free base compound of Formula (I) in anisole;
b1) evaporating anisole at ambient temperature; and
c1) isolating the solid to afford Free Base Form J;
or
a2) obtaining Free Base Form F and storing under ambient conditions (including a temperature of from 15° C. to 30° C.) for 4-7 days;
b2) isolating solid to afford Free Base Form J.

### Additional Salt Forms

Also provided herein is a salt form of a compound of Formula (I) selected from the group consisting of a fumarate salt, a maleate salt, and a p-toluenesulfonic acid (PTSA) salt.

In one or more embodiments, provided herein is a salt form of a compound of Formula (I) selected from the group consisting of a hydrochloric acid salt (HCl salt), a sulfuric acid salt, a methanesulfonic acid salt, a phosphoric acid salt, a tartaric acid salt, a citric acid salt, a hippuric acid salt, and a gluconic acid salt.

In one or more embodiments, provided herein is a salt form of a compound of Formula (I) selected from the group consisting of Formula (I-A), Formula (I-B), and Formula (I-C):

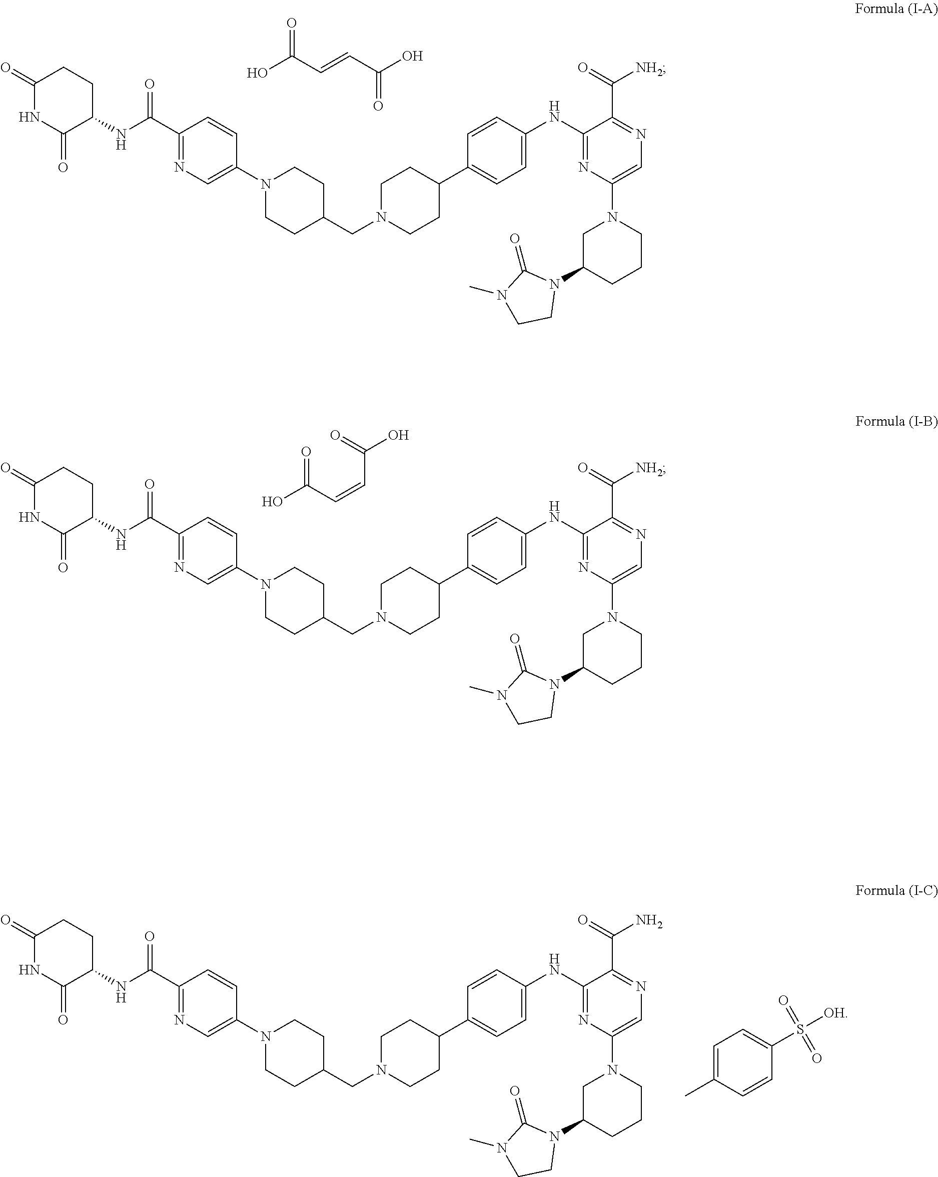

The salt forms of a compound of Formula (I) described herein can be in a form that is at least 90% free of the opposite compound of Formula (I) enantiomer (excluding the weight of the salt). In one or more embodiments, the salt form of a compound of Formula (I) is at least 95%, 98%, 99%, or even 100% free of the opposite compound of Formula (I) enantiomer (excluding the weight of the salt).

For example, in one or more embodiments, the compound of Formula (I-A) is at least 90%, 95%, 98%, 99%, or even 100% free of the opposite compound of Formula (I-A) (excluding the weight of the salt). For example, in one or more embodiments, the compound of Formula (I-B) is at least 90%, 95%, 98%, 99%, or even 100% free of the opposite compound of Formula (I-B) (excluding the weight of the salt). For example, in one or more embodiments, the compound of Formula (I-C) is at least 90%, 95%, 98%, 99%, or even 100% free of the opposite compound of Formula (I-C) (excluding the weight of the salt).

### Fumarate Salt Form A

In one or more embodiments, provided herein is a salt of a compound of Formula (I). In one or more embodiments, the salt of the compound of Formula (I) is a fumarate salt. In one or more embodiments, the fumarate salt is Fumarate Salt Form A.

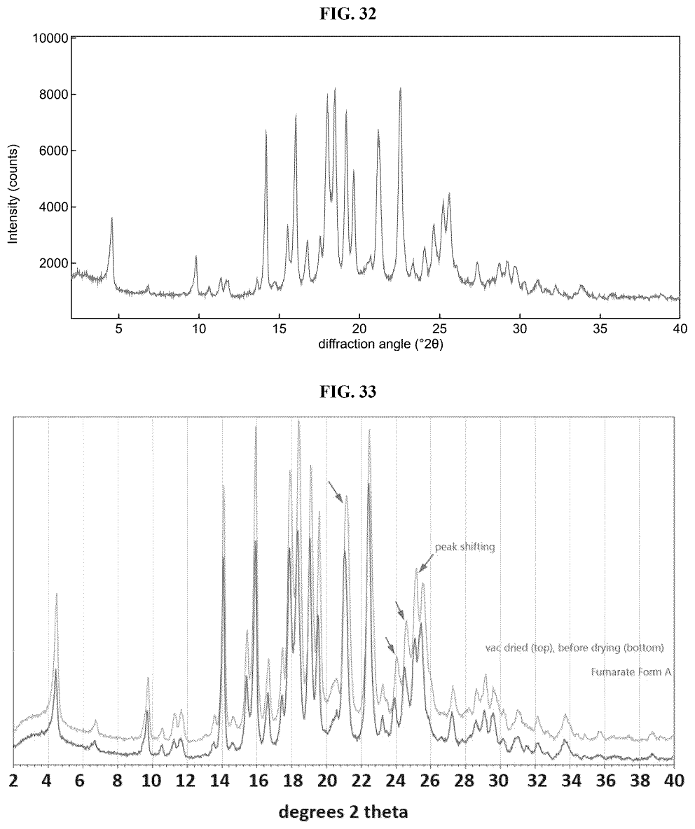

In one or more embodiments, Fumarate Salt Form A is characterized by an X-ray powder diffraction (XRPD) pattern substantially similar to that set forth in FIG. 32. In one or more embodiments, Fumarate Salt Form A is characterized by an X-ray powder diffraction (XRPD) pattern comprising peaks substantially similar to those set forth in Table 30. In one or more embodiments, Fumarate Salt Form A is characterized by an XRPD pattern comprising:
a) 2θ values of about 4.58, 14.18, 16.04, 18.00, 18.48, 19.18, 19.64, 21.16, 22.56, 25.22, and 25.58±0.2° 2θ;
b) 2θ values of about 4.58, 9.82, 14.18, 15.52, 16.04, 18.00, 18.48, 19.18, 19.64, 21.16, 22.56, 24.64, 25.22, and 25.58±0.2° 2θ;
c) 2θ values including at least or selected from about 4.58, 6.84, 9.82, 10.64, 11.38, 11.72, 13.62, 14.18, 14.72, 15.52, 16.04, 16.76, 17.54, 18.00, 18.48, 19.18, 19.64, 20.70 21.16, 22.56, 23.36, 24.06, 24.64, 25.22, 25.58, 26.06, 27.36, 27.98, 28.72, 29.18, 29.68, 30.30, 31.08, 31.58, 32.22, and 33.76±0.2° 2θ;
d) at least two, three, four, or five 2θ values selected from about 4.58, 14.18, 15.52, 16.04, 16.76, 17.54, 18.00, 18.48, 19.18, 19.64, 21.16, 22.56, 24.06, 24.64, 25.22, 25.58±0.2° 2θ;
e) at least six, seven, or eight 2θ values selected from about 4.58, 9.82, 14.18, 15.52, 16.04, 16.76, 17.54, 18.00, 18.48, 19.18, 19.64, 20.70, 21.16, 22.56, 23.36, 24.06, 24.64, 25.22, 25.58, 26.06, 27.36, 28.72, 29.18, 29.68±0.2° 2θ;
f) 2θ values of about 14.18, 16.04, 18.00, 18.48, 19.18, 21.16, and 22.56±0.2° 2θ;
g) 2θ values including at least or selected from about 14.18, 16.04, 18.00, 18.48, 19.18, 21.16, and 22.56±0.2° 2θ;
h) 2θ values of about 18.00, 18.48, 19.18, 22.56±0.2° 2θ;
i) 2θ values including at least or selected from about 18.00, 18.48, 19.18, 22.56±0.2° 2θ;
j) 2θ values of about 18.00, 18.48, 22.56±0.2° 2θ;
k) 2θ values including at least or selected from about 18.00, 18.48, 22.56±0.2° 2θ;
l) 2θ values of about 14.18, 16.04, 18.00, 18.48, 19.18, 19.64, 21.16, 22.56, 25.22, 25.58±0.2° 2θ;
m) 2θ values including at least or selected from about 14.18, 16.04, 18.00, 18.48, 19.18, 19.64, 21.16, 22.56, 25.22, 25.58±0.2° 2θ;
n) 2θ values of about 14.18, 16.04, 18.00, 18.48, 19.18, 19.64, 21.16, and 22.56±0.2° 2θ;
o) 2θ values including at least or selected from about 14.18, 16.04, 18.00, 18.48, 19.18, 19.64, 21.16, and 22.56±0.2° 2θ;
p) 2θ values of about 14.18, 18.00, 18.48, and 22.56±0.2° 2θ;
q) 2θ values including at least or selected from about 14.18, 18.00, 18.48, and 22.56±0.2° 2θ;
r) 2θ values of about 16.04, 18.00, 18.48, and 22.56±0.2° 2θ;
s) 2θ values including at least or selected from about 16.04, 18.00, 18.48, and 22.56±0.2° 2θ;
t) 2θ values of about 18.00, 18.48, 19.18, and 22.56±0.2° 2θ;
u) 2θ values including at least or selected from about 18.00, 18.48, 19.18, and 22.56±0.2° 2θ;
v) 2θ values of about 18.00, 18.48, 21.16, and 22.56±0.2° 2θ; and
w) 2θ values including at least or selected from about 18.00, 18.48, 21.16, and 22.56±0.2° 2θ.

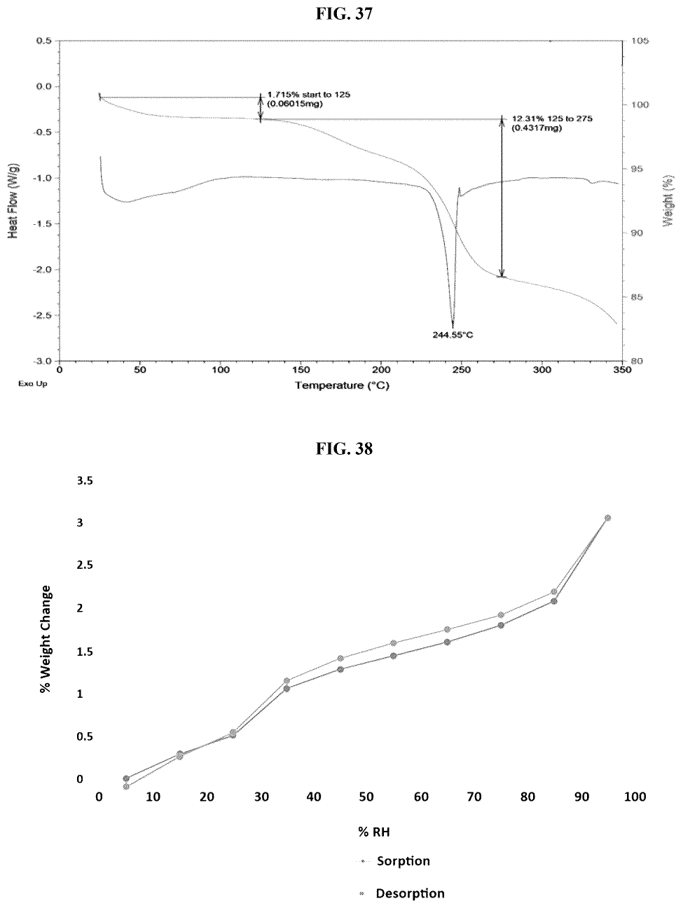

In one or more embodiments, Fumarate Salt Form A is characterized by a differential scanning calorimetry (DSC) thermogram having one endotherm with an endotherm maxima between about 240° C. and about 250° C. In one or more embodiments, Fumarate Salt Form A is characterized by a differential scanning calorimetry (DSC) thermogram having one endotherm with an endotherm maxima at about 245° C. In one or more embodiments, Fumarate Salt Form A is characterized by a DSC thermogram substantially similar to that set forth in FIG. 37.

In one or more embodiments, Fumarate Salt Form A is characterized by a weight loss in the range of about 0.1% to about 5% when heated from about 25° C. to about 125° C., and in the range of about 10% to about 15% when heated from about 125° C. to about 275° C. in a thermogravimetric analysis (TGA). In one or more embodiments, Fumarate Salt Form A is characterized by a weight loss of about 1.7% when heated from about 25° C. to about 125° C. in a TGA, and from about 10.0% to about 12.3% when heated from about 125° C. to about 275° C. In one or more embodiments, Fumarate Salt Form A is characterized by a TGA substantially similar to that set forth in FIG. 37.

A DVS analysis of Fumarate Salt Form A is shown in FIG. 38.

Fumarate Salt Form A can be synthesized as described in Synthetic Example 11. In one or more embodiments, Fumarate Salt Form A is synthesized by mixing free base compound of Formula (I) and fumaric acid in a solvent. Compound of Formula (I) can be synthesized as described in Synthetic Example A, in its free form (which is called a free base or free base starting material). In one or more embodiments, the solvent is a protic solvent. In one or more embodiments, the solvent is an aprotic solvent. In one or more embodiments, the solvent is selected from the group consisting of methanol, ethanol, isopropanol, formic acid, acetic acid, acetone, and DCM. In one or more embodiments, the mixture comprises water. In one or more embodiments, the mixture does not comprise water. After stirring, for instance overnight or a week and under heat, the precipitate is centrifuged, decanted, and dried to afford Fumarate Salt Form A.

In one or more embodiments, Fumarate Salt Form A is synthesized by a method comprising:
a) mixing free base compound of Formula (I) and fumaric acid in methanol and water;
b) stirring and heating to afford a precipitate; and
c) isolating the precipitate to afford Fumaric Salt Form A.

In one or more embodiments, the mixture is allowed to stir and heat for at least 12 hours, at least 14 hours, at least 16 hours, at least 18 hours, at least 20 hours, at least 24 hours, at least 2 days, at least 3 days, at least 4 days, at least 5 days, or at least 6 days in step (b).

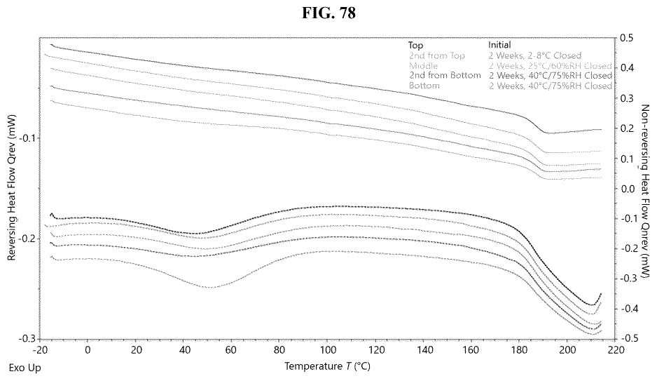

In one or more embodiments, an amorphous solid dispersion (ASD) prepared from Fumarate Salt Form A is characterized by an X-ray powder diffraction (XRPD) pattern substantially similar to that set forth in FIG. 77. In one or more embodiments, an amorphous solid dispersion (ASD) prepared from Fumarate Salt Form A is characterized by a differential scanning calorimetry (DSC) pattern substantially similar to that set forth in FIG. 78. In one or more embodiments, when a compound of Formula (I) (e.g., Fumarate Salt Form A) is converted and presented in the amorphous solid dispersion, the form is amorphous.

### Fumarate Salt Amorphous Form

In one or more embodiments, provided herein is a salt a compound of Formula (I). In one or more embodiments, the salt of the compound of Formula (I) is a fumarate salt. In one or more embodiments, the salt of the compound of Formula (I) is Fumarate Salt Amorphous Form. In one or more embodiments, the Fumarate Salt Amorphous Form is a hydrate.

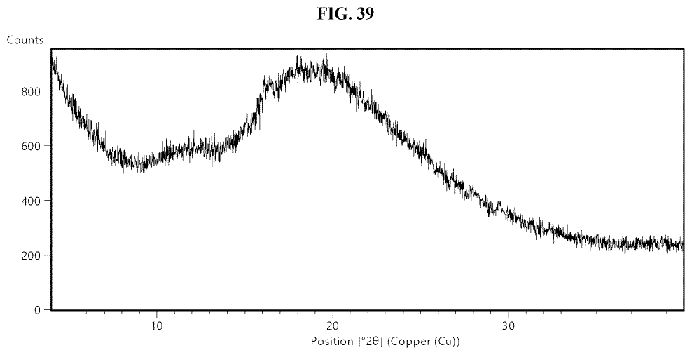

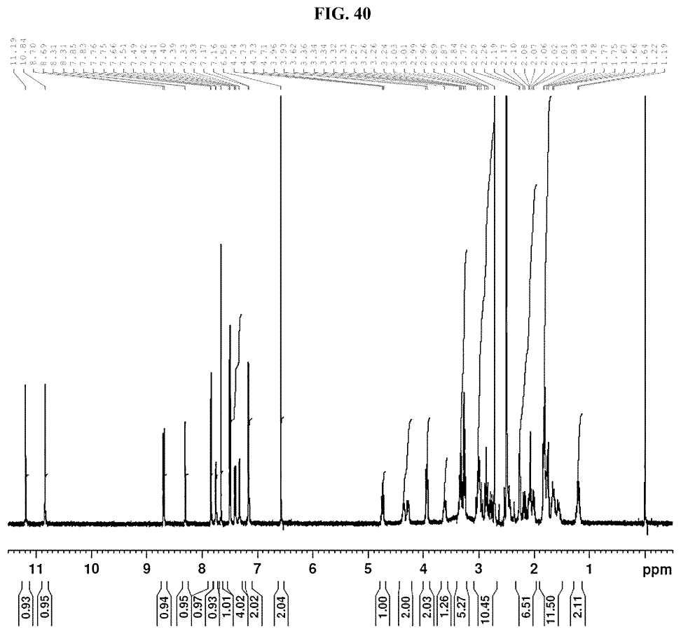

In one or more embodiments, Fumarate Salt Amorphous Form is characterized by an X-ray powder diffraction (XRPD) pattern substantially similar to that set forth in FIG. 39. In one or more embodiments, Fumarate Salt Amorphous Form is shown to be amorphous by XRPD.

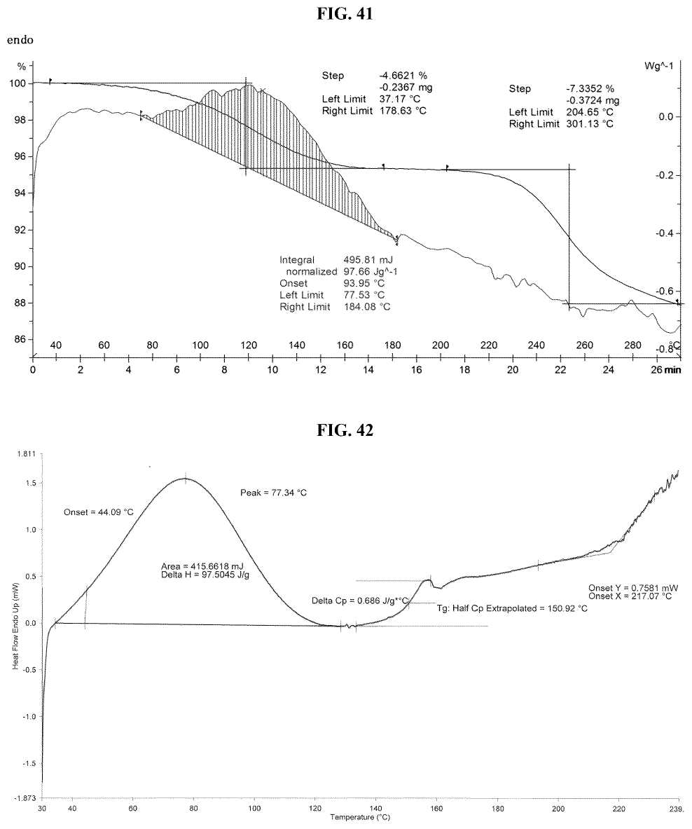

In one or more embodiments, Fumarate Salt Amorphous Form is characterized by a differential thermal analysis (DTA) thermogram having an endotherm with an endotherm maxima between about 75° C. and about 190° C. In one or more embodiments, Fumarate Salt Amorphous Form is characterized by a differential thermal analysis (DTA) thermogram having an endotherm with endotherm maxima between about 77° C. and about 184° C. In one or more embodiments, Fumarate Salt Amorphous Form is characterized by DTA thermogram substantially similar to that set forth in FIG. 41.

In one or more embodiments, Fumarate Salt Amorphous Form is characterized by a weight loss in the range of about 1% to about 10% when heated from about 35° C. to about 180° C. in a thermogravimetric analysis (TGA). In one or more embodiments, Fumarate Salt Amorphous Form is characterized by a weight loss of about 4.66% when heated from about 37° C. to about 179° C. in a TGA. In one or more embodiments, Fumarate Salt Amorphous Form is characterized by a TGA substantially similar to that set forth in FIG. 41.

Fumarate Salt Amorphous Form can be synthesized as described in Synthetic Example 12. In one or more embodiments, Fumarate Salt Amorphous Form is synthesized by heating and lyophilizing (freeze drying) a solution of free base compound of Formula (I) (Fumarate Salt Form A). In one or more embodiments, the solution includes acetonitrile and water. In one or more embodiments, the heating is at 80° C. In one or more embodiments, the lyophilizing (freeze drying) is in liquid nitrogen.

In one or more embodiments, Fumarate Salt Amorphous Form is synthesized by a method comprising:
a) mixing Fumarate Salt Form A in acetonitrile/water (1:3) to form a suspension;
b) heating the suspension to 80° C. to afford a solution; and
c) removing from heat and freezing in liquid nitrogen, lyophilizing (freeze drying) for 24 hours.

In one or more embodiments, Fumarate Salt Amorphous Form is characterized by a differential scanning calorimetry (DSC) thermogram having an endotherm with an endotherm maxima between about 30° C. and about 130° C. In one or more embodiments, Fumarate Salt Amorphous Form is characterized by a differential scanning calorimetry (DSC) thermogram having an endotherm with endotherm maxima at about 77° C. In one or more embodiments, Fumarate Salt Amorphous Form is characterized by DSC thermogram substantially similar to that set forth in FIG. 42.

A DVS analysis of Fumarate Salt Amorphous Form is shown in FIG. 44.

### Fumarate Salt Form B

In one or more embodiments, provided herein is a salt of a compound of Formula (I). In one or more embodiments, the salt of the compound of Formula (I) is a hemi-fumarate salt. In one or more embodiments, the hemi-fumarate salt is Fumarate Salt Form B.

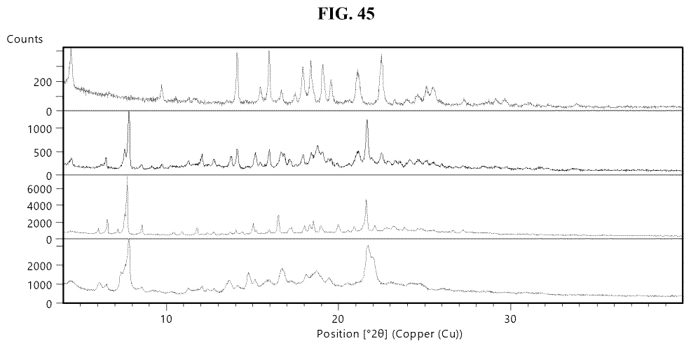

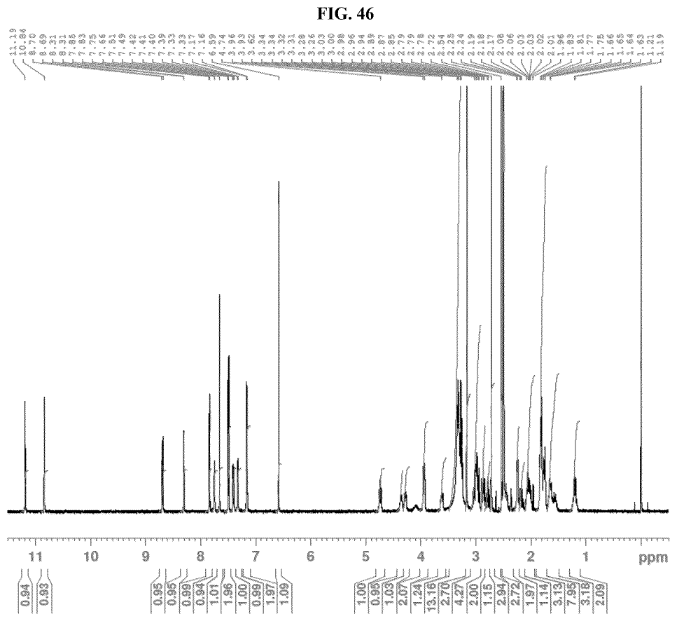

In one or more embodiments, Fumarate Salt Form B is characterized by an X-ray powder diffraction (XRPD) pattern substantially similar to that set forth in FIG. 45 (2^{nd} from bottom). In one or more embodiments, Fumarate Salt Form B (hemi-fumarate) is characterized by an X-ray powder diffraction (XRPD) pattern comprising peaks substantially similar to those set forth in Table 31. In one or more embodiments, Fumarate Salt Form B (hemi-fumarate) is characterized by an XRPD pattern comprising:
a) 2θ values of about 6.60, 7.54, 7.67, 8.57, 16.42, 18.18, and 21.56±0.2° 2θ;
b) 2θ values of about 6.02, 6.60, 7.54, 7.67, 8.57, 11.74, 14.97, 16.42, 17.19, 18.18, 18.49, and 21.56±0.2° 2θ;
c) 2θ values including at least or selected from about 4.54, 6.02, 6.60, 7.15, 7.54, 7.67, 8.57, 8.88, 10.44, 10.82, 11.74, 12.04, 12.42, 12.71, 13.63, 13.95, 14.35, 14.97, 15.17, 15.90, 16.42, 17.19, 17.83, 18.18, 18.49, 18.96, 19.86, 20.09, 20.43, 20.80, 21.56, 22.09, 22.76, 23.21, 23.74, 24.78, 25.35, 26.52, 27.31, 28.53, 30.47, 31.29, 31.89, 33.51, 35.56, and 38.03±0.2° 2θ;
d) at least two, three, four, or five 2θ values selected from about 6.02, 6.60, 7.54, 7.67, 8.57, 11.74, 14.97, 16.42, 17.19, 18.18, 18.49, 21.56, 23.21±0.2° 2θ;
e) at least six, seven, or eight 2θ values selected from about 6.02, 6.60, 7.15, 7.54, 7.67, 8.57, 11.74, 14.97, 16.42, 17.19, 18.18, 18.49, 18.96, 19.89, 21.56, 22.09, 23.21±0.2° 2θ;
f) 2θ values of about 6.02, 6.60, 7.54, 7.67, 16.42, 18.18, and 21.56±0.2° 2θ;
g) 2θ values including at least or selected from about 20 values of about 6.02, 6.60, 7.54, 7.67, 16.42, 18.49, and 21.56±0.2° 2θ;
h) 2θ values of about 6.60, 7.54, 7.67, 16.42, and 21.56±0.2° 2θ;
i) 2θ values including at least or selected from about 7.54, 7.67, 16.42, and 21.56±0.2° 2θ;
j) 2θ values of about 6.60, 7.67, 16.42, and 21.56; and
k) 2θ values including at least or selected from about 7.67, 16.42, and 21.56±0.2° 2θ.

Fumarate Salt Form B can be synthesized as described in Synthetic Example 13.

In one or more embodiments, Fumarate Salt Form B is synthesized by a method comprising:
a) mixing Fumarate Salt Amorphous Form (hemi-fumarate salt, a compound of Formula (I)) with methanol;
b) stirring to form a slurry; and
c) isolating the precipitate to afford Fumarate Salt Form B.

In one or more embodiments, Fumarate Salt Form B (hemi-fumarate salt) is synthesized by a method comprising:
a) mixing Free Base Form A (a compound of Formula (I)) with methanol and 0.5 mol. equivalents of fumaric acid;
b) stirring to form a slurry; and
c) isolating the precipitate to afford Fumarate Salt Form B.

In one or more embodiments, the mixture is allowed to stir and heat for at least 12 hours, at least 14 hours, at least 16 hours, at least 18 hours, at least 20 hours, at least 24 hours, at least 2 days, at least 3 days, at least 4 days, at least 5 days, or at least 6 days in step (b).

### Fumarate Salt Form C

In one or more embodiments, provided herein is a salt of a compound of Formula (I). In one or more embodiments, the salt of the compound of Formula (I) is a hemi-fumarate salt. In one or more embodiments, the hemi-fumarate salt is Fumarate Salt Form C.

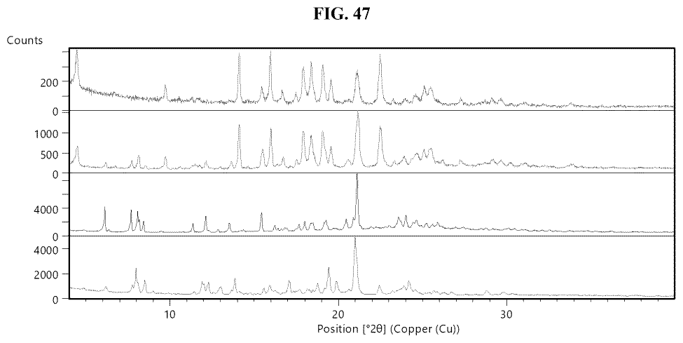

In one or more embodiments, Fumarate Salt Form C is characterized by an X-ray powder diffraction (XRPD) pattern substantially similar to that set forth in FIG. 47 (2^{nd} from bottom). In one or more embodiments, Fumarate Salt Form C (hemi-fumarate) is characterized by an X-ray powder diffraction (XRPD) pattern comprising peaks substantially similar to those set forth in Table 32. In one or more embodiments, Fumarate Salt Form C (hemi-fumarate) is characterized by an XRPD pattern comprising:
a) 2θ values of about 6.14, 7.69, 8.08, 8.18, 12.14, 15.44, 20.89, 21.13, and 24.03±0.2° 2θ;
b) 2θ values of about 6.14, 8.44, 7.69, 8.08, 8.18, 11.37, 12.14, 13.53, 15.44, 19.28, 20.47, 20.89, 21.13, 23.59, and 24.03±0.2° 2θ;
c) 2θ values including at least or selected from about 4.89, 6.14, 6.38, 7.69, 8.08, 8.18, 8.44, 9.51, 11.37, 12.14, 12.86, 13.53, 14.21, 14.37, 15.44, 16.23, 16.42, 16.61, 16.68, 16.95, 17.51, 17.67, 18.01, 18.35, 18.51, 19.16, 19.28, 19.71, 20.47, 20.89, 21.13, 21.96, 22.30, 22.84, 23.04, 23.59, 23.74, 24.03, 24.43, 24.65, 24.93, 25.25, 25.61, 25.91, 26.14, 26.99, 27.46, 28.49, 28.78, 29.18, 30.14, 30.47, 30.91, 31.18, 31.68, 32.14, 32.59, 32.95, 33.77, 34.86, 35.30, 36.27, 38.05, 39.50 and ±0.2° 2θ;
d) at least two, three, four, or five 2θ values selected from about 6.14, 8.44, 7.69, 8.08, 8.18, 11.37, 12.14, 13.53, 15.44, 19.28, 20.47, 20.89, 21.13, 23.59, and 24.03±0.2° 2θ;
e) at least six, seven, or eight 2θ values selected from about 6.14, 8.44, 7.69, 8.08, 8.18, 11.37, 12.14, 13.53, 15.44, 16.23, 17.67, 18.01, 18.35, 18.51, 19.16, 19.28, 20.47, 20.89, 21.13, 23.59, 23.74, 24.03, 24.65, and 25.91±0.2° 2θ;
f) 2θ values of about 6.14, 7.69, 8.08, 15.44, and 21.13±0.2° 2θ;
g) 2θ values including at least or selected from about 2θ values of about 6.14, 7.69, 8.08, 12.14, 15.44, and 21.13±0.2° 2θ;
h) 2θ values of about 6.14, 7.69, 8.08, 15.44, 20.89, and 21.13±0.2° 2θ;
i) 2θ values including at least or selected from about 6.14, 7.69, 8.08, 15.44, 20.47, and 21.13±0.2° 2θ;
j) 2θ values of about 6.14, 7.69, 8.08, and 21.13; and
k) 2θ values including at least or selected from about 6.14, 7.69, 15.44, and 21.13±0.2° 2θ.

Fumarate Salt Form C can be synthesized as described in Synthetic Example 14.

In one or more embodiments, Fumarate Salt Form C (hemi-fumarate salt) is synthesized by a method comprising:
a) mixing Free Base Form A (a compound of Formula (I)) with 0.5 mol. equivalent of fumaric acid in methanol/water;
b) stirring; and
c) isolating the precipitate to afford Fumarate Salt Form C.

In one or more embodiments, Fumarate Salt Form C (hemi-fumarate salt) is synthesized by a method comprising:
a) mixing Fumarate Salt Form B (hemi-fumarate salt, a compound of Formula (I)) in methanol/water;
b) stirring; and
c) isolating the precipitate to afford Fumarate Salt Form C.

In one or more embodiments, the methanol/water in step (a) is in a ratio of from about 99/1 to about 50/50, such as from 99/1 to 60/40, from 99/1 to 70/30, from 99/1 to 80/20, from 90/10 to 50/50, from 90/10 to 60/40, from 90/10 to 70/30, or from 90/10 to 80/20. In one or more embodiments, the methanol/water in step (a) is in a ratio of 84/16.

In one or more embodiments, the mixture is allowed to stir and heat for at least 12 hours, at least 14 hours, at least 16 hours, at least 18 hours, at least 20 hours, at least 24 hours, at least 2 days, at least 3 days, at least 4 days, at least 5 days, or at least 6 days in step (b).

### Fumarate Salt Form D

In one or more embodiments, provided herein is a salt of a compound of Formula (I). In one or more embodiments, the salt of the compound of Formula (I) is a mono-fumarate salt. In one or more embodiments, the mono-fumarate salt is Fumarate Salt Form D.

In one or more embodiments, Fumarate Salt Form D is characterized by an X-ray powder diffraction (XRPD) pattern substantially similar to that set forth in FIG. 49.

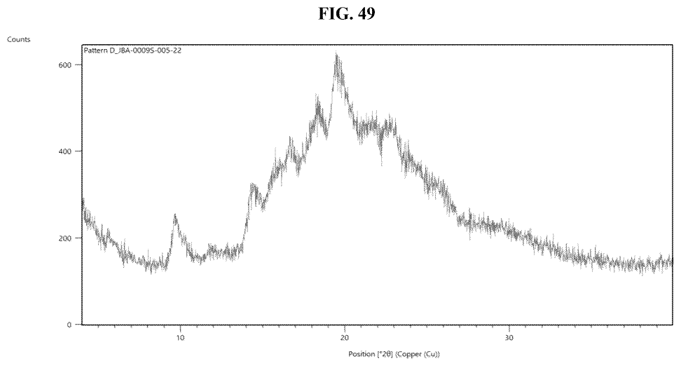

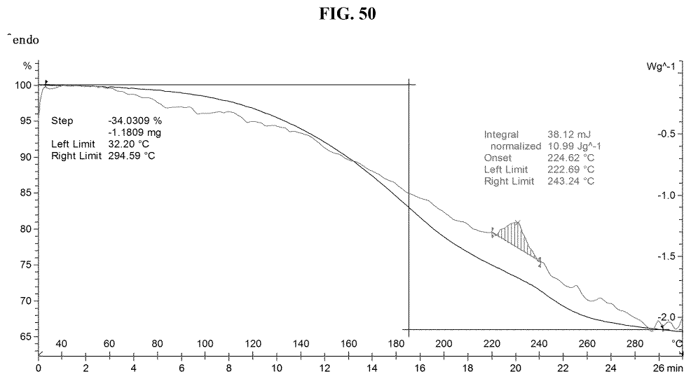

In one or more embodiments, Fumarate Salt Form D is characterized by a differential thermal analysis (DTA) thermogram having an endotherm with an endotherm maxima between about 220° C. and about 250° C. In one or more embodiments, Fumarate Salt Form D is characterized by a differential thermal analysis (DTA) thermogram having an endotherm with endotherm maxima at about 222° C. and about 243° C. In one or more embodiments, Fumarate Salt Form D is characterized by DTA thermogram substantially similar to that set forth in FIG. 50.

In one or more embodiments, Fumarate Salt Form D is characterized by a weight loss in the range of about 30% to about 40% when heated from about 30° C. to about 300° C. in a thermogravimetric analysis (TGA). In one or more embodiments, Fumarate Salt Form D is characterized by a weight loss of about 34.0% when heated from about 32° C. to about 295° C. in a TGA. In one or more embodiments, Fumarate Salt Form D is characterized by a TGA substantially similar to that set forth in FIG. 50.

Fumarate Salt Form D can be synthesized as described in Synthetic Example 15.

In one or more embodiments, Fumarate Salt Form D is synthesized by a method comprising:
a) mixing Fumarate Salt Amorphous Form (a compound of Formula (I)) in DMSO or DMSO/water;
b) stirring and temperature cycling; and
c) isolating the precipitate to afford Fumarate Salt Form D.

In one or more embodiments, the DMSO/water in step (a) is in a ratio of from about 70/30 to about 95/5, such as from about 75/25 to about 90/10, or from about 80/20 to about 90/10, or about 85/15. In one or more embodiments, the DMSO/water in step (a) is in a ratio of 86/14.

In one or more embodiments, the mixture is allowed to stir and heat for at least 12 hours, at least 14 hours, at least 16 hours, at least 18 hours, at least 20 hours, at least 24 hours, at least 2 days, at least 3 days, at least 4 days, at least 5 days, or at least 6 days in step (b).

In one or more embodiments, the temperature cycling is between 5° C. to 100° C. and back to 5° C. in step (b).

### Fumarate Salt Form E

In one or more embodiments, provided herein is a salt of a compound of Formula (I). In one or more embodiments, the salt of the compound of Formula (I) is a mono-fumarate salt. In one or more embodiments, the mono-fumarate salt is Fumarate Salt Form E.

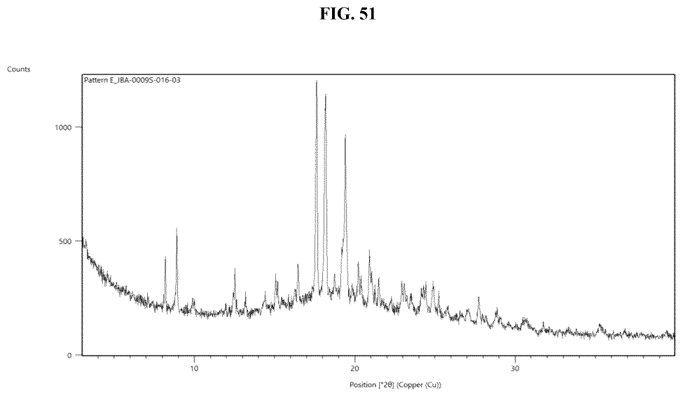

In one or more embodiments, Fumarate Salt Form E is characterized by an X-ray powder diffraction (XRPD) pattern substantially similar to that set forth in FIG. 51.

Fumarate Salt Form E can be synthesized as described in Synthetic Example 16.

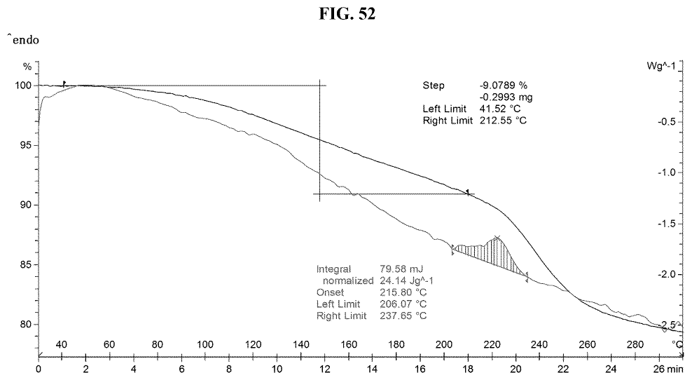

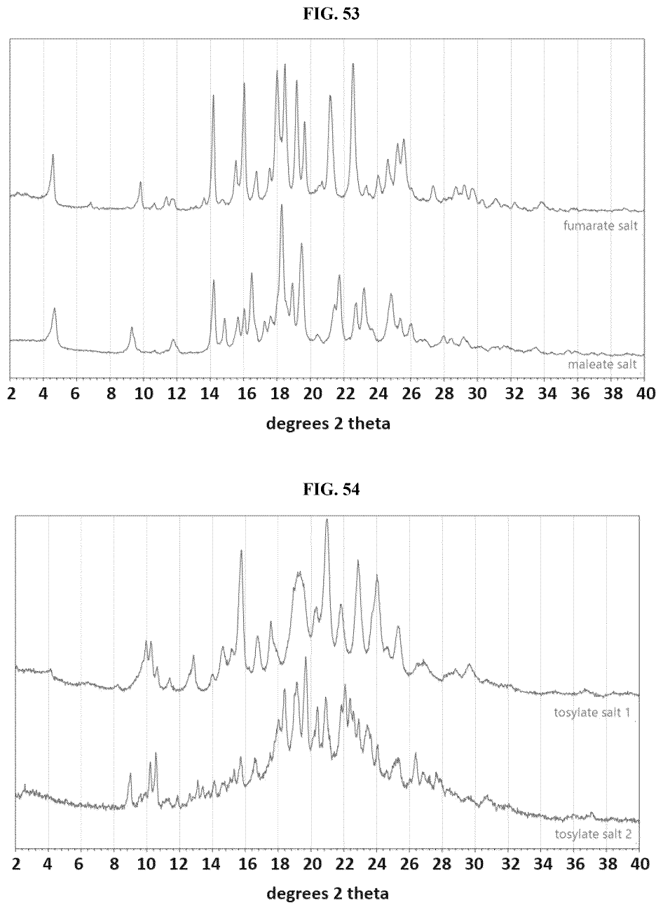

In one or more embodiments, Fumarate Salt Form E is characterized by a differential thermal analysis (DTA) thermogram having an endotherm with an endotherm maxima between about 200° C. and about 240° C. In one or more embodiments, Fumarate Salt Form E is characterized by a differential thermal analysis (DTA) thermogram having an endotherm with endotherm maxima at about 206° C. and about 238° C. In one or more embodiments, Fumarate Salt Form E is characterized by DTA thermogram substantially similar to that set forth in FIG. 52.

In one or more embodiments, Fumarate Salt Form E is characterized by a weight loss in the range of about 5% to about 15% when heated from about 40° C. to about 215° C. in a thermogravimetric analysis (TGA). In one or more embodiments, Fumarate Salt Form E is characterized by a weight loss of about 9.08% when heated from about 41° C. to about 213° C. in a TGA. In one or more embodiments, Fumarate Salt Form E is characterized by a TGA substantially similar to that set forth in FIG. 52.

In one or more embodiments, Fumarate Salt Form E is synthesized by a method comprising:
a) mixing Fumarate Salt Amorphous Form (a compound of Formula (I)) in dimethylacetamide;
b) stirring to form a slurry;
c) isolating the precipitate to afford Fumarate Salt Form E.

In one or more embodiments, the mixture is allowed to stir and heat for at least 12 hours, at least 14 hours, at least 16 hours, at least 18 hours, at least 20 hours, at least 24 hours, at least 2 days, at least 3 days, at least 4 days, at least 5 days, or at least 6 days in step (b).

### Maleate Salt Form A (Maleate Form 1 or Maleate Salt Form 1)

In one or more embodiments, provided herein is a salt of a compound of Formula (I). In one or more embodiments, the salt of the compound of Formula (I) is a maleate salt. In one or more embodiments, the maleate salt is Maleate Salt Form A.

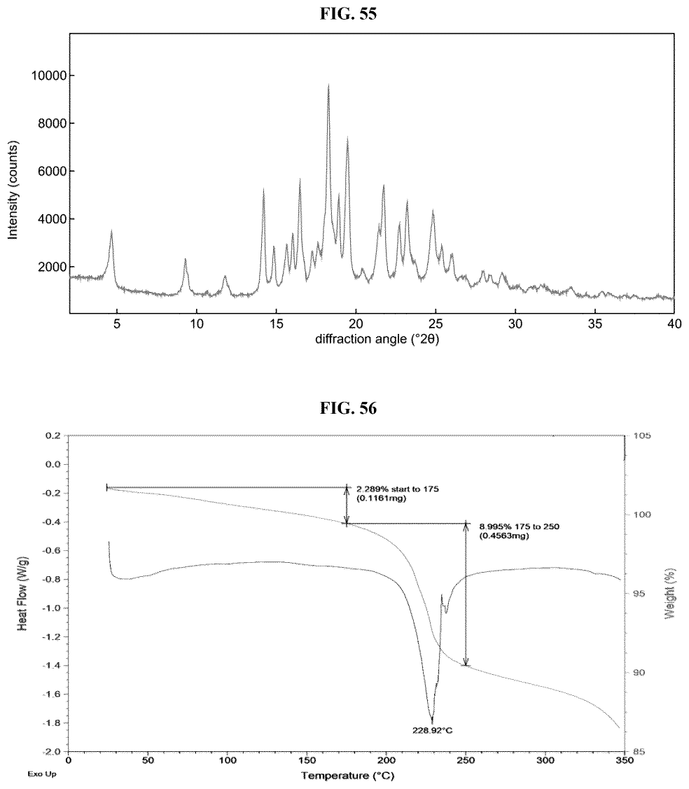

In one or more embodiments, Maleate Salt Form A is characterized by an X-ray powder diffraction (XRPD) pattern substantially similar to that set forth in FIG. 55. In one or more embodiments, Maleate Salt Form A is characterized by an X-ray powder diffraction (XRPD) pattern comprising peaks substantially similar to those set forth in Table 33. In one or more embodiments, Maleate Salt Form A is characterized by an XRPD pattern comprising:
a) 2θ values of about 14.20, 16.48, 18.28, 19.46, and 21.74±0.2° 2θ;
b) 2θ values of about 4.66, 9.28, 14.20, 16.48, 18.28, 18.92, 19.46, 21.44, 21.74, 22.74, 23.22, and 24.82±0.2° 2θ;
c) 2θ values including at least or selected from about 4.66, 9.28, 11.78, 14.20, 14.84, 15.64, 16.02, 16.48, 17.26, 17.60, 18.28, 18.92, 19.46, 20.42, 21.44, 21.74, 22.74, 23.22, 23.70, 24.82, 25.36, 26.04, 26.60, 26.80, 28.02, 28.40, 29.16, 30.04, 30.96, 31.58, 33.50, 35.46, and 35.82±0.2° 2θ;
d) at least two, three, four, or five 2θ values selected from about 14.20, 16.48, 18.28, 18.92, 19.46, 21.44, 21.74, 22.74, 23.22, and 24.82±0.2° 2θ;
e) at least six, seven, or eight 2θ values selected from about 4.66, 9.28, 14.20, 14.84, 15.64, 16.02, 16.48, 17.26, 17.60, 18.28, 18.92, 19.46, 20.42, 21.44, 21.74, 22.74, 23.22, 23.70, 24.82, 25.36, and 26.04±0.2° 2θ;
f) 2θ values of about 14.20, 16.48, 18.28, 18.92, 19.46, 21.74, 23.22, and 24.82±0.2° 2θ;
g) 2θ values including at least or selected from about 14.20, 16.48, 18.28, 18.92, 19.46, 21.74, 23.22, and 24.82±0.2° 2θ;
h) 2θ values of about 14.20, 16.48, 18.28, and 19.46±0.2° 2θ;
i) 2θ values including at least or selected from about 14.20, 16.48, 18.28, and 19.46±0.2° 2θ;
j) 2θ values of about 16.48, 18.28, and 19.46±0.2° 2θ;
k) 2θ values including at least or selected from about 16.48, 18.28, and 19.46±0.2° 2θ;
l) 2θ values of about 14.20, 16.48, 18.28, 18.92, 19.46, 21.44, 21.74, 23.22, and 24.82±0.2° 2θ;
m) 2θ values including at least or selected from about 14.20, 16.48, 18.28, 18.92, 19.46, 21.44, 21.74, 23.22, and 24.82±0.2° 2θ;
n) 2θ values of about 14.20, 16.48, 18.28, 18.92, 19.46, 21.44, and 21.74±0.2° 2θ;
o) 2θ values including at least or selected from about 14.20, 16.48, 18.28, 18.92, 19.46, 21.44, and 21.74±0.2° 2θ;
p) 2θ values of about 16.48, 18.28, 18.92, and 19.46±0.2° 2θ;
q) 2θ values including at least or selected from about 16.48, 18.28, 18.92, and 19.46±0.2° 2θ;
r) 2θ values of about 16.48, 18.28, 19.46, and 21.74±0.2° 2θ;
s) 2θ values including at least or selected from about 16.48, 18.28, 19.46, and 21.74±0.2° 2θ;
t) 2θ values of about 16.48, 18.28, 19.46, and 23.22±0.2° 2θ;
u) 2θ values including at least or selected from about 16.48, 18.28, 19.46, and 23.22±0.2° 2θ;
v) 2θ values of about 16.48, 18.28, 19.46, and 24.82±0.2° 2θ; and
w) 2θ values including at least or selected from about 16.48, 18.28, 19.46, and 24.82±0.2° 2θ.

In one or more embodiments, Maleate Salt Form A is characterized by a differential scanning calorimetry (DSC) thermogram having one endotherm with an endotherm maxima between about 225° C. and about 235° C. In one or more embodiments, Maleate Salt Form A is characterized by a differential scanning calorimetry (DSC) thermogram having one endotherm with an endotherm maxima at about 229° C. In one or more embodiments, Maleate Salt Form A is characterized by a DSC thermogram substantially similar to that set forth in FIG. 56.

In one or more embodiments, Maleate Salt Form A is characterized by a weight loss in the range of about 0.1% to about 5% when heated from about 25° C. to about 175° C., and in the range of about 5% to about 10% when heated from about 175° C. to about 250° C. in a thermogravimetric analysis (TGA). In one or more embodiments, Maleate Salt Form A is characterized by a weight loss of about 2.3% when heated from about 25° C. to about 175° C. in a TGA, and about 9.0% when heated from about 175° C. to about 250° C. In one or more embodiments, Maleate Salt Form A is characterized by a TGA substantially similar to that set forth in FIG. 56.

Maleate Salt Form A can be synthesized as described in Synthetic Example 17. In one or more embodiments, Maleate Salt Form A is synthesized by mixing free base compound of Formula (I) with maleic acid in an aprotic solvent (wet). In one or more embodiments, the solvent is selected from the group consisting of tetrahydrofuran, acetone, ethyl acetate, diethyl ether, toluene, hexane, dichloromethane, methyl ethyl ketone, dimethylsulfoxide, and dimethylformamide. In one or more embodiments, the mixture comprises tetrahydrofuran. In one or more embodiments, the mixture does not comprise water. After stirring, for instance overnight or a week, the precipitate is centrifuged, decanted, and dried to afford Maleate Salt Form A.

In one or more embodiments, Maleate Salt Form A is synthesized by a method comprising:
a) mixing free base compound of Formula (I) and maleic acid in tetrahydrofuran;
b) stirring to afford a precipitate;
c) isolating the precipitate to afford Maleate Salt Form A.

In one or more embodiments, the mixture is allowed to stir for at least 12 hours, at least 14 hours, at least 16 hours, at least 18 hours, at least 20 hours, at least 24 hours, at least 2 days, at least 3 days, at least 4 days, at least 5 days, or at least 6 days in step (b).

### Tosylate Salt Form A (Tosylate Form 1 or Tosylate Salt Form 1)

In one or more embodiments, provided herein is a salt of a compound of Formula (I). In one or more embodiments, the salt of the compound of Formula (I) is a tosylate salt. In one or more embodiments, the tosylate salt is Tosylate Salt Form A.

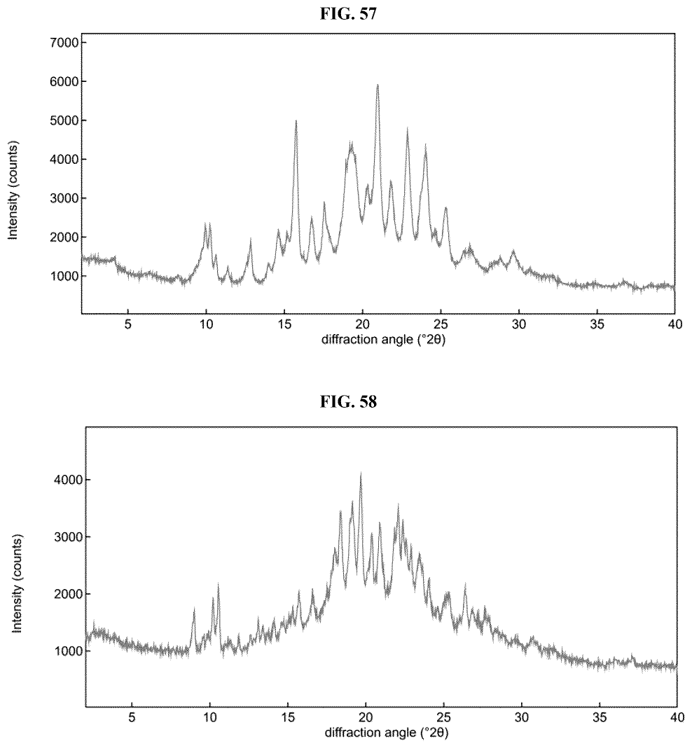

In one or more embodiments, Tosylate Salt Form A is characterized by an X-ray powder diffraction (XRPD) pattern substantially similar to that set forth in FIG. 57. In one or more embodiments, Tosylate Salt Form A is characterized by an X-ray powder diffraction (XRPD) pattern comprising peaks substantially similar to those set forth in Table 34. In one or more embodiments, Tosylate Salt Form A is characterized by an XRPD pattern comprising:
a) 2θ values of about 15.76, 19.34, 20.96, 22.86, and 24.02±0.2° 2θ;
b) 2θ values of about 9.94, 10.24, 15.76, 16.74, 17.54, 19.34, 20.32, 20.96, 21.84, 22.86, 24.02, and 25.32±0.2° 2θ;
c) 2θ values including at least or selected from about 4.14, 8.20, 9.94, 10.24, 10.62, 11.40, 12.84, 13.96, 14.64, 15.14, 15.76, 16.74, 17.54, 19.34, 20.32, 20.96, 21.84, 22.86, 24.02, 24.64, 25.32, 26.84, 28.38, 28.78, 29.64, 30.68, 32.14, and 36.66±0.2° 2θ;
d) at least two, three, four, or five 2θ values selected from about 9.94, 10.24, 14.64, 15.14, 15.76, 16.74, 17.54, 19.34, 20.32, 20.96, 21.84, 22.86, 24.02, 24.64, and 25.32±0.2° 2θ;
e) at least six, seven, or eight 2θ values selected from about 9.94, 10.24, 12.84, 14.64, 15.14, 15.76, 16.74, 17.54, 19.34, 20.32, 20.96, 21.84, 22.86, 24.02, 24.64, 25.32, and 26.84±0.2° 2θ;
f) 20 values of about 15.76, 17.54, 19.34, 20.32, 20.96, 21.84, 22.86, 24.02, and 25.32±0.2° 2θ;
g) 2θ values including at least or selected from about 15.76, 17.54, 19.34, 20.32, 20.96, 21.84, 22.86, 24.02, and 25.32±0.2° 2θ;
h) 2θ values of about 15.76, 19.34, 20.96, and 22.86±0.2° 2θ;
i) 2θ values including at least or selected from about 15.76, 19.34, 20.96, and 22.86±0.2° 2θ;
j) 2θ values of about 15.76, 20.96, and 22.86±0.2° 2θ;
k) 2θ values including at least or selected from about 15.76, 20.96, and 22.86±0.2° 2θ;
l) 2θ values of about 15.76, 17.54, 19.34, 20.32, 20.96, 21.84, 22.86, and 24.02±0.2° 2θ;
m) 2θ values including at least or selected from about 15.76, 17.54, 19.34, 20.32, 20.96, 21.84, 22.86, and 24.02±0.2° 2θ;
n) 2θ values of about 15.76, 19.34, 20.32, 20.96, 21.84, 22.86, and 24.02±0.2° 2θ;
o) 2θ values including at least or selected from about 15.76, 19.34, 20.32, 20.96, 21.84, 22.86, and 24.02±0.2° 2θ;
p) 2θ values of about 15.76, 20.96, 22.86, and 24.02±0.2° 2θ;
q) 2θ values including at least or selected from about 15.76, 20.96, 22.86, and 24.02±0.2° 2θ;
r) 2θ values of about 15.76, 17.54, 20.96, and 22.86±0.2° 2θ;
s) 2θ values including at least or selected from about 15.76, 17.54, 20.96, and 22.86±0.2° 2θ;
t) 2θ values of about 15.76, 20.32, 20.96, and 22.86±0.2° 2θ;
u) 2θ values including at least or selected from about 15.76, 20.32, 20.96, and 22.86±0.2° 2θ;
v) 2θ values of about 15.76, 20.96, 21.84, and 22.86±0.2° 2θ; and
w) 2θ values including at least or selected from about 15.76, 20.96, 21.84, and 22.86±0.2° 2θ.

Tosylate Salt Form A can be synthesized as described in Synthetic Example 18. In one or more embodiments, Tosylate Salt Form A is synthesized by mixing free base compound of Formula (I) with p-toluenesulfonic acid in an aprotic solvent (wet). In one or more embodiments, the solvent is selected from the group consisting of tetrahydrofuran, acetone, ethyl acetate, diethyl ether, toluene, hexane, dichloromethane, methyl ethyl ketone, dimethyl sulfoxide, and dimethylformamide. In one or more embodiments, the mixture comprises tetrahydrofuran. In one or more embodiments, the mixture does not comprise water. After stirring, for instance overnight or a week, the precipitate is centrifuged, decanted, and dried to afford Tosylate Salt Form A.

In one or more embodiments, Tosylate Salt Form A is synthesized by a method comprising:
a) mixing free base compound of Formula (I) and p-toluenesulfonic acid in tetrahydrofuran;
b) stirring to afford a precipitate; and
c) isolating the precipitate to afford Tosylate Salt Form A.

In one or more embodiments, the mixture is allowed to stir for at least 12 hours, at least 14 hours, at least 16 hours, at least 18 hours, at least 20 hours, at least 24 hours, at least 2 days, at least 3 days, at least 4 days, at least 5 days, or at least 6 days in step (b).

### Tosylate Salt Form B (Tosylate Form 2 or Tosylate Salt Form 2)

In one or more embodiments, provided herein is a salt of a compound of Formula (I). In one or more embodiments, the salt of the compound of Formula (I) is a tosylate salt. In one or more embodiments, the tosylate salt is Tosylate Salt Form B.

In one or more embodiments, Tosylate Salt Form B is characterized by an X-ray powder diffraction (XRPD) pattern substantially similar to that set forth in FIG. 58. In one or more embodiments, Tosylate Salt Form B is characterized by an X-ray powder diffraction (XRPD) pattern comprising peaks substantially similar to those set forth in Table 35. In one or more embodiments, Tosylate Salt Form B is characterized by an XRPD pattern comprising:
a) 2θ values of about 18.40, 19.12, 19.68, and 22.08±0.2° 2θ;
b) 2θ values of about 9.00, 10.20, 10.54, 18.00, 18.40, 19.12, 19.68, 20.38, 20.88, 21.86, 22.08, 22.36, 22.58, 22.90, and 26.38±0.2° 2θ;
c) 2θ values including at least or selected from about 9.00, 9.64, 9.90, 10.20, 10.54, 11.36, 11.86, 12.58, 12.80, 13.10, 13.42, 13.78, 14.12, 14.64, 15.06, 15.32, 15.72, 16.56, 17.50, 18.00, 18.40, 19.12, 19.68, 20.38, 20.88, 21.86, 22.08, 22.36, 22.58, 22.90, 23.42, 24.08, 24.60, 25.28, 26.38, 26.86, 27.24, and 27.64±0.2° 2θ;
d) at least two, three, four, or five 2θ values selected from about 15.72, 16.56, 17.50, 18.40, 19.12, 19.68, and 22.08±0.2° 2θ;
e) at least six, seven, or eight 2θ values selected from about 10.20, 10.54, 15.72, 16.56, 17.50, 18.00, 18.40, 19.12, 19.68, 20.38, 20.88, 21.86, 22.08, 22.36, 22.58, 22.90, 23.42, 24.08, 25.28, and 26.38±0.2° 2θ;
f) 2θ values of about 18.40, 19.12, 19.68, 20.38, 20.88, 21.86, 22.08, 22.36, 22.58, and 22.90±0.2° 2θ;
g) 2θ values including at least or selected from about 18.40, 19.12, 19.68, 20.38, 20.88, 21.86, 22.08, 22.36, 22.58, and 22.90±0.2° 2θ;
h) 2θ values of about 18.40, 19.12, 19.68, and 22.08±0.2° 2θ;
i) 2θ values including at least or selected from about 18.40, 19.12, 19.68, and 22.08±0.2° 2θ;
j) 2θ values of about 19.12, 19.68, and 22.08±0.2° 2θ;
k) 2θ values including at least or selected from about 19.12, 19.68, and 22.08±0.2° 2θ;
l) 2θ values of about 18.00, 18.40, 19.12, 19.68, 20.38, 20.88, 21.86, 22.08, 22.36, 22.58, and 22.90±0.2° 2θ;
m) 2θ values including at least or selected from about 18.00, 18.40, 19.12, 19.68, 20.38, 20.88, 21.86, 22.08, 22.36, 22.58, and 22.90±0.2° 2θ;
n) 2θ values of about 18.00, 18.40, 19.12, 19.68, 20.38, 20.88, 21.86, 22.08, 22.36, and 22.58±0.2° 2θ;
o) 2θ values including at least or selected from about 18.00, 18.40, 19.12, 19.68, 20.38, 20.88, 21.86, 22.08, 22.36, and 22.58±0.2° 2θ;
p) 2θ values of about 19.12, 19.68, 20.38, and 22.08±0.2° 2θ;
q) 2θ values including at least or selected from about 19.12, 19.68, 20.38, and 22.08±0.2° 2θ;
r) 2θ values of about 19.12, 19.68, 20.88, and 22.08±0.2° 2θ;
s) 2θ values including at least or selected from about 19.12, 19.68, 20.88, and 22.08±0.2° 2θ;
t) 2θ values of about 19.12, 19.68, 21.86, and 22.08±0.2° 2θ;
u) 2θ values including at least or selected from about 19.12, 19.68, 21.86, and 22.08±0.2° 2θ;
v) 2θ values of about 19.12, 19.68, 22.08, and 22.36±0.2° 2θ; and
w) 2θ values including at least or selected from about 19.12, 19.68, 22.08, and 22.36±0.2° 2θ.

Tosylate Salt Form B can be synthesized as described in Synthetic Example 19. In one or more embodiments, Tosylate Salt Form B is synthesized by mixing free base compound of Formula (I) and p-toluenesulfonic acid in a protic solvent. In one or more embodiments, the solvent is a polar protic solvent. In one or more embodiments, the solvent is selected from the group consisting of methanol, ethanol, isopropanol, formic acid, and acetic acid. In one or more embodiments, the mixture comprises water. In one or more embodiments, the mixture does not comprise water. After stirring, for instance overnight or a week and under heat, the precipitate is centrifuged, decanted, and dried to afford Tosylate Salt Form B.

In one or more embodiments, Tosylate Salt Form B is synthesized by a method comprising:
a) mixing free base compound of Formula (I) and p-toluenesulfonic acid in methanol and water;
b) stirring and heating to afford a precipitate;
c) isolating the precipitate to afford Tosylate Salt Form B.

In one or more embodiments, the mixture is allowed to stir and heat for at least 12 hours, at least 14 hours, at least 16 hours, at least 18 hours, at least 20 hours, at least 24 hours, at least 2 days, at least 3 days, at least 4 days, at least 5 days, or at least 6 days in step (b).

### Besylate Salt Form A

In one or more embodiments, provided herein is a salt of a compound of Formula (I). In one or more embodiments, the salt is a besylate salt. In one or more embodiments, the besylate salt is Besylate Salt Form A.

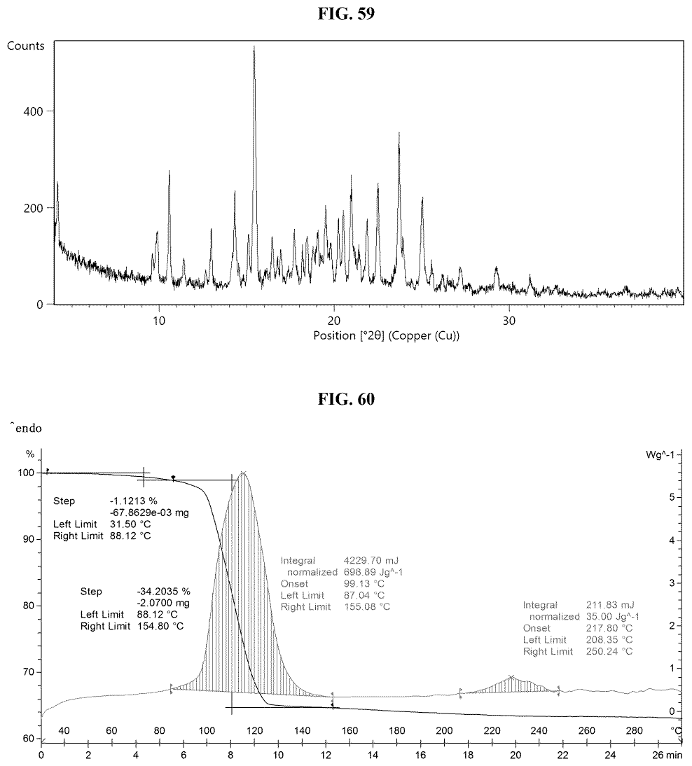

In one or more embodiments, Besylate Salt Form A is characterized by an X-ray powder diffraction (XRPD) pattern substantially similar to that set forth in FIG. 59 (2^{nd} from top). Besylate Salt Form A can be synthesized as described in Synthetic Example 20.

In one or more embodiments, Besylate Salt Form A is characterized by a differential thermal analysis (DTA) thermogram having two endotherms with a first endotherm maxima between about 85° C. and about 160° C., and a second endotherm maxima between about 205° C. and about 255° C. In one or more embodiments, Besylate Salt Form A is characterized by a differential thermal analysis (DTA) thermogram having two endotherms with a first endotherm maxima at about 87° C. and about 155° C., and a second endotherm maxima at about 208° C. and about 205° C. In one or more embodiments, Besylate Salt Form A is characterized by DTA thermogram substantially similar to that set forth in FIG. 60.

In one or more embodiments, Besylate Salt Form A is characterized by a weight loss in the range of about 0.1% to about 5% when heated from about 30° C. to about 90° C., and a weight loss in the range of about 30% to about 40% when heated from about 85° C. to about 160° C. in a thermogravimetric analysis (TGA). In one or more embodiments, Besylate Salt Form A is characterized by a weight loss of about 1.12% when heated from about 31° C. to about 88° C., and a weight loss of about 34.2% when heated from about 88° C. to about 155° C. in a TGA. In one or more embodiments, Besylate Salt Form A is characterized by a TGA substantially similar to that set forth in FIG. 60.

In one or more embodiments, Besylate Salt Form A is synthesized by a method comprising:
a) mixing Free Base Form A (a compound of Formula (I)) in tetrahydrofuran/water and 1 mol. equivalent of benzenesulfonic acid;
b) stirring and temperature cycling;
c) isolating the precipitate to afford Besylate Salt Form A.

In one or more embodiments, the tetrahydrofuran/water in step (a) is in a ratio of from about 1:1 to about 10:1, such as from 1:1 to 9:1, from 1:1 to 8:1, from 1:1 to 7:1, from 1:1 to 6:1, from 1:1 to 5:1, from 1:1 to 4:1, or from 1:1 to 3:1. In one or more embodiments, the tetrahydrofuran/water in step (a) is in a ratio of 2:1 (12 vols).

In one or more embodiments, the mixture is allowed to stir and temperature cycle for at least 12 hours, at least 14 hours, at least 16 hours, at least 18 hours, at least 20 hours, at least 24 hours, at least 2 days, at least 3 days, at least 4 days, at least 5 days, or at least 6 days in step (b). In one or more embodiments, the mixture is allowed to stir and temperature cycle for 75-80 hours.

In one or more embodiments, the temperature cycling is between ambient temperature (of from 15° C. to 30° C.) to 50° C. and back to ambient temperature in step (b). In one or more embodiments, the temperature cycling is between ambient temperature to 50° C. at 60 hours, returned to ambient temperature, and stirred for another 18 hours.

In one or more embodiments, the isolating is by centrifugation in step (c).

### Cyclamate Salt Form A

In one or more embodiments, provided herein is a salt of a compound of Formula (I). In one or more embodiments, the salt of the compound of Formula (I) is a cyclamate salt. In one or more embodiments, the cyclamate salt is Cyclamate Salt Form A.

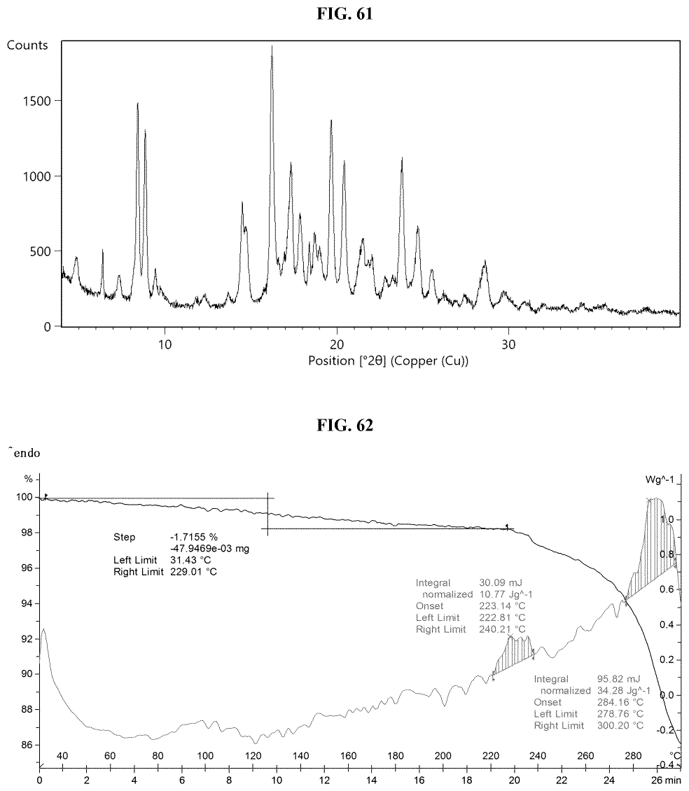

In one or more embodiments, Cyclamate Salt Form A is characterized by an X-ray powder diffraction (XRPD) pattern substantially similar to that set forth in FIG. 61.

Cyclamate Salt Form A can be synthesized as described in Synthetic Example 21.

In one or more embodiments, Cyclamate Salt Form A is characterized by a differential thermal analysis (DTA) thermogram having two endotherms with a first endotherm maxima between about 220° C. and about 245° C., and a second endotherm maxima between about 275° C. to about 305° C. In one or more embodiments, Cyclamate Salt Form A is characterized by a differential thermal analysis (DTA) thermogram having an endotherm with endotherm maxima at about 222° C. and about 240° C., and at about 278° C. and about 300° C. In one or more embodiments, Cyclamate Salt Form A is characterized by DTA thermogram substantially similar to that set forth in FIG. 62.

In one or more embodiments, Cyclamate Salt Form A is characterized by a weight loss in the range of about 1% to about 5% when heated from about 30° C. to about 235° C. in a thermogravimetric analysis (TGA). In one or more embodiments, Cyclamate Salt Form A is characterized by a weight loss of about 1.72% when heated from about 31° C. to about 229° C. in a TGA. In one or more embodiments, Cyclamate Salt Form A is characterized by a TGA substantially similar to that set forth in FIG. 62.

In one or more embodiments, Cyclamate Salt Form A is synthesized by a method comprising:
a) mixing Free Base Form A (a compound of Formula (I)) and cyclamic acid (1 mol. equivalent) in THF;
b) stirring and temperature cycling;
c) isolating the precipitate to afford Cyclamate Salt Form A; optionally drying under vacuum for 1 hour to afford Cyclamate Salt Form A.

In one or more embodiments, the mixture is allowed to stir and temperature cycle for at least 12 hours, at least 14 hours, at least 16 hours, at least 18 hours, at least 20 hours, at least 24 hours, at least 2 days, at least 3 days, at least 4 days, at least 5 days, or at least 6 days in step (b). In one or more embodiments, the mixture is allowed to stir and temperature cycle for 75-80 hours.

In one or more embodiments, the temperature cycling is between ambient temperature (of from 15° C. to 30° C.) to 40° C. and back to 5° C. in step (b). In one or more embodiments, the temperature cycling is between ambient temperature to 40° C. at 60 hours, then cooled to 5° C. and stirred at this temperature for another 18 hours.

In one or more embodiments, the isolating is by centrifugation in step (c).

### Malate Salt Form A

In one or more embodiments, provided herein is a salt of a compound of Formula (I). In one or more embodiments, the salt of the compound of Formula (I) is a malate salt. In one or more embodiments, the malate salt is Malate Salt Form A.

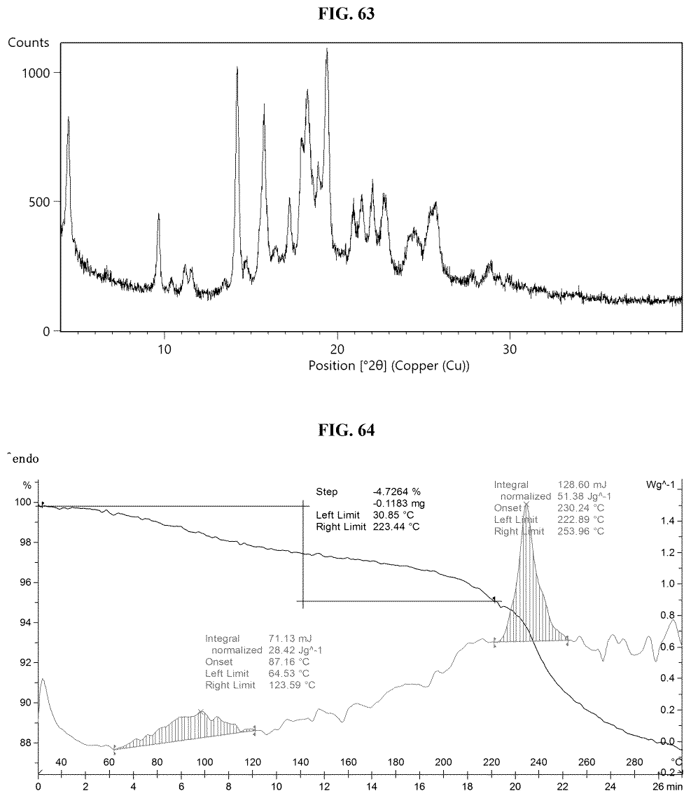

In one or more embodiments, Malate Salt Form A is characterized by an X-ray powder diffraction (XRPD) pattern substantially similar to that set forth in FIG. 63.

Malate Salt Form A can be synthesized as described in Synthetic Example 22.

In one or more embodiments, Malate Salt Form A is characterized by a differential thermal analysis (DTA) thermogram having an endotherm with two endotherm maximas between about 60° C. and about 130° C., and between about 220° C. and about 260° C. In one or more embodiments, Malate Salt Form A is characterized by a differential thermal analysis (DTA) thermogram having two endotherms with endotherm maxima at about 64° C. and about 124° C., and about 222° C. and about 254° C. In one or more embodiments, Malate Salt Form A is characterized by DTA thermogram substantially similar to that set forth in FIG. 64.

In one or more embodiments, Malate Salt Form A is characterized by a weight loss in the range of about 1% to about 10% when heated from about 30° C. to about 225° C. in a thermogravimetric analysis (TGA). In one or more embodiments, Malate Salt Form A is characterized by a weight loss of about 4.73% when heated from about 30° C. to about 223° C. in a TGA. In one or more embodiments, Malate Salt Form A is characterized by a TGA substantially similar to that set forth in FIG. 64.

In one or more embodiments, Malate Salt Form A is synthesized by a method comprising:
a) mixing Free Base Form A (a compound of Formula (I)) and malic acid (1 mol. equivalent) in tetrahydrofuran;
b) stirring and temperature cycling;
c) isolating the precipitate to afford Malate Salt Form A; optionally drying under vacuum for 1 hour to afford Malate Salt Form A.

In one or more embodiments, the mixture is allowed to stir and temperature cycle for at least 12 hours, at least 14 hours, at least 16 hours, at least 18 hours, at least 20 hours, at least 24 hours, at least 2 days, at least 3 days, at least 4 days, at least 5 days, or at least 6 days in step (b). In one or more embodiments, the mixture is allowed to stir and temperature cycle for 75-80 hours.

In one or more embodiments, the temperature cycling is between ambient temperature (of from 15° C. to 30° C.) to 40° C. and back to 5° C. in step (b). In one or more embodiments, the temperature cycling is between ambient temperature to 40° C. at 60 hours, then cooled to 5° C. and stirred at this temperature for another 18 hours.

In one or more embodiments, the isolating is by centrifugation in step (c).

### Malonate Salt Form A

In one or more embodiments, provided herein is a salt of a compound of Formula (I). In one or more embodiments, the salt of the compound of Formula (I) is a malonate salt. In one or more embodiments, the malonate salt is Malonate Salt Form A.

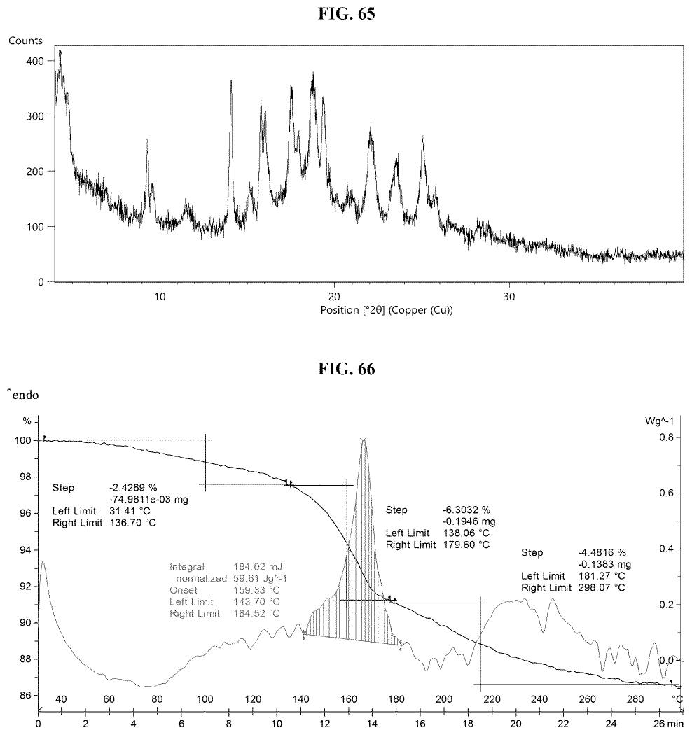

In one or more embodiments, Malonate Salt Form A is characterized by an X-ray powder diffraction (XRPD) pattern substantially similar to that set forth in FIG. 65.

Malonate Salt Form A can be synthesized as described in Synthetic Example 23.

In one or more embodiments, Malonate Salt Form A is characterized by a differential thermal analysis (DTA) thermogram having an endotherm with an endotherm maxima between about 140° C. and about 190° C. In one or more embodiments, Malonate Salt Form A is characterized by a differential thermal analysis (DTA) thermogram having an endotherm with endotherm maxima at about 143° C. and about 185° C. In one or more embodiments, Malonate Salt Form A is characterized by DTA thermogram substantially similar to that set forth in FIG. 66.

In one or more embodiments, Malonate Salt Form A is characterized by a weight loss in the range of about 1% to about 10% when heated from about 30° C. to about 140° C., about 1% to about 10% when heated from about 135° C. to about 180° C., and about 1% to about 10% when heated from about 180° C. to about 305° C. in a thermogravimetric analysis (TGA). In one or more embodiments, Malonate Salt Form A is characterized by a weight loss of about 2.43% when heated from about 31° C. to about 137° C., 6.30% when heated from about 138° C. to about 180° C., and 4.48% when heated from about 181° C. to about 298° C. in a TGA. In one or more embodiments, Malonate Salt Form A is characterized by a TGA substantially similar to that set forth in FIG. 66.

In one or more embodiments, Malonate Salt Form A is synthesized by a method comprising:
a) mixing Free Base Form A (a compound of Formula (I)) with malonic acid (1 mol. equivalent) in THF/water;
b) stirring and temperature cycling;
c) isolating the precipitate as an amorphous solid;
d) mixing the amorphous solid with acetone (15-20 vols) to form a slurry;
e) temperature cycling;
f) isolating the solid to afford Malonate Salt Form A.

In one or more embodiments, the tetrahydrofuran/water in step (a) is in a ratio of from about 1:1 to about 10:1, such as from 1:1 to 9:1, from 1:1 to 8:1, from 1:1 to 7:1, from 1:1 to 6:1, from 1:1 to 5:1, from 1:1 to 4:1, or from 1:1 to 3:1. In one or more embodiments, the tetrahydrofuran/water in step (a) is in a ratio of 2:1 (12 vols).

In one or more embodiments, the mixture is allowed to stir and temperature cycle for at least 12 hours, at least 14 hours, at least 16 hours, at least 18 hours, at least 20 hours, at least 24 hours, at least 2 days, at least 3 days, at least 4 days, at least 5 days, or at least 6 days in step (b) and (e). In one or more embodiments, the mixture is allowed to stir and temperature cycle for 75-80 hours in step (b). In one or more embodiments, the mixture is allowed to stir and temperature cycle for 45-55 hours in step (e).

In one or more embodiments, the temperature cycling is between ambient temperature (of from 15° C. to 30° C.) to 50° C. and back to ambient temperature in step (b). In one or more embodiments, the temperature cycling is between ambient temperature to 50° C. at 60 hours, then cooled to ambient and stirred at this temperature for another 18 hours.

In one or more embodiments, the temperature cycling is between ambient temperature (of from 15° C. to 30° C.) to 40° C. and back to ambient temperature in step (e). In one or more embodiments, the temperature cycling is between ambient temperature to 40° C. at 48 hours, then cooled to ambient temperature in step (e).

In one or more embodiments, the isolating is by centrifugation in steps (c) and (f).

### Napsylate Salt Form A

In one or more embodiments, provided herein is a salt of a compound of Formula (I). In one or more embodiments, the salt of the compound of Formula (I) is a napsylate salt. In one or more embodiments, the napsylate salt is Napsylate Salt Form A.

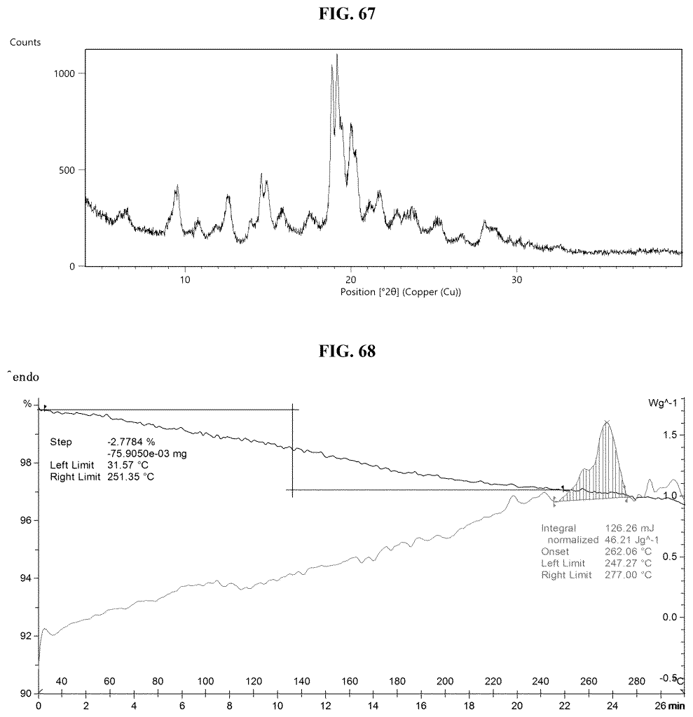

In one or more embodiments, Napsylate Salt Form A is characterized by an X-ray powder diffraction (XRPD) pattern substantially similar to that set forth in FIG. 67.

Napsylate Salt Form A can be synthesized as described in Synthetic Example 24.

In one or more embodiments, Napsylate Salt Form A is characterized by a differential thermal analysis (DTA) thermogram having an endotherm with an endotherm maxima between about 240° C. and about 280° C. In one or more embodiments, Napsylate Salt Form A is characterized by a differential thermal analysis (DTA) thermogram having an endotherm with endotherm maxima at about 247° C. and about 277° C. In one or more embodiments, Napsylate Salt Form A is characterized by DTA thermogram substantially similar to that set forth in FIG. 68.

In one or more embodiments, Napsylate Salt Form A is characterized by a weight loss in the range of about 1% to about 5% when heated from about 30° C. to about 255° C. in a thermogravimetric analysis (TGA). In one or more embodiments, Napsylate Salt Form A is characterized by a weight loss of about 2.78% when heated from about 31° C. to about 251° C. in a TGA. In one or more embodiments, Napsylate Salt Form A is characterized by a TGA substantially similar to that set forth in FIG. 68.

In one or more embodiments, Napsylate Salt Form A is synthesized by a method comprising:
a) mixing Free Base Form A (a compound of Formula (I)) and naphthalene-2-sulfonic acid (1 mol. equivalent) in THF;
b) stirring and temperature cycling;
c) isolating the precipitate to afford Napsylate Salt Form A.

In one or more embodiments, the mixture is allowed to stir and temperature cycle for at least 12 hours, at least 14 hours, at least 16 hours, at least 18 hours, at least 20 hours, at least 24 hours, at least 2 days, at least 3 days, at least 4 days, at least 5 days, or at least 6 days in step (b). In one or more embodiments, the mixture is allowed to stir and temperature cycle for 75-80 hours.

In one or more embodiments, the temperature cycling is between ambient temperature (of from 15° C. to 30° C.) to 40° C. and back to 5° C. in step (b). In one or more embodiments, the temperature cycling is between ambient temperature to 40° C. at 60 hours, then cooled to 5° C. and stirred at this temperature for another 18 hours.

In one or more embodiments, the isolating is by centrifugation in step (c).

### Napsylate Salt Form B, Napsylate Salt Form C, and Napsylate Salt Form D

In one or more embodiments, provided herein is a salt of a compound of Formula (I). In one or more embodiments, the salt of the compound of Formula (I) is a napsylate salt. In one or more embodiments, the napsylate salt is Napsylate Salt Form B. In one or more embodiments, the napsylate salt is Napsylate Salt Form C. In one or more embodiments, the napsylate salt is Napsylate Salt Form D.

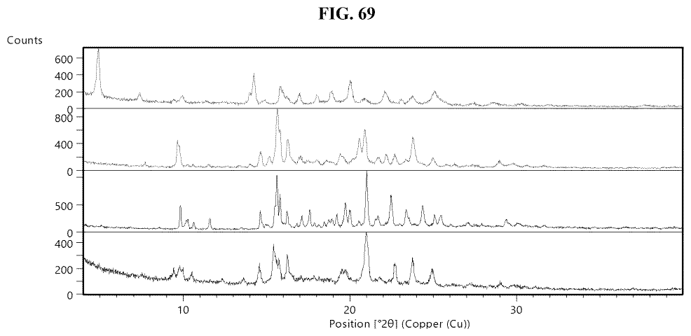

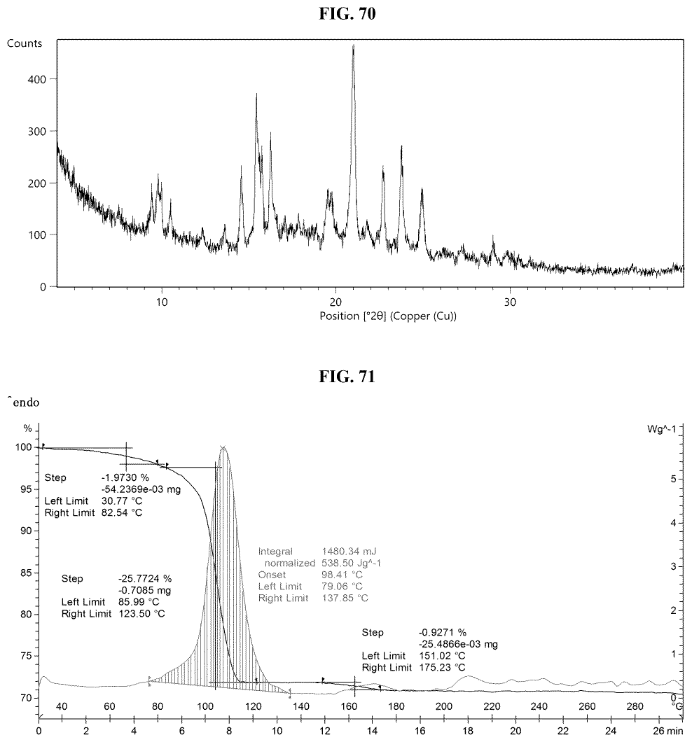

In one or more embodiments, Napsylate Salt Form B is characterized by an X-ray powder diffraction (XRPD) pattern substantially similar to that set forth in FIG. 69 (2^{nd} from top). In one or more embodiments, Napsylate Salt Form C is characterized by an X-ray powder diffraction (XRPD) pattern substantially similar to that set forth in FIG. 69 (2^{nd} from bottom). In one or more embodiments, Napsylate Salt Form D is characterized by an X-ray powder diffraction (XRPD) pattern substantially similar to that set forth in FIG. 69 (bottom) and FIG. 70.

Napsylate Salt Form B can be synthesized as described in Synthetic Example 25. Napsylate Salt Form C can be synthesized as described in Synthetic Example 26. Napsylate Salt Form D can be synthesized as described in Synthetic Example 27.

In one or more embodiments, Napsylate Salt Form D is characterized by a differential thermal analysis (DTA) thermogram having an endotherm with an endotherm maxima between about 75° C. and about 140° C. In one or more embodiments, Napsylate Salt Form D is characterized by a differential thermal analysis (DTA) thermogram having an endotherm with endotherm maxima at about 79° C. and about 138° C. In one or more embodiments, Napsylate Salt Form D is characterized by DTA thermogram substantially similar to that set forth in FIG. 71.

In one or more embodiments, Napsylate Salt Form D is characterized by a weight loss in the range of about 1% to about 5% when heated from about 25° C. to about 85° C., a range of about 20% to about 30% when heated from about 85° C. to about 125° C., and a range of about 0.1% to 5% when heated from about 150° C. to about 180° C. in a thermogravimetric analysis (TGA). In one or more embodiments, Napsylate Salt Form D is characterized by a weight loss of about 1.97% when heated from about 30° C. to about 83° C., a range of about 25% when heated from about 86° C. to about 124° C., and a range of about 0.9% when heated from about 151° C. to about 175° C. in a TGA. In one or more embodiments, Napsylate Salt Form D is characterized by a TGA substantially similar to that set forth in FIG. 71.

In one or more embodiments, Napsylate Salt Form B is synthesized by a method comprising:
a) mixing Free Base Form A (a compound of Formula (I)) with naphthalene-2-sulfonic acid (1 mol. equivalent) in THF/water (12 vols);
b) stirring and temperature cycling;
c) isolating the precipitate to afford Napsylate Salt Form B.

In one or more embodiments, the mixture is allowed to stir and temperature cycle for at least 12 hours, at least 14 hours, at least 16 hours, at least 18 hours, at least 20 hours, at least 24 hours, at least 2 days, at least 3 days, at least 4 days, at least 5 days, or at least 6 days in step (b). In one or more embodiments, the mixture is allowed to stir and temperature cycle for 75-80 hours.

In one or more embodiments, the temperature cycling is between ambient temperature (of from 15° C. to 30° C.) to 50° C. and back to ambient temperature in step (b). In one or more embodiments, the temperature cycling is between ambient temperature to 50° C. at 60 hours, then cooled to ambient temperature and stirred at this temperature for another 18 hours.

In one or more embodiments, the isolating is by centrifugation in step (c).

In one or more embodiments, Napsylate Salt Form C is synthesized by a method comprising storing Napsylate Salt Form B under ambient conditions (including a temperature of from 15° C. to 30° C.) to convert to Napsylate Salt Form C.

In one or more embodiments, Napsylate Salt Form D is synthesized by a method comprising drying Napsylate Salt Form B under vacuum for 1 hour to convert to Napsylate Salt Form D.

### Succinate Salt Forms A and B

In one or more embodiments, provided herein is a salt of a compound of Formula (I). In one or more embodiments, the salt of the compound of Formula (I) is a succinate salt. In one or more embodiments, the succinate salt is Succinate Salt Form A. In one or more embodiments, the succinate salt is Succinate Salt Form B.

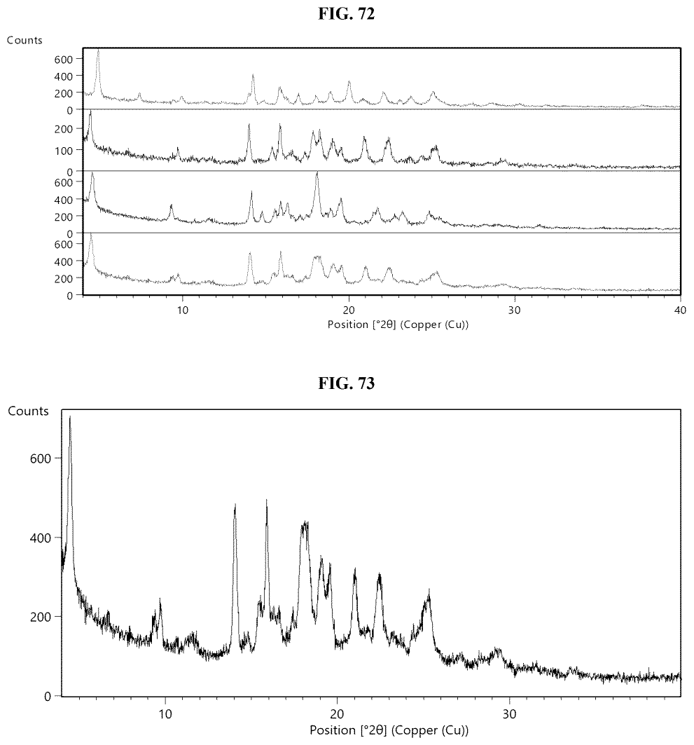

In one or more embodiments, Succinate Salt Form A is characterized by an X-ray powder diffraction (XRPD) pattern substantially similar to that set forth in FIG. 72 (2^{nd} from top, and bottom) and FIG. 73.

Succinate Salt Form A can be synthesized as described in Synthetic Example 28.

In one or more embodiments, Succinate Salt Form B is characterized by an X-ray powder diffraction (XRPD) pattern substantially similar to that set forth in FIG. 72 (2^{nd} from bottom).

Succinate Salt Form B can be synthesized as described in Synthetic Example 29.

In one or more embodiments, Succinate Salt Form B is characterized by a differential thermal analysis (DTA) thermogram having an endotherm with two endotherm maxima, the first endotherm maxima between about 75° C. and about 125° C., and between about 200° C. and about 230° C. In one or more embodiments, Succinate Salt Form B is characterized by a differential thermal analysis (DTA) thermogram having an endotherm with endotherm maxima at about 77° C. and about 122° C., and at about 202° C. and about 226° C. In one or more embodiments, Succinate Salt Form B is characterized by DTA thermogram substantially similar to that set forth in FIG. 74.

In one or more embodiments, Succinate Salt Form B is characterized by a weight loss in the range of about 1% to about 5% when heated from about 30° C. to about 215° C. in a thermogravimetric analysis (TGA). In one or more embodiments, Succinate Salt Form B is characterized by a weight loss of about 1.55% when heated from about 34° C. to about 214° C. in a TGA. In one or more embodiments, Succinate Salt Form B is characterized by a TGA substantially similar to that set forth in FIG. 74.

In one or more embodiments, Succinate Salt Form A and Succinate Salt Form B are synthesized by a method comprising:
a) mixing Free Base Form A (a compound of Formula (I)) with succinic acid (1 mol. equivalent) in THF;
b) stirring and temperature cycling;
c) isolating the precipitate to afford Succinate Salt Form A;
d) drying the precipitate under vacuum 1 hour to afford Succinate Salt Form B.

In one or more embodiments, the mixture is allowed to stir and temperature cycle for at least 12 hours, at least 14 hours, at least 16 hours, at least 18 hours, at least 20 hours, at least 24 hours, at least 2 days, at least 3 days, at least 4 days, at least 5 days, or at least 6 days in step (b). In one or more embodiments, the mixture is allowed to stir and temperature cycle for 75-80 hours.

In one or more embodiments, the temperature cycling is between ambient temperature (of from 15° C. to 30° C.) to 40° C. and back to 5° C. in step (b). In one or more embodiments, the temperature cycling is between ambient temperature to 40° C. at 60 hours, then cooled to 5° C. and stirred at this temperature for another 18 hours.

In one or more embodiments, the isolating is by centrifugation in step (c).

### Succinate Salt Form C

In one or more embodiments, provided herein is a salt of a compound of Formula (I). In one or more embodiments, the salt of the compound of Formula (I) is a succinate salt. In one or more embodiments, the succinate salt is Succinate Salt Form C.

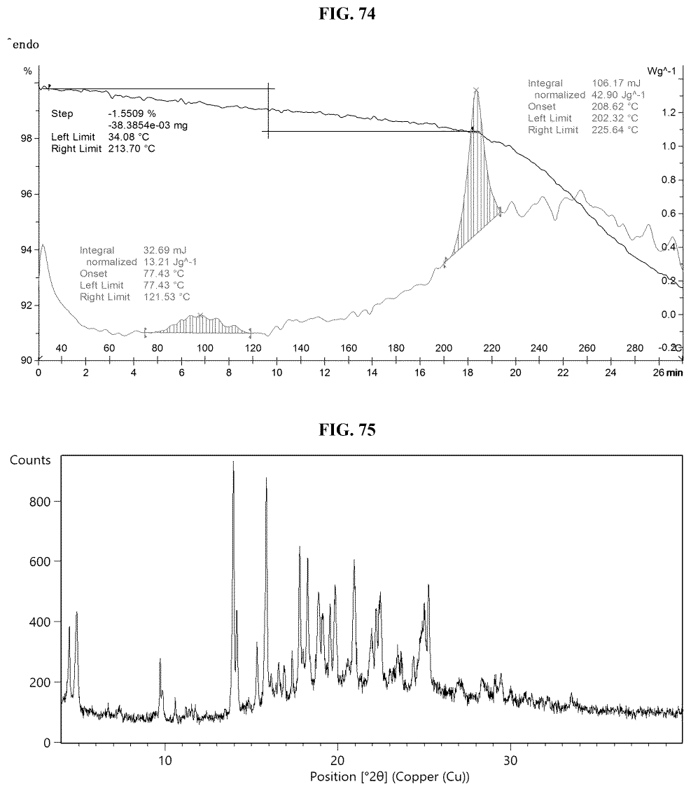

In one or more embodiments, Succinate Salt Form C is characterized by an X-ray powder diffraction (XRPD) pattern substantially similar to that set forth in FIG. 75.

Succinate Salt Form C can be synthesized as described in Synthetic Example 30.

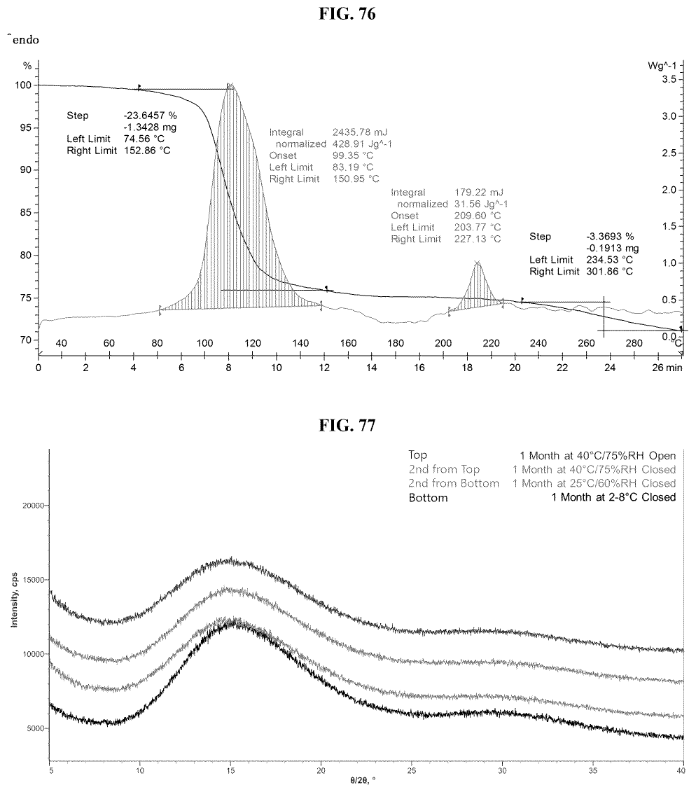

In one or more embodiments, Succinate Salt Form C is characterized by a differential thermal analysis (DTA) thermogram having an endotherm with two endotherms, the first endotherm maxima between about 80° C. and about 155° C., and the second endotherm maxima between about 200° C. and about 235° C. In one or more embodiments, Succinate Salt Form C is characterized by a differential thermal analysis (DTA) thermogram having two endotherms, the first endotherm maxima at about 83° C. and about 151° C., and the second endotherm maxima at about 203° C. to about 227° C. In one or more embodiments, Succinate Salt Form C is characterized by DTA thermogram substantially similar to that set forth in FIG. 76.

In one or more embodiments, Succinate Salt Form C is characterized by a weight loss in the range of about 20% to about 30% when heated from about 70° C. to about 155° C. in a thermogravimetric analysis (TGA). In one or more embodiments, Succinate Salt Form C is characterized by a weight loss of about 23.6% when heated from about 74° C. to about 153° C. in a TGA. In one or more embodiments, Succinate Salt Form C is characterized by a TGA substantially similar to that set forth in FIG. 76.

In one or more embodiments, Succinate Salt Form C is synthesized by a method comprising:
a) mixing Free Base Form A (a compound of Formula (I)) with succinic acid (1 mol. equivalent) in THF/water (12 vols);
b) stirring and temperature cycling;
c) isolating the precipitate to afford Succinate Salt Form C.

In one or more embodiments, the mixture is allowed to stir and temperature cycle for at least 12 hours, at least 14 hours, at least 16 hours, at least 18 hours, at least 20 hours, at least 24 hours, at least 2 days, at least 3 days, at least 4 days, at least 5 days, or at least 6 days in step (b). In one or more embodiments, the mixture is allowed to stir and temperature cycle for 75-80 hours.

In one or more embodiments, the temperature cycling is between ambient temperature (of from 15° C. to 30° C.) to 50° C. and back to ambient temperature in step (b). In one or more embodiments, the temperature cycling is between ambient temperature to 50° C. at 60 hours, then cooled to ambient temperature and stirred at this temperature for another 18 hours.

In one or more embodiments, the isolating is by centrifugation in step (c).

### Pharmaceutical Compositions

The compounds described herein can be formulated into pharmaceutical compositions that further comprise a pharmaceutically acceptable carrier, diluent, adjuvant, or vehicle. In one or more embodiments, this disclosure provides a pharmaceutical composition comprising a compound described above, and a pharmaceutically acceptable carrier, diluent, adjuvant, or vehicle. In one or more embodiments, this disclosure is a pharmaceutical composition comprising an effective amount of a compound of this disclosure or a pharmaceutically acceptable salt thereof and a pharmaceutically acceptable carrier, diluent, adjuvant, or vehicle. Pharmaceutically acceptable carriers include, for example, pharmaceutical diluents, excipients, or carriers suitably selected with respect to the intended form of administration, and consistent with conventional pharmaceutical practices.

According to one or more embodiments, the description provides a composition comprising a compound herein or a pharmaceutically acceptable salt thereof and a pharmaceutically acceptable carrier, adjuvant, or vehicle.

Pharmaceutical compositions of this description comprise a therapeutically effective amount of a salt or free base compound of Formula (I), Free Base Form A, Free Base Amorphous Form, Free Base Form B, Free Base Form C, Free Base Form D, Free Base Form E, Free Base Form F, Free Base Form G, Free Base Form H, Free Base Form J, Fumarate Salt Form A, Fumarate Salt Amorphous Form, Fumarate Salt Form B, Fumarate Salt Form C, Fumarate Salt Form D, Fumarate Salt Form E, Maleate Salt Form A, Tosylate Salt Form A, Tosylate Salt Form B, Besylate Salt Form A, Cyclamate Salt Form A, Malate Salt Form A, Malonate Salt Form A, Napsylate Salt Form A, Napsylate Salt Form B, Napsylate Salt Form C, Napsylate Salt Form D, Succinate Salt Form A, Succinate Salt Form B, and Succinate Salt Form C.

Pharmaceutical compositions of this description comprise a therapeutically effective amount of a salt or free base compound of Formula (I), Fumarate Salt Form A, Maleate Salt Form A, Tosylate Salt Form A, Tosylate Salt Form B, Free Base Form A, Free Base Form B, Free Base Form C, Free Base Form D, Free Base Form E, Free Base Form F, Free Base Form G, Free Base Form H, Free Base Form J, Fumarate Salt Amorphous Form, and/or Free Base Amorphous Form.

Pharmaceutical compositions of this description comprise a therapeutically effective amount of a salt or free base compound of Formula (I), Fumarate Salt Form A, Maleate Salt Form A, Tosylate Salt Form A, Tosylate Salt Form B, Free Base Form A, Free Base Form B, Free Base Form C, and/or Free Base Form D.

A “therapeutically effective amount” is an amount that is (a) effective to measurably degrade BTK (or reduce the amount of BTK) in a biological sample or in a patient; or (b) effective in treating and/or ameliorating a disease, disorder, or condition that is mediated by BTK.

The term “patient,” as used herein, means an animal, alternatively a mammal, and alternatively a human.

A pharmaceutically acceptable carrier may contain inert ingredients that do not unduly inhibit the biological activity of the compounds. The pharmaceutically acceptable carriers should be biocompatible, for example, non-toxic, non-inflammatory, non-immunogenic, or devoid of other undesired reactions or side-effects upon the administration to a subject. Standard pharmaceutical formulation techniques can be employed.

The pharmaceutically acceptable carrier, adjuvant, or vehicle, as used herein, includes any and all solvents, diluents, or other liquid vehicle, dispersion or suspension aids, surface active agents, isotonic agents, thickening or emulsifying agents, preservatives, solid binders, lubricants, and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds described herein, such as by producing any undesirable biological effect or otherwise interacting in a deleterious manner with any other component(s) of the pharmaceutically acceptable composition, the use of such conventional carrier medium is contemplated to be within the scope of this description. As used herein, the phrase “side effects” encompasses unwanted and adverse effects of a therapy (e.g., a prophylactic or therapeutic agent). Side effects are always unwanted, but unwanted effects are not necessarily adverse. An adverse effect from a therapy (e.g., prophylactic or therapeutic agent) might be harmful, uncomfortable, or risky. Side effects include, but are not limited to, fever, chills, lethargy, gastrointestinal toxicities (including gastric and intestinal ulcerations and erosions), nausea, vomiting, neurotoxicities, nephrotoxicities, renal toxicities (including such conditions as papillary necrosis and chronic interstitial nephritis), hepatic toxicities (including elevated serum liver enzyme levels), myelotoxicities (including leukopenia, myelosuppression, thrombocytopenia and anemia), dry mouth, metallic taste, prolongation of gestation, weakness, somnolence, pain (including muscle pain, bone pain, and headache), hair loss, asthenia, dizziness, extra-pyramidal symptoms, akathisia, cardiovascular disturbances, and sexual dysfunction.

In one or more embodiments, an amorphous solid dispersion includes a pharmaceutically acceptable carrier, adjuvant, or vehicle. In one or more embodiments, an amorphous solid dispersion comprises a dispersion aid. In one or more embodiments, the dispersion aid is a polymer. In one or more embodiments, the dispersion aid is a polymer selected from the group consisting of hydroxypropyl methylcellulose acetate succinate (HPMCAS), an anionic copolymer consisting of methacrylic acid/ethyl acrylate, or a random copolymer of N-vinyl pyrrolidone and vinyl acetate (PVP/VA). In one or more embodiments, the HPMCAS is type L (HPCMAS-L), type M (HPMCAS-M), or type H (HPMCAS-H). In one or more embodiments, the HPMCAS is type L (HPCMAS-L) or type H (HPMCAS-H). In one or more embodiments, the anionic copolymer consisting of methacrylic acid/ethyl acrylate is Eudragit L100. In one or more embodiments, the random copolymer of N-vinyl pyrrolidone and vinyl is a 6:4 linear random copolymer of N-vinyl pyrrolidone and vinyl acetate (PVP-VA64).

Some examples of materials that can serve as pharmaceutically acceptable carriers include, but are not limited to, ion exchangers, alumina, aluminum stearate, lecithin, serum proteins (such as human serum albumin), buffer substances (such as tween 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring, and perfuming agents. Preservatives and antioxidants can also be present in the composition, according to the judgment of the formulator.

As used herein, the term “measurably degrade,” means a measurable reduction in (a) BTK activity, between a sample comprising a compound of this description and a BTK and an equivalent sample comprising a BTK in the absence of said compound; or (b) the concentration of the BTK in a sample over time.

### Administration

The compositions of this disclosure may be administered orally. The pharmaceutically acceptable compositions of this description may be orally administered in any orally acceptable dosage form including, but not limited to, capsules, tablets, aqueous suspensions, or solutions. In the case of tablets for oral use, carriers commonly used include lactose and corn starch. Lubricating agents, such as magnesium stearate, are also typically added. For oral administration in a capsule form, useful diluents include lactose and dried cornstarch. When an aqueous suspension is for oral use, the active ingredient is combined with emulsifying and suspending agents. If desired, sweetening, flavoring, or coloring agents also may be added.

Liquid dosage forms for oral administration include, but are not limited to, pharmaceutically acceptable emulsions, microemulsions, solutions, suspensions, syrups, and clixirs. In addition to the active compounds herein, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions also can include adjuvants such as wetting agents, emulsifying and suspending agents, sweetening, flavoring, and perfuming agents.

Solid dosage forms for oral administration include capsules, tablets, pills, powders, and granules. In such solid dosage forms, the active compound herein is mixed with at least one inert, pharmaceutically acceptable excipient or carrier such as sodium citrate or dicalcium phosphate and/or a) fillers or extenders such as starches, lactose, sucrose, glucose, mannitol, and silicic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, one or more silicates, and sodium carbonate; c) solution retarding agents such as paraffin; f) absorption accelerators such as quaternary ammonium compounds; g) wetting agents such as, for example, cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets, and pills, the dosage form also may comprise buffering agents.

Solid compositions of a similar type also may be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. Solid dosage forms optionally may contain opacifying agents. These solid dosage forms also can be of a composition such that they release the active ingredient(s) only, for example, in a part of the intestinal tract, optionally, in a delayed manner. Examples of embedding compositions that can be used include polymeric substances and waxes. Solid compositions of a similar type also may be employed as fillers in soft and hard-filled gelatin capsules using such excipients as lactose or milk sugar as well as high molecular weight polethylene glycols and the like.

The active compounds herein also can be in micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active compound may be admixed with at least one inert diluent such as sucrose, lactose, or starch. Such dosage forms also may comprise, as is normal practice, additional substances other than inert diluents, for example, tableting lubricants and other tableting aids such a magnesium stearate and microcrystalline cellulose. In the case of capsules, tablets and pills, the dosage forms also may comprise buffering agents. They may optionally contain opacifying agents and also can be of a composition such that they release the active ingredient(s) only, for example, in a part of the intestinal tract, optionally, in a delayed manner. Examples of embedding compositions that can be used include polymeric substances and waxes.

The compounds of the description are formulated in dosage unit form for case of administration and uniformity of dosage. As used herein, the phrase “dosage unit form” refers to a physically discrete unit of agent appropriate for the patient to be treated. It will be understood, however, that the total daily usage of the compounds and compositions of this disclosure will be decided by the attending physician within the scope of sound medical judgment. The specific effective dose level for any particular patient or organism will depend upon a variety of factors including the disease, disorder, or condition being treated and the severity of the disease, disorder, or condition; the activity of the specific compound employed; the specific composition employed; the age, body weight, general health, sex, and diet of the patient; the time of administration, route of administration, and rate of excretion of the specific compound employed; the duration of the treatment; drugs used in combination or coincidental with the specific compound employed, and like factors well known in the medical arts.

The amount of the compounds of this disclosure that may be combined with the carrier materials to produce a composition in a single dosage form will vary depending upon the host treated, the particular mode of administration, and other factors. The compositions should be formulated so that a dosage of between 0.01-100 mg/kg body weight/day of the compound or inhibitor can be administered to a patient receiving these compositions.

Depending upon the particular disease, disorder, or condition to be treated or prevented, additional therapeutic agents, which are normally administered to treat or prevent that condition, also may be present in the compositions of this disclosure. As used herein, additional therapeutic agents that are normally administered to treat or prevent a particular disease, disorder, or condition, are known as “appropriate for the disease, disorder, or condition being treated.”

For example, chemotherapeutic agents or other anti-proliferative agents may be combined with the compounds of this disclosure to treat proliferative diseases and cancer. Examples of known chemotherapeutic agents include, but are not limited to, PI3K inhibitors (e.g., idelalisib and copanlisib), BCL-2 inhibitors (e.g., venetoclax), BTK inhibitors (e.g., ibrutinib and acalabrutinib), etoposide, CD20 antibodies (e.g., rituximab, ocrelizumab, obinutuzumab, ofatumumab, ibritumomab tiuxctan, tositumomab, and ublituximab), aletuzumab, bendamustine, cladribine, doxorubicin, chlorambucil, prednisone, midostaurin, lenalidomide, pomalidomide, checkpoint inhibitors (e.g., ipilimumab, nivolumab, pembolizumab, atezolizumab, avelumab, durvalumab), engineered cell therapy (e.g., CAR-T therapy-Kymriah®, Yescarta®), Gleevec™, adriamycin, dexamethasone, vincristine, cyclophosphamide, fluorouracil, topotecan, taxol, interferons, and platinum derivatives.

And, in some instances, radiation therapy is administered during the treatment course wherein a compound of this disclosure (or a pharmaceutically acceptable salt thereof) is administered to a patient in need thereof.

Other examples of agents with which the compounds or inhibitors of this disclosure also may be combined include, without limitation, treatments for Alzheimer's Disease such as Aricept® and Excelon®; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif®), Copaxone®, and mitoxantrone; treatments for asthma such as albuterol and Singulair®; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; and agents for treating immunodeficiency disorders such as gamma globulin.

The amount of additional therapeutic agent present in the compositions of this disclosure will be no more than the amount that would normally be administered in a composition comprising that therapeutic agent as the only active agent. The amount of additional therapeutic agent in the presently disclosed compositions will range from about 50% to 100% of the amount normally present in a composition comprising that agent as the only therapeutically active agent.

### Methods of Treatment

In one or more embodiments, a form of compound of Formula (I) provided herein, including salt forms and crystalline forms thereof and the Fumarate Salt Form A, Maleate Salt Form A, Tosylate Salt Form A, and Tosylate Salt Form B, is used to treat a disease, disorder, or condition caused by oxidative stress and/or inflammation useful for degrading BTK in biological samples or in patients via a ubiquitin proteolytic pathway.

In one or more embodiments, a form of compound of Formula (I) provided herein, including free bases and crystalline forms thereof and the Free Base Form A, Free Base Form B, Free Base Form C, and Free Base Form D, is used to treat a disease, disorder, or condition caused by oxidative stress and/or inflammation useful for degrading BTK in biological samples or in patients via a ubiquitin proteolytic pathway.

In one or more embodiments, a form of compound of Formula (I) provided herein, including free bases and crystalline forms thereof and the Free Base Form A, Free Base Form B, Free Base Form C, Free Base Form D, Free Base Form E, Free Base Form F, Free Base Form G, Free Base Form H, and Free Base Form J, is used to treat a disease, disorder, or condition caused by oxidative stress and/or inflammation useful for degrading BTK in biological samples or in patients via a ubiquitin proteolytic pathway.

In one or more embodiments, a form of compound of Formula (I) provided herein, including free bases and crystalline forms thereof and the Free Base Form A, Free Base Amorphous Form, Free Base Form B, Free Base Form C, Free Base Form D, Free Base Form E, Free Base Form F, Free Base Form G, Free Base Form H, Free Base Form J, Fumarate Salt Form A, Fumarate Salt Amorphous Form, Fumarate Salt Form B, Fumarate Salt Form C, Fumarate Salt Form D, Fumarate Salt Form E, Maleate Salt Form A, Tosylate Salt Form A, Tosylate Salt Form B, Besylate Salt Form A, Cyclamate Salt Form A, Malate Salt Form A, Malonate Salt Form A, Napsylate Salt Form A, Napsylate Salt Form B, Napsylate Salt Form C, Napsylate Salt Form D, Succinate Salt Form A, Succinate Salt Form B, and Succinate Salt Form C, is used to treat a disease, disorder, or condition caused by oxidative stress and/or inflammation useful for degrading BTK in biological samples or in patients via a ubiquitin proteolytic pathway.

In one or more embodiments, a compound of Formula (I) provided herein, including amorphous forms thereof and the Fumarate Salt Amorphous Form and Free Base Amorphous Form, is used to treat a disease, disorder, or condition caused by oxidative stress and/or inflammation useful for degrading BTK in biological samples or in patients via a ubiquitin proteolytic pathway.

The forms of compounds of the present disclosure are useful for degrading BTK in biological samples or in patients via a ubiquitin proteolytic pathway. Thus, an embodiment of the present disclosure provides a method of treating a BTK-mediated disease, disorder, or condition. As used herein, the term “BTK-mediated disease, disorder, or condition” means any disease, disorder, or other deleterious condition in which a BTK is known to play a role. In some instances, a BTK-mediated disease, disorder, or condition is a proliferative disorder or an autoimmune disorder. Examples of proliferative disorders include cancer.

The term “cancer” includes, but is not limited to, the following cancers: epidermoid Oral: buccal cavity, lip, tongue, mouth, pharynx; Cardiac: sarcoma (angiosarcoma, fibrosarcoma, rhabdomyosarcoma, liposarcoma), yxo a, rhabdomyoma, fibroma, lipoma, and teratoma; Lung: bronchogenic carcinoma (squamous cell or epidermoid, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, larynx, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel or small intestines (adenocarcinoma, lymphoma, carcinoid tumors, Karposi's sarcoma, leiomyoma, hemangioma, lipoma, neurofibroma, fibroma), large bowel or large intestines (adenocarcinoma, tubular adenoma, villous adenoma, hamartoma, leiomyoma), colon, colon-rectum, colorectal, rectum; Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma, biliary passages; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (ostcoma, hemangioma, granuloma, xanthoma, osteitis deformans), meninges (meningioma, meningiosarcoma, gliomatosis), brain (astrocytoma, medulloblastoma, glioma, ependymoma, germinoma (pincaloma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast; Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma) hairy cell; lymphoid disorders (e.g., mantle cell lymphoma, Waldenstrom's macroglobulinemia, Marginal zone lymphoma, and Follicular lymphoma); Skin: malilymphgnant melanoma, basal cell carcinoma, squamous cell carcinoma, Karposi's sarcoma, keratoacanthoma, moles dysplastic nevi, lipoma, angioma, dermatofibroma, keloids, psoriasis, Thyroid gland: papillary thyroid carcinoma, follicular thyroid carcinoma; medullary thyroid carcinoma, undifferentiated thyroid cancer, multiple endocrine neoplasia type 2A, multiple endocrine neoplasia type 2B, familial medullary thyroid cancer, phcochromocytoma, paraganglioma; and Adrenal glands: neuroblastoma.

Examples of autoimmune diseases, disorders, or conditions include uticaria, graft-versus-host disease, pemphigus vulgaris, achalasia, Addison's disease, Adult Still's disease, agammaglobulinemia, alopecia arcata, amyloidosis, ankylosing spondylitis, anti-GBM/anti-TBM nephritis, antiphospholipid syndrome, autoimmune angioedema, autoimmune dysautonomia, autoimmune encephalomyelitis, autoimmune hepatitis, autoimmune inner car disease (AIED), autoimmune myocarditis, autoimmune oophoritis, autoimmune orchitis, autoimmune pancreatitis, autoimmune retinopathy, axonal and neuronal neuropathy (AMAN), Balo disease, Behcet's disease, benign mucosal pemphigoid, bullous pemphigoid, Castleman disease (CD), Celiac disease, Chagas disease, chronic inflammatory demyelinating polyneuropathy (CIDP), chronic recurrent multifocal osteomyelitis (CRMO), Churg-Strauss Syndrome (CSS) or Eosinophilic Granulomatosis (EGPA), cicatricial pemphigoid, Cogan's syndrome, cold agglutinin disease, congenital heart block, coxsackie myocarditis, CREST syndrome, Crohn's disease, dermatitis herpetiformis, dermatomyositis, Devic's disease (neuromyelitis optica), discoid lupus, Dressler's syndrome, endometriosis, cosinophilic esophagitis (EoE), cosinophilic fasciitis, crythema nodosum, essential mixed cryoglobulinemia, evans syndrome, fibromyalgia, fibrosing alveolitis, giant cell arteritis (temporal arteritis), giant cell myocarditis, glomerulonephritis, goodpasture's syndrome, granulomatosis with polyangiitis, Graves' disease, Guillain-Barre syndrome, Hashimoto's thyroiditis, hemolytic anemia, Henoch-Schonlein purpura (HSP), herpes gestationis or pemphigoid gestationis (PG), hidradenitis suppurativa (HS) (Acne Inversa), hypogammalglobulinemia, IgA nephropathy, IgG4-related sclerosing disease, immune thrombocytopenia purpura (ITP), inclusion body myositis (IBM), interstitial cystitis (IC), juvenile arthritis, juvenile diabetes (Type 1 diabetes), juvenile myositis (JM), Kawasaki disease, Lambert-Eaton syndrome, leukocytoclastic vasculitis, lichen planus, lichen sclerosus, ligneous conjunctivitis, linear IgA disease (LAD), lupus, lyme disease chronic, Meniere's disease, microscopic polyangiitis (MPA), mixed connective tissue disease (MCTD), Mooren's ulcer, Mucha-Habermann disease, Multifocal Motor Neuropathy (MMN) or MMNCB, multiple sclerosis, myasthenia gravis, myositis, narcolepsy, neonatal lupus, neuromyelitis optica, neutropenia, ocular cicatricial pemphigoid, optic neuritis, palindromic rheumatism (PR), PANDAS, paraneoplastic cerebellar degeneration (PCD), paroxysmal nocturnal hemoglobinuria (PNH), Parry Romberg syndrome, pars planitis (peripheral uveitis), Parsonnage-Tumer syndrome, pemphigus, peripheral neuropathy, perivenous encephalomyelitis, pernicious anemia (PA), POEMS syndrome, polyarteritis nodosa, polyglandular syndromes type I, II, III, polymyalgia rheumatica, polymyositis, postmyocardial infarction syndrome, postpericardiotomy syndrome, primary biliary cirrhosis, primary sclerosing cholangitis, progesterone dermatitis, psoriasis, psoriatic arthritis, pure red cell aplasia (PRC A), pyoderma gangrenosum, Raynaud's phenomenon, reactive Arthritis, reflex sympathetic dystrophy, relapsing polychondritis, restless legs syndrome (RLS), retroperitoneal fibrosis, rheumatic fever, rheumatoid arthritis, sarcoidosis, Schmidt syndrome, scleritis, scleroderma, Sjogren's syndrome, sperm and testicular autoimmunity, stiff person syndrome (SPS), subacute bacterial endocarditis (SBE), Susac's syndrome, sympathetic ophthalmia (SO), Takayasu's arteritis, temporal arteritis (giant cell arteritis), thrombocytopeni purpura (TTP), Tolosa-Hunt syndrome (THS), transverse myelitis, Type 1 diabetes, ulcerative colitis (UC), undifferentiated connective tissue disease (UCTD), uveitis, vasculitis, vitiligo, Vogt-Koyanagi-Harada Disease, and Wegener's granulomatosis (or Granulomatosis with Polyangiitis (GPA)).

In one or more embodiments, the disease, disorder, or condition is diffuse large B cell lymphoma (DLBCL), chronic lymphocytic leukemia (CLL), small lymphocytic lymphoma (SLL), follicular lymphoma (FL), mantle cell lymphoma (MCL), marginal zone lymphoma (MZL), Waldenström macroglobulinemia (WM), including those with secondary CNS involvement in any disease, disorder, or condition listed, or primary central nervous system lymphoma (PCNSL).

In one or more embodiments, DLBCL is non-GCM DLBCL: non-Germinal center (non-GCB) B-cell like subtype DLBCL. In one or more embodiments, the MCL is non-GCB MCL.

In one or more embodiments, the disease, disorder, or condition includes secondary CNS involvement in the disease, disorder, or condition.

In one or more embodiments, the disease, disorder, or condition is in a patient (or adult patient) with relapsed/refractory (R/R) B cell malignancy who has received at least 2 prior lines of therapy and for whom no other therapies are known to provide clinical benefit.

### EXAMPLES

### Methods

For Fumarate Salt Form A, Maleate Salt Form A, Tosylate Salt Form A, Tosylate Salt Form B, Free Base Form A, Free Base Form B, Free Base Form C, and Free Base Form D: XRPD analyses were performed using a Panalytical Empyrean diffractometer equipped with a Cu X-ray tube and a PIXcel ID-Medipix3 detector system. The samples were analyzed at ambient temperature (of from 15° C. to 30° C.) in transmission mode on a 48 well plate and held between mylar polymer film. The Almac default XRPD program was used (range 4-40° 20, step size 0.01313°, counting time 24 sec, ˜6 min run time). Diffraction peaks were detected using the peak picking tool of HighScore Plus v4.9 software and fitted using the “default profile fit” routine of HighScore Plus v4.9 software. A typical variability of ±0.2° 2θ in peak positions (USP-941) applies to this data. XRPD peak positions were determined using Panalytical X′Pert HighScore Plus software V4.9c with default peak picking and fitting parameters. Peak positions were added for a few small peaks that were not found using the default peak search. Peak positions can be difficult to determine for small intensity peaks, peaks with an unusual profile shape, broad peaks (disorder) or peaks that were poorly resolved.

Powder samples for XRPD analysis were prepared in a low background silicon holder using light manual pressure to keep the sample surfaces flat and level with the reference surface of the sample holder. Each sample was analysed from 2 to 40° 2θ using a continuous scan of 6° 2θ per minute with an effective step size of 0.02° 2θ.

For Free Base Form E, Free Base Form F, Free Base Form G, Free Base Form H, and Free Base Form J: two different instruments were used for characterizing the materials by XRPD. Samples were analysed dry with a Malvern Panalytical Xpert Pro diffractometer, except for Free Base Form F. Free Base Form F was analysed using a Malvern Panalytical Empyrean diffractometer. Free Base Form F was characterised by XRPD as it was a wet sample, which converted on drying at ambient conditions (including a temperature of from 15° C. to 30° C.) to Free Base Form J.

XRPD patterns of Free Base Form E, Free Base Form F, Free Base Form G, Free Base Form H, and Free Base Form J were collected with a Malvern Panalytical Xpert Pro diffractometer equipped with a Cu X-ray tube and a PIXcel detector system. The isothermal samples were analysed in transmission mode and held on low density PVC films. The Almac default program was used (range 3-40° 2θ, step size 0.013°, counting time 99 seconds, about 22 minutes runtime.

XRPD patterns of Free Base Form E, Free Base Form F, Free Base Form G, Free Base Form H, and Free Base Form J were collected with a Malvern Panalytical Empyrean diffractometer equipped with a Cu X-ray tube as described previously, and a Malvern Panalytical PIXcelID detector with Medipix3. Powder samples for XRPD analysis were analysed at ambient temperature in transmission mode on a 96 well plate and held on mylar polymer film. The default XRPD program was used (range 4-40° 20, step size 0.01313°, counting time 24 sec, ˜5 min run time).

Diffraction peaks were detected using the peak picking tool of HighScore Plus v4.9 software and fitted using the “default profile fit” routine of HighScore Plus v4.9 software. A typical variability of +0.1° 20 in peak positions (USP-941) applies to this data for Free Base Form E, Free Base Form F, Free Base Form G, Free Base Form H, and Free Base Form J.

For Fumarate Salt Amorphous Form and Free Base Amorphous Form: X-ray powder diffraction (XRPD) patterns were collected with were collected with a Malvern Panalytical Empyrean diffractometer equipped with a Cu X-ray tube as described previously, and a Malvern Panalytical PIXcel1D detector with Medipix3. The samples for XRPD analysis were analysed at ambient temperature in transmission mode on a 48 well plate and held on mylar polymer film. The default XRPD program was used (range 4-40° 2θ, step size 0.01313°, counting time 24 sec, ˜5 min run time).

For Fumarate Salt Form A, Maleate Salt Form A, Tosylate Salt Form A, Tosylate Salt Form B, Free Base Form A, Free Base Form B, Free Base Form C, and Free Base Form D: differential scanning calorimetry (DSC) analyses were performed using a TA Instruments Q2500 Discovery Series instrument. The instrument temperature calibration was performed using indium. The DSC cell was kept under a nitrogen purge of ˜50 mL per minute during each analysis. The sample was placed in a standard, crimped, aluminum pan and was heated from approximately 25° C. to 350° C. at a rate of 10° C. per minute.

For Fumarate Salt Form A, Maleate Salt Form A, Tosylate Salt Form A, Tosylate Salt Form B, Free Base Form A, Free Base Form B, Free Base Form C, and Free Base Form D: thermogravimetric analysis (TGA) was performed using a TA Instruments Q5500 Discovery Series instrument. The instrument balance was calibrated using class M weights and the temperature calibration was performed using alumel. The nitrogen purge was ˜40 mL per minute at the balance and ˜60 mL per minute at the furnace. Each sample was placed into a pre-tared platinum pan and heated from approximately 25° C. to 350° C. at a rate of 10° C. per minute.

For Free Base Form E, Free Base Form F, Free Base Form G, Free Base Form H, and Free Base Form J: Thermogravimetric analyses were carried out on a Mettler Toledo TGA/DSC1 STARe. Samples were placed in an aluminium sample pan, accurately weighed and pin hole lid crimped into position before inserting into the TG furnace. Under a stream of nitrogen at a rate of 10° C./minute, the heat flow signal was stabilised for one minute at 30° C., prior to heating to 300° C. DTA signal is calibrated against indium and zinc standards.

For Fumarate Salt Amorphous Form and Free Base Amorphous Form: thermogravimetric analysis and differential thermal analysis (TGA/DTA) was performed using a Mettler Toledo TGA/DSC1 STARe. Samples were placed on an aluminum sample pan, accurately weighed as previously described in the preceding paragraph, and inserted into the TG furnace. Under a stream of nitrogen at a rate of 10° C./minute, the heat flow signal was stabilized for 1 minute at 30° C., prior to heating to 300° C. The DTA signal was calibrated against indium and zinc standards.

For Fumarate Salt Amorphous Form and Free Base Amorphous Form: differential scanning calorimetry (DSC) analyses were performed using a Perkin Elmer DSC8500. The samples were accurately weighed and were placed in aluminium pans, covered with lid, and crimped (closed but not gas tight). Each sample was heated under nitrogen at a rate of 10° C./minute to a maximum of 240° C. for Fumarate Salt Amorphous Form and 300° C. for Free Base Amorphous Form. Indium and Zinc metals were used as the calibration standard. Temperatures were reported at the transition onset to the nearest 0.01 degree.

In one or more examples, Karl Fischer titrations (water content analyses) for Free base(s) were carried out using a Mettler Toledo model D03080 drying oven interfaced to a Mettler-Toledo C20 Coulometric KF titrator. The titrator was equipped a DM143-SC sensor and filled with HYDRANAL®-Coulomat AD fritless methanol solution. The oven and titrator were calibrated using Apura® Water Standard Oven 1%. For each measurement, the sample was accurately weighed into an aluminum weigh boat and placed into the oven. The sample was titrated using a maximum starting drift of 25 μg/min, a 120 second mix time, and a 35% stirring speed. The oven was purged with nitrogen at a flow rate of 40 mL/min and the temperature was set to 150° C. Duplicate analyses were performed per sample.

In another one or more examples, Karl Fischer titrations (water content analysis) for fumarate salt(s) (e.g., Fumarate Salt Form A) were carried out using a Mettler-Toledo C20 Coulometric KF titrator. The instrument was calibrated using a Hydranal water standard containing 1% water. The titrant was a Hydranal methanol solution.

For Fumarate Salt Form A, Dynamic vapor sorption (DVS) analysis was carried out using a TA Instruments Q5000 Dynamic Vapor Sorption analyser. The instrument was calibrated with standard weights and a sodium bromide standard for humidity. Approximately 10-25 mg of sample was loaded into a metal coated quartz pan for analysis. The sample was analysed at 25° C. with a maximum equilibration time of one hour in 10% relative humidity (RH) steps from 5 to 95% RH (adsorption cycle) and from 95 to 5% RH (desorption cycle). The movement from one step to the next occurred either after satisfying the equilibrium criterion of 0.01% weight change, or when the equilibrium criterion was not met after one hour. The percent weight change values were then calculated.

For Fumarate Salt Amorphous Form and Free Base Amorphous Form: Dynamic Vapour Sorption (DVS) was performed using a SMS DVS Intrinsic Vapour Sorption Balance. Approximately 19.75 mg of Frec Base Amorphous Form and 15.47 mg of Fumarate Salt Amorphous Form, respectively, were placed into an aluminium balance pan, loaded into the vapour sorption balance and held at 25° C.±0.1° C. The sample was subjected to a step profile in 10% increments from 40-90% relative humidity (RH), followed by desorption from 90-0% RH and a second sorption cycle from 0-90% RH, a second desorption from 90-0% and finished by a last sorption step from 0-40% relative humidity (RH). The equilibrium criterion was set as ∂m/∂t=0.002%/min with a minimum of 60 minutes and a maximum of 5 hours for each increment. The weight change during the sorption cycle was monitored, allowing for the hygroscopic nature of the sample to be determined. The data collection interval was in seconds.

Optical microscopy samples were observed under a Leica DM 2500 P compound microscope. Images were captured using a QImaging MicroPublisher 3.3 RTV camera. Images were collected at 10× magnification.

The ¹H NMR spectra were acquired on a Bruker Avance II 400 MHz or 500 MHZ spectrometer. Samples were prepared by dissolving material in DMSO-d6. The solutions were filtered and placed into individual 5 mm NMR tubes for subsequent spectral acquisition. The temperature controlled (295 K) ¹H NMR spectra were acquired on the Bruker Avance II 400 utilized a 5 mm cryoprobe operating at an observing frequency of 400.18 MHZ.

Fourier transform infrared spectroscopy (FT-IR) analyses were performed on a Nicolet 6700 FT-IR system. Samples were analysed using a Nicolet SMART iTR attenuated total reflectance device.

Fourier transform Raman spectroscopy were performed on a Nicolet model 6700 spectrometer interfaced to a Nexus Raman accessory module. This instrument is configured with a Nd: YAG laser operating at 1024 nm, a CaF2 beamsplitter, and an indium gallium arsenide detector. OMNIC 8.1 software was used for control of data acquisition and processing of the spectra. Samples were packed into a 3-inch glass NMR tube for analysis.

### Example 1: Free Base Compound of Formula (I), Starting Material Characterization and Studies

Polymorph screening of compounds of Formula (I) were conducted. Compounds in these studies included four polymorphic salt forms of compounds of Formula (I): fumarate, maleate, and two tosylate salts. Compounds in these studies also included four polymorphic free bases of compounds of Formula (I): Free Base Forms A, B, C, and D.

The free base compound of Formula (I) was used as a starting material. The compound of Formula (I) (MW 806.973 g/mol) is a chimeric targeting molecule that has two ligands connected by a linker. The pKa (base) of the free base compound of Formula (I) is about 6.5; permeability is 2.3/16.3 or 9.6/6.2 (with and without Aloe vera, respectively), and kinetic solubility at pH 7.4 is 10 μM.

Solubilities of the compound of Formula (I), free base, in a few solvents were estimated. The experiments were carried out by adding the test solvent in aliquots to weighed portions of solid. Whether dissolution had occurred was judged by visual inspection after addition of each solvent aliquot. The results are shown in Tables 1 and 2. Solubility numbers were calculated by dividing the weight of the sample by total amount of solvent used to dissolve the sample. The actual solubilities may be greater than the numbers calculated because of the use of solvent aliquots that were too large or because of slow dissolution rates. The solubility number is expressed as “less than” if dissolution did not occur during the experiment. The solubility number is expressed as “greater than or equal to” if dissolution occurred on addition of the first solvent aliquot.

TABLE 1
Solubilities of Compounds of Formula
(I), free base, in selected solvents

| Solvent | Solubility (mg/mL) |
|---|---|
| acetone | <1^{a} |
| acetonitrile (ACN) | <1^{a} |
| anisole | <1 at 60° C. |
| cyclohexane | <2 at 60° C. |
| dichloromethane (DCM) | 17^{a} |
| 1,4-dioxane | 6^{a} |
| 1,2-dichloroethane | 2 at 45° C. |
| dimethylformamide (DMF) | 21^{a} |
| dimethylsulfoxide (DMSO) | 9^{a} |
| ethanol (EtOH) | <1^{a} |
| ethyl acetate (EtOAc) | <1^{a} |
| diethyl ether (Et₂O) | <1^{a} |
| heptane | <2 at 60° C. |
| methanol (MeOH) | <1^{a} |
| methyl ethyl ketone (MEK) | <1^{a} |
| methyl acetate (MeOAc) | <1 at 60° C. |
| Methyl tert-butyl ether (MTBE) | <1 at 60° C. |
| 2-methyltetrahydrofuran (2-MeTHF) | <1^{a} |
| 2-propanol (2-PrOH) | <1^{a} |
| tetrahydrofuran (THF) | 9^{a} |
| toluene | <2 at 60° C. |
| water | <1^{a} |

^{a}solubility without temperature

TABLE 2
Solubilities and water activity of compounds of Formula
(I), free base, in aqueous binary mixtures

| Solvent mixture | Water activity, a_{w} | Solubility (mg/mL) |
|---|---|---|
| acetone/H₂O (70/30) | 0.85 | <2 at 60° C. |
| acetonitrile/H₂O (45/55) | 0.9 | <2 at 60° C. |
| 1,4-dioxane/H₂O (60/40) | 1.0 | 2 at 60° C. |
| ethanol/H₂O (70/30) | 0.8 | <2 at 60° C. |
| methanol/H₂O (50/50) | 0.75 | <2 at 60° C. |
| 1-pentanol/H₂O (saturated) | — | <3 at 60° C. |
| (80:20 - 2 phase system) | | |
| 2-propanol/H₂O (80/20) | 0.9 | <2 at 60° C. |
| tetrahydrofuran/H₂O (80/20) | 1 | 5 at RT |

“—” is not applicable or not tested

### Example 2: Salt Screening

The free base compound of Formula (I) was mixed with different acids (counterions) under various conditions to generate crystalline salts. Samples generated and analysed are listed in Table 3. Experiments were performed using 1 mol. equivalent of acid, except sulfuric (0.5 mol eq.).

TABLE 3
Samples generated and analysed

| Base | Conditions | XRPD pattern |
|---|---|---|
| citric | cool, THF, −15° C.; hexane | non-crystalline |
| | antisolvent, −15° C. | |
| | slurry, 95:5 MeOH/H₂O, 40° C. | non-crystalline |
| | precipitation, DMF, Et₂O | non-crystalline + peak |
| | antisolvent, RT | |
| fumaric | cool, THF, −15° C. | non-crystalline + peaks (broad) |
| | slurry, 95:5 MeOH/H₂O, 40° C. | not previously identified |
| | precipitation, DMF, ACN | non-crystalline + peaks (broad) |
| | antisolvent, RT | |
| D-gluconic | cool, THF, −15° C.; hexane | compound of Formula (I), Free Base |
| | antisolvent, −15° C. | Form A (low crystallinity) |
| | slurry, 95:5 MeOH/H₂O, 40° C. | compound of Formula (I), Free Base |
| | | Form A + non-crystalline |
| | precipitation, DMF, acetone | non-crystalline |
| | antisolvent, RT | |
| hippuric | cool, THF, −15° C.; hexane | non-crystalline + peaks (broad) |
| | antisolvent, −15° C. | |
| | slurry, 95:5 MeOH/H₂O, 40° C. | compound of Formula (I), Free Base |
| | | Form A + non-crystalline |
| | precipitation, DMF, EtOAc | non-crystalline |
| | antisolvent, RT; −15° C. | |
| hydrochloric | precipitation, THF, RT | non-crystalline + peak (broad) |
| | cool, DCM, −15° C. | non-crystalline + peaks (broad) |
| | precipitation, DMF, Et₂O | non-crystalline + peak (broad) |
| | antisolvent, RT | |
| maleic | precipitation, THF, RT | not previously identified |
| | cool, DCM, −15° C. | non-crystalline + peaks (broad) |
| | precipitation, DMF, acetone | not previously identified |
| | antisolvent, RT | |
| methanesulfonic | precipitation, THF, RT | non-crystalline + peaks (broad) |
| | cool, DCM, RT | non-crystalline |
| | precipitation, DMF, ACN | non-crystalline |
| | antisolvent, RT; −15° C. | |
| phosphoric | precipitation, THF, RT | non-crystalline + peaks (broad) |
| | cool, DCM, −15° C. | non-crystalline + peaks (broad) |
| | precipitation, DMF, EtOAc | non-crystalline + peaks (broad) |
| | antisolvent, RT | |
| sulfuric (0.5 mol | precipitation, THF, RT | compound of Formula (I), Free Base |
| eq.) | | Form D (low crystallinity) |
| | cool, DCM, −15° C. | non-crystalline + peaks (broad) |
| | precipitation, DMF, Et₂O | non-crystalline + peaks (broad) |
| | antisolvent, RT | |
| L-tartaric | cool, THF, −15° C.; hexane | non-crystalline |
| | antisolvent, −15° C. | |
| | slurry, 95:5 MeOH/H₂O, 40° C. | non-crystalline |
| | precipitation, DMF, acetone | non-crystalline |
| | antisolvent, RT | |
| p-toluenesulfonic | precipitation, THF, RT | not previously identified (tosylate 1) |
| | | (low crystallinity) |
| | slurry, 95:5 MeOH/H₂O, 40° C. | not previously identified (tosylate 2) + |
| | | non-crystalline |
| | precipitation, DMF, EtOAc | non-crystalline + |
| | antisolvent, RT | peaks (broad) |

Two crystalline salts were identified: fumarate and maleate salt forms; and two non-crystalline or low crystalline salts were identified: two tosylate salt forms. Poorly crystalline material was obtained from experiments involving p-toluenesulfonic acid. Overlay plots of the XRPD patterns are shown in FIGS. 1 and 2.

All samples having a unique XRPD pattern were characterized further. The results are summarized in Table 4.

TABLE 4
Characterization of unique phases

| Salt | Test | Results |
|---|---|---|
| Fumarate | NMR | Consistent with 1:1 salt |
| (Fumarate | | No organic solvents observed |
| Salt | DSC | Endo 244.6° C. |
| Form A) | TGA | 1.7% start to 125° C. (0.9 moles water) |
| | | 12.3% 125 to 275° C. (7.2 moles water) |
| | Water | <0.3 mg/ mL |
| | solubility^{a} | |
| | DSC | Endo 241.8° C. |
| | TGA | 1.7% start to 125° C. |
| | DVS | Moderately hygroscopic |
| Maleate | NMR | Consistent with a 1:1 salt |
| | | 0.7 moles of THF |
| | DSC | Endo 228.9° C. |
| | TGA | 2.3% start to 175° C. (0.3 moles THF) |
| | | 9.0% 175 to 250° C. (1.3 moles THF) |
| | Water | <0.3 mg/ mL |
| | solubility^{a} | |
| | ¹H NMR, post- | Not consistent with 1:1 salt (0.4 |
| | water slurry^{b} | moles maleic acid) |
| | ¹H NMR, post- | Consistent with a 1:1 salt |
| | EtOH slurry^{b} | 0.6 moles of THF |

^{a}Visual solubility estimation using pipet method
^{b}NMR analysis of solids recovered from slurry. The slurry was stirred overnight in the given solvent. The slurry was then centrifuged, mother liquor decanted, and the solids allowed to air dry

### Example 3: Scale Up of Fumarate Salt (Fumarate Salt Form A)

The fumarate salt (Fumarate Salt Form A) was made at a larger scale for further characterization. The experiments are summarized in Table 5.

TABLE 5
Scale up of fumarate salt (Fumarate Salt Form A)

| Experimental Details | XRPD Results |
|---|---|
| Combined 203 mg of starting material (free base compound of Formula (I)) | Fumarate Salt |
| and 1 equivalent of fumaric acid (31 mg) in a 20 mL glass vial. Add 5 mL of | Form A |
| 95:5 MeOH:water and stir magnetically at 40° C. for 3 days. Solids recovered | |
| by vacuum filtration and allowed to air dry overnight. | |
| Spatula full of fumarate salt (compound of Formula (I)) was added to an HPLC | Fumarate Salt |
| vial with 1 mL water, and stirred magnetically on 60° C. plate overnight. Solids | Form A |
| were recovered by vacuum filtration. | |
| 1 mL of 95:5 MeOH:water added to solids of fumarate salt (compound of | low crystallinity + |
| Formula (I)), stirred on 80° C. plate, and solids dissolved. Plate set to ambient. | peaks |
| Upon cooling, no solids form. Seeded with a fumarate salt crystal (compound | |
| of Formula (I)). Solids formed within 4 hours, let stirred 2 days at ambient. | |
| Solids were recovered by vacuum filtration. | |

The fumarate salt (Fumarate Salt Form A) was made successfully at larger scale and was characterized. The data is summarized in Table 6. The thermal data was consistent with the previous lot (small scale) of fumarate salt (Fumarate Salt Form A) that was characterized, showing a weight loss of 1.7% below 125° C. followed by melting around 240° C. The weight loss in the TGA suggests the material is solvated, and approximately 0.5 moles of water were observed by Karl Fischer analysis. This suggests that the fumarate salt (Fumarate Salt Form A) may be a hydrate. The fumarate salt (Fumarate Salt Form A) was moderately hygroscopic, showing a weight gain of approximately 4% up to 95% RH. The absorbed water was lost on the desorption step and the resulting material was unchanged by XRPD.

TABLE 6
Characterization of fumarate salt (Fumarate Salt Form A)

| Test | Results |
|---|---|
| XRPD | Fumarate A |
| DSC | Endo 241.8° C. |
| TGA | 1.7% start to 125° C. (0.9 moles water) |
| | 10.0% 125 to 275° C. |
| KF | 1.00% water (0.52 moles water) |
| DVS | Moderately hygroscopic |
| Post-DVS | Unchanged |
| XRPD | |

### Example 4: Fumarate Salt Polymorph Screen (Fumarate Salt Form A)

A polymorph screen of the compound of Formula (I) as a fumarate salt was conducted. The fumarate salt (Fumarate Salt Form A) polymorph screen experiments used wet solvents (class 2 or 3 solvents, e.g., wet DCM, wet EtOAc), and aqueous binary systems with high water activities (aw). Fumarate Salt Form A was prepared at approximately 800 mg scale and characterized by DSC, TGA, Karl Fisher, DVS analysis, FT-IR, Raman, and ¹H NMR.

The free base compound of Formula (I) was combined with fumaric acid under various conditions to generate a crystalline fumarate salt (Fumarate Salt Form A). Samples generated and analysed from this polymorph screening via salt formation in situ are listed in Table 7.

TABLE 7
Samples generated and analysed (Fumarate Salt Form A)

| Method | Solvent | Conditions | XRPD Pattern |
|---|---|---|---|
| ball milling | MeOH/H₂O (95/5) | 15 μL, 35 min/100 Hz | Form A (low |
| | | | crystallinity) |
| cooling | acetone/H₂O | 45° C. to RT to −15° C.: | — |
| | (70/30) | clear solution to slow | |
| | | evaporation | |
| | EtOH/H₂O (70/30) | 45° C. to RT to −15° C.: | — |
| | | dark yellow oil in | |
| | | solution to slow | |
| | | evaporation | |
| | THF/H₂O (80/20) | 45° C. to RT to −15° C.: | — |
| | | clear solution to slow | |
| | | evaporation | |
| evaporation | acetone/H₂O | slow evaporation to fast | non-crystalline + |
| | (70/30) | evaporation: orange oil | weak 30°peak |
| | | to glassy solid | |
| | ACN/H₂O (45/55) | slow evaporation: yellow | — |
| | | oil in solution | |
| | | fast evaporation: orange | |
| | | oil to glassy solid | |
| | 1,4-dioxane/ | slow evaporation: | non-crystalline + broad |
| | H₂O(60/40) | yellow, glassy solid; | peaks |
| | | vac dried | |
| | EtOH/H₂O (70/30) | slow evaporation to fast | — |
| | | evaporation: orange oil | |
| | | to glassy solid | |
| | THF/H₂O (80/20) | slow evaporation: | non-crystalline |
| | | yellow, tacky oil; vac | |
| | | dried: glassy solid | |
| freeze | dioxane/H₂O | ~100-mg scale | non-crystalline |
| drying | (60/40) | ~300-mg scale | non-crystalline |
| precipitation | DMF | dissolved at 45° C.; | non-crystalline + |
| | | acetone antisolvent, RT | broad peaks |
| | | slurry, 1 d | |
| | acetone/H₂O (60/40 | 2-PrOH antisolvent, | non-crystalline |
| | to ~70/30) | 60° C. to RT/overnight to | |
| | | 8° C., slurry, 5 hr | |
| | dioxane/H₂O | ACN antisolvent, RT | Form A |
| | (60/40) | (yellow oil) to 45° C., | |
| | | slurry, 3 d | |
| | | 1-PrOH antisolvent, RT | Form A (low |
| | | (tacky, yellow solid); | crystallinity) |
| | | 45° C. to 60° C. to | |
| | | RT(overnight) to 8° C., | |
| | | 6 d | |
| slurry | acetone | 45° C., 4 d | Form A (low |
| | | | crystallinity) |
| | DCE | 45° C., 1 d | Form A (low |
| | | | crystallinity) + |
| | | | acid |
| | DCM (wet) | RT/4 d: oily material to | Form A (low |
| | | yellow solid | crystallinity) + |
| | | | acid peaks + |
| | | | non-crystalline |
| | EtOH | 45° C., 4 d | Form A (low |
| | | | crystallinity) |
| slurry | EtOAc (wet) | 45° C., 1 d | Form A |
| | BuOAc | 45° C., 4 d | Form A (low crystallinity) |
| | MEK | 45° C., 4 d | Form A (low crystallinity) |
| | 1-pentanol | 45° C., 4 d | Form A (low crystallinity) |
| | acetone/H₂O | 45° C., 3 d | Form A |
| | (90/10) (a_{w} 0.7) | | |
| | acetone/H₂O | 45° C., clear solution | — |
| | (70/30) (a_{w} 0.85) | with stirring | |
| | ACN/H₂O (45/55) | 45° C., clear solution | — |
| | (a_{w} 0.9) | with stirring | |
| | 2-BuOH/H₂O | 45° C., 3 d | Form A |
| | (90/10) | | |
| | 1,4-dioxane/ | 45° C., 3 d | Form A |
| | H₂O (95/5) | | |
| | (a_{w} 0.4) | | |
| | 1,4-dioxane/H₂O | 45° C., clear solution | — |
| | (60/40) (a_{w} 1.0) | with stirring | |
| | DMF/H₂O (50/50) | 45° C. to RT, 2 d | Form A |
| | (a_{w} 0.8) | | |
| | DMSO/H₂O | 45° C., 2 d | Form A (disordered) + |
| | (20/80) (a_{w} 0.9) | | non-crystalline |
| | EtOH/H₂O | 45° C., 3 d | Form A |
| | (90/10) (a_{w} 0.5) | | |
| | EtOH/H₂O | 45° C., clear solution | — |
| | (70/30) (a_{w} 0.8) | with stirring | |
| | MeOH/H₂O | 45° C., 2 d | Form A (low crystallinity) |
| | (50/50) | | |
| | (a_{w} 0.75) | | |
| | MEK/H₂O (90/10) | 45° C., 3 d | Form A |
| | (a_{w} unknown) | | |
| | 1-PrOH/H₂O | 45° C., 3 d | Form A |
| | (90/10) (a_{w} 0.65) | | |
| | 2-PrOH/H₂O | 45° C., 2 d | Form A |
| | (80/20) (a_{w} 0.9) | | |
| | THF/H₂O (80/20) | 45° C., clear solution | — |
| | (a_{w} 1) | with stirring | |
| | H₂O | 45° C., 3 d | Form A |
| solvent | 2-PrOH/H₂O | RT, 1 d: tacky, orange | — |
| vapor stress | (80/20) (a_{w} 0.9) | oil | |
| | EtOH | 40° C., 1 d to 1 week: | non-crystalline + |
| | | partially solidified | Form A (broad peaks) |
| | | with some | |
| | | birefringence; air-dried | |

“—” is not applicable or not tested

The “Form A” or “A” (or Fumarate Salt Form A) designation regarding XRPD pattern of Fumarate Salt Form A is used herein to describe XRPD patterns that exhibited a higher degree of crystallinity; “low crystallinity” was used to describe XRPD patterns showing a higher degree of disorder. Form A (higher crystallinity) in these studies also exhibited crystalline disorder by XRPD, as evident by some diffuse scattering (amorphous halo) and peak broadening at higher angles (top pattern, FIG. 31). XRPD patterns showing a varying degree of crystallinity in Form A are compared in an overlay plot (FIG. 31).

Initial polymorph screening was conducted by salt formation in situ (Table 7). The fumarate salt (Fumarate Salt Form A) was found to be soluble in aqueous solvents with high water activities; however, oily or glassy materials resulted from evaporation or cooling in those solvent systems. A crystalline fumarate salt (Fumarate Salt Form A) was obtained by slurry in various organic solvents and aqueous solvent systems (Table 7). A higher crystallinity Fumarate Salt Form A was obtained in wet EtOAc and solvent systems with high water activities (Table 7).

Based on XRPD analysis, crystalline Fumarate Salt Form A appeared to be stable upon preparing a slurry in aqueous binary mixtures with high water activities (50° C. for 1 week). The slurries in non-aqueous and some of the low water activity solvents (e.g., dioxane/water 95:5, aw 0.4) resulted in disordered/low crystalline materials.

Fumarate Salt Form A (crystalline compound of Formula (I), fumarate salt) was prepared by obtaining a slurry of the free base and fumaric acid in 90:10 acetone/water, at approximately 45° C. A detailed procedure to prepare the salt at approximately 800-mg scale is described herein.

Initial physical stability evaluation of Fumarate Salt Form A was conducted at various relative humidity conditions and room temperature for approximately 1 week. While no changes were observed by XPRD analysis, the water content in the samples varied from approximately 0.6 moles at 0% relative humidity (RH) to 2.1 moles at 97% RH. The water content was found to be unchanged in the relative humidity range from 40 to 75% RH. This observation was consistent with a lower water uptake observed for that RH range by DVS analysis (FIG. 38).

Solubilities of Fumarate Salt Form A are shown in Tables 8 and 9, in selected solvents and aqueous binary mixtures.

TABLE 8
Solubilities of Fumarate Salt Form A in selected solvents

| Solvent | Solubility (mg/mL) |
|---|---|
| acetone | <1 at 60° C. |
| acetonitrile (ACN) | <1 at 60° C. |
| dichloromethane (DCM) | <1 at 60° C. |
| dimethylformamide (DMF) | >50 at RT |
| 1,4-dioxane | 1 at 60° C. |
| ethanol (EtOH) | <1 at 60° C. |
| ethyl formate | <1 at 60° C. |
| methanol (MeOH) | 1 at RT |
| methyl ethyl ketone (MEK) | <1 at 60° C. |
| tetrahydrofuran (THF) | <1 at 60° C. |
| water | <1 at 60° C. |

TABLE 9
Solubilities of Fumarate Salt Form A in aqueous binary mixtures

| Solvent mixture | Water activity, a_{w} | Solubility (mg/mL) |
|---|---|---|
| acetone/H₂O (70/30) | 0.85 | 6^{a} |
| acetonitrile/H₂O (45/55) | 0.9 | >10^{a} |
| 1,4-dioxane/H₂O (60/40) | 1.0 | 10^{a} |
| 1,4-dioxane/H₂O (95/5) | 0.4 | 1^{a} |
| ethanol/H₂O (70/30) | 0.8 | 4^{a} |
| methanol/H₂O (50/50) | 0.75 | 2^{a} |
| 2-propanol/H₂O (80/20) | 0.9 | <1^{a} |
| | | >1 at 45° C. |
| tetrahydrofuran/H₂O (80/20) | 1 | >9^{a} |

^{a}solubility without temperature

Regarding the use of crystalline Fumarate Salt Form A in polymorph screens, crystalline Fumarate Salt Form A as a starting material was mixed with different solvents under various conditions in attempts to generate polymorphs. Samples generated and analyzed are listed in Table 10.

TABLE 10
Samples Generated and analysed: Fumarate Salt Form A as starting material

| Method | Solvent | Conditions | XRPD Pattern |
|---|---|---|---|
| evaporation | DMF | Fast | Form A (low |
| | | evaporation: | crystallinity) |
| | | yellow solid | |
| slurry | acetone/H₂O (70/30) | RT, 7 d | Form A |
| | (a_{w} 0.85) | | |
| | 1,4-dioxane | 60° C., 7 d | Form A (low |
| | | | crystallinity) |
| | 1,4-dioxane/H₂O | RT, 7 d | Form A |
| | (60/40) (a_{w} 1.0) | | |
| | 1,4-dioxane/H₂O | 50° C., 7 d | Form A (low |
| | (95/5) (a_{w} 0.4) | | crystallinity) |
| | EtOH/H₂O (70/30) | RT, 7 d | Form A |
| | (a_{w} 0.8) | | |
| | MeOH | 50° C., 7 d | Form A (low |
| | | | crystallinity) |
| | MeOH/ACN (50/50) | 50° C., 7 d | Form A |
| | MeOH/i-PrOAc | 50° C., 7 d | Form A (low |
| | (50/50) | | crystallinity) |
| | MeOH/MEK (50/50) | 50° C., 7 d | Form A (low |
| | | | crystallinity) |
| | MeOH/MTBE | 50° C., 7 d | Form A (low |
| | (50/50) | | crystallinity) |
| | MeOH/H₂O (50/50) | 50° C., 7 d | Form A (low |
| | (a_{w} 0.75) | | crystallinity) |
| | 2-PrOH/H₂O (80/20) | 50° C., 7 d | Form A |
| | (a_{w} 0.9) | | |
| vapor diffusion | DMSO/acetone | RT | non-crystalline + broad |
| (solvent/antisolvent) | | | peaks |
| | DMSO/BuOAc | RT: tacky | non-crystalline (low |
| | | solid, vac | signal, small sample) |
| | | dried | |
| | DMSO/EtOH | RT | non-crystalline + broad |
| | | | peaks |

Turning to the use of the non-crystalline Fumarate Salt Form A in polymorph screens, a non-crystalline Fumarate Salt Form A was generated by freeze-drying a solution in 60:40 dioxane/water (Table 7). The non-crystalline fumarate salt (Fumarate Salt Form A) remained unchanged by XRPD analysis at 40° C./75% RH for 2 days. The non-crystalline Fumarate Salt Form A was used as starting material in additional polymorph screen experiments shown in Table 11. Samples generated and analyzed are listed in Table 11. Higher crystallinity Fumarate Salt Form A was obtained by stressing the non-crystalline material under acetone/water 90:10 and wet EtOAc vapor, i.e., higher water activity conditions.

TABLE 11
Samples generated and analysed: non-crystalline material
with Fumarate Salt Form A as starting material

| Method | Solvent | Conditions | XRPD Pattern |
|---|---|---|---|
| slurry | acetone | 50° C., 7 d | Form A (low crystallinity) |
| | 2-BuOH | 60° C., 7 d | Form A (low crystallinity) |
| | 1,4-dioxane | 60° C., 7 d | non-crystalline + broad peaks |
| | BuOAc | 60° C., 7 d | Form A (low crystallinity) |
| | 1-pentanol | 60° C., 7 d | Form A (low crystallinity) |
| | EtOH | 60° C., 7 d | Form A (low crystallinity) |
| | EtOH/H₂O | RT, 2 d: tacky oil | — |
| | (90:10) | 8° C., 1 d: tacky oil | |
| | (a_{w} 0.5) | | |
| | H₂O | 60° C., 2 d: tacky | — |
| | | oil 8° C., 1 d: tacky | |
| | | solid | |
| solvent | acetone | RT/1 hr, 40° C./2 d: | Form A (low crystallinity) |
| vapor | | wet paste, vac | |
| stress | | dried | |
| | 2-BuOH | RT/1 hr, 40° C./2 d | non-crystalline |
| | | yellow solid | |
| | DCM (wet) | RT/1 hr, 40° C./2 d: | Form A (low crystallinity) |
| | | wet paste, vac | |
| | | dried | |
| | EtOH | RT/1 hr, 40° C./11 d | non-crystalline + broad peaks |
| | EtOAc (wet) | RT/1 hr, 40° C./2 d: | Form A |
| | | wet paste, vac | |
| | | dried | |
| | acetone/H₂O | RT/1 hr, 40° C./2 d: | Form A |
| | (90:10) (a_{w} 0.7) | wet paste, vac | |
| | | dried | |
| | EtOH/H₂O | RT/1 hr, 40° C./2 d: | Form A (low crystallinity) |
| | (90/10) (a_{w} 0.5) | wet paste, vac | |
| | | dried | |
| relative | — | 40° C./75% RH, 2 d: | non-crystalline |
| humidity | | yellow solid | |
| stress | | | |

“—” is not applicable or not tested

In sum, polymorph screens showed that Fumarate Salt Form A exhibited a varying degree of crystallinity. and non-crystalline materials were observed in the polymorph screen.

Characterization of compound of Formula (I), fumarate salt (Fumarate Salt Form A) The compound of Formula (I), fumarate salt (Fumarate Salt Form A), was prepared at approximately 800-mg scale by elevated temperature slurry (45° C.) in acetone/water (9:1) (Table 12). The material was characterized by various analytical techniques as summarized in Table 13.

TABLE 12
Preparation of compounds of Formula (I),
fumarate salt (Fumarate Salt Form A)

| Method | Solvent | Conditions | XRPD Pattern |
|---|---|---|---|
| ET | acetone/H₂O | 45° C., 3 d (subsample) | Form A |
| slurry | (90/10) | 45° C., 5 d, vac dried (2 d) | Form A, high angle |
| | (a_{w} 0.7) | | peak shifting |
| | | 45° C., 3 d (subsample) | Form A, high angle |
| | | | peak shifting |
| | | 45° C., 3 d, vac dried (2 d) | Form A |
| | | | Form A |

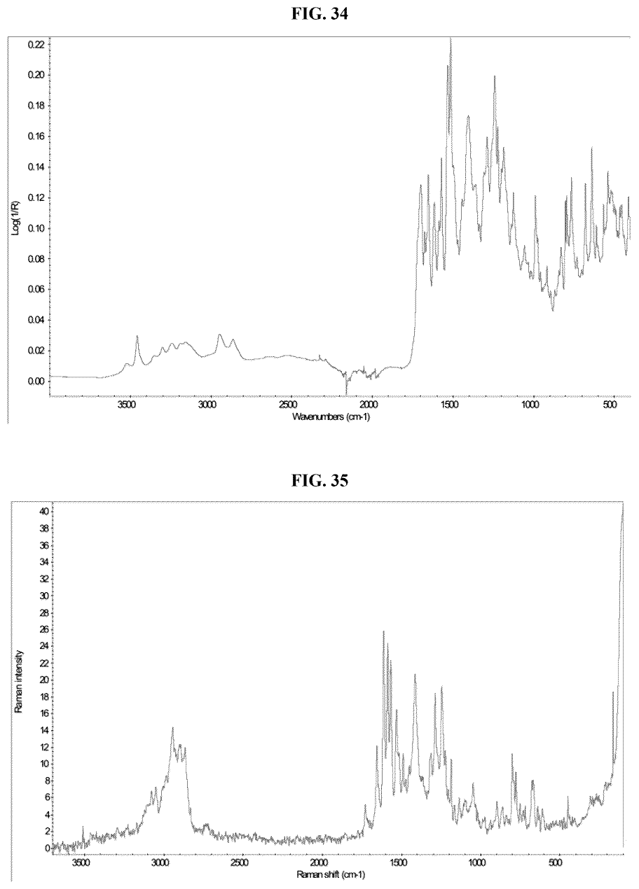

TABLE 13
Characterization of compounds of Formula
(I), fumarate salt (Fumarate Salt Form A)

| Salt | Analysis | Results |
|---|---|---|
| Fumarate | XRPD | Form A |
| Salt | | Form A, high angle peak shifting |
| Form A | DSC | Endo 47 (small, broad) |
| | | 244° C. (ΔH ~83 J/g) |
| | TGA | 1.7% wt loss from ambient to 120° C. |
| | | 2.2% wt loss from ambient to 200° C. |
| | KF-Oven | 1.49% or ~0.8 moles water |
| | | 1.45% or ~0.8 moles water |
| | DVS | 0% loss upon drying at 5% RH |
| | | 3% gain from 5 to 95% RH |
| | | 3% loss from 95 to 5% RH |
| | Post-DVS XRPD | Form A, high angle peak shifting |
| | FT-IR | FIG. 34 |
| | Raman | FIG. 35 |
| Salt | Analysis | Results |
| | NMR | Consistent with 1:1 salt; trace acetone |
| | | present |

By XRPD analysis, the fumarate salt (Fumarate Salt Form A) was crystalline with some disorder and was consistent with Fumarate Salt Form A observed in the polymorph screen study and a previously conducted salt screen. The proton NMR (¹H NMR) spectrum confirmed a 1:1 salt stoichiometry with a trace amount of acetone present (0.04 moles). Fumarate Salt Form A exhibited some peak shifting (XRPD) at higher angles after 2-day vacuum drying, as shown in FIG. 33, likely indicating variable solvent content.

By thermal analysis, Fumarate Salt Form A showed initial dissolving/dehydrating based on a small, broad endotherm at approximately 47° C.; the corresponding weight loss in TGA (approximately 1.7%) was consistent with the water content found by Karl Fischer analysis (1.5%). An additional weight loss of approximately 0.5% was observed in the range between 100 to 200° C. A probable melt (sharp, intense endotherm) was observed at approximately 244° C., immediately followed by decomposition.

By DVS analysis, the fumarate salt (Fumarate Salt Form A) was moderately hygroscopic and exhibited approximately 3% water uptake between 5-95% RH. Approximately 1% of water (or one third of the total uptake) was absorbed over each of the following relative humidity ranges: 5 to 35%; 35 to 85%; and 85 to 95% RH. The desorption curve showed a continuous weight loss from 95 to 5% RH; all of the absorbed water was lost during desorption. A hysteresis was not observed, indicating no stable hydrate was formed; however, based on continuous sorption/desorption, a variable hydrate system is possible. The XRPD pattern of post-DVS material was consistent with Fumarate Salt Form A exhibiting some peak shifting at higher angles (degrees 2Theta or ° 2θ).

FT-IR and Raman spectra of the fumarate salt (Fumarate Salt Form A) are included in FIGS. 5 and 6, respectively.

### Initial Physical Stability Evaluation of Compound of Formula (I), Fumarate Salt (Fumarate Salt Form A)

For initial physical stability evaluation, Fumarate Salt Form A was stored at various relative humidity conditions and room temperature for approximately 1 week (Table 14). Subsamples of Fumarate Salt Form A (approximately 90 mg) were weighed and placed inside relative humidity (RH) jars; after 1 week the samples were analyzed by XRPD and Karl Fisher analysis; the results are summarized in Table 14.

TABLE 14
Physical stability of compound of Formula
(I), fumarate salt (Fumarate Salt Form A)

| Starting | | XRPD | KF-Oven % Water |
|---|---|---|---|
| Form | Conditions | Result* | (moles) |
| Form A | RT, ~0% RH, ~1 week | Form A | 1.14; 1.01 |
| | | | Av: 1.08% (0.6) |
| | RT, 20% RH, ~1 week | Form A | 1.22; 1.24 |
| | | | Av: 1.23% (0.7) |
| | RT, 39% RH, ~1 week | Form A | 1.67; 1.50 |
| | | | Av: 1.59% (0.9) |
| | RT, 58% RH, ~1 week | Form A | 1.53; 1.63 |
| | | | Av: 1.58% (0.9) |
| | RT, 75% RH, ~1 week | Form A | 1.82; 1.63 |
| | | | Av: 1.73% (0.9) |
| | RT, 97% RH, ~1 week | Form A | 3.77; 3.94 |
| | | | Av: 3.86% (2.1) |

*Samples were analyzed at approximately 26-30% RH

Solubilities of the compound of Formula (I), Fumarate Salt Form A, are shown in Tables 15 and 16, in selected solvents and aqueous binary mixtures.

TABLE 15
Solubilities of Fumarate Salt Form A in selected solvents

| Solvent | Solubility (mg/mL) |
|---|---|
| acetone | <1 at 60° C. |
| acetonitrile (ACN) | <1 at 60° C. |
| dichloromethane (DCM) | <1 at 60° C. |
| dimethylformamide (DMF) | >50 at RT |
| 1,4-dioxane | 1 at 60° C. |
| ethanol (EtOH) | <1 at 60° C. |
| ethyl formate | <1 at 60° C. |
| methanol (MeOH) | 1 at RT |
| methyl ethyl ketone (MEK) | <1 at 60° C. |
| tetrahydrofuran (THF) | <1 at 60° C. |
| water | <1 at 60° C. |

TABLE 16
Solubilities of Fumarate Salt Form A in aqueous binary mixtures

| Solvent mixture | Water activity, a_{w} | Solubility (mg/mL) |
|---|---|---|
| acetone/H₂O (70/30) | 0.85 | 6^{a} |
| acetonitrile/H₂O (45/55) | 0.9 | >10 |
| 1,4-dioxane/H₂O (60/40) | 1.0 | 10^{a} |
| 1,4-dioxane/H₂O (95/5) | 0.4 | 1^{a} |
| ethanol/H₂O (70/30) | 0.8 | 4^{a} |
| methanol/H₂O (50/50) | 0.75 | 2^{a} |
| 2-propanol/H₂O (80/20) | 0.9 | <1^{a} |
| | | >1 at 45° C. |
| tetrahydrofuran/H₂O (80/20) | 1 | >9^{a} |

^{a}solubility without temperature

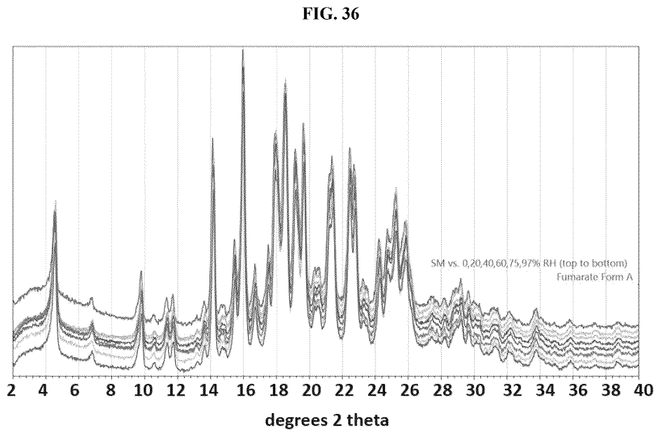

By XRPD comparison, no changes were observed between the starting material and samples after stressing at various relative humidities (FIG. 36). XRPD data in these studies were acquired at approximately 26-30% RH (laboratory RH).

Based on Karl Fisher data, the water content varied from approximately 0.6 moles at 0% RH to 2.1 moles at 97% RH; some of which may be due to surface water. In the relative humidity range from 40-75% RH, the water content was found to be unchanged (approximately 0.9 moles). This observation was consistent with a lower water uptake (approximately 0.5%) observed for that RH range by DVS analysis.

In sum, the polymorph screen revealed that Fumarate Salt Form A may have a varying degree of crystallinity depending on how it (the polymorph, etc.) is prepared, from non-crystalline to highly crystalline.

### Acid Selection

At least 11 different acids were used in the salt screen (Table 17).

TABLE 17
Acids (counterions) used in salt screen

| Acid | MW (g/mol) | pKa | Stahl Class |
|---|---|---|---|
| HCl | 36 | −6 | — |
| sulfuric (0.5 mol | 98 | −3 | 1 |
| eq.) | | | |
| p-toluenesulfonic | 172 | −1.34 | 2 |
| methanesulfonic | 96 | −1.2 | 2 |
| maleic | 116 | 1.92 | 1 |
| phosphoric | 98 | 1.96 | 1 |
| L-tartaric | 150 | 3.02 | 1 |
| fumaric | 116 | 3.03 | 1 |
| citric | 192 | 3.13 | 1 |
| hippuric | 179 | 3.55 | 1 |
| D-gluconic | 196 | 3.76 | 1 |

### Example 5: Free Base Polymorph Screen (Free Base Forms a, B, C, and D)

These studies characterized the compound of Formula (I) free base (starting material), and screened and characterized polymorphs of the free base compound of Formula (I).

Characterization data of the free base of the compound of Formula (I) are previously described. The free base compound of Formula (I) starting material was the same free base used in the, e.g., fumarate salt screen. The free base compound of Formula (I) was crystalline, although the XRPD pattern exhibited some disorder. The TGA results showed a 3.5% weight loss which corresponds to 1.6 moles of water; so, the free base compound of Formula (I) starting material may be a hydrate. No organic solvents were observed in the 1HNMR. The free base compound of Formula (I) starting material was moderately hygroscopic, exhibiting a weight gain of approximately 7% up to 95% relative humidity (DVS). The water absorbed during the experiment was lost on the desorption test, and the resulting material was unchanged by XRPD. DSC displayed the following thermograms: Endo 63.7, 99.9, 213.0, and 231.4° C.

### Polymorph Screening

The free base compound of Formula (I) was mixed with solvents under various conditions in attempts to generate crystalline material. Samples generated and analysed are listed in Table 18.

TABLE 18
Samples generated and analysed (free base of compound of Formula (I))

| Method | Solvent | Conditions | XRPD Pattern |
|---|---|---|---|
| cooling | DCM | 40° C. to −15° C.; hexane | non-crystalline + 4 peak |
| | | antisolvent, −15° C. | |
| | 1,4-dioxane | 100° C. to −15° C.; hexane | non-crystalline |
| | | antisolvent, −15° C. | |
| | DMF | 120° C. to −15° C.; toluene | low crystallinity |
| | | antisolvent, −15° C. | |
| | DMSO | 120° C. to −15° C.; toluene | non-crystalline + Free Base |
| | | antisolvent, −15° C. | Form B |
| | THF | 65° C. to −15° C.; hexane | low crystallinity |
| | | antisolvent, −15° C. | |
| | 95:5 dioxane/H₂O | 100° C. to −15° C.; hexane | low crystallinity |
| | | antisolvent, −15° C.; E | |
| | 95:5 MeOH/H₂O | 65° C. to −15° C.; Et₂O | low crystallinity |
| | | antisolvent, −15° C. | |
| | 95:5 THF/H₂O | 65° C. to −15° C.; hexane | Free Base Form A |
| | | antisolvent, −15° C. | |
| evaporation | DCM | foil with 3 pin holes, RT | non-crystalline + 4 peak |
| | 1,4-dioxane | foil with 3 pin holes, RT | low crystallinity |
| | DMF | Open vial, RT | Free Base Form C |
| | DMSO | Open vial, RT | Free Base Form B |
| | THF | foil with 3 pin holes, RT | low crystallinity |
| | 95:5 dioxane/H₂O | foil with 3 pin holes, RT | Free Base Form A |
| | 95:5 MeOH/H₂O | foil with 3 pin holes, RT | low crystallinity |
| | 95:5 THF/H₂O | foil with 3 pin holes, RT | Free Base Form A |
| precipitation | DCM | acetone antisolvent, RT | Free Base Form A |
| | | to −15° C.; | |
| | | EtOAc antisolvent, RT | Free Base Form A + non- |
| | | to −15° C.; evaporation | crystalline |
| | | Et₂O antisolvent, RT | low crystallinity |
| | | hexane antisolvent, RT | Free Base Form A (low |
| | | | crystallinity) + 4 peak |
| | | MEK antisolvent, RT | Free Base Form A |
| | | to −15° C.; evaporation | |
| | | 2-PrOH antisolvent, RT | low crystallinity |
| precipitation | DMF | ACN antisolvent, 60° C. | Free Base Form A + non- |
| | | to −15° C.; evaporation | crystalline |
| | | EtOAc antisolvent, 60° C. | Free Base Form A + non- |
| | | to −15° C.; evaporation | crystalline |
| | | MEK antisolvent, 60° C. | Free Base Form A |
| | | to −15° C.; evaporation | |
| | | 2-PrOH antisolvent, 60° C. | low crystallinity |
| | | water antisolvent, 60° C. | Free Base Form A + non- |
| | | | crystalline |
| | DMSO | EtOAc antisolvent, 60° C. | low crystallinity |
| | | to −15° C.; evaporation | |
| | | 2-PrOH antisolvent, 60° C. | low crystallinity |
| | | water antisolvent, 60° C. | low crystallinity |
| | THF | acetone antisolvent, 60° C. | Free Base Form A |
| | | to −15° C.; evaporation | |
| | | EtOAc antisolvent, 60° C. | Free Base Form A |
| | | to −15° C.; evaporation | |
| | | Et₂O antisolvent, 60° C. | low crystallinity |
| | | MEK antisolvent, 60° C. | Free Base Form A |
| | | to −15° C.; evaporation | |
| | | 2-PrOH antisolvent, 60° C. | low crystallinity |
| | | water antisolvent, 60° C. | Free Base Form A |
| slurry | acetone | 40° C., 6 days | Free Base Form A (low |
| | | | crystallinity) |
| | ACN | 40° C., 6 days | non-crystalline |
| | DCM | 40° C., 6 days | low crystallinity |
| | 1,4-dioxane | 40° C., 6 days | Free Base Form D (low |
| | | | crystallinity) |
| | DMF | 40° C., 6 days | Free Base Form A |
| | DMSO | 40° C., 6 days | non-crystalline |
| | EtOH | 40° C., 6 days | low crystallinity |
| | EtOAc | 40° C., 6 days | Free Base Form A |
| | Et₂O | 40° C., 6 days | Free Base Form D (low |
| | | | crystallinity) |
| | MeOH | 40° C., 6 days | Free Base Form A (low |
| | | | crystallinity) |
| | MEK | 40° C., 6 days | non-crystalline |
| | 2-MeTHF | 40° C., 6 days | Free Base Form A |
| | 2-PrOH | 40° C., 6 days | low crystallinity |
| | THF | 40° C., 6 days | Free Base Form D (low |
| | | | crystallinity) |
| | water | 40° C., 6 days | Free Base Form A (low |
| | | | crystallinity) |
| | 95:5 acetone/H₂O | 40° C., 6 days | Free Base Form A |
| | 95:5 ACN/H₂O | 40° C., 6 days | Free Base Form A |
| slurry | 95:5 dioxane/H₂O | 40° C., 6 days | non-crystalline + min |
| | | | Free Base Form A |
| | 95:5 EtOH/H₂O | 40° C., 6 days | Free Base Form A |
| | 95:5 2-PrOH/H₂O | 40° C., 6 days | Free Base Form A |
| | 95:5 THF/H₂O | 40° C., 6 days | Free Base Form A |

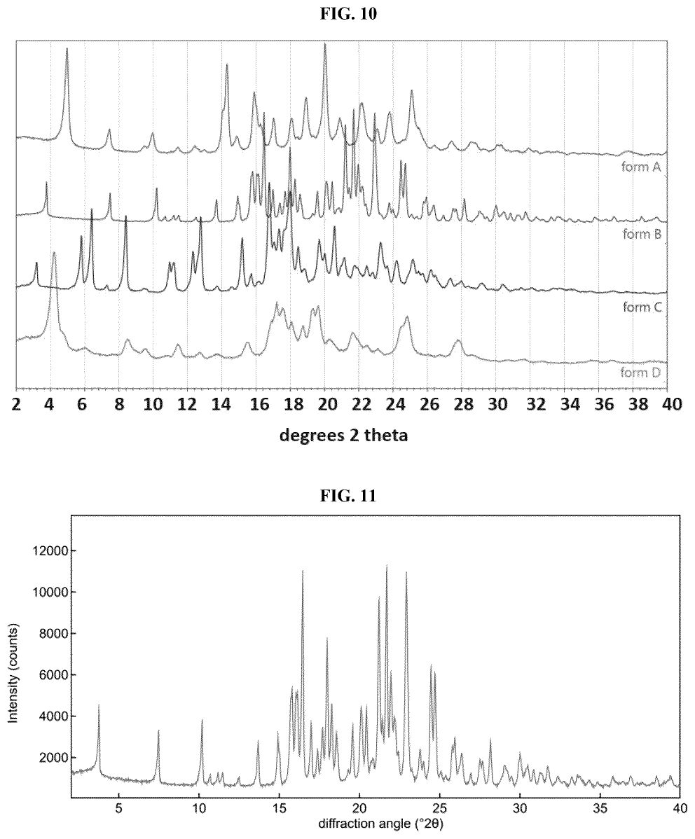

Four crystalline forms were identified during the screen, designated as Free Base Forms “A”, “B”, “C”, and “D”. An overlay plot of the XRPD patterns is shown in FIG. 10. Free Base Form A was the form most often seen in the screen. Free Base Form D was obtained as disordered (poorly crystalline) material.

The new forms were characterized by DSC and TGA. The results are summarized in Table 19.

TABLE 19
Characterization of Free Base Forms B, C, and D

| Free Base Form | Test | Result |
|---|---|---|
| B | DSC | Endo 123.9, 225.8, 236.4° C. |
| | TGA | 13.4% start to 175° C. |
| | | (1.6 moles DMSO or 6.9 moles water) NMR |
| | | shows ~2.5 moles DMSO |
| C | DSC | Endo 63.9, 202.0° C. |
| | TGA | 11.4% start to 205° C. |
| | | (1.4 moles DMF or 5.8 moles water) NMR |
| | | shows ~1 mole DMF |
| D | DSC | Endo 56.7, 162.1, 176.9° C. |
| | TGA | 1.9% start to 80° C. |
| | | (0.2 moles ether or 0.9 moles water) No |
| | | organics observed in NMR |

Samples of each form were heat stressed on a TGA. Samples were heated to the specified temperature and held at that temperature for approximately 1 minute. The material was then recovered from the TGA and analyzed by XRPD. The results are summarized in Table 20.

TABLE 20
Heat stress of Free Base Forms A, B, C, and D

| Free Base Form | Test | Result |
|---|---|---|
| A | Post-XRPD (100° C.) | Free Base Form A |
| | | (low crystallinity) |
| B | Post-XRPD (150° C.) | Free Base Form A |
| | | (low crystallinity) |
| C | Post-XRPD (150° C.) | Free Base Form C |
| D | Post-XRPD (100° C.) | Free Base Form D |

### SYNTHETIC EXAMPLES

Synthetic Example A. Synthesis of compound of Formula (I) (starting material, free form, or free base compound of Formula (I))

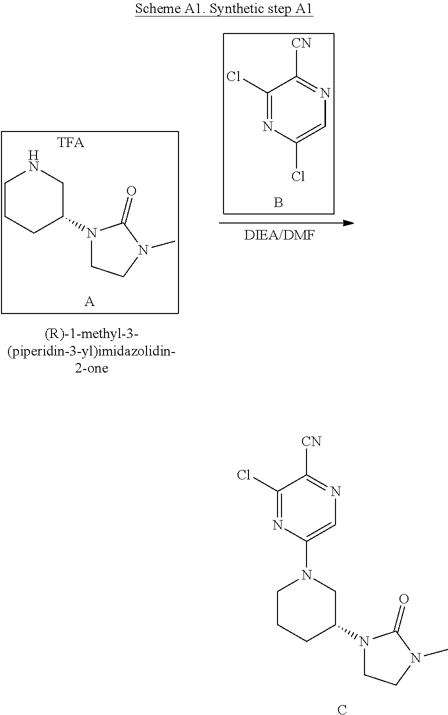

TABLE A1
Material usage, step A1

| Compound | MW | Amount | | Equiv. |
|---|---|---|---|---|
| B | 173.98 | 4.4 kg | (25.2 mol) | 1.00 |
| A | 297.28 | 13 kg | (43.7 mol) | 1.73 |
| DMF | 73.09 | 44 L + 10 L + 3.5 L | | — |
| DIEA | 129.25 | 16.3 kg | (126 mol) | 5.00 |
| sat. NH₄Cl solution | — | 100 | L | — |
| ethyl acetate | 88.11 | 120 L + 40 L | | — |
| Brine (solution) | — | 50 | L | — |
| Na₂SO₄ | — | — | | — |
| petroleum ether | 86.178 | 20 | L | — |

| | | | |
|---|---|---|---|
| MTBE | 88.15 | 21 L + 40 L | — |

“—” is not applicable

Step A1 was performed in a reaction kettle, 44.0 L of DMF was added at 20° C. Compound A (13.0 kg, 1.73 equiv., TFA) was added at 25° C. The mixture was cooled to 0° C. DIEA (22.0 L, 5.00 equiv.) was added drop-wise at 0˜5° C. over approximately 1 hr. The mixture was stirred at 0° C. for 0.5 hr. Compound B (4.40 kg, 1.00 equiv.) in dimethyl formamide (10.0 L) was added drop-wise over approximately 1 hr. The mixture was heated to 15° C. and stirred for 1.5 hrs. TLC showed that compound B was consumed.

The mixture was poured into saturated NH₄Cl solution, extracted with ethyl acetate, the combined organic layers were washed with brine, dried over Na₂SO₄, filtered, and concentrated to give crude product C.

Ethyl acetate was added to crude product C (16.2 kg, 1.00 equiv.), heated to 80° C. and stirred for 1 hr. Petroleum ether (20.0 L) was added to the mixture and stirred at 80° C. for 0.5 hr, then cooled to 25° C. and stirred for 8 hrs. The mixture was filtered. The filter cake was dried under vacuum to give the crude product C (10.5 kg, 1.00 equiv.).

A mixture of crude product C (10.3 kg, 1.00 equiv.) in dimethyl formamide (3.50 L) was stirred at 70° C. After dissolved clarification, to the mixture was added methyl tert-butyl ether (21.0 L) slowly at 70° C. The mixture was cooled to 25° C. and stirred for 12 hrs. The mixture was filtered to give a crude product. The crude product was purified by triturated with methyl tert-butyl ether (40.0 L) at 50° C. for 1 hr. After cooling to 25° C., the mixture was filtered. The filter cake was dried by vacuum drier to give the product C (7.84 kg).

Characterization of product C from Step A1. LCMS: Rt=1.909 min, m/z=321.1 (M+H)⁺. HPLC: Rt=7.807 mins, under 220 nm. ¹H NMR: (400 MHZ, CDCl₃) δ 8.06 (s, 1H), 4.40 (s, 2H), 3.80-3.73 (m, 1H), 3.42-3.30 (m, 4H), 3.18-3.12 (m, 1H), 3.02-2.95 (m, 1H), 2.81 (s, 3H), 2.05-1.92 (m, 2H), 1.84-1.75 (m, 1H), 1.72-1.64 (m, 1H).

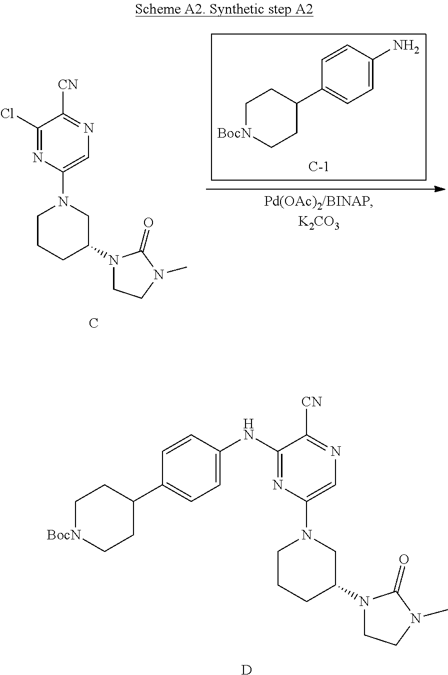

TABLE A2
Material usage, step A2

| Compound | MW | Amount | | Equiv. |
|---|---|---|---|---|
| C | 320.78 | 19.5 | kg | 1.0 |
| C-1 | 276.37 | 16.9 | kg | 1.0 |
| 2-MeTHF | 86.13 | 179 kg + 138 kg + 54 | | — |
| | | kg + 38 kg | | |
| Potassium carbonate | 138.21 | 16.8 | kg | 2.0 |
| Palladium (II) Acetate | 224.51 | 0.149 | kg | 0.01 |
| BINAP | 622.67 | 0.38 | kg | 0.01 |
| Water | 18.02 | 104 | kg | — |
| MTBE | 88.15 | 145 | kg | — |

“—” is not applicable

For step A2, A reactor was prepared as follows. Nitrogen was charged into a reactor, then 2-MeTHF (179 kg) was charged into the reactor via SS charging device, with SS diaphragm pump. Refluxed at 70-90° C. for 1 to 3 h. Lowered temperature to 20-40° C. Removed material from reactor, vacuumed and charged reactor with nitrogen.

The materials were charged as follows. 2-MeTHF (138 kg) was charged via a charging device. Compound C (19.5 kg), Compound C-1 (16.9 kg), and K₂CO₃ were charged (16.8 kg) into the reactor via a flexible isolator. The reactor was purged/charged with vacuum and nitrogen. Next, 2-MeTHF (54 kg) was charged into the reactor via charging device (SS diaphragm pump and spray ball). The reactor temperature was adjusted to 15-25° C. under nitrogen; bubbling with nitrogen continued for 0.5 to 1 hr. Palladium (II) acetate (0.149 kg) and BINAP (0.38 kg) were charged into the reactor. The reactor was purged and charged again with 2-MeTHF (38 kg). The reactor was purged/charged with vacuum and nitrogen and temperature was adjusted to 15-25° C. under nitrogen. Next, the reaction was stirred in the reactor at 20-25° C. for 0.5-1.0 h under nitrogen; the temperature was raised to 40-60° C., and then 70-80° C. and held at this temperature while stirred for 12-16 h under nitrogen. The reactor temperature was adjusted to 35 to 45° C. under nitrogen. An aliquot was removed and tested; the reaction was deemed complete.

To workup, the reactor temperature was lowered to 20-30° C. over 1.5 hours and then charged with water (104 kg) in portions over 1 hr via peristaltic pump under nitrogen, then stirred for 1-2 hours. Next, MTBE (145 kg) was charged into the reactor in portions over 0.5 to 1 hr at 20-30° C., and stirred for 1-2 hours under nitrogen at the same temperature. An aliquot was removed, and the residual crude D was confirmed to be present. To isolate compound D, the crude product was transferred to a centrifuge bag in portions, centrifuged, and then dried overnight under vacuum at 45-55° C. to obtain compound D (17.90 kg).

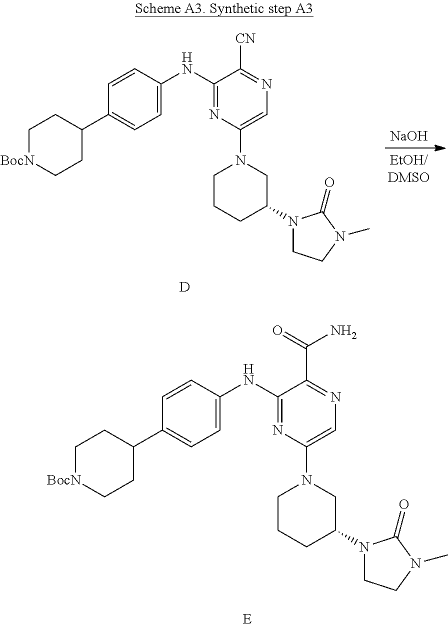

TABLE A3
Material usage, step A3

| Compound | MW | Amount | | Equiv. |
|---|---|---|---|---|
| D | 560.70 | 22.8 | kg | 1.0 |
| EtOH | 46.07 | 60 + 108 | kg | — |
| DMSO | 78.13 | 50 | kg | — |
| NaOH | 40.0 | 3.45 | kg | 2.1 |
| Water | 18.02 | 183 + 44 + 45 | kg | — |

“—” is not applicable

For step A3, the reactor was prepared by charging EtOH (60 kg), heated, distilled, refluxed, cleared, and charged with nitrogen and purged. Next, compound D (22.8 kg), DMSO (50 kg), and EtOH (108 kg) were charged into a reactor, adjusted to 20-30° C. under nitrogen. NaOH (3.45 kg) was charged, and the mixture was stirred for 1 hr. Temperature was adjusted to 40° C. for 1 hr, 50° C. for 1 hr, 60° C. for 1 hr, and 70° C. for 6-8 hrs. Next, the temperature was adjusted to 45-50° C. to remove a sample for testing, and back to 70° C. for 2-5 hours. This process was repeated until the reaction was found to be complete.

For workup, water (183 kg) was charged, and the temperature was lowered to 0-10° C., with stirring for 1 hr. The temperature was adjusted to 20-30° C. for 2-6 hours and the contents of the reactor were transferred to centrifuge bags and centrifuged. The material was dried under vacuum for 18-24 hours at 50-60° C. to obtain compound E (21.80 kg).

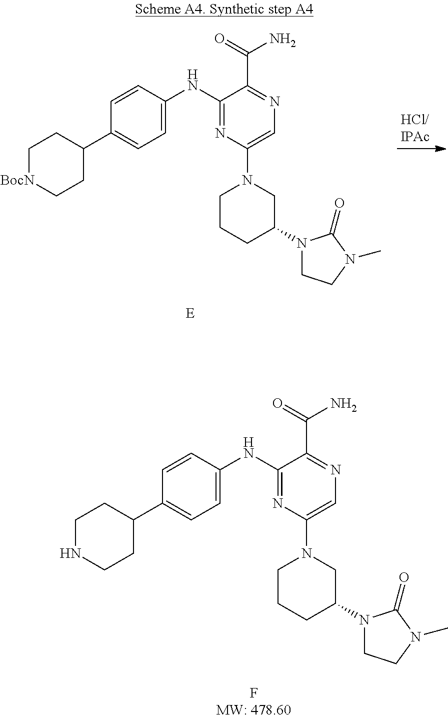

TABLE A4
Material usage, step A4

| Compound | MW | Amount | | Equiv. |
|---|---|---|---|---|
| E | 578.72 | 21.8 | kg | 1.0 |
| HCl Gas | 36.50 | 12.5 | kg | 9.0 |
| IPAc | 102.13 | 88 + 88 | kg | — |
| Water | 18.01 | 259 | kg | — |
| 10% NaOH aqueous | — | 55 | kg | — |
| solution | | | | |

“—” is not applicable

For step A4, 3M HCl IPAc solution was prepared by charging IPAc (88 kg) into the reactor, the temperature was adjusted to −10 to 10° C. under nitrogen, and then the reactor was scrubbed, and pH was confirmed. HCl gas (12.5 kg) was added to the reactor at −10 to 10° C., and stirred at −10 to 10° C. for 10-20 minutes under nitrogen. 3M HCl/IPA was obtained (100.1 kg).

The reactor was purged and charged, then compound E was charged (21.8 kg) into the reactor, followed by nitrogen, then the IPAc (88 kg) was charged to the reactor, and temperature was adjusted to 20-30° C. Next, 3M HCl IPAc (97 kg) was charged in the reactor slowly at 20-30° C. under nitrogen, and stirring continue for 3-6 hr. Crude product F was obtained and confirmed by analysis. The crude was centrifuged in centrifuge bags, processed, and dried to obtain product F (17.76 kg).

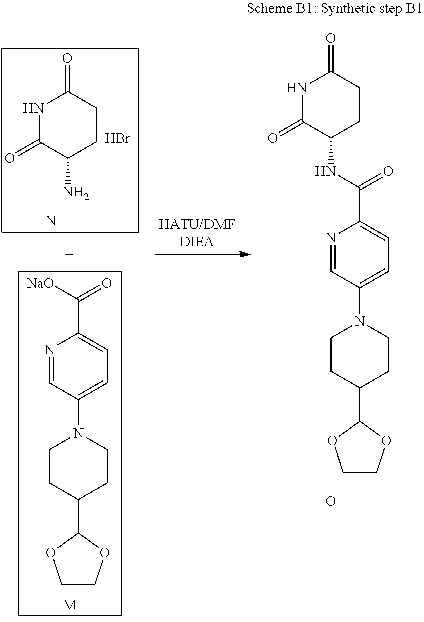

TABLE B1
Material usage, step B1

| Compound | MW | Amount | | Equiv. |
|---|---|---|---|---|
| M (Na salt) | 300.11 | 2.35 | kg | 1.0 |
| DMF | 73.09 | 35.2 | L | — |
| DIPEA | 129.25 | 2.528 | kg | 2.5 |
| N | 209.04 | 1.472 | kg | 0.9 |
| HATU | 380.23 | 4.463 | kg | 1.5 |
| KH₂PO₄ | 136.086 | 5.325 | kg | — |
| water | 18.02 | 95 + 15 + 10 | L | — |

“—” is not applicable

For step B1, compound M (2350 g, 1.0 equiv.) and DMF (35.2 L) were added to a 50 L jacketed reactor at 15˜25° C. under N₂ protection. DIPEA (2528.5 g, 2.5 equiv.) was added. Cooled to 0˜5° C. HATU (4463.4 g, 1.5 equiv.) was added in portions at 0˜5° C. Stirred at 0˜5° C. for an extra 1 h. Compound N (1472.3 g, 0.9 equiv.) was added in portions at 0˜5° C. over 30 min. Stirred at 15˜25° C. for extra 16 h.

The reaction mixture was poured into KH₂PO₄ aq. (5325 g, 5 eq., in 94.5 L H₂O) at 0˜10° C. with stirring (pH=6˜7). Next, the mixture was stirred at 0˜10° C. for extra 1 h, filtered, and the cake washed with H₂O (5 L×3). This was combined with the crude solid from another batch with similar result (2350 g×1). The batch mixture was added to water (15 L) at 15˜20° C. for 1 h to form another slurry. The slurry was filtered, and cake was washed with H₂O (2 L×2). The cake was dried under N₂ flow for 16 h to obtain 7900 g of Compound O. Purity was confirmed by HPLC, and ¹H NMR: consistent and clean.

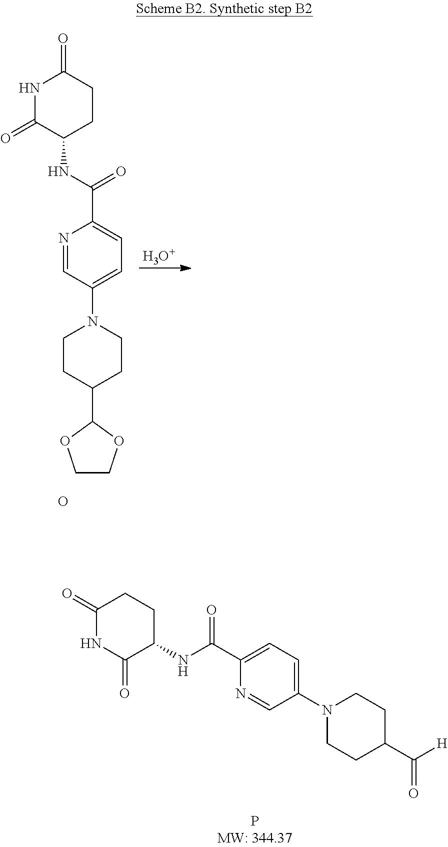

TABLE B2
Material usage, step B2

| Compound | MW | Amount | | Equiv. |
|---|---|---|---|---|
| O | 388.42 | 1.316 | kg | 1.0 |
| THF | 72.11 | 7.6 | L | — |
| water | 18.02 | 26 | L | — |
| HCl aqueous solution | — | 1.7 | L | — |
| K₂HPO₄ | 174.2 | 1.8 | kg | 5.26 |
| DCM/iPrOH (10/1) | — | 7.5 + 7 | L | — |

| | | | |
|---|---|---|---|
| Na₂SO₄ | 142.04 | — | — |
| heptane | 100.21 | — | ~3x |

“—” is not applicable

For step B2, compound O (1316 g, 1.0 equiv.) and THF (7.6 L) were added into 50 L jacketed reactor at 25˜30° C. DI water (26 L) was added. 3 M aq. HCl (1700 mL; 425 mL 12M aq. HCl+1275 mL DI H₂O) was added. The mixture was heated to 50˜55° C. and stirred for 2 h. The pH was adjusted to 7-8 by addition of K₂HPO₄ (1800 g, 5.26 eq.) portion-wise into the reaction mixture at 15˜20° C. Next, DCM/iPrOH (10/1, 7.5 L) was added. The mixture was separated, and the aqueous phase was back extracted with DCM/iPrOH (10/1, 3.5 L×2). The organic phase was dried over Na₂SO₄, filtered, and concentrated via rotary evaporation to afford 676 g of crude product P. These above steps were repeated with the crude to increase conversion.

7 batches were combined (about 6 kg); heptane (21.4 L) was added to the final precipitate and stirred at 25˜30° C. for 1 h. After filtering, the filter cake was washed with heptane and dried under air for 24 h to obtain 5300 g of product P. LC-MS: [M+H]⁺=345.5; [M+NH₄+H]⁺=363.5. ¹HNMR: Consistent and clean (with 0.122 wt % DCM; 0.209 wt % THF; 0.174 wt % iPrOH; heptanes-ND). ROA: −26.859° (1.0133 g/100 mL, MeCN).

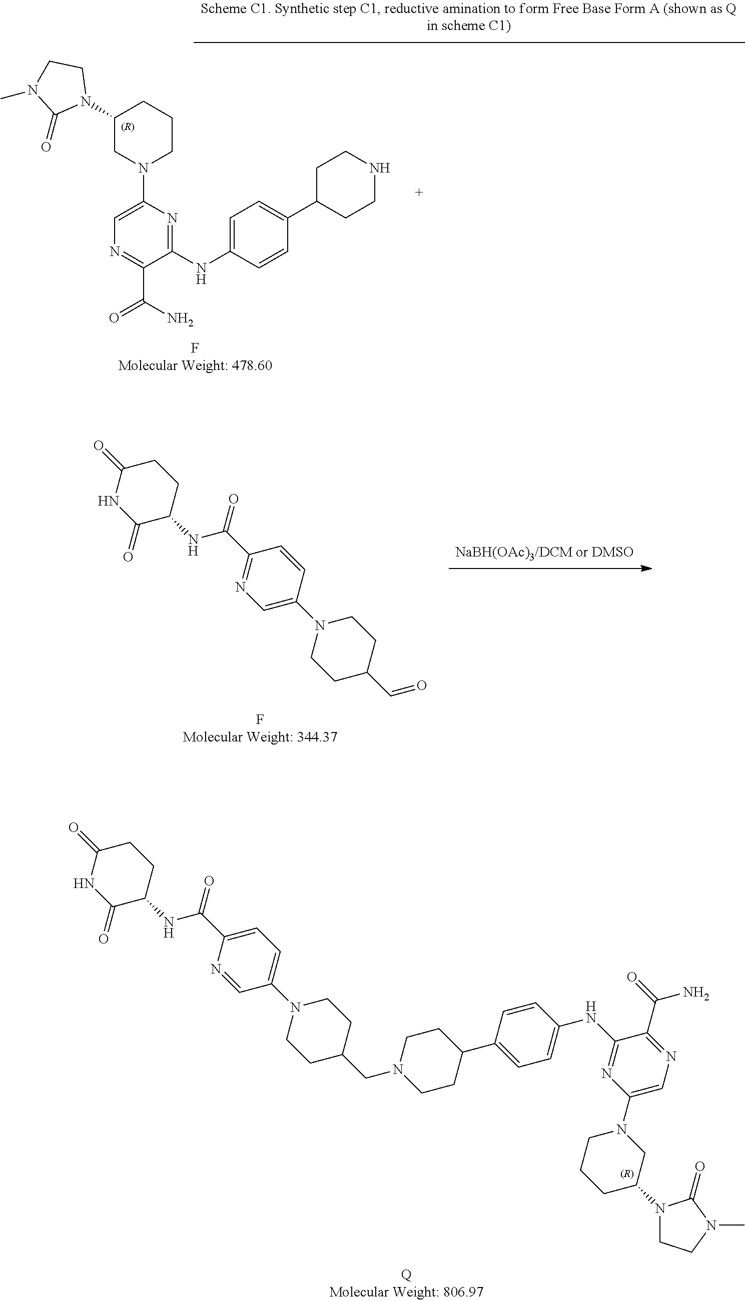

TABLE C1
Material usage, step C1, reductive amination

| Compound | MW | Amount | | Equiv. | |
|---|---|---|---|---|---|
| F (amine) | 478.60 | 1.50 kg* | (1.58 kg) | Limiting reagent | |
| P (aldehyde) | 344.37 | 1.30 | kg | 1.20 | |
| DMSO | 78.13 | 15.13 | kg | 9 | V |
| DCM | 84.93 | 1.97 kg | (1.48 L) | 0.9 | V |
| AcOH | 60.05 | 189 | g | 1.0 | eq |
| NaBH(OAc)₃ | 211.94 | 1.66 | kg | 2.5 | eq |
| Water | 18.0 | — | | 36.5 | V |
| NaOH solution (2N) | — | — | | ~4 | eq |
| ACN | 41.05 | — | | 48.5 | V |
| EtOAc | 88.11 | 13.55 | kg | 30 | V |

*corrected by assay
“—” is not applicable

For step C1, a 100 L reactor was charged with DMSO (7.68 kg), DCM (0.98 kg), compound F (amine, 1.58 kg), compound P (aldehyde, 1.30 kg, 1.20 equiv.), and again with DMSO (7.45 kg) and DCM (0.99 kg). The suspension was stirred for 1 h and charged with AcOH (189 g, 1.0 equiv.). Next, the solution was charged with NaBH (OAc) 3 (1.66 kg, 2.5 equiv.) in one equal portions at T<30° C. Stirring continued at 15-25° C. for 4 to 24 h. When the product Q was obtained, the wet cake was blown dry under nitrogen for 1 hour, and dried at ambient temperature under vacuum. Product Q (2.35 kg) was confirmed by HPLC.

Alternatively for step C1, in a round bottom flask, compound F (30.5 g) was charged with dimethylsulfoxide (DMSO, 153 mL, 5 vol), acetic acid (AcOH, 1.9 g, 0.5 eq), and the line was washed with DMSO (3 mL, 0.1 vol). The slurry was stirred for not less than (NLT) 1 hour at 25° C. A solution of compound P (24.6 g, 1.20 eq) in DMSO (153 mL, 5 vol) was prepared at 25° C. The solutions were stirred for not less than 1 hour. Visual inspection ensured that the mixture was fully dissolved. Compound P was charged in DMSO solution to reaction at 25° C. The line was washed with DMSO (30 mL, 1 vol). The slurry was stirred for 1 hour at 25° C. A solution of sodium triacetoxyborohydride (STAB, 20.2 g, 1.5 eq) in DMSO (183 mL, 6 vol) was prepared at 25° C. The solution was stirred for not less than 10 minutes. The sodium triacetoxyborohydride was fully solubilized in DMSO solution, and was charged to the reaction mixture at 20-30° C. The line was washed with DMSO (91 mL, 3 vol). The slurry was stirred for not less than 12 hours at 25° C.

In a second reactor, water (426 mL, 14 V) and THF (61 mL, 2 vol) were charged and the temperature was adjusted to 25° C. The reaction mixture was dosed over a water/THE mixture in not less than 1 hour. The internal temperature was controlled at 20-35 C. The line was washed with DMSO (30 mL, 1 vol). The mixture was stirred at 25° C. for not less than 2 hours, but for not more than 4 hours. Solid crystallization was observed toward the end of the addition or within few minutes from the end of addition; the aging time was required to crystallize the product before pH adjustment.

The pH was adjusted to 6.5-7.0 (or between 6.5 to 7.0) using NaOH (aq. 2N, ˜ 1.2 vol) at 25° C. The pH was be measured on the supernatant and not on the entire mixture. The mixture was stirred at room temperature for not less than 15 min at 25° C. and the pH was confirmed in the range of 6.5-7.0. The slurry was aged for 2-20 hours at 20-25° C. The slurry was filtered. The wet cake was washed with a mixture of THF/H₂O 1/4 (5 vol). The wet cake was washed with a mixture of tetrahydrofuran/water 1/4 (5 vol). The wet cake was washed with water (5 vol); and again with water (5 vol). The wet cake was washed with ethanol (5 vol) by reslurry. The wet cake was blown dry under nitrogen and vacuum for not less than 1 hour. The wet cake was dried at 25° C. under vacuum for not less than 23 hours. Product Q as Free Base Form A was obtained in about 85% molar yield and close to or above 99.0% a/a purity.

### Synthetic Example 1. Synthesis and Characterization of Free Base Form A

The compound of Formula (I) (16.7 mg, free base) was placed into a 20 mL glass vial. A magnetic stir bar was added, and the vial was placed on a hotplate set to 60° C. with stirring on. Tetrahydrofuran (2 mL) was slowly added until the solid dissolved. Methyl ethyl ketone (10 mL) was then slowly added until the solution became turbid. The head was turned off and the sample was cooled to room temperature. No solid was observed the next day and an additional 5 mL of methyl ethyl ketone was added. Minimal solids were observed after 6 days and the sample was placed in a freezer at −15° C. Minimal solids were observed after 2 days in the freezer and the sample was removed from the freezer, uncapped, and placed in a fume hood to allow the solvent to evaporate. The resulting solid was analysed by XRPD.

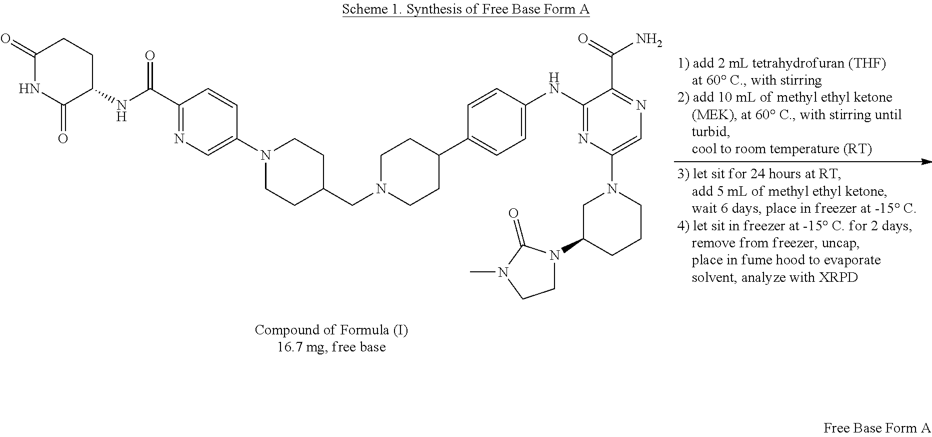 [SMILES: CN1CCN([C@@H]2CCCN(c3cnc(C(N)=O)c(Nc4ccc(C5CCN(CC6CCN(c7ccc(C(=O)N[C@H]8CCC(=O)NC8=O)nc7)CC6)CC5)cc4)n3)C2)C1=O]

The XRPD pattern of Free Base Form A is shown in FIG. 1. Table 21 is a list of the XRPD peaks for the Free Base Form A.

TABLE 21
Peaks of the XRPD pattern of Free Base Form A

| °2θ | d value (or d space, Å) | Intensity (%) |
|---|---|---|
| 4.96 ± 0.20 | 17.8158 | 74.5236 |
| 7.44 ± 0.20 | 11.8818 | 22.7838 |
| 9.42 ± 0.20 | 9.3883 | 13.4217 |
| 9.92 ± 0.20 | 8.9162 | 24.9793 |
| 11.42 ± 0.20 | 7.7483 | 13.3941 |
| 12.42 ± 0.20 | 7.1266 | 14.1259 |
| 14.00 ± 0.20 | 6.3256 | 41.3146 |
| 14.26 ± 0.20 | 6.2109 | 84.2999 |
| 14.86 ± 0.20 | 5.9614 | 21.7758 |
| 15.86 ± 0.20 | 5.5877 | 59.8730 |
| 16.24 ± 0.20 | 5.4578 | 31.8835 |
| 17.00 ± 0.20 | 5.2155 | 38.0006 |
| 18.04 ± 0.20 | 4.9171 | 36.8268 |
| 18.90 ± 0.20 | 4.6953 | 55.3714 |
| 19.98 ± 0.20 | 4.4438 | 100.0000 |
| 20.82 ± 0.20 | 4.2664 | 33.2643 |
| 22.12 ± 0.20 | 4.0185 | 55.4681 |
| 23.06 ± 0.20 | 3.8568 | 29.3289 |
| 23.68 ± 0.20 | 3.7572 | 41.0108 |
| 25.00 ± 0.20 | 3.5617 | 56.0204 |
| 26.36 ± 0.20 | 3.3810 | 15.3549 |
| 27.32 ± 0.20 | 3.2643 | 17.2880 |
| 28.50 ± 0.20 | 3.1318 | 17.3847 |
| 30.06 ± 0.20 | 2.9727 | 14.3331 |
| 31.88 ± 0.20 | 2.8070 | 12.6208 |
| 32.32 ± 0.20 | 2.7698 | 10.2458 |

The thermograms for Free Base Form A are provided in FIG. 2. The TGA (right side y-axis) showed 3.455% weight loss up to 75° C. As shown in FIG. 2 (left side y-axis), four DSC endotherms in the TGA exhibited peak maxima of 63.73° C., 99.88° C., 212.98° C., and 231.37° C.

A DVS analysis of Free Base Form A is shown in FIG. 3.

### Synthetic Example 2. Synthesis and Characterization of Free Base Amorphous Form

Free Base Amorphous Form was prepared by melt/quench of Free Base Form A (a compound of Formula (I)) (25-50 mg). An amber glass sample vial was flushed with nitrogen, and Free Base Form A was weighted into the vial. The vial was placed on a hot plate and heated to 260° C. After 45-60 seconds, when the material appeared fully molten, the vial was removed from the hot plate and quenched in liquid nitrogen. The material obtained was a light brown solid (glassy appearance). The resulting solid was analysed by XRPD analysis, ¹H NMR spectroscopy, and TGA-DSC, and DVS analysis.

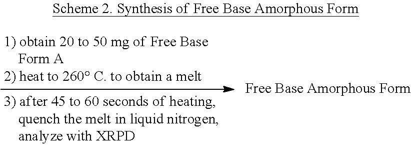

The XRPD pattern of Free Base Amorphous Form is shown in FIG. 4. Free Base Amorphous Form was analysed by XRPD and was shown to be amorphous, with an XRPD pattern as expected from an amorphous solid (FIG. 4).

The ¹H NMR spectrum (500 MHz, DMSO-d₆) of Free Base Amorphous Form is shown in FIG. 5. Analysis by 1H-NMR spectroscopy (FIG. 5) conformed to the molecular structure with no degradation observed.

The thermograms for Free Base Amorphous Form are provided in FIG. 6. The TGA (right side y-axis) showed 0.5% weight loss between 36° C. and 184° C. As shown in FIG. 6 (left side y-axis), a DTA endotherm in the TGA exhibited peak onset at 145.17° C. (in the DTA signal).

The DSC thermogram for Free Base Amorphous Form is provided in FIG. 7. The DSC thermogram (FIG. 7) concurred with the thermal behaviour seen by TGA/DTA with an initial broad endotherm between 3° and 100° C. likely due to dehydration, which was followed by a possible glass transition at 148.15° C.

Free Base Amorphous Form was physically stable to stressing at 40° C./75% relative humidity (RH) for 7 days (FIG. 8, top), and to the DVS cycling experiment (FIG. 8, bottom). DVS showed that Free Base Amorphous Form was hygroscopic with an average weight gain between 40-80% relative humidity (RH) of 3.2% (FIG. 9).

### Synthetic Example 3. Synthesis and Characterization of Free Base Form B

The compound of Formula (I) (20.0 mg, Free Base Form A) was placed into a 1-dram glass vial and of dimethyl sulfoxide (0.5 mL) was added. A magnetic stir bar was added, and the vial was placed on a stir plate at room temperature with stirring turned on. The slurry was stirred for 7 days at room temperature. The vial was centrifuged, the mother liquor was decanted, and the solid was air dried at room temperature. The resulting solid was analysed by XRPD analysis.

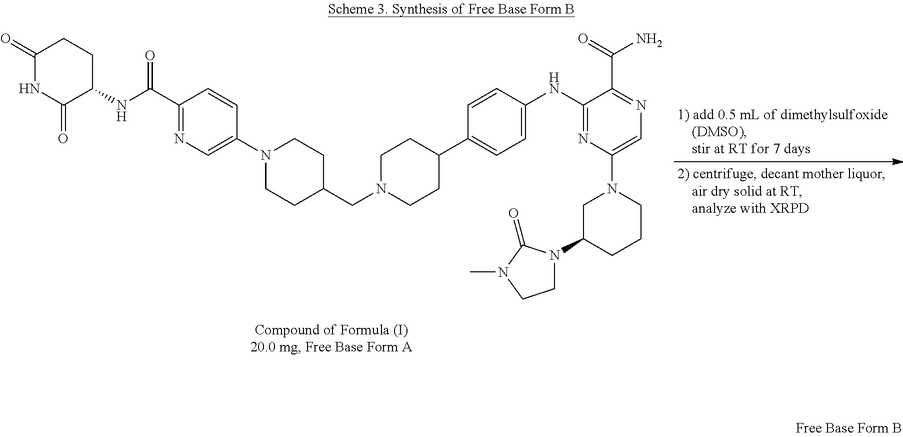 [SMILES: CN1CCN([C@@H]2CCCN(c3cnc(C(N)=O)c(Nc4ccc(C5CCN(CC6CCN(c7ccc(C(=O)N[C@H]8CCC(=O)NC8=O)nc7)CC6)CC5)cc4)n3)C2)C1=O]

The XRPD pattern of Free Base Form B is shown in FIG. 11. Table 22 is a list of the XRPD peaks for the Free Base Form B.

TABLE 22
Peaks of the XRPD pattern of Free Base Form B

| °2θ | d value (or d space, Å) | Intensity (%) |
|---|---|---|
| 3.76 ± 0.20 | 23.4985 | 39.1387 |
| 7.48 ± 0.20 | 11.8184 | 29.6874 |
| 10.20 ± 0.20 | 8.6721 | 34.1923 |
| 10.70 ± 0.20 | 8.2680 | 10.4604 |
| 11.18 ± 0.20 | 7.9140 | 11.5055 |
| 11.46 ± 0.20 | 7.7213 | 11.4695 |
| 12.50 ± 0.20 | 7.0811 | 9.5684 |
| 13.68 ± 0.20 | 6.4729 | 24.3626 |
| 14.92 ± 0.20 | 5.9376 | 27.4980 |
| 15.80 ± 0.20 | 5.6088 | 47.8782 |
| 16.04 ± 0.20 | 5.5254 | 45.9231 |
| 16.14 ± 0.20 | 5.4914 | 46.0492 |
| 16.46 ± 0.20 | 5.3854 | 97.3331 |
| 16.98 ± 0.20 | 5.2216 | 32.8498 |
| 17.38 ± 0.20 | 5.1023 | 21.5064 |
| 17.70 ± 0.20 | 5.0108 | 31.0569 |
| 17.98 ± 0.20 | 4.9334 | 68.1413 |
| 18.28 ± 0.20 | 4.8531 | 41.3821 |
| 18.56 ± 0.20 | 4.7805 | 28.9035 |
| 19.30 ± 0.20 | 4.5988 | 12.9471 |
| 19.58 ± 0.20 | 4.5337 | 31.4353 |
| 20.10 ± 0.20 | 4.4176 | 39.6973 |
| 20.44 ± 0.20 | 4.3449 | 39.3459 |
| 20.82 ± 0.20 | 4.2664 | 17.7674 |
| 21.22 ± 0.20 | 4.1869 | 86.8186 |
| 21.42 ± 0.20 | 4.1482 | 34.5166 |
| 21.70 ± 0.20 | 4.0953 | 100.0000 |
| 21.96 ± 0.20 | 4.0474 | 53.9148 |
| 22.18 ± 0.20 | 4.0078 | 35.5618 |
| 22.42 ± 0.20 | 3.9654 | 20.6145 |
| 22.92 ± 0.20 | 3.8800 | 96.8376 |
| 23.78 ± 0.20 | 3.7416 | 21.6686 |
| 24.00 ± 0.20 | 3.7078 | 16.1636 |
| 24.46 ± 0.20 | 3.6391 | 57.1493 |
| 24.70 ± 0.20 | 3.6043 | 54.4554 |
| 25.08 ± 0.20 | 3.5506 | 12.1813 |
| 25.36 ± 0.20 | 3.5120 | 10.2982 |
| 25.78 ± 0.20 | 3.4557 | 22.8579 |
| 25.94 ± 0.20 | 3.4348 | 26.6510 |
| 26.38 ± 0.20 | 3.3785 | 19.4612 |
| 26.94 ± 0.20 | 3.3095 | 11.3253 |
| 27.50 ± 0.20 | 3.2434 | 17.1547 |
| 27.68 ± 0.20 | 3.227 | 16.4970 |
| 28.16 ± 0.20 | 3.1688 | 25.1104 |
| 29.06 ± 0.20 | 3.0727 | 14.6680 |
| 29.48 ± 0.20 | 3.0299 | 9.9919 |
| 30.00 ± 0.20 | 2.9785 | 19.5964 |
| 30.48 ± 0.20 | 2.9327 | 14.7040 |
| 30.86 ± 0.20 | 2.8975 | 12.8570 |
| 31.24 ± 0.20 | 2.8631 | 11.6137 |
| 31.72 ± 0.20 | 2.8208 | 14.3526 |
| 32.36 ± 0.20 | 2.7665 | 9.9468 |
| 33.24 ± 0.20 | 2.6952 | 9.7036 |
| 33.60 ± 0.20 | 2.6672 | 10.2892 |
| 34.28 ± 0.20 | 2.6158 | 8.2530 |
| 34.82 ± 0.20 | 2.5765 | 7.4601 |
| 35.78 ± 0.20 | 2.5095 | 9.7937 |
| 36.38 ± 0.20 | 2.4695 | 7.6674 |
| 36.90 ± 0.20 | 2.4359 | 10.0910 |
| 38.52 ± 0.20 | 2.3371 | 10.0279 |
| 39.40 ± 0.20 | 2.2869 | 9.9198 |

The thermograms for Free Base Form B are provided in FIG. 12. The TGA (right side y-axis) showed 13.40% weight loss up to 175° C. As shown in FIG. 12, three DSC endotherms in the TGA exhibited peak maxima of 123.92° C., 225.75° C., and 236.42° C.

### Synthetic Example 4. Synthesis and Characterization of Free Base Form C

The compound of Formula (I) (16.1 mg, Free Base Form A) was placed into a 1-dram glass vial and N,N-dimethylformamide (0.5 mL) was added. A magnetic stir bar was added, and the vial was placed on a stir plate at room temperature with stirring turned on. The slurry was stirred for 7 days. The vial was centrifuged, the mother liquor was decanted, and the solid was air dried at room temperature. The resulting solid was analysed by XRPD analysis.

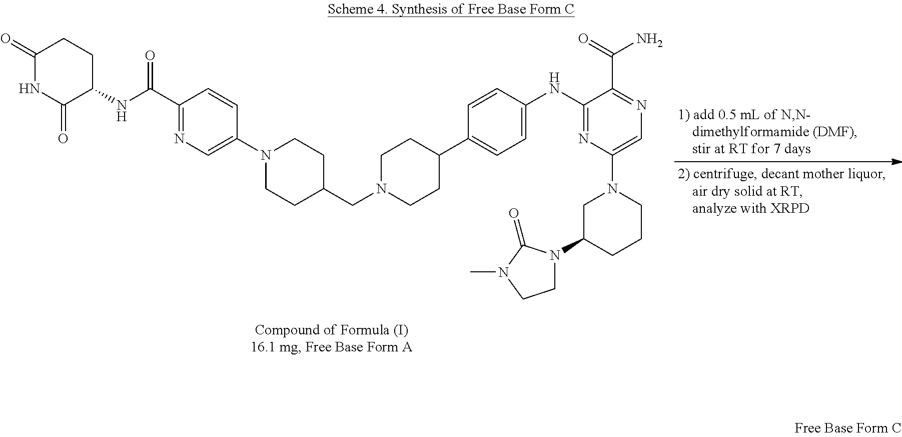 [SMILES: CN1CCN([C@@H]2CCCN(c3cnc(C(N)=O)c(Nc4ccc(C5CCN(CC6CCN(c7ccc(C(=O)N[C@H]8CCC(=O)NC8=O)nc7)CC6)CC5)cc4)n3)C2)C1=O]

The XRPD pattern of Free Base Form C is shown in FIG. 13. Table 23 is a list of the XRPD peaks for the Free Base Form C.

TABLE 23
Peaks of the XRPD pattern of Free Base Form C

| °2θ | d value (or d space, Å) | Intensity (%) |
|---|---|---|
| 3.18 ± 0.20 | 27.7830 | 33.4736 |
| 5.80 ± 0.20 | 15.2373 | 55.5606 |
| 6.40 ± 0.20 | 13.8101 | 78.1324 |
| 7.30 ± 0.20 | 12.1094 | 14.5327 |
| 8.40 ± 0.20 | 10.5259 | 72.2151 |
| 9.44 ± 0.20 | 9.3685 | 11.7157 |
| 10.96 ± 0.20 | 8.0724 | 33.3272 |
| 11.18 ± 0.20 | 7.9140 | 32.9980 |
| 12.30 ± 0.20 | 7.1958 | 42.6194 |
| 12.76 ± 0.20 | 6.9374 | 71.4103 |
| 13.68 ± 0.20 | 6.4729 | 13.4169 |
| 14.54 ± 0.20 | 6.0919 | 13.1974 |
| 15.18 ± 0.20 | 5.8365 | 53.5943 |
| 15.72 ± 0.20 | 5.6372 | 24.1540 |
| 16.16 ± 0.20 | 5.4847 | 17.7428 |
| 16.76 ± 0.20 | 5.2896 | 100.0000 |
| 17.06 ± 0.20 | 5.1973 | 50.5213 |
| 17.34 ± 0.20 | 5.1140 | 61.3042 |
| 17.98 ± 0.20 | 4.9334 | 92.7931 |
| 18.44 ± 0.20 | 4.8113 | 46.5155 |
| 18.82 ± 0.20 | 4.7150 | 27.9038 |
| 19.68 ± 0.20 | 4.5109 | 52.6797 |
| 20.02 ± 0.20 | 4.4350 | 40.5433 |
| 20.58 ± 0.20 | 4.3156 | 63.5358 |
| 21.16 ± 0.20 | 4.1986 | 37.3605 |
| 21.76 ± 0.20 | 4.0842 | 30.7664 |
| 22.46 ± 0.20 | 3.9584 | 30.3549 |
| 22.80 ± 0.20 | 3.9002 | 24.9497 |
| 23.28 ± 0.20 | 3.8208 | 50.1463 |
| 23.64 ± 0.20 | 3.7635 | 30.6201 |
| 24.20 ± 0.20 | 3.6776 | 34.8089 |
| 25.16 ± 0.20 | 3.5394 | 35.7600 |
| 25.48 ± 0.20 | 3.4957 | 26.2210 |
| 26.20 ± 0.20 | 3.4013 | 27.1630 |
| 26.46 ± 0.20 | 3.3684 | 22.6175 |
| 27.34 ± 0.20 | 3.2620 | 20.1939 |
| 27.98 ± 0.20 | 3.1888 | 17.9074 |
| 29.18 ± 0.20 | 3.0603 | 16.0691 |
| 30.40 ± 0.20 | 2.9402 | 14.9716 |
| 31.48 ± 0.20 | 2.8418 | 12.1547 |
| 32.06 ± 0.20 | 2.7917 | 12.0359 |
| 33.30 ± 0.20 | 2.6905 | 11.8529 |
| 33.70 ± 0.20 | 2.6595 | 11.8438 |
| 34.56 ± 0.20 | 2.5953 | 10.7554 |
| 36.84 ± 0.20 | 2.4397 | 9.3744 |

The thermograms for Free Base Form C are provided in FIG. 14. The TGA (right side y-axis) showed 11.36% weight loss up to 205° C. As shown in FIG. 14 (left side y-axis), two DSC endotherms in the TGA exhibited peak maxima of 63.89° C. and 201.97° C.

### Synthetic Example 5. Synthesis and Characterization of Free Base Form D

The compound of Formula (I) (17.8 mg, Free Base Form A) was placed into a 20 mL glass vial and diethyl ether (15 mL) was added. A magnetic stir bar was added, and the vial was placed on a hotplate set to 40° C. with stirring turned on. The slurry was stirred for 6 days at 40° C. The vial was centrifuged, the mother liquor was decanted, and the solid was air dried at room temperature. The resulting solid was analysed by XRPD analysis, TGA-DSC, and TGA/DTA.

Alternatively, Free Base Form D was prepared by temperature cycling Free Base Form A in acetonitrile or tetrahydrofuran. For example, Free Base Form D was prepared by temperature cycling Free Base Form A in acetonitrile between 20-79° C. with two cycles of: heating at a rate of 0.5° C./min, and cooling at a rate of 0.2° C./min.

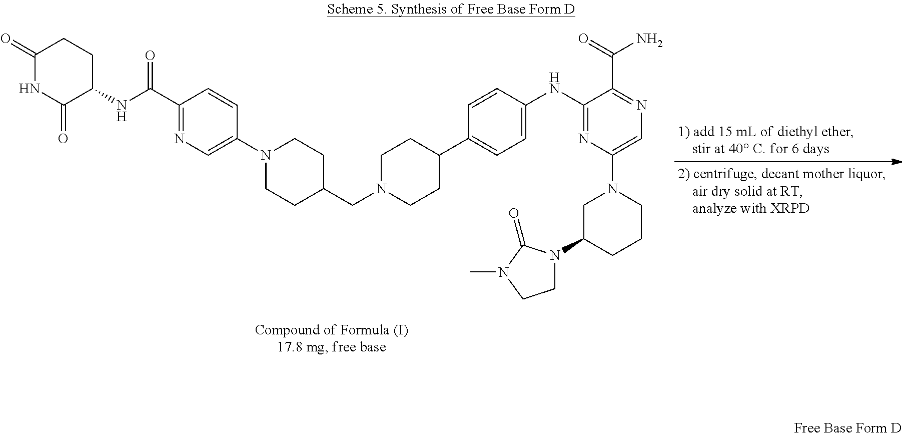 [SMILES: CN1CCN([C@@H]2CCCN(c3cnc(C(N)=O)c(Nc4ccc(C5CCN(CC6CCN(c7ccc(C(=O)N[C@H]8CCC(=O)NC8=O)nc7)CC6)CC5)cc4)n3)C2)C1=O]

The XRPD pattern of Free Base Form D is shown in FIG. 15. Table 24 is a list of the XRPD peaks for the Free Base Form D.

TABLE 24
Peaks of the XRPD pattern of Free Base Form D

| °2θ | d value (or d space, Å) | Intensity (%) |
|---|---|---|
| 4.18 ± 0.20 | 21.1383 | 100.0000 |
| 5.98 ± 0.20 | 14.7791 | 20.1933 |
| 8.48 ± 0.20 | 10.4268 | 27.3173 |
| 9.54 ± 0.20 | 9.2705 | 19.3988 |
| 11.42 ± 0.20 | 7.7483 | 23.0800 |
| 12.68 ± 0.20 | 6.9810 | 16.1149 |
| 13.78 ± 0.20 | 6.4261 | 15.0821 |
| 15.48 ± 0.20 | 5.7240 | 24.5233 |
| 17.20 ± 0.20 | 5.1553 | 58.3289 |
| 17.52 ± 0.20 | 5.0619 | 53.4560 |
| 18.06 ± 0.20 | 4.9117 | 42.0816 |
| 18.74 ± 0.20 | 4.7350 | 38.7050 |
| 19.32 ± 0.20 | 4.5941 | 52.7145 |
| 19.64 ± 0.20 | 4.5200 | 55.3099 |
| 20.26 ± 0.20 | 4.3830 | 27.4894 |
| 21.62 ± 0.20 | 4.1103 | 32.5344 |
| 22.44 ± 0.20 | 3.9619 | 22.3252 |
| 23.12 ± 0.20 | 3.8469 | 19.3194 |
| 24.84 ± 0.20 | 3.5843 | 46.3586 |
| 27.78 ± 0.20 | 3.2113 | 26.9597 |
| 30.74 ± 0.20 | 2.9085 | 10.0636 |
| 31.68 ± 0.20 | 2.8243 | 10.4608 |
| 32.68 ± 0.20 | 2.7401 | 9.6266 |
| 35.84 ± 0.20 | 2.5055 | 9.4544 |
| 36.84 ± 0.20 | 2.4397 | 9.5339 |

The thermograms for Free Base Form D are provided in FIG. 16 (TGA-DSC) and FIG. 17 (TGA/DTA). The TGA (right side y-axis) showed 1.944% weight loss up to 80° C. As shown in FIG. 16 (left side y-axis), three DSC endotherms in the TGA exhibited peak maxima of 56.73° C., 162.07° C., and 176.92° C. As shown in FIG. 17, the TGA (right side y-axis) showed 2.97% weight loss between 43° C. and 193° C. As shown in FIG. 17 (left side y-axis), one DTA endotherm in the TGA exhibited a peak onset at 173.68° C. (in the DTA signal).

### Synthetic Example 6. Synthesis and Characterization of Free Base Form E

Compound of Formula (I) (Free Base Form A) was temperature cycled in ethanol, ethyl acetate, methanol, 2-methyl-THF, or isopropanol solvent systems to prepare Free Base Form E; or Free Base Form E was prepared by evaporation of dioxane from the compound of Formula (I) (Free Base Form A).

The samples analysed here were prepared by temperature cycling Formula (I) (Free Base Form A) in ethanol between 20° C. to 75° C., with two temperature cycles. The two temperature cycles were as follows: heat at a rate of 0.5° C./min (heating rate) from 20° C. to 75° C., and cool at a rate of 0.2° C./min (cooling rate) from 75° C. to 20° C.; repeat the same heating and cooling temperature cycle. The resulting solid was analysed by XRPD analysis, ¹H NMR spectroscopy, and TGA/DTA.

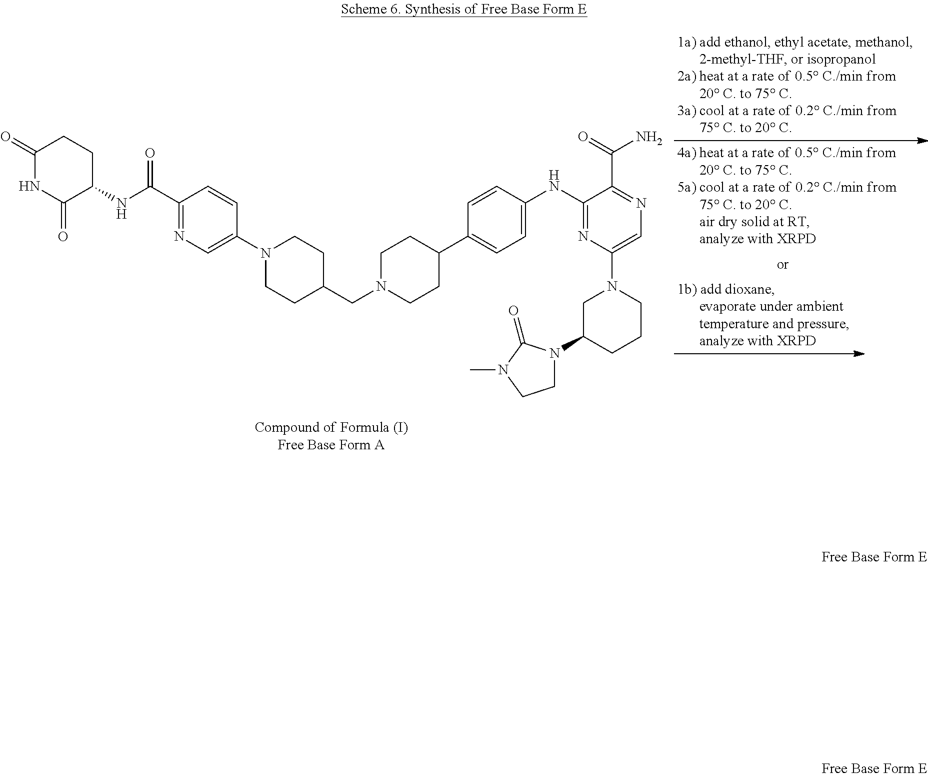 [SMILES: CN1CCN([C@@H]2CCCN(c3cnc(C(N)=O)c(Nc4ccc(C5CCN(CC6CCN(c7ccc(C(=O)N[C@H]8CCC(=O)NC8=O)nc7)CC6)CC5)cc4)n3)C2)C1=O]

The XRPD pattern of Free Base Form E is shown in FIG. 18. Table 25 is a list of the XRPD peaks for the Free Base Form E.

TABLE 25
Peaks of the XRPD pattern of Free Base Form E

| °2θ | Intensity (%) |
|---|---|
| 4.6 ± 0.20 | 100.00 |
| 6.9 ± 0.20 | 18.95 |
| 9.4 ± 0.20 | 18.79 |
| 12.3 ± 0.20 | 4.47 |
| 14.6 ± 0.20 | 46.08 |
| 15.7 ± 0.20 | 28.46 |
| 17.4 ± 0.20 | 45.58 |
| 18.6 ± 0.20 | 11.45 |
| 19.2 ± 0.20 | 10.32 |
| 20.0 ± 0.20 | 31.7 |
| 22.1 ± 0.20 | 27.92 |
| 24.5 ± 0.20 | 37.85 |

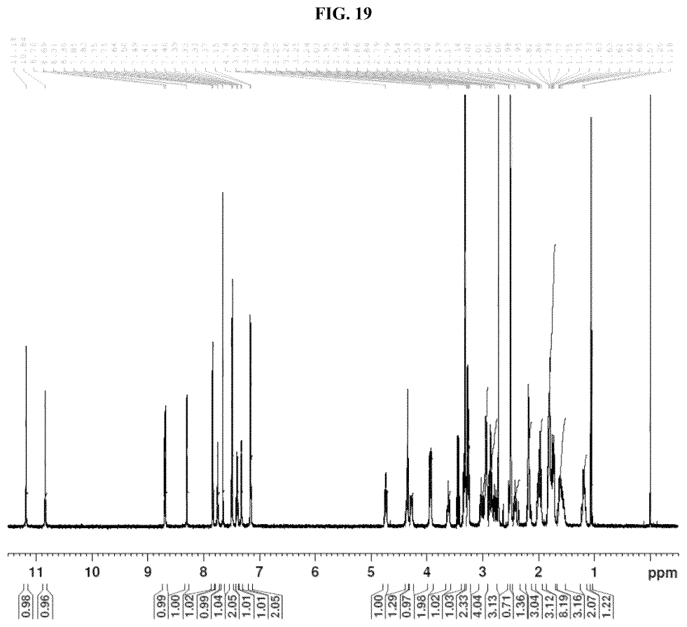

The ¹H NMR spectrum (500 MHz, DMSO-d₆) of Free Base Form E is shown in FIG. 19. An ethanol peak is shown in the ¹H NMR at 1.06 ppm, with an integration of 1.22 (ethanol in sample was further confirmed by TGA/DTA analysis).

The thermograms for Free Base Form E are provided in FIG. 20. The TGA (right side y-axis) showed 2.42% weight loss between 44° C. and 150° C. As shown in FIG. 20 (left side y-axis), a DTA endotherm in the TGA exhibited peak onset at 169.9° C. (in the DTA signal).

Free Base Form E was physically stable to stressing at 40° C./75% relative humidity (RH) for 7 days (stability study).

In one or more embodiments, Free Base Form E is an isostructural solvate (dioxane, ethanol, ethyl acetate, methanol, 2-methyl-THF, or isopropanol).

### Synthetic Example 7. Synthesis and Characterization of Free Base Form F

The compound of Formula (I) (Free Base Form A) was temperature cycled in toluene solvent system to prepare Free Base Form F.

The samples analysed here were prepared by temperature cycling Formula (I) (Free Base Form A) in toluene between 20° C. to 100° C., with two temperature cycles. The two temperature cycles were as follows: heat at a rate of 0.5° C./min (heating rate) from 20° C. to 100° C., and cool at a rate of 0.2° C./min (cooling rate) from 100° C. to 20° C.; repeat the same heating and cooling temperature cycle. The resulting solid was analysed by XRPD analysis.

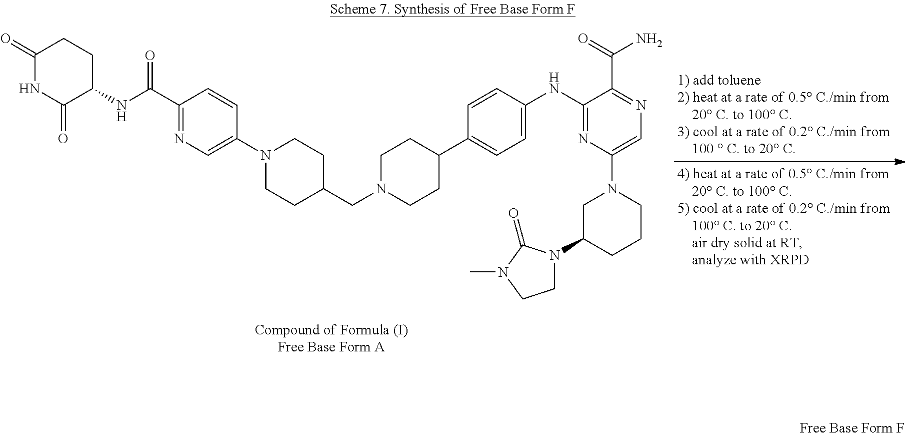 [SMILES: CN1CCN([C@@H]2CCCN(c3cnc(C(N)=O)c(Nc4ccc(C5CCN(CC6CCN(c7ccc(C(=O)N[C@H]8CCC(=O)NC8=O)nc7)CC6)CC5)cc4)n3)C2)C1=O]

The XRPD pattern of Free Base Form F is shown in FIG. 21. Table 26 is a list of the XRPD peaks for the Free Base Form F.

TABLE 26
Peaks of the XRPD pattern of Free Base Form F

| °2θ | Intensity (%) |
|---|---|
| 4.3 ± 0.20 | 100.00 |
| 6.6 ± 0.20 | 7.75 |
| 8.8 ± 0.20 | 2.9 |
| 9.5 ± 0.20 | 20.37 |
| 9.9 ± 0.20 | 2.46 |
| 10.1 ± 0.20 | 2.94 |
| 11.0 ± 0.20 | 3.95 |
| 11.9 ± 0.20 | 1.4 |
| 12.4 ± 0.20 | 4.14 |
| 13.2 ± 0.20 | 2.85 |
| 13.5 ± 0.20 | 5.28 |
| 14.0 ± 0.20 | 13.61 |
| 14.1 ± 0.20 | 7.61 |
| 14.2 ± 0.20 | 8.3 |
| 14.6 ± 0.20 | 5.24 |
| 14.8 ± 0.20 | 5.98 |
| 15.1 ± 0.20 | 5.71 |
| 15.6 ± 0.20 | 15.28 |
| 15.9 ± 0.20 | 3.64 |
| 16.4 ± 0.20 | 5.57 |
| 16.8 ± 0.20 | 17.87 |
| 17.4 ± 0.20 | 15.52 |
| 18.5 ± 0.20 | 33.75 |
| 18.9 ± 0.20 | 15.36 |
| 19.2 ± 0.20 | 12.71 |
| 19.6 ± 0.20 | 6.65 |
| 19.8 ± 0.20 | 18.88 |
| 20.6 ± 0.20 | 5.03 |
| 21.3 ± 0.20 | 10.54 |
| 21.6 ± 0.20 | 11.41 |
| 22.3 ± 0.20 | 7.46 |
| 22.9 ± 0.20 | 17.05 |
| 23.6 ± 0.20 | 2.33 |
| 24.2 ± 0.20 | 3.8 |
| 24.4 ± 0.20 | 11.43 |
| 24.9 ± 0.20 | 3.74 |
| 25.7 ± 0.20 | 5.99 |
| 25.9 ± 0.20 | 3.33 |
| 26.6 ± 0.20 | 2.32 |
| 27.0 ± 0.20 | 1.95 |
| 27.5 ± 0.20 | 1.92 |
| 28.4 ± 0.20 | 3.67 |
| 29.4 ± 0.20 | 2.66 |
| 30.0 ± 0.20 | 0.92 |
| 30.6 ± 0.20 | 1.64 |
| 31.7 ± 0.20 | 1.02 |

Free Base Form F was not physically stable to stressing at 40° C./75% relative humidity (RH) for 7 days or to ambient conditions (stability studies) including a temperature of from 15° C. to 30° C.

In one or more embodiments, Free Base Form F is a toluene solvate, which converts to Free Base Form J, to be described.

### Synthetic Example 8. Synthesis and Characterization of Free Base Form G

Free Base Form G was prepared by evaporation from a solution of the compound of Formula (I) (Free Base Form A) in dimethylacetamide (evaporation under vacuum at about 50° C.). The resulting solid was analysed by XRPD analysis, ¹H NMR spectroscopy, and TGA/DTA.

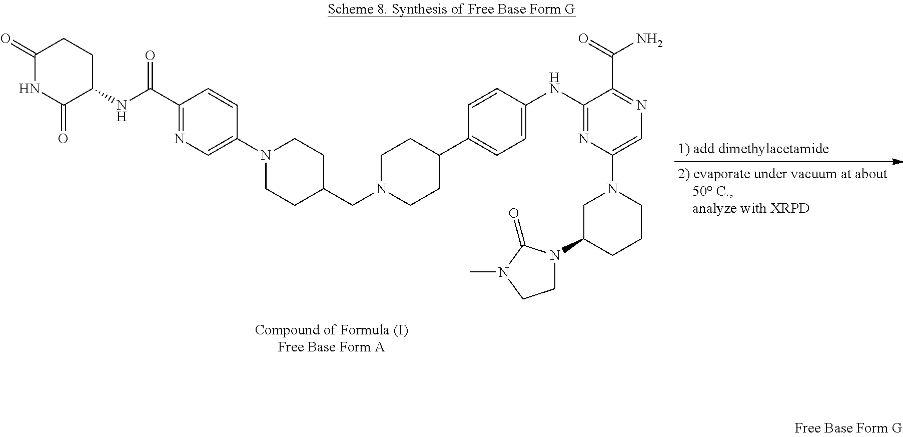 [SMILES: CN1CCN([C@@H]2CCCN(c3cnc(C(N)=O)c(Nc4ccc(C5CCN(CC6CCN(c7ccc(C(=O)N[C@H]8CCC(=O)NC8=O)nc7)CC6)CC5)cc4)n3)C2)C1=O]

The XRPD pattern of Free Base Form G is shown in FIG. 22. Table 27 is a list of the XRPD peaks for the Free Base Form G.

TABLE 27
Peaks of the XRPD pattern of Free Base Form G

| °2θ | Intensity (%) |
|---|---|
| 3.9 ± 0.20 | 41.22 |
| 7.8 ± 0.20 | 54.89 |
| 10.1 ± 0.20 | 49.48 |
| 10.6 ± 0.20 | 21.21 |
| 11.1 ± 0.20 | 32.19 |
| 12.4 ± 0.20 | 25.87 |
| 13.2 ± 0.20 | 30.34 |
| 15.1 ± 0.20 | 32.67 |
| 15.4 ± 0.20 | 36.89 |
| 15.7 ± 0.20 | 14.16 |
| 16.0 ± 0.20 | 51.21 |
| 16.4 ± 0.20 | 28.9 |
| 16.5 ± 0.20 | 100 |
| 17.2 ± 0.20 | 37.47 |
| 17.6 ± 0.20 | 36.4 |
| 18.2 ± 0.20 | 73.05 |
| 19.1 ± 0.20 | 7.93 |
| 19.3 ± 0.20 | 40.69 |
| 19.7 ± 0.20 | 12.85 |
| 20.5 ± 0.20 | 30.69 |
| 21.4 ± 0.20 | 26.27 |
| 21.6 ± 0.20 | 42.47 |
| 21.9 ± 0.20 | 95.3 |
| 22.9 ± 0.20 | 16.41 |
| 23.5 ± 0.20 | 41.87 |
| 24.9 ± 0.20 | 38.78 |
| 25.5 ± 0.20 | 48.21 |
| 26.5 ± 0.20 | 5.94 |
| 28.1 ± 0.20 | 21.33 |
| 29.0 ± 0.20 | 7.78 |
| 30.3 ± 0.20 | 7.81 |
| 30.8 ± 0.20 | 5.57 |

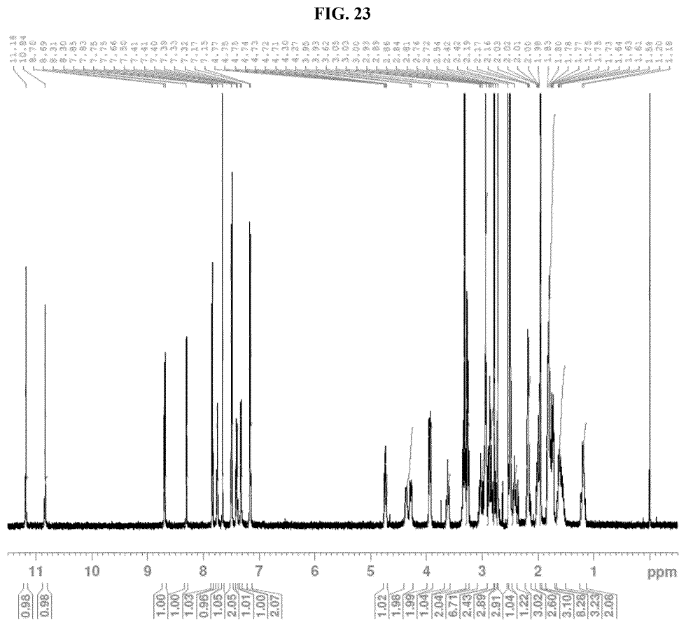

The ¹H NMR spectrum (500 MHZ, DMSO-d₆) of Free Base Form G is shown in FIG. 23. A dimethylacetamide peak is shown in the ¹H NMR at 1.96 ppm, with an integration of 3.10 (dimethylacetamide in sample was further confirmed by TGA/DTA analysis).

The thermograms for Free Base Form G are provided in FIG. 24. The TGA (right side y-axis) showed 9% weight loss between 40° C. and 300° C. As shown in FIG. 24 (left side y-axis), two DTA endotherms in the TGA exhibited peak onsets at 131.94° C. and 214.27° C. (in the DTA signal).

Free Base Form G was not physically stable to stressing at 40° C./75% relative humidity (RH) for 7 days (stability study).

In one or more embodiments, Free Base Form G is a dimethylacetamide solvate.

### Synthetic Example 9. Synthesis and Characterization of Free Base Form H

Free Base Form H was prepared by evaporation from a solution of the compound of Formula (I) (Free Base Form A) in dimethylsulfoxide (evaporation under vacuum at about 50° C.). The resulting solid was analysed by XRPD analysis, ¹H NMR spectroscopy, and TGA/DTA.

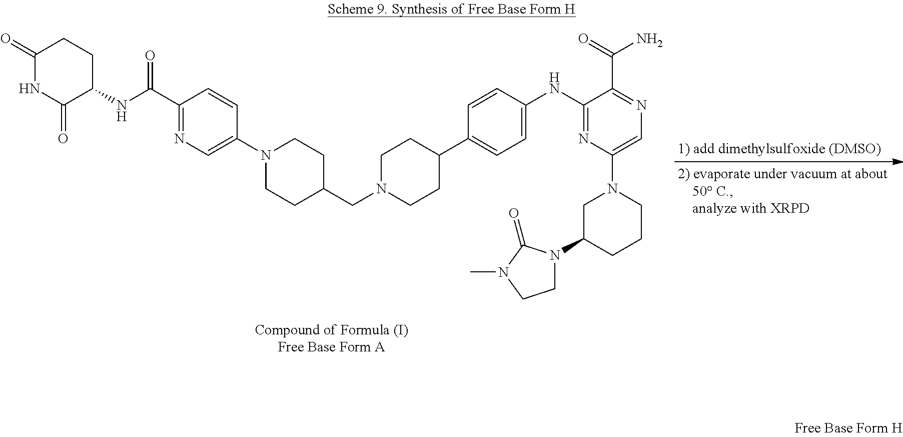 [SMILES: CN1CCN([C@@H]2CCCN(c3cnc(C(N)=O)c(Nc4ccc(C5CCN(CC6CCN(c7ccc(C(=O)N[C@H]8CCC(=O)NC8=O)nc7)CC6)CC5)cc4)n3)C2)C1=O]

The XRPD pattern of Free Base Form H is shown in FIG. 25. Table 28 is a list of the XRPD peaks for the Free Base Form H.

TABLE 28
Peaks of the XRPD pattern of Free Base Form H

| °2θ | Intensity (%) |
|---|---|
| 4.2 ± 0.20 | 63.25 |
| 6.4 ± 0.20 | 7.67 |
| 8.4 ± 0.20 | 15.69 |
| 10.2 ± 0.20 | 71.27 |
| 15.1 ± 0.20 | 29.44 |
| 15.7 ± 0.20 | 100 |
| 16.5 ± 0.20 | 64.37 |
| 16.9 ± 0.20 | 13.52 |
| 17.2 ± 0.20 | 25.98 |
| 18.1 ± 0.20 | 33.07 |
| 19.0 ± 0.20 | 11.85 |
| 20.6 ± 0.20 | 25.13 |
| 20.9 ± 0.20 | 43.32 |
| 21.9 ± 0.20 | 20.55 |
| 23.4 ± 0.20 | 34.68 |
| 25.6 ± 0.20 | 9.82 |
| 26.4 ± 0.20 | 2.99 |
| 27.0 ± 0.20 | 1.94 |
| 28.6 ± 0.20 | 6.91 |
| 28.8 ± 0.20 | 5.7 |
| 30.0 ± 0.20 | 6.14 |
| 39.7 ± 0.20 | 3.81 |

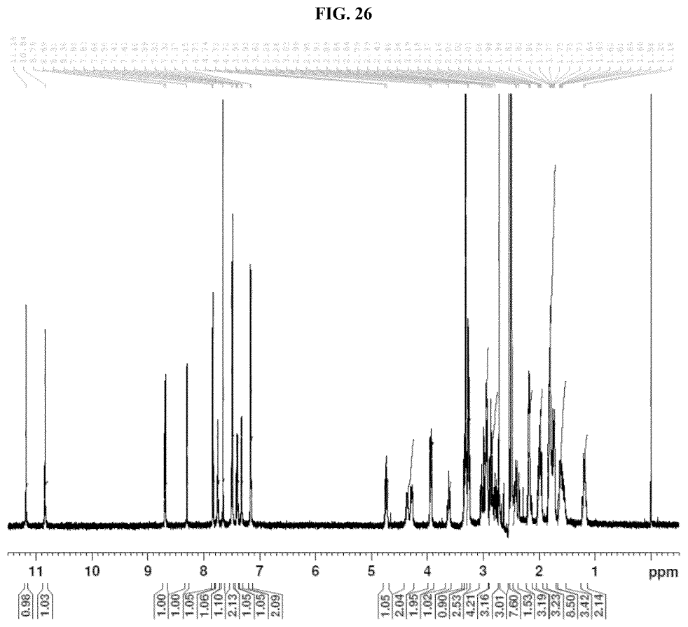

The ¹H NMR spectrum (500 MHZ, DMSO-d₆) of Free Base Form H is shown in FIG. 26. A DMSO peak is shown in the ¹H NMR.

The thermograms for Free Base Form H are provided in FIG. 27. The TGA (right side y-axis) showed 8.8% weight loss between 45° C. and 301° C. As shown in FIG. 27 (left side y-axis), two DTA endotherms in the TGA exhibited peak onsets at 175.7° C. and 231.7° C. (in the DTA signal).

Free Base Form H was physically stable to stressing at 40° C./75% relative humidity (RH) for 7 days (stability study).

In one or more embodiments, Free Base Form H is a DMSO solvate.

### Synthetic Example 10. Synthesis and Characterization of Free Base Form J

Free Base Form J was prepared by evaporation from a solution of the compound of Formula (I) (Free Base Form A) in anisole (evaporation from anisole); or Free Base Form J was prepared by via conversion of Free Base Form F stored under ambient conditions (including a temperature of from 15° C. to 30° C.). The resulting solid was analysed by XRPD analysis, ¹H NMR spectroscopy, and TGA/DTA.

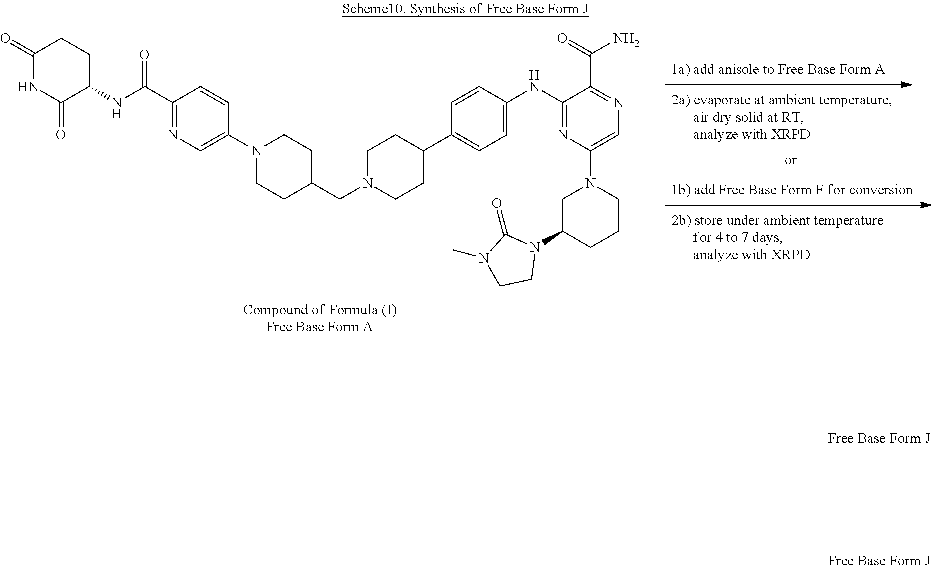 [SMILES: CN1CCN([C@@H]2CCCN(c3cnc(C(N)=O)c(Nc4ccc(C5CCN(CC6CCN(c7ccc(C(=O)N[C@H]8CCC(=O)NC8=O)nc7)CC6)CC5)cc4)n3)C2)C1=O]

The XRPD pattern of Free Base Form J is shown in FIG. 28. Table 29 is a list of the XRPD peaks for the Free Base Form J.

TABLE 29
Peaks of the XRPD pattern of Free Base Form J

| °2θ | Intensity (%) |
|---|---|
| 4.6 ± 0.20 | 100 |
| 6.9 ± 0.20 | 13.19 |
| 9.9 ± 0.20 | 32.14 |
| 12.1 ± 0.20 | 3.37 |
| 14.2 ± 0.20 | 38.18 |
| 14.9 ± 0.20 | 15.41 |
| 15.7 ± 0.20 | 46.1 |
| 17.0 ± 0.20 | 34.69 |
| 17.8 ± 0.20 | 24.4 |
| 18.4 ± 0.20 | 24.62 |
| 18.8 ± 0.20 | 12.13 |
| 19.6 ± 0.20 | 30.3 |
| 20.3 ± 0.20 | 26.6 |
| 22.0 ± 0.20 | 19.8 |
| 22.9 ± 0.20 | 21.25 |
| 24.7 ± 0.20 | 14.49 |

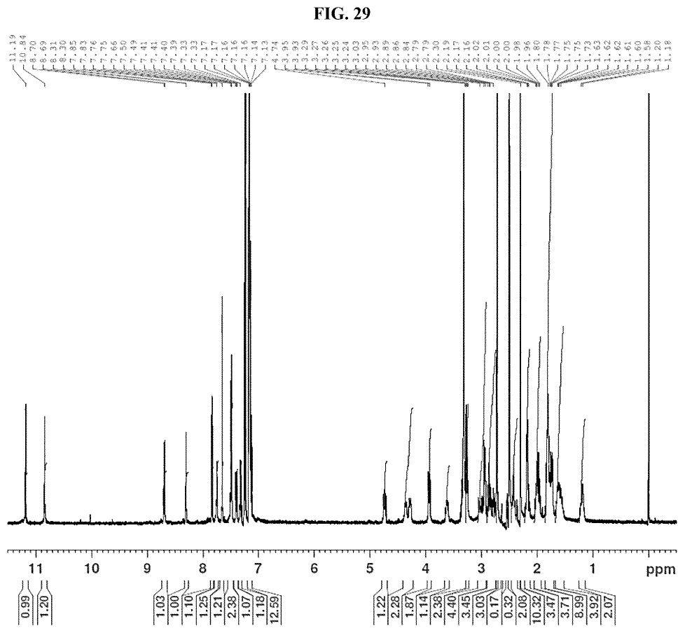

The ¹H NMR spectrum (500 MHZ, DMSO-d₆) of Free Base Form J is shown in FIG. 29.

The thermograms for Free Base Form J are provided in FIG. 30. The TGA (right side yaxis) showed 7.8% weight loss between 53° C. and 299° C. As shown in FIG. 30 (left side y-axis), a DTA endotherm in the TGA exhibited peak onset at 205.5° C. (in the DTA signal).

Free Base Form J was physically stable to stressing at 40° C./75% relative humidity (RH) for 7 days (stability study).

In one or more embodiments, Free Base Form J is an isostructural solvate (toluene or anisole).

### Synthetic Example 11. Synthesis and Characterization of Fumarate Salt Form A

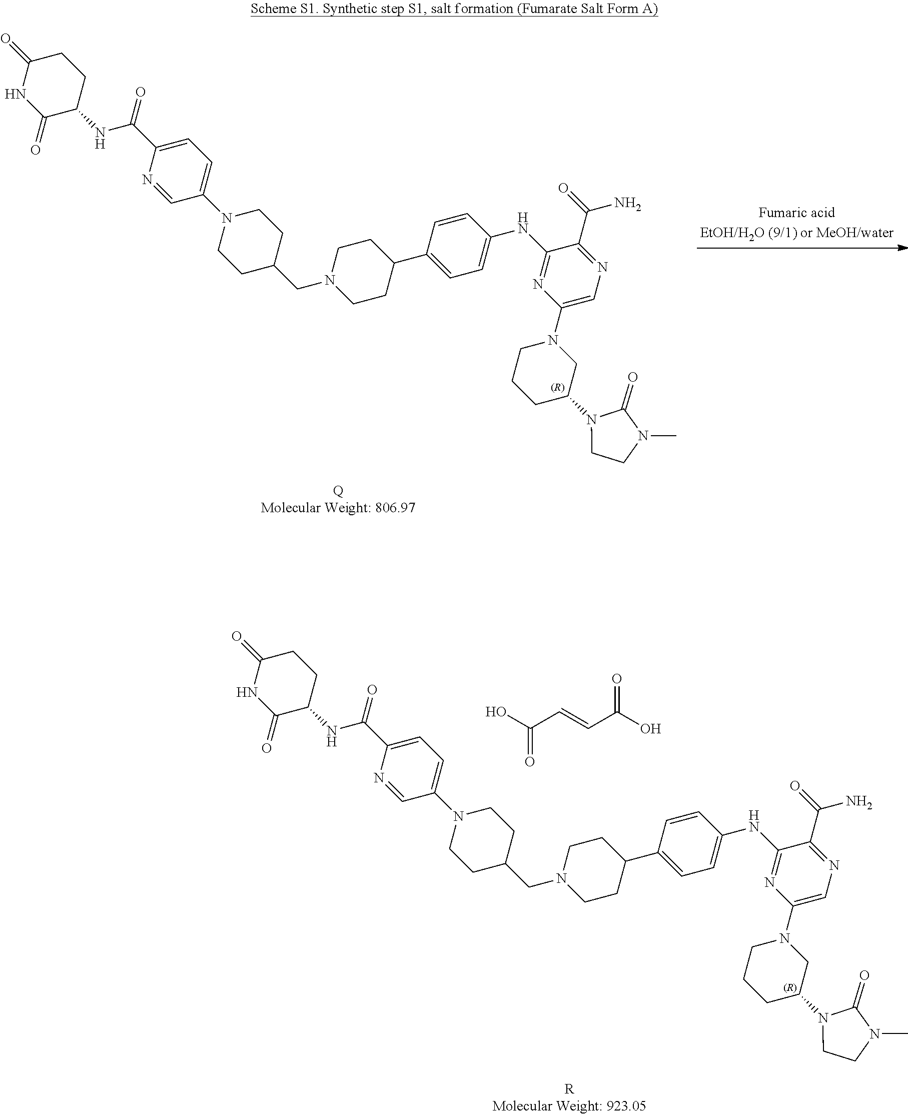

TABLE S1
Material usage, salt formation (Fumarate Salt Form A)

| Compound | MW | Amount | Equiv. | |
|---|---|---|---|---|
| Q | 806.97 | 2.35 kg | Limiting reagent | |
| EtOH | 46.07 | — | 25.2 | V |
| Water | 18.02 | — | 2.8 | V |
| fumaric acid | 116.07 | 0.378 kg | 1.12 | eq |

“—” is not applicable

For salt formation, a 100 L reactor was charged with EtOH (30.2 kg), water (4.24 kg), compound Q (2.35 kg), and fumaric acid (1.12 equiv., 0.378 kg). The mixture was stirred and warmed to 35±5° C., for 3 days. XRPD of the suspension was consistent with Fumarate Salt Form A. The heating was turned off and reaction mixture cooled to ambient, then the suspension was filtered and washed (EtOH/H₂O, 9/1 volume ratio). The filter cake was blown dry under nitrogen for 1 h. The product R was dried in a vacuum oven at about 35° C. until constant weight obtained. The solid was sieved with 20 mesh (830 μm) stainless steel sieve. Product R (2.4 kg) was obtained, and purity was confirmed by HPLC. The Fumarate Salt Form A was also obtained as follows.

### Synthetic Example 11 (Continued). Synthesis and Characterization of Fumarate Salt Form A

The compound of Formula (I) (16.9 mg, free base) and fumaric acid (2.9 mg) were placed into a 1-dram glass vial. To the vial, 0.5 mL of 95:5 methanol: water (v: v) and a magnetic stir bar were added. The vial was placed on a hotplate set to 40° C. with stirring turned on. The slurry was stirred and heated at 40° C. for 7 days. The vial was centrifuged, the mother liquor was decanted, and the solid was air dried at room temperature. The resulting solid was analysed by XRPD.

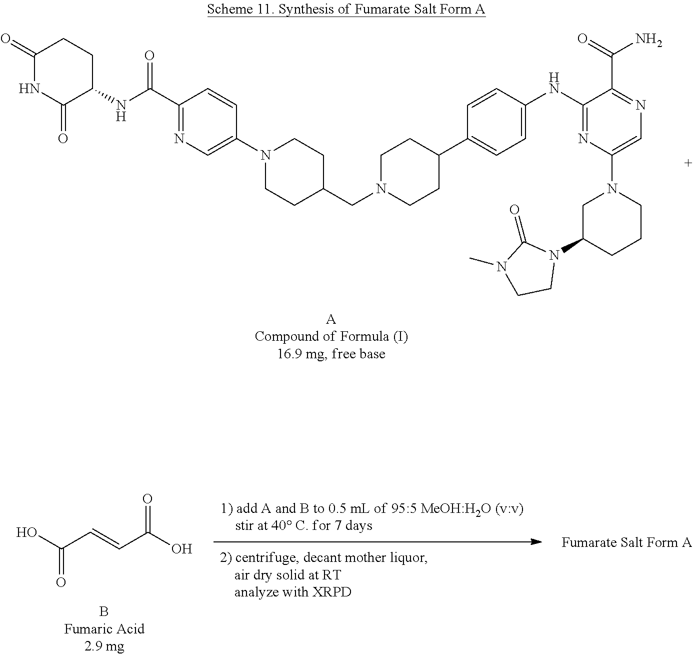 [SMILES: CN1CCN([C@@H]2CCCN(c3cnc(C(N)=O)c(Nc4ccc(C5CCN(CC6CCN(c7ccc(C(=O)N[C@H]8CCC(=O)NC8=O)nc7)CC6)CC5)cc4)n3)C2)C1=O ; O=C(O)/C=C/C(=O)O]

The XRPD pattern of Fumarate Salt Form A is shown in FIG. 32. Table 30 is a list of the XRPD peaks for the Fumarate Salt Form A.

TABLE 30
Peaks of the XRPD pattern of Fumarate Salt Form A

| °2θ | d value (or d space, Å) | Intensity (%) |
|---|---|---|
| 4.58 ± 0.20 | 19.2930 | 44.0961 |
| 6.84 ± 0.20 | 12.9227 | 14.8329 |
| 9.82 ± 0.20 | 9.0068 | 27.3847 |
| 10.64 ± 0.20 | 8.3144 | 14.2596 |
| 11.38 ± 0.20 | 7.7754 | 18.1630 |
| 11.72 ± 0.20 | 7.5506 | 16.7846 |
| 13.62 ± 0.20 | 6.5012 | 17.6141 |
| 14.18 ± 0.20 | 6.2457 | 80.6416 |
| 14.72 ± 0.20 | 6.0178 | 16.4552 |
| 15.52 ± 0.20 | 5.7094 | 40.4123 |
| 16.04 ± 0.20 | 5.5254 | 87.9239 |
| 16.76 ± 0.20 | 5.2896 | 34.1059 |
| 17.54 ± 0.20 | 5.0561 | 35.7648 |
| 18.00 ± 0.20 | 4.9279 | 95.7917 |
| 18.48 ± 0.20 | 4.8010 | 99.6585 |
| 19.18 ± 0.20 | 4.6273 | 89.6926 |
| 19.64 ± 0.20 | 4.5200 | 64.1864 |
| 20.70 ± 0.20 | 4.2909 | 27.7141 |
| 21.16 ± 0.20 | 4.1986 | 80.5318 |
| 22.56 ± 0.20 | 3.9411 | 100.0000 |
| 23.36 ± 0.20 | 3.8079 | 25.2623 |
| 24.06 ± 0.20 | 3.6987 | 31.2759 |
| 24.64 ± 0.20 | 3.6129 | 41.1930 |
| 25.22 ± 0.20 | 3.5312 | 50.9149 |
| 25.58 ± 0.20 | 3.4823 | 53.7204 |
| 26.06 ± 0.20 | 3.4192 | 23.4447 |
| 27.36 ± 0.20 | 3.2596 | 24.7011 |
| 27.98 ± 0.20 | 3.1888 | 17.0285 |
| 28.72 ± 0.20 | 3.1083 | 24.1766 |
| 29.18 ± 0.20 | 3.0603 | 25.3354 |
| 29.68 ± 0.20 | 3.0099 | 23.0666 |
| 30.30 ± 0.20 | 2.9497 | 16.4674 |
| 31.08 ± 0.20 | 2.8774 | 17.0285 |
| 31.58 ± 0.20 | 2.8330 | 13.2837 |
| 32.22 ± 0.20 | 2.7782 | 15.0646 |
| 33.76 ± 0.20 | 2.6549 | 14.9183 |

The solution ¹H NMR spectrum for Fumarate Salt Form A was consistent with the chemical structure of the compound of Formula (I) and contained peaks attributed to fumaric acid.

The thermograms for Fumarate Salt Form A are provided in FIG. 37. The TGA (right side y-axis) showed 1.715% weight loss up to 125° C. and an additional 12.31% weight loss from 125° C. to 275° C. As shown in FIG. 37, a DSC endotherm in the TGA exhibited a peak maximum of 244.55° C.

A DVS analysis of Fumarate Salt Form A is shown in FIG. 38.

### Synthetic Example 12. Synthesis and Characterization of Fumarate Salt Amorphous Form

Fumarate Salt Amorphous Form was prepared by lyophilisation of a suspension of Fumarate Salt Form A (a compound of Formula (I)) (15 mg/mL) in acetonitrile/water (1:3). The suspension was heated to 80° C. to afford a (hot) solution. The hot solution was then frozen in liquid nitrogen and freeze dried (lyophilized, 24 hours) to afford Fumarate Salt Amorphous Form. The resulting solid was analysed by XRPD analysis, ¹H NMR spectroscopy, and TGA/DTA, DSC, and DVS analysis.

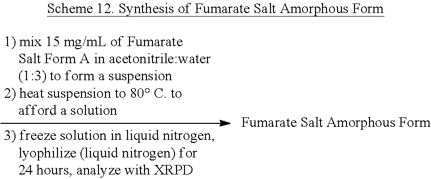

The XRPD pattern of Fumarate Salt Amorphous Form is shown in FIG. 39. Fumarate Salt Amorphous Form was analysed by XRPD and was shown to be amorphous, with an XRPD pattern as expected from an amorphous solid (FIG. 39).

The ¹H NMR spectrum (500 MHZ, DMSO-d₆) of Fumarate Salt Amorphous Form is shown in FIG. 40. Analysis by 1H-NMR spectroscopy (FIG. 40) conformed to the molecular structure and minimal residual acetonitrile content (max 0.07 mol. eq.).

The thermograms for Fumarate Salt Amorphous Form are provided in FIG. 41. The TGA (right side y-axis) showed 4.66% weight loss between about 35° C. and about 180° C. As shown in FIG. 41 (left side y-axis), a DTA endotherm in the TGA exhibited peak onset at 93.95° C. (in the DTA signal). Decomposition of the Fumarate Salt Amorphous Form was observed at ˜220° C.

The DSC thermogram for Free Base Amorphous Form is provided in FIG. 42. The DSC thermogram (FIG. 42) concurred with the thermal behaviour seen by TGA/DTA with an initial broad endotherm between 3° and 130° C. likely due to dehydration, which was followed by a possible glass transition at 150.92° C.

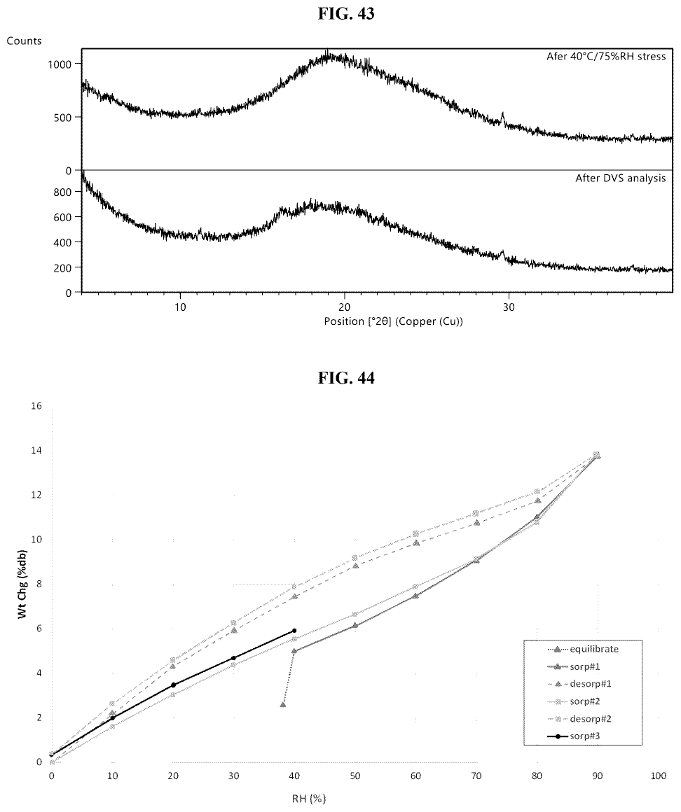

Fumarate Salt Amorphous Form was physically stable to stressing at 40° C./75% relative humidity (RH) for 7 days (FIG. 43, top), and to the DVS cycling experiment (FIG. 43, bottom). However, very small diffraction peaks seem to appear in both diffractograms. DVS showed that Fumarate Salt Amorphous Form was hygroscopic with an average weight gain between 40-80% relative humidity (RH) of 5.6% (FIG. 44).

### Synthetic Example 13. Synthesis and Characterization of Fumarate Salt Form B

Fumarate Salt Form B was prepared by isolation from a slurry of Fumarate Salt Amorphous Form in methanol (MeOH). Alternatively, Fumarate Salt Form B was prepared by salt formation in methanol with Free Base Form A and 0.5 mol. equivalents of fumaric acid. The resulting solid was analysed by XRPD analysis and ¹H NMR spectroscopy.

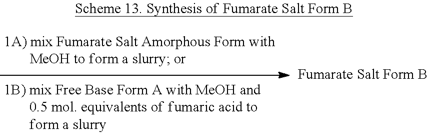

The XRPD pattern of Fumarate Salt Forms A and B are shown in FIG. 45: Fumarate Salt Form A (top); Fumarate Salt Form A+Fumarate Salt Form B (2^{nd} from top); Fumarate Salt Form B (2^{nd} from bottom); and Fumarate Salt Form B after stressing for one week at 40° C. and 75% relative humidity (bottom). Table 31 is a list of the XRPD peaks for the Fumarate Salt Form B.

TABLE 31
Peaks of the XRPD pattern of Fumarate
Salt Form B (hemi-fumarate salt)

| °2θ | Intensity (%) |
|---|---|
| 4.54 ± 0.20 | 1.31 |
| 6.02 ± 0.20 | 14.33 |
| 6.60 ± 0.20 | 23.34 |
| 7.15 ± 0.20 | 9.77 |
| 7.54 ± 0.20 | 25.61 |
| 7.67 ± 0.20 | 100.0 |
| 8.57 ± 0.20 | 15.20 |
| 8.88 ± 0.20 | 1.60 |
| 10.44 ± 0.20 | 5.87 |
| 10.82 ± 0.20 | 7.32 |
| 11.74 ± 0.20 | 12.08 |
| 12.04 ± 0.20 | 2.79 |
| 12.42 ± 0.20 | 2.17 |
| 12.71 ± 0.20 | 7.17 |
| 13.63 ± 0.20 | 5.11 |
| 13.95 ± 0.20 | 6.14 |
| 14.35 ± 0.20 | 3.76 |
| 14.97 ± 0.20 | 13.6 |
| 15.17 ± 0.20 | 4.09 |
| 15.90 ± 0.20 | 5.00 |
| 16.42 ± 0.20 | 29.47 |
| 17.19 ± 0.20 | 13.71 |
| 17.83 ± 0.20 | 6.78 |
| 18.18 ± 0.20 | 15.94 |
| 18.49 ± 0.20 | 13.49 |
| 18.96 ± 0.20 | 9.91 |
| 19.89 ± 0.20 | 9.80 |
| 20.09 ± 0.20 | 3.34 |
| 20.43 ± 0.20 | 2.14 |
| 20.80 ± 0.20 | 7.45 |
| 21.56 ± 0.20 | 71.79 |
| 22.09 ± 0.20 | 9.42 |
| 22.76 ± 0.20 | 4.86 |
| 23.21 ± 0.20 | 10.16 |
| 23.74 ± 0.20 | 7.85 |
| 24.78 ± 0.20 | 4.11 |
| 25.35 ± 0.20 | 2.98 |
| 26.52 ± 0.20 | 2.04 |
| 27.31 ± 0.20 | 4.97 |
| 28.53 ± 0.20 | 1.97 |
| 30.47 ± 0.20 | 1.17 |
| 31.29 ± 0.20 | 1.07 |
| 31.89 ± 0.20 | 1.69 |
| 33.51 ± 0.20 | 0.93 |
| 35.56 ± 0.20 | 0.79 |
| 38.03 ± 0.20 | 0.86 |

The ¹H NMR spectrum (500 MHZ, DMSO-d₆) of Fumarate Salt Form B is shown in FIG. 46. Analysis by ¹H-NMR spectroscopy (FIG. 46) conformed to the molecular structure and showed that Fumarate Salt Form B is a hemi-fumarate salt.

### Synthetic Example 14. Synthesis and Characterization of Fumarate Salt Form C

Fumarate Salt Form C was prepared by isolation from a salt formation with Fee Base Form A and 0.5 mol. equivalent of fumaric acid in methanol/water (84/16). Alternatively, Fumarate Salt Form C was prepared by temperature cycling between 5° C. and 60° C. Fumarate Salt Form B in methanol/water (84/16). The resulting solid was analysed by XRPD analysis and ¹H NMR spectroscopy.

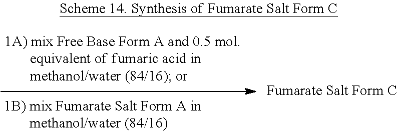

The XRPD pattern of Fumarate Salt Forms A and C are shown in FIG. 47: Fumarate Salt Form A (top); Fumarate Salt Form A+Fumarate Salt Form C (2^{nd} from top); Fumarate Salt Form C (2^{nd} from bottom); and Fumarate Salt Form C after stressing for one week at 40° C. and 75% relative humidity (bottom). Table 32 is a list of the XRPD peaks for the Fumarate Salt Form C.

TABLE 32
Peaks of the XRPD pattern of Fumarate
Salt Form C (hemi-fumarate salt)

| °2θ | Intensity (%) |
|---|---|
| 4.89 ± 0.20 | 1.79 |
| 6.14 ± 0.20 | 44.78 |
| 6.38 ± 0.20 | 3.84 |
| 7.69 ± 0.20 | 40.03 |
| 8.08 ± 0.20 | 36.83 |
| 8.18 ± 0.20 | 21.28 |
| 8.44 ± 0.20 | 19.89 |
| 9.51 ± 0.20 | 2.1 |
| 11.37 ± 0.20 | 16.36 |
| 12.14 ± 0.20 | 28.25 |
| 12.86 ± 0.20 | 4.87 |
| 13.53 ± 0.20 | 16.16 |
| 14.21 ± 0.20 | 1.95 |
| 14.37 ± 0.20 | 3.67 |
| 15.44 ± 0.20 | 34.56 |
| 16.23 ± 0.20 | 10.7 |
| 16.42 ± 0.20 | 4.62 |
| 16.61 ± 0.20 | 3.54 |
| 16.80 ± 0.20 | 6.01 |
| 16.95 ± 0.20 | 5.26 |
| 17.51 ± 0.20 | 3.47 |
| 17.67 ± 0.20 | 11.47 |
| 18.01 ± 0.20 | 15.94 |
| 18.35 ± 0.20 | 12.16 |
| 18.51 ± 0.20 | 13.00 |
| 19.16 ± 0.20 | 13.28 |
| 19.28 ± 0.20 | 17.02 |
| 19.71 ± 0.20 | 2.60 |
| 20.47 ± 0.20 | 18.61 |
| 20.89 ± 0.20 | 20.81 |
| 21.13 ± 0.20 | 100.00 |
| 21.96 ± 0.20 | 3.89 |
| 22.30 ± 0.20 | 1.75 |
| 22.84 ± 0.20 | 2.26 |
| 23.04 ± 0.20 | 4.57 |
| 23.59 ± 0.20 | 19.13 |
| 23.74 ± 0.20 | 11.29 |
| 24.03 ± 0.20 | 22.12 |
| 24.43 ± 0.20 | 9.28 |
| 24.65 ± 0.20 | 13.49 |
| 24.93 ± 0.20 | 3.72 |
| 25.25 ± 0.20 | 7.64 |
| 25.61 ± 0.20 | 7.05 |
| 25.91 ± 0.20 | 10.65 |
| 26.14 ± 0.20 | 4.05 |
| 26.99 ± 0.20 | 3.63 |
| 27.46 ± 0.20 | 3.51 |
| 28.49 ± 0.20 | 2.08 |
| 28.78 ± 0.20 | 3.26 |
| 29.18 ± 0.20 | 2.76 |
| 30.14 ± 0.20 | 4.36 |
| 30.47 ± 0.20 | 3.66 |
| 30.91 ± 0.20 | 2.48 |
| 31.18 ± 0.20 | 2.00 |
| 31.68 ± 0.20 | 1.60 |
| 32.14 ± 0.20 | 1.67 |
| 32.59 ± 0.20 | 2.23 |
| 32.95 ± 0.20 | 1.53 |
| 33.77 ± 0.20 | 1.75 |
| 34.86 ± 0.20 | 2.02 |
| 35.30 ± 0.20 | 1.09 |
| 36.27 ± 0.20 | 1.77 |
| 38.05 ± 0.20 | 1.13 |
| 39.50 ± 0.20 | 0.61 |

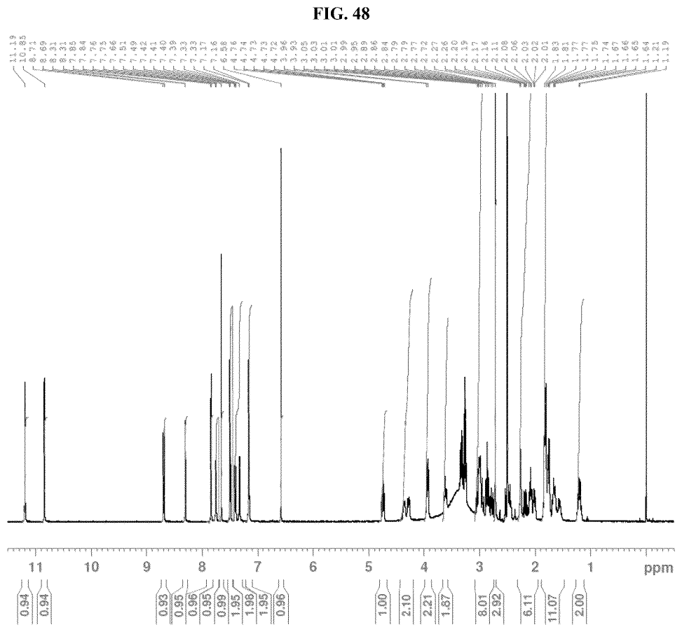

The ¹H NMR spectrum (500 MHZ, DMSO-d₆) of Fumarate Salt Form C is shown in FIG. 48. Analysis by 1H-NMR spectroscopy (FIG. 48) conformed to the molecular structure and showed that Fumarate Salt Form C is a hemi-fumarate salt.

### Synthetic Example 15. Synthesis and Characterization of Fumarate Salt Form D

Fumarate Salt Form D was prepared by temperature cycling Fumarate Salt Amorphous Form in DMSO or DMSO/water. The resulting solid was analysed by XRPD and TGA-DSC analysis. The analysis showed that Fumarate Salt Form D is a mono salt.

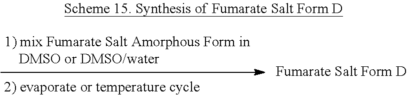

The XRPD pattern of Fumarate Salt Form D is shown in FIG. 49.

The thermograms for Fumarate Salt Form D are provided in FIG. 50. The TGA (right side y-axis) showed 34.0% weight loss from 32° C. to 295° C. As shown in FIG. 50 (left side y-axis), the DTA endotherm in the TGA exhibited a peak onset at 224.6° C. (in the DTA signal).

### Synthetic Example 16. Synthesis and Characterization of Fumarate Salt Form E

Fumarate Salt Form E was prepared by isolation from a slurry of Fumarate Salt Amorphous Form in dimethylacetamide (DMAc). The resulting solid was analysed by XRPD and TGA-DSC analysis. The analysis showed that Fumarate Salt Form E is a mono salt.

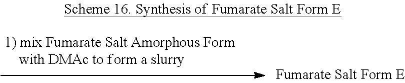

The XRPD pattern of Fumarate Salt Form E is shown in FIG. 51.

The thermograms for Fumarate Salt Form E are provided in FIG. 52. The TGA (right side y-axis) showed 9.08% weight loss from 41° C. to 213° C. As shown in FIG. 52 (left side y-axis), the DTA endotherm in the TGA exhibited a peak onset at 215.8° C. (in the DTA signal).

### Synthetic Example 17. Synthesis and Characterization of Maleate Salt Form A

The compound of Formula (I) (17.8 mg, free base) was dissolved in 2.0 mL of tetrahydrofuran. In a separate container, maleic acid (2.7 mg) was dissolved in 0.5 mL of tetrahydrofuran. The solutions were combined, and a precipitate was observed. A magnetic stir bar was added, and the vial was placed on a stir plate at room temperature with stirring turned on. The slurry was stirred for 6 days at room temperature. The vial was centrifuged, the mother liquor was decanted, and the solid was air dried at room temperature. The resulting solid was analysed by XRPD.

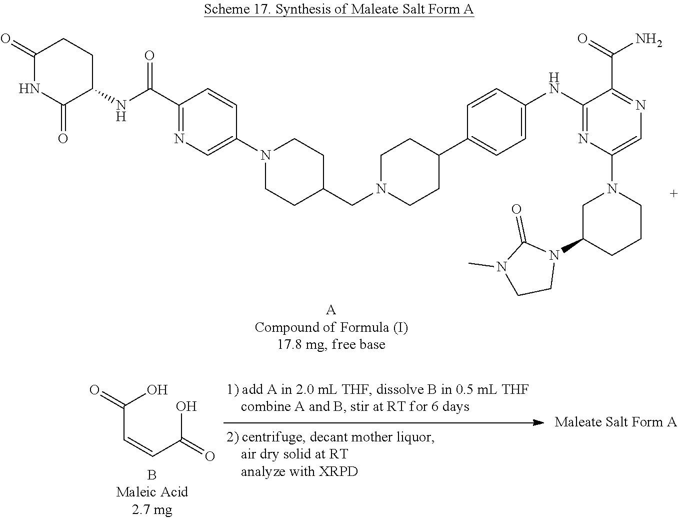 [SMILES: CN1CCN([C@@H]2CCCN(c3cnc(C(N)=O)c(Nc4ccc(C5CCN(CC6CCN(c7ccc(C(=O)N[C@H]8CCC(=O)NC8=O)nc7)CC6)CC5)cc4)n3)C2)C1=O ; O=C(O)/C=C\C(=O)O]

The XRPD pattern of Maleate Salt Form A is shown in FIG. 55. Table 33 is a list of the XRPD peaks for the Malcate Salt Form A.

TABLE 33
Peaks of the XRPD pattern of Maleate Salt Form A

| °2θ | d value (or d space, Å) | Intensity (%) |
|---|---|---|
| 4.66 ± 0.20 | 18.9620 | 36.3655 |
| 9.28 ± 0.20 | 9.5297 | 24.5336 |
| 11.78 ± 0.20 | 7.5123 | 17.0300 |
| 14.20 ± 0.20 | 6.2370 | 53.7309 |
| 14.84 ± 0.20 | 5.9694 | 29.9413 |
| 15.64 ± 0.20 | 5.6658 | 30.6959 |
| 16.02 ± 0.20 | 5.5323 | 35.6110 |
| 16.48 ± 0.20 | 5.3789 | 57.9333 |
| 17.26 ± 0.20 | 5.1375 | 27.8453 |
| 17.60 ± 0.20 | 5.0390 | 31.3037 |
| 18.28 ± 0.20 | 4.8531 | 100.0000 |
| 18.92 ± 0.20 | 4.6903 | 51.8130 |
| 19.46 ± 0.20 | 4.5614 | 76.2838 |
| 20.42 ± 0.20 | 4.3491 | 20.1425 |
| 21.44 ± 0.20 | 4.1444 | 38.5768 |
| 21.74 ± 0.20 | 4.0879 | 56.5709 |
| 22.74 ± 0.20 | 3.9103 | 39.5515 |
| 23.22 ± 0.20 | 3.8306 | 48.6795 |
| 23.70 ± 0.20 | 3.7541 | 23.6324 |
| 24.82 ± 0.20 | 3.5872 | 45.1897 |
| 25.36 ± 0.20 | 3.5120 | 30.1719 |
| 26.04 ± 0.20 | 3.4218 | 26.6506 |
| 26.60 ± 0.20 | 3.3510 | 16.9147 |
| 26.80 ± 0.20 | 3.3265 | 16.9566 |
| 28.02 ± 0.20 | 3.1843 | 19.1469 |
| 28.40 ± 0.20 | 3.1426 | 17.3025 |
| 29.16 ± 0.20 | 3.0624 | 18.5077 |
| 30.04 ± 0.20 | 2.9747 | 12.6808 |
| 30.96 ± 0.20 | 2.8883 | 12.3873 |
| 31.58 ± 0.20 | 2.8330 | 13.1838 |
| 33.50 ± 0.20 | 2.6749 | 11.9681 |
| 35.46 ± 0.20 | 2.5314 | 10.2285 |
| 35.82 ± 0.20 | 2.5068 | 9.7883 |

The solution ¹H NMR spectrum for Maleate Salt Form A was consistent with the chemical structure of the compound of Formula (I) and contained peaks attributed to maleic acid.

The thermograms for Maleate Salt Form A are provided in FIG. 56. The TGA (right side y-axis) showed 2.289% weight loss up to 175° C. and an additional 8.995% weight loss from 175° C. to 250° C. As shown in FIG. 56 (left side y-axis), a broad DSC endotherm exhibited a peak maximum of 228.92° C.

### Synthetic Example 18. Synthesis and Characterization of Tosylate Salt Form A

The compound of Formula (I) (16.9 mg, free base) was dissolved in 2.0 mL of tetrahydrofuran. p-Toluenesulfonic acid (4.0 mg, hydrate) was dissolved in 0.5 mL of tetrahydrofuran. The solutions were combined, and a precipitate was observed. A magnetic stir bar was added, and the vial was placed on a stir plate at room temperature with stirring turned on. The slurry was stirred for 6 days at room temperature. The vial was centrifuged, the mother liquor was decanted, and the solid was air dried at room temperature. The resulting solid was analysed by XRPD.

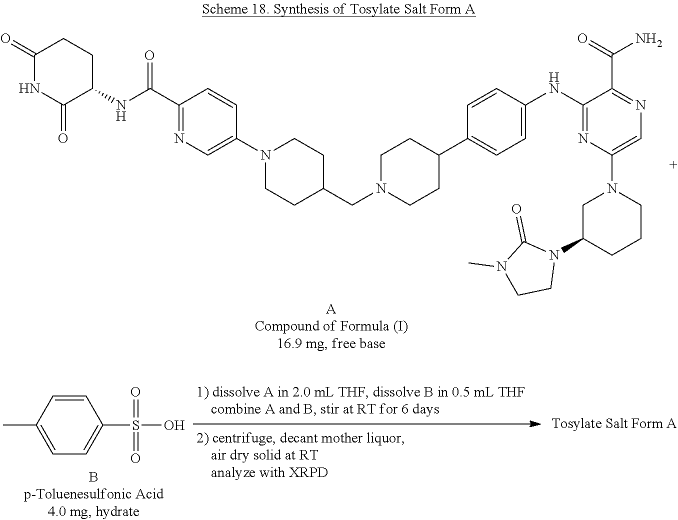 [SMILES: CN1CCN([C@@H]2CCCN(c3cnc(C(N)=O)c(Nc4ccc(C5CCN(CC6CCN(c7ccc(C(=O)N[C@H]8CCC(=O)NC8=O)nc7)CC6)CC5)cc4)n3)C2)C1=O ; Cc1ccc(S(=O)(=O)O)cc1]

The XRPD pattern of Tosylate Salt Form A is shown in FIG. 57. Table 34 is a list of the XRPD peaks for the Tosylate Salt Form A.

TABLE 34
Peaks of the XRPD pattern of Tosylate Salt Form A

| °2θ | d value (or d space, Å) | Intensity (%) |
|---|---|---|
| 4.14 ± 0.20 | 21.3425 | 25.2112 |
| 8.20 ± 0.20 | 10.7822 | 17.2525 |
| 9.94 ± 0.20 | 8.8983 | 39.1517 |
| 10.24 ± 0.20 | 8.6383 | 38.9659 |
| 10.62 ± 0.20 | 8.3301 | 26.1913 |
| 11.40 ± 0.20 | 7.7618 | 20.7503 |
| 12.84 ± 0.20 | 6.8944 | 32.1730 |
| 13.96 ± 0.20 | 6.3437 | 22.3217 |
| 14.64 ± 0.20 | 6.0505 | 36.3467 |
| 15.14 ± 0.20 | 5.8518 | 35.3329 |
| 15.76 ± 0.20 | 5.6230 | 84.4373 |
| 16.74 ± 0.20 | 5.2959 | 41.7709 |
| 17.54 ± 0.20 | 5.0561 | 48.9692 |
| 19.34 ± 0.20 | 4.5894 | 73.9777 |
| 20.32 ± 0.20 | 4.3702 | 56.2352 |
| 20.96 ± 0.20 | 4.2382 | 100.0000 |
| 21.84 ± 0.20 | 4.0694 | 57.4518 |
| 22.86 ± 0.20 | 3.8901 | 79.6553 |
| 24.02 ± 0.20 | 3.7048 | 72.3217 |
| 24.64 ± 0.20 | 3.6129 | 36.3129 |
| 25.32 ± 0.20 | 3.5174 | 46.5022 |
| 26.84 ± 0.20 | 3.3216 | 30.0101 |
| 28.38 ± 0.20 | 3.1448 | 23.2004 |
| 28.78 ± 0.20 | 3.1020 | 25.2957 |
| 29.64 ± 0.20 | 3.0139 | 27.6783 |
| 30.68 ± 0.20 | 2.9140 | 20.0743 |
| 32.14 ± 0.20 | 2.7849 | 17.2693 |
| 36.66 ± 0.20 | 2.4513 | 15.3937 |

The solution ¹H NMR spectrum for Tosylate Salt Form A was consistent with the chemical structure of the compound of Formula (I) and contained peaks attributed to ptoluenesulfonic acid.

### Synthetic Example 19. Synthesis and Characterization of Tosylate Salt Form B

The compound of Formula (I) (19.1 mg, free base) and p-toluenesulfonic acid (5.0 mg, hydrate) were placed into a 1-dram glass vial. To the vial, 0.5 mL of 95:5 methanol:water (v:v) and a magnetic stir bar were added. The vial was placed on a hotplate set to 40° C. with stirring turned on. The slurry was stirred for 7 days at 40° C. The vial was centrifuged, the mother liquor was decanted, and the solid was air dried at room temperature. The resulting solid was analysed by XRPD.

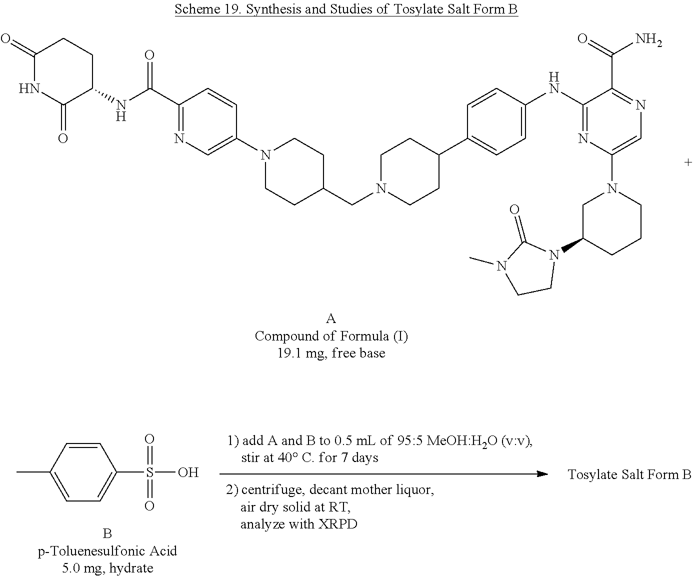 [SMILES: CN1CCN([C@@H]2CCCN(c3cnc(C(N)=O)c(Nc4ccc(C5CCN(CC6CCN(c7ccc(C(=O)N[C@H]8CCC(=O)NC8=O)nc7)CC6)CC5)cc4)n3)C2)C1=O ; Cc1ccc(S(=O)(=O)O)cc1]

The XRPD pattern of Tosylate Salt Form B is shown in FIG. 58. Table 35 is a list of the XRPD peaks for the Tosylate Salt Form B.

TABLE 35
Peaks of the XRPD pattern of Tosylate Salt Form B

| °2θ | d value (or d space, Å) | Intensity (%) |
|---|---|---|
| 9.00 ± 0.20 | 9.8255 | 41.8240 |
| 9.64 ± 0.20 | 9.1746 | 31.5519 |
| 9.90 ± 0.20 | 8.9342 | 32.2138 |
| 10.20 ± 0.20 | 8.6721 | 47.2665 |
| 10.54 ± 0.20 | 8.3931 | 52.1942 |
| 11.36 ± 0.20 | 7.7890 | 29.1983 |
| 11.86 ± 0.20 | 7.4618 | 30.7919 |
| 12.58 ± 0.20 | 7.0363 | 31.5028 |
| 12.80 ± 0.20 | 6.9158 | 29.9093 |
| 13.10 ± 0.20 | 6.7581 | 38.2937 |
| 13.42 ± 0.20 | 6.5977 | 35.2047 |
| 13.78 ± 0.20 | 6.4261 | 32.9983 |
| 14.12 ± 0.20 | 6.2721 | 38.0240 |
| 14.64 ± 0.20 | 6.0505 | 36.8473 |
| 15.06 ± 0.20 | 5.8827 | 39.7156 |
| 15.32 ± 0.20 | 5.7834 | 43.7362 |
| 15.72 ± 0.20 | 5.6372 | 50.0368 |
| 16.56 ± 0.20 | 5.3531 | 49.3748 |
| 17.50 ± 0.20 | 5.0676 | 52.1696 |
| 18.00 ± 0.20 | 4.9279 | 68.4236 |
| 18.40 ± 0.20 | 4.8217 | 83.8931 |
| 19.12 ± 0.20 | 4.6417 | 87.3008 |
| 19.68 ± 0.20 | 4.5109 | 100.0000 |
| 20.38 ± 0.20 | 4.3575 | 74.8958 |
| 20.88 ± 0.20 | 4.2543 | 79.6764 |
| 21.86 ± 0.20 | 4.0657 | 76.0481 |
| 22.08 ± 0.20 | 4.0257 | 86.1731 |
| 22.36 ± 0.20 | 3.9759 | 79.1370 |
| 22.58 ± 0.20 | 3.9377 | 72.6404 |
| 22.90 ± 0.20 | 3.8834 | 68.9385 |
| 23.42 ± 0.20 | 3.7983 | 65.9966 |
| 24.08 ± 0.20 | 3.6957 | 55.8961 |
| 24.60 ± 0.20 | 3.6187 | 43.4420 |
| 25.28 ± 0.20 | 3.5229 | 49.5710 |
| 26.38 ± 0.20 | 3.3785 | 52.0961 |
| 26.86 ± 0.20 | 3.3192 | 42.3143 |
| 27.24 ± 0.20 | 3.2737 | 40.6227 |
| 27.64 ± 0.20 | 3.2272 | 42.1672 |
| 27.84 ± 0.20 | 3.2045 | 39.1027 |
| 28.26 ± 0.20 | 3.1578 | 33.8563 |
| 28.76 ± 0.20 | 3.1041 | 32.4099 |
| 29.62 ± 0.20 | 3.0159 | 30.1544 |
| 30.74 ± 0.20 | 2.9085 | 30.8654 |
| 32.06 ± 0.20 | 2.7917 | 26.5751 |
| 37.04 ± 0.20 | 2.4270 | 22.1868 |

The solution ¹H NMR spectrum for Tosylate Salt Form B was consistent with the chemical structure of the compound of Formula (I) and contained peaks attributed to ptoluenesulfonic acid.

### Synthetic Example 20. Synthesis and Characterization of Besylate Salt Form A

Besylate Salt Form A was prepared by a salt formation reaction with Free Base Form A in tetrahydrofuran/water (2:1, 12 vols) and 1 mol. equivalent of benzenesulfonic acid. The reaction mixture was stirred, and temperature cycled between ambient temperature (of from 15° C. to 30° C.) and 50° C. for 60 hours before being returned to ambient temperature and stirred for another 18 hours. Besylate Salt Form A was obtained as a solid, isolated by centrifugation. The resulting solid was analysed by XRPD and TGA-DSC analysis.

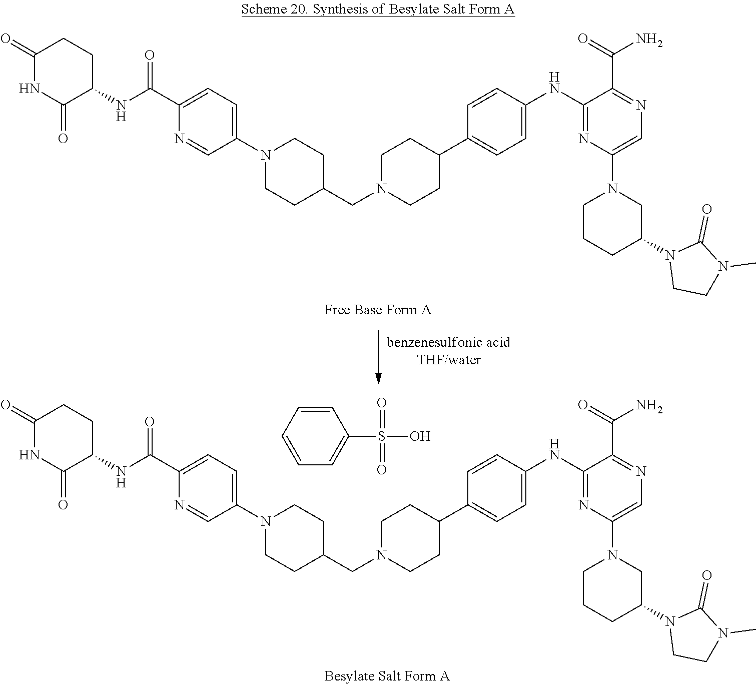

The XRPD pattern of Besylate Salt Form A is shown in FIG. 59.

The thermograms for Besylate Salt Form A are provided in FIG. 60. The TGA (right side y-axis) showed 1.12% weight loss from 31° C. to 88° C., and from 34.20% weight loss from 88° C. to 155° C. As shown in FIG. 60 (left side y-axis), the DTA endotherm in the TGA exhibited a peak onset at 99.13° C. and 217.8° C. (in the DTA signal).

### Synthetic Example 21. Synthesis and Characterization of Cyclamate Salt Form A

Cyclamate Salt Form A was prepared by a salt formation reaction with Free Base Form A and cyclamic acid (1 mol. equivalent) in THF. The reaction mixture was stirred at 40° C. for 60 hours then cooled to 5° C. and stirred at this lower temperature for 18h before the solids were isolated by centrifugation. The solids were dried under vacuum for 1 hour to afford Cyclamate Salt Form A. The resulting solid was analysed by XRPD and TGA-DSC analysis.

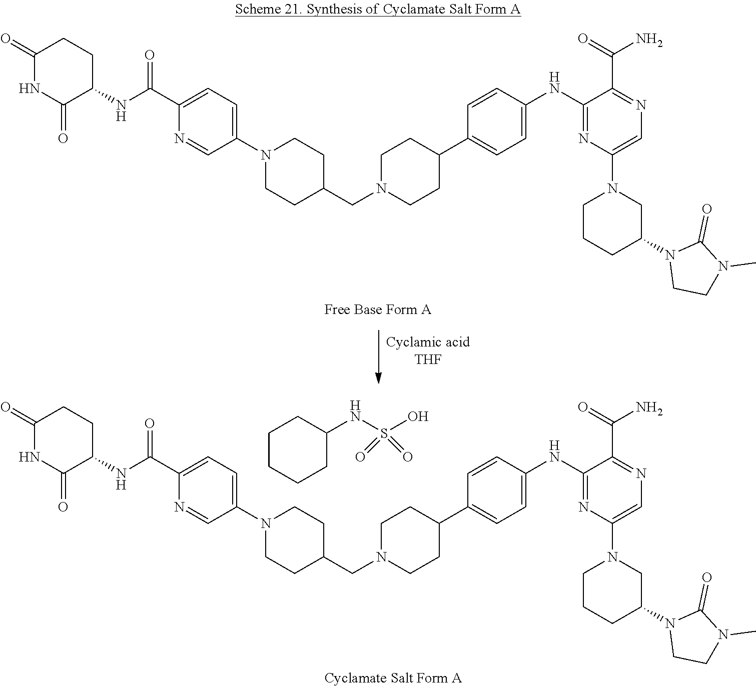

The XRPD pattern of Cyclamate Salt Form A is shown in FIG. 61.

The thermograms for Cyclamate Salt Form A are provided in FIG. 62. The TGA (right side y-axis) showed 1.72% weight loss from 31° C. to 229° C. As shown in FIG. 62 (left side y-axis), the DTA endotherm in the TGA exhibited peak onsets at 223.1° C. and 284.2° C. (in the DTA signal).

### Synthetic Example 22. Synthesis and Characterization of Malate Salt Form A

Malate Salt Form A was prepared by a salt formation reaction with Free Base Form A and malic acid (1 mol. eq.) in THF. The reaction mixture was stirred at 40° C. for 60 hours then cooled to 5° C. and stirred at this lower temperature for 18 hours before the solids were isolated by centrifugation. The solids were dried for 1 hour under vacuum to afford Malate Salt Form A. The resulting solid was analysed by XRPD and TGA-DSC analysis.

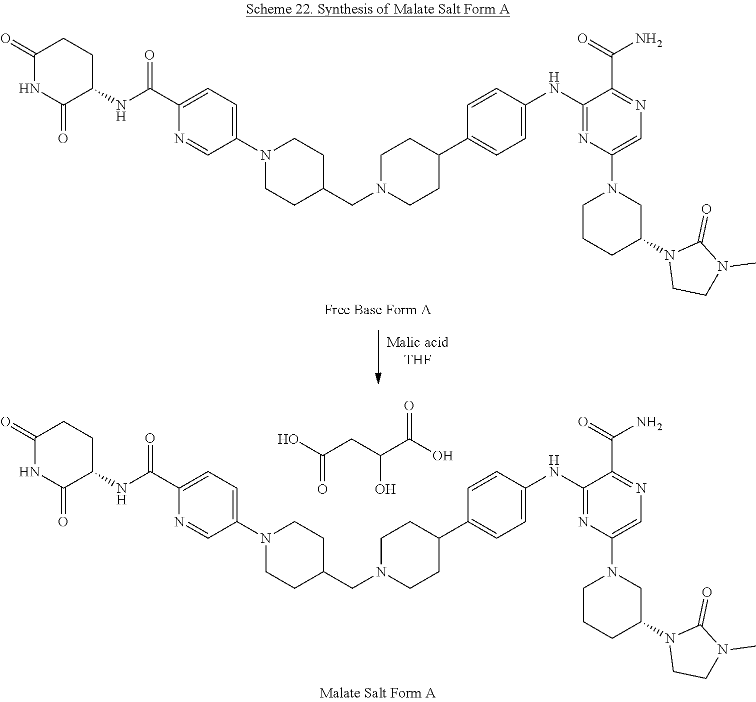

The XRPD pattern of Malate Salt Form A is shown in FIG. 63.

The thermograms for Malate Salt Form A are provided in FIG. 64. The TGA (right side y-axis) showed 4.73% weight loss from 30° C. to 223° C. As shown in FIG. 64 (left side y-axis), the DTA endotherm in the TGA exhibited peak onsets at 87.2° C. and 230.2° C. (in the DTA signal).

### Synthetic Example 23. Synthesis and Characterization of Malonate Salt Form A

Malonate Salt Form A was prepared by salt formation with Free Base Form A in THF/water (2:1, 12 vols) using 1 mol. equivalent of malonic acid. The reaction mixture was stirred, and temperature cycled between ambient temperature (of from 15° C. to 30° C.) and 50° C. for 60 hours before being returned to ambient temperature and stirred for a further 18 hours. Amorphous solids were isolated by centrifugation. These amorphous solids were mixed in acetone (15-20 vols) to form a slurry and heated at 40° C. for a minimum of 48 hours before returning to ambient temperature. The solids were isolated by centrifugation and air dried for 1 hour before analysis to afford Malonate Salt Form A. The resulting solid was analysed by XRPD and TGA-DSC analysis.

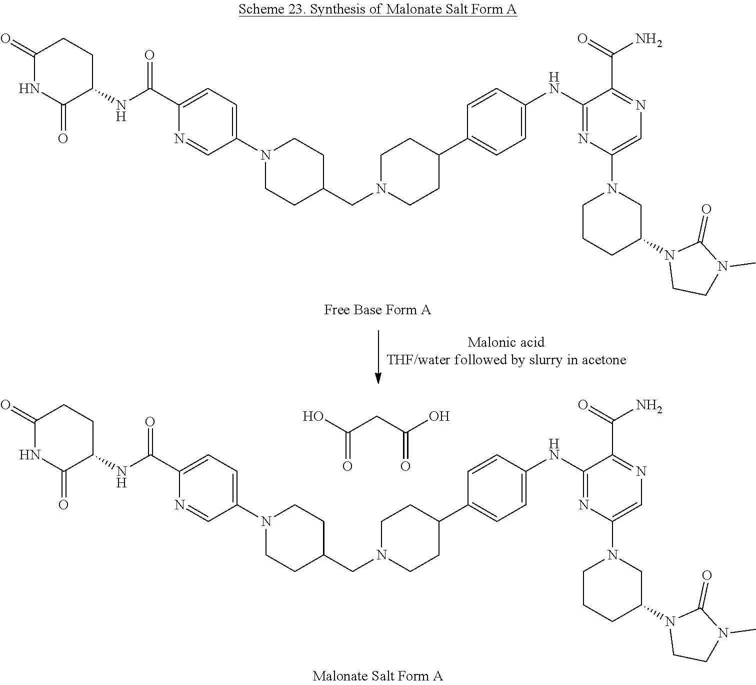

The XRPD pattern of Malonate Salt Form A is shown in FIG. 65.

The thermograms for Malonate Salt Form A are provided in FIG. 66. The TGA (right side y-axis) showed 2.43% weight loss from 31° C. to 137° C., 6.30% weight loss from 138° C. to 180° C., and 4.48% weight loss from 181° C. to 298° C. As shown in FIG. 66 (left side y-axis), the DTA endotherm in the TGA exhibited a peak onset at 159.3° C. (in the DTA signal).

### Synthetic Example 24. Synthesis and Characterization of Napsylate Salt Form A

Napsylate Salt Form A was prepared by a salt formation reaction with Free Base Form A and naphthalene-2-sulfonic acid (1 mol. equivalent) in THF. The reaction mixture was stirred at 40° C. for 60 hours then cooled to 5° C. and stirred at this lower temperature for 18 hours before the solids were isolated by centrifugation to afford Napsylate Salt Form A. The resulting solid was analysed by XRPD and TGA-DSC analysis.

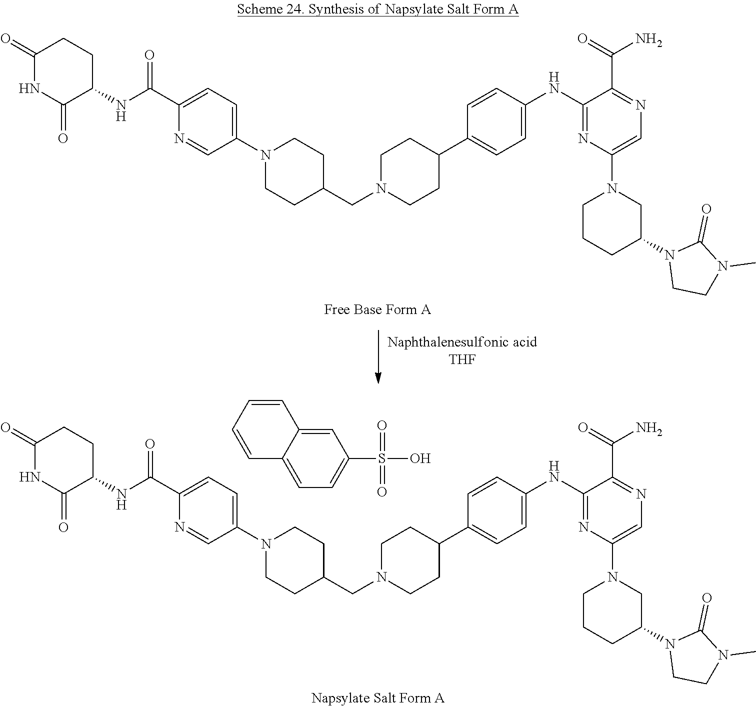

The XRPD pattern of Napsylate Salt Form A is shown in FIG. 67.

The thermograms for Napsylate Salt Form A are provided in FIG. 68. The TGA (right side y-axis) showed 2.78% weight loss from 31° C. to 251° C. As shown in FIG. 68 (left side y-axis), the DTA endotherm in the TGA exhibited a peak onset at 262.1° C. (in the DTA signal).

### Synthetic Examples 25-27. Synthesis and Characterization of Napsylate Salt Form B (Synthetic Example 25), Napsylate Salt Form C (Synthetic Example 26), and Napsylate Salt Form D (Synthetic Example 27)

Napsylate Salt Form B was prepared by salt formation with Free Base Form A in THF/water (2:1, 12 vols) using 1 mol. equivalent of naphthalene-2-sulfonic acid. The reaction mixture was stirred, and temperature cycled between ambient temperature (of from 15° C. to 30° C.) and 50° C. for 60 hours before returning to ambient temperature. The mixture was stirred for a further 18 hours. Solids were isolated by centrifugation to afford Napsylate Salt Form B, analysed by XRPD.

Napsylate Salt Form C was prepared by storing Napsylate Salt Form B under ambient conditions, which completed the conversion to Napsylate Salt Form C. Napsylate Salt Form C was analysed by XRPD.

Napsylate Salt Form D was prepared by drying Napsylate Salt Form B under vacuum for 1 hour, which completed the conversion to Napsylate Salt Form D. Napsylate Salt Form D was analysed by XRPD and TGA-DSC analysis.

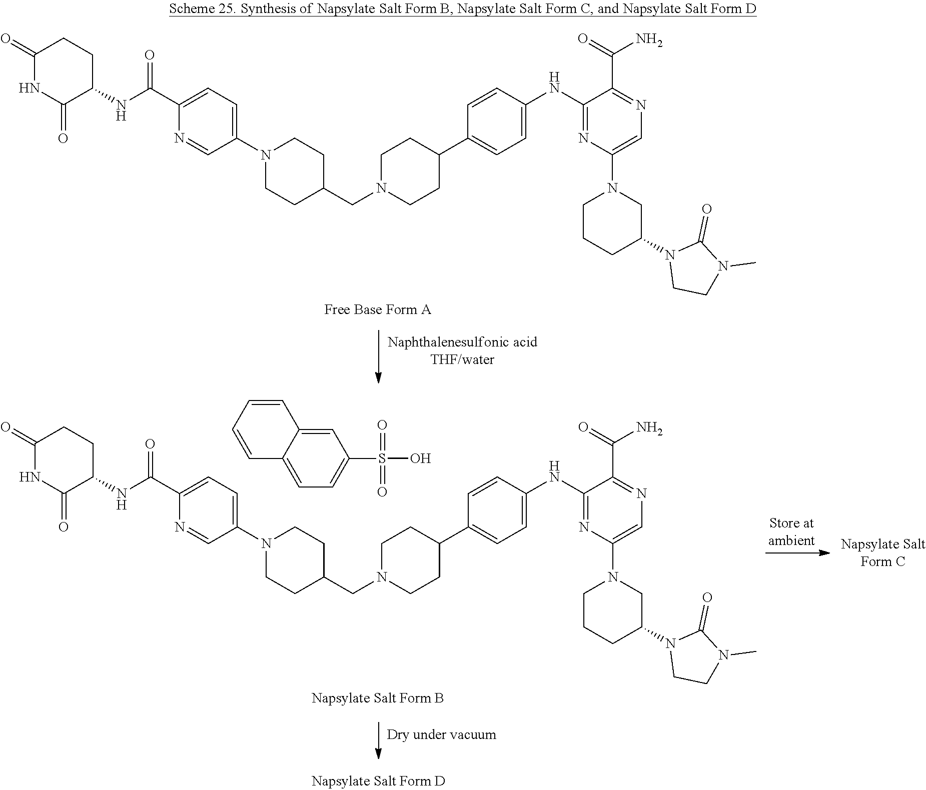

The XRPD patterns of Napsylate Salt Forms A, B, C, and D are shown in FIG. 69: Napsylate Salt Form A (top); Napsylate Salt Form B (2ªd from top); Napsylate Salt Form C (2^{nd} from bottom); and Napsylate Salt Form D (bottom).

The XRPD pattern of Napsylate Salt Form D is shown in FIG. 70.

The thermograms for Napsylate Salt Form D are provided in FIG. 71. The TGA (right side y-axis) showed 1.97% weight loss from 30° C. to 83° C., 25.77% weight loss from 86° C. to 124° C., and 0.93% weight loss from 151° C. to 175° C. As shown in FIG. 71 (left side y-axis), the DTA endotherm in the TGA exhibited a peak onset at 98.4° C. (in the DTA signal).

### Synthetic Examples 28-29. Synthesis and Characterization of Succinate Salt Form A (Synthetic Example 28) and Succinate Salt Form B (Synthetic Example 29)

Succinate Salt Form A and Succinate Salt Form B were prepared by salt formation reactions with Free Base Form A and succinic acid (1 mol. equivalent) in THF. The reaction mixture was stirred at 40° C. for 60 hours then cooled to 5° C. and stirred at this lower temperature for 18 hours before Succinate Salt Form A solids were isolated by centrifugation. After drying the solids for 1 hour under vacuum, XRPD analysis showed that Succinate Salt Form A converted to Succinate Salt Form B. Succinate Salt Form B was stressed at 40° C./75% relative humidity (RH) and was converted back to Succinate Salt Form A material. Succinate Salt Form A was analysed by XRPD. Succinate Salt Form B was analysed by XRPD and TGA-DSC.

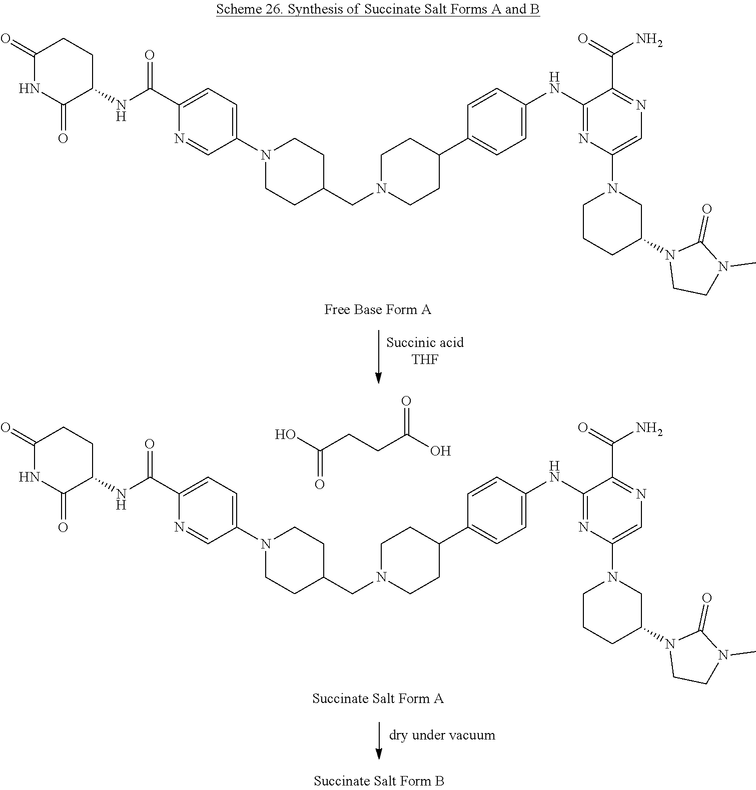 [SMILES: CN1CCN([C@@H]2CCCN(c3cnc(C(N)=O)c(Nc4ccc(C5CCN(CC6CCN(c7ccc(C(=O)N[C@H]8CCC(=O)NC8=O)nc7)CC6)CC5)cc4)n3)C2)C1=O ; CN1CCN([C@@H]2CCCN(c3cnc(C(N)=O)c(Nc4ccc(C5CCN(CC6CCN(c7ccc(C(=O)N[C@H]8CCC(=O)NC8=O)nc7)CC6)CC5)cc4)n3)C2)C1=O ; O=C(O)CCC(=O)O]

The XRPD patterns of Succinate Salt Forms A and B are shown in FIG. 72: Free Base Form A (top); Succinate Salt Form A obtained after stressing Succinate Salt Form B at 40° C./75% RH for 1 week (2^{nd} from top); Succinate Salt Form B (2^{nd} from bottom); and Succinate Salt Form A formed from the reaction in THF (bottom).

The XRPD pattern of Succinate Salt Form A is shown in FIG. 73.

The thermograms for Succinate Salt Form B are provided in FIG. 74. The TGA (right side y-axis) showed 1.55% weight loss from 34° C. to 214° C. As shown in FIG. 74 (left side y-axis), the DTA endotherm in the TGA exhibited peak onsets at 77.4° C. and 208.6° C. (in the DTA signal).

### Synthetic Example 30. Synthesis and Characterization of Succinate Salt Form C

Succinate Salt Form C was prepared by salt formation with Free Base Form A in THF/water (2:1, 12 vols) using 1 mol. equivalent of succinic acid. The reaction mixture was stirred, and the temperature was cycled between ambient temperature (of from 15° C. to 30° C.) and 50° C. for 60 hours before being returned to ambient temperature and stirred for a further 18 hours. Solids were isolated by centrifugation and air dried under vacuum for 1 hour to afford Succinate Salt Form C. Succinate Salt Form C was analysed by XRPD and TGA-DSC.

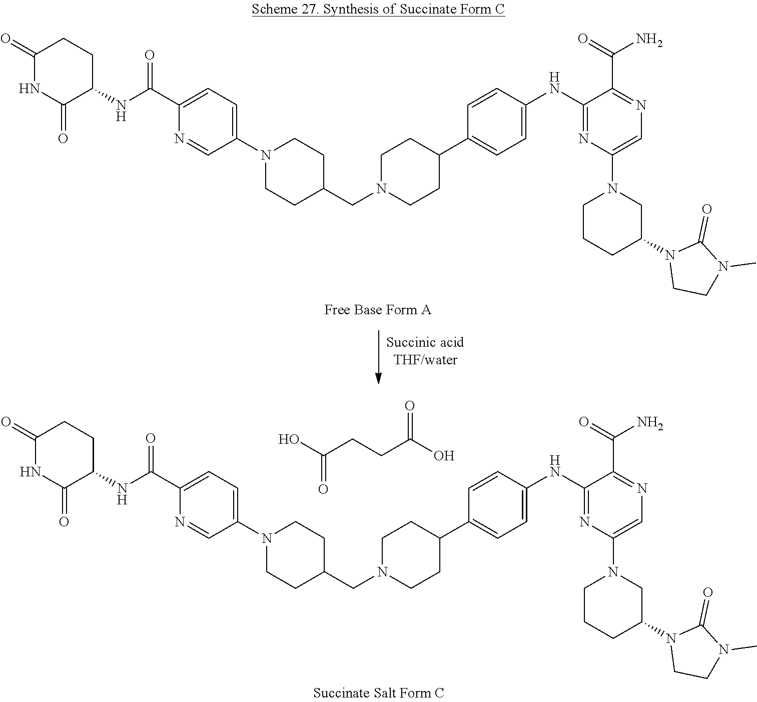

The XRPD pattern of Succinate Salt Form C is shown in FIG. 75.

The thermograms for Succinate Salt Form C are provided in FIG. 76. The TGA (right side y-axis) showed 23.6% weight loss from 75° C. to 153° C., and 3.37% weight loss from 235° C. to 302° C. As shown in FIG. 76 (left side y-axis), the DTA endotherm in the TGA exhibited peak onsets at 99.4° C. and 209.6° C. (in the DTA signal).

Scheme 28. Preparation of Amorphous Solid Dispersion with HPMCAS-H

The components of amorphous solid dispersion with HPMCAS-H can be found below. The quantities of Fumarate salt Form A and HPMCAS-H are adjusted according to target a 20% (w/w) as free base concentration in the amorphous solid dispersion.

| | Theoretical | Batch Quantity, 2 kg |
|---|---|---|
| | Composition | Basis |
| Components | (w/w %) | (g) |
| Fumarate Salt Form A | 20 (as free base) | 400 (as free base) |
| HPMCAS-H | 80 | 1600 |

The manufacture of the amorphous solid dispersion includes the following processing steps:
1. Dispensing a total of 6% (w/w) solids of Fumarate Salt Form A and HPMCAS-H in a solvent mixture of DCM: MeOH (4:1 ratio)
2. Mixing to effect dissolution of Fumarate Salt Form A and HPMCAS-H in a solvent mixture of DCM: MeOH (4:1 ratio)
3. Spray-drying
4. Secondary drying
   Scheme 29. Preparation of Amorphous Solid Dispersion with Eudragit L-100

The components of amorphous solid dispersion with Eudragit L-100 can be found below. The quantities of Fumarate salt Form A and Eudragit L-100 are adjusted according to target a 20% (w/w) as free base concentration in the amorphous solid dispersion.

| | Theoretical | Batch Quantity, 2 kg |
|---|---|---|
| | Composition | Basis |
| Components | (w/w %) | (g) |
| Fumarate Salt Form A | 20 (as free base) | 400 (as free base) |
| Eudragit L-100 | 80 | 1600 |

1. Dispensing a total of 4% (w/w) solids of Fumarate Salt Form A and Eudragit L-100 in a solvent mixture of DCM: MeOH (4:1 ratio)
2. Mixing to effect dissolution of Fumarate Salt Form A and Eudragit L-100 in a solvent mixture of DCM: MeOH (4:1 ratio)
3. Spray-drying
4. Secondary drying

The spray drying was conducted using BüChi B-290 (ID: RD-038). The secondary drying was conducted using a VWM tray dryer (1370FM). The spray drying and secondary drying parameters can be found below.

| | |
|---|---|
| Nozzle Type | Two-Fluid −1.5 mm Air Cap, 0.7 mm |
| | Liquid Tip |
| Drying Gas Mode | Recycle |
| Condenser Temperature (° C.) | Approximately −20 |
| Solution Flow Rate (g/min) | 20 |
| Atomization Pressure (psi) | 26 |
| Inlet Temperature (° C.) | 85-90 |
| Outlet Temperature (° C.) | 40-45 |
| Secondary Drying | 24 hr at 40° C. |

XRPD was performed using a Rigaku Miniflex 6G X-ray diffractometer to evaluate the crystallinity of bulk Fumarate Salt Form A and amorphous solid dispersion. Amorphous materials give an “amorphous halo” diffraction pattern, absent of discrete peaks that are found in crystalline material. See FIG. 77 for XRPD diffractograms of amorphous solid dispersion of Fumarate Salt Form A with Eudragit L-100 under different storage conditions over one month period. Samples were irradiated with monochromatized Cu Kα radiation and analyzed from 5° and 40° with a continuous scanning mode. Samples were rotated during analysis to minimize preferred orientation effects.

DSC was performed using a TA Instruments Discovery DSC2500 or X3 differential scanning calorimeter equipped with a TA instruments Refrigerated Cooling System (RCS) 90 operating in either modulated or ramp mode. DSC was used to measure thermodynamic properties of amorphous solid dispersion, including the following: glass transition temperature (Tg) defined as the temperature at which amorphous materials transition from a low mobility glassy state to a high mobility rubbery state, cold crystallization temperature (Tc), defined as a crystallization event at a temperature lower than the melt temperature, and melting temperature (Tm). The system was purged by nitrogen flow at 50 mL/min to ensure inert atmosphere through the course of measurement. See summary of modulated DSC parameters below:

| Parameter | Value | |
|---|---|---|
| Instrument | TA Discovery DSC2500 or X3 with RCS90 | |
| | chiller | |
| Sample Pans | Tzero AI, non-hermetic | |
| Temperature Range | −20° C.-200° C. | |
| Heating Rate | 2° | C./min |
| Scanning Mode | Modulated | |
| Modulation Frequency | 60 | s |
| Modulation Amplitude | 1° | C. |

| | |
|---|---|
| Patheon Method | DM-0046 |

See FIG. 78 for thermograms of amorphous solid dispersion of Fumarate Salt Form A with Eudragit L-100 under different storage conditions over a two-week period.

The embodiments and examples described above are intended to be merely illustrative and non-limiting. Those skilled in the art will recognize or will be able to ascertain using no more than routine experimentation, numerous equivalents of specific compounds, materials and procedures. All such equivalents are considered to be within the scope and are encompassed by the appended claims.

1. A free base of a compound of Formula (I):

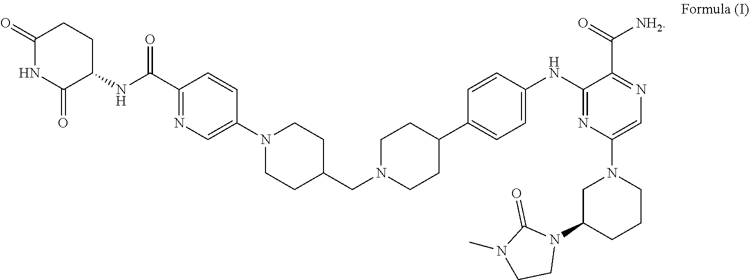 [SMILES: CN1CCN([C@@H]2CCCN(c3cnc(C(N)=O)c(Nc4ccc(C5CCN(CC6CCN(c7ccc(C(=O)N[C@H]8CCC(=O)NC8=O)nc7)CC6)CC5)cc4)n3)C2)C1=O]

2. The free base of claim 1, wherein the free base is crystalline, semi-crystalline, amorphous, a hydrate, or a solvate.

3. The free base of claim 1, wherein the free base exhibits at least one diffraction peak at about 5.0−40° 2θ in its X-ray powder diffraction pattern.

4.-9. (canceled)

10. The free base of claim 1, wherein the free base is a solvate and the solvate is a tetrahydrofuran (THF) solvate, a methyl ethyl ketone (MEK) solvate, a dimethyl sulfoxide (DMSO) solvate, a dimethylformamide (DMF) solvate, a diethyl ether solvate, an acetone solvate, a dimethylacetamide (DMAc) solvate, or a combination thereof.

11. The free base of claim 1, wherein the free base is Free Base Form A.

12. The Free Base Form A of claim 11, wherein:
the X-ray powder diffraction pattern exhibits diffraction peaks at about 4.96, 14.26, 15.86, 18.90, 19.98, 22.12, 23.68, and 25.00±0.2° 2θ; or
the differential scanning calorimetry thermogram exhibits four endotherms, wherein the first endotherm has an endotherm maximum between about 60° C. and about 70° C., the second endotherm has an endotherm maximum between about 95° C. and about 105° C., the third endotherm has an endotherm maximum between about 210° C. and about 220° C., and the fourth endotherm has an endotherm maximum between about 225° C. and about 235° C.; or
the Free Base Form A exhibits a weight loss in a thermogravimetric analysis (TGA) in the range of about 0.1% to about 5% when heated from about 25° C. to about 75° C.

13. The Free Base Form A of claim 11, wherein:
the X-ray powder diffraction pattern is substantially similar to that of FIG. 1; or
the differential scanning calorimetry thermogram is substantially similar to that of FIG. 2; or
the TGA pattern is substantially similar to that of FIG. 2.

14.-20. (canceled)

21. An amorphous solid dispersion comprising the free base of claim 11.

22.-24. (canceled)

25. The free base of claim 1, wherein the free base is Free Base Amorphous Form.

26. The Free Base Amorphous Form of claim 25, wherein:
the Free Base Amorphous Form is shown to be amorphous by X-ray powder diffraction; or
the differential scanning calorimetry thermogram exhibits one endotherm, where the endotherm has an endotherm maximum between about 30° C. and about 100° C.; or
the Free Base Amorphous Form exhibits a weight loss in a thermogravimetric analysis (TGA) in the range of about 0.1% to about 1% when heated from about 35° C. to about 190° C.

27. The Free Base Amorphous Form of claim 25, wherein;
the X-ray powder diffraction pattern is substantially similar to that of FIG. 4; or
the differential scanning calorimetry thermogram is substantially similar to that of FIG. 7.

28.-32. (canceled)

33. An amorphous solid dispersion comprising the free base of claim 25.

34.-35. (canceled)

36. The free base of claim 1, wherein the free base is Free Base Form B.

37. The Free Base Form B, wherein:
the Free Base Form B is a DMSO solvate; or
the X-ray powder diffraction pattern exhibits diffraction peaks at about 16.46, 17.98, 21.22, 21.70, 22.92, and 24.46±0.2°2θ; or
the differential scanning calorimetry thermogram exhibits three endotherms, wherein the first endotherm has an endotherm maximum between about 120° C. and about 130° C., the second endotherm has an endotherm maximum between about 220° C. and about 230° C., and the third endotherm has an endotherm maximum between about 230° C. and about 240° C.; or
the Free Base Form B exhibits a weight loss in a thermogravimetric analysis (TGA) in the range of about 10% to about 15% when heated from about 25° C. to about 175° C.

38. The Free Base Form B of claim 36, wherein:
the X-ray powder diffraction pattern is substantially similar to that of FIG. 11; or
the differential scanning calorimetry thermogram is substantially similar to that of FIG. 12; or
the TGA pattern is substantially similar to that of FIG. 12.

39.-43. (canceled)

44. An amorphous solid dispersion comprising the free base of claim 36.

45.-46. (canceled)

47. The free base of claim 1, wherein the free base is Free Base Form C.

48. The Free Base Form C of claim 47, wherein;
the Free Base Form C is a DMF solvate; or
the X-ray powder diffraction pattern exhibits diffraction peaks at about 6.40, 8.40, 12.76, 16.76, and 17.98±0.2°2θ; or
the differential scanning calorimetry thermogram exhibits two endotherms, wherein the first endotherm has an endotherm maximum between about 60° C. and about 70° C., and the second endotherm has an endotherm maximum between about 195° C. and about 205° C.; or
the Free Base Form C exhibits a weight loss in a thermogravimetric analysis (TGA) in the range of about 10% to about 15% when heated from about 25° C. to about 200° C.

49. The Free Base Form C of claim 47, wherein:
the X-ray powder diffraction pattern is substantially similar to that of FIG. 13; or
the differential scanning calorimetry thermogram is substantially similar to that of FIG. 14; or
the TGA pattern is substantially similar to that of FIG. 14.

50.-54. (canceled)

55. An amorphous solid dispersion comprising the free base of claim 47.

56.-57. (canceled)

58. The free base of claim 1, wherein the free base is Free Base Form D.

59. The Free Base Form D of claim 58, wherein:
the X-ray powder diffraction pattern exhibits diffraction peaks at about 4.18, 17.20, 17.52, and 19.64±0.2° 2θ; or
the differential scanning calorimetry thermogram exhibits three endotherms, wherein the first endotherm has an endotherm maximum between about 50° C. and about 60° C., the second endotherm has an endotherm maximum between about 155° C. and about 165° C., and the third endotherm has an endotherm maximum between about 170° C. and about 180° C.; or
the Free Base Form D exhibits a weight loss in a thermogravimetric analysis (TGA) in the range of about 0.1% to about 5% when heated from about 25° C. to about 75° C.

60. The Free Base Form D of claim 58, wherein:
the X-ray powder diffraction pattern is substantially similar to that of FIG. 15; or
the differential scanning calorimetry thermogram is substantially similar to that of FIG. 16; or
the TGA pattern is substantially similar to that of FIG. 16 or FIG. 17.

61.-64. (canceled)

65. An amorphous solid dispersion comprising the free base of claim 58.

66.-74. (canceled)

75. The free base of claim 1, wherein the free base is a solvate and the solvate is an acetonitrile solvate, a tetrahydrofuran solvate, a dioxane solvate, an ethanol solvate, an ethyl acetate solvate, a methanol solvate, a 2-methyl-tetrahydrofuran solvate, an isopropanol solvate, a toluene solvate, a dimethylacetamide solvate, a dimethylsulfonamide solvate, or an anisole solvate.

76. The free base of claim 1, wherein the free base is Free Base Form E.

77. The Free Base Form E of claim 76, wherein:
the Free Base Form E is an ethanol solvate, an ethyl acetate solvate, a methanol solvate, a 2-methyl-tetrahydrofuran solvate, an isopropanol solvate, or a dioxane solvate; or
the X-ray powder diffraction pattern exhibits diffraction peaks at about 4.6, 14.6, 17.4, 20.0, and 24.5±0.2° 2θ; or
the differential thermal analysis thermogram exhibits an endotherm, wherein the endotherm has an endotherm maximum between about 169° C. and 190° C.; or
the Free Base Form E exhibits a weight loss in a thermogravimetric analysis (TGA) in the range of about 0.1% to about 5% when heated from about 40° C. to about 150° C.

78. The Free Base Form E of claim 76, wherein:
the X-ray powder diffraction pattern is substantially similar to that of FIG. 18; or
the differential thermal analysis thermogram is substantially similar to that of FIG. 20; or
the TGA pattern is substantially similar to that of FIG. 20.

79.-83. (canceled)

84. An amorphous solid dispersion comprising the free base of claim 76.

85.-86. (canceled)

87. The free base of claim 1, wherein the free base is Free Base Form F.

88. The Free Base Form F of claim 87, wherein;
the Free Base Form F is a toluene solvate; or
the X-ray powder diffraction pattern exhibits diffraction peaks at about 4.3, 9.5, 16.8, 18.5, and 19.8±0.2°2θ.

89. The Free Base Form F of claim 87, wherein:
the X-ray powder diffraction pattern is substantially similar to that of FIG. 21.

90. An amorphous solid dispersion comprising the free base of claim 87.

91.-92. (canceled)

93. The free base of claim 1, wherein the free base is Free Base Form G.

94. The Free Base Form G of claim 93, wherein:
the Free Base Form G is a dimethylacetamide solvate; or
the X-ray powder diffraction pattern exhibits diffraction peaks at about 7.8, 16.0, 16.5, 18.2, and 21.9±0.2° 2θ; or
the differential thermal analysis thermogram exhibits two endotherms, wherein the first endotherm has an endotherm maximum between about 120° C. and about 167° C., and the second endotherm has an endotherm maximum between about 209° C. and about 247° C.; or
the Free Base Form G exhibits a weight loss in a thermogravimetric analysis (TGA) in the range of about 5% to about 10% when heated from about 65° C. to about 295° C.

95. The Free Base Form G of claim 93, wherein:
the X-ray powder diffraction pattern is substantially similar to that of FIG. 22; or
the differential thermal analysis thermogram is substantially similar to that of FIG. 24; or
the TGA pattern is substantially similar to that of FIG. 24.

96.-99. (canceled)

100. An amorphous solid dispersion comprising the free base of claim 93.

101.-102. (canceled)

103. The free base of claim 1, wherein the free base is Free Base Form H.

104. The Free Base Form H of claim 103, wherein:
the X-ray powder diffraction pattern exhibits diffraction peaks at about 4.2, 10.2, 15.7, 16.5, and 20.9±0.2° 2θ; or
the differential thermal analysis thermogram exhibits two endotherms, wherein the first endotherm has an endotherm maximum between about 174° C. and 194° C., and the second endotherm has an endotherm maximum between about 231° C. and 261° C.; or
the Free Base Form H exhibits a weight loss in a thermogravimetric analysis (TGA) in the range of about 5% to about 10% when heated from about 44° C. to about 302° C.

105. The Free Base Form H of claim 103:
the X-ray powder diffraction pattern is substantially similar to that of FIG. 25; or
the differential thermal analysis thermogram is substantially similar to that of FIG. 27; or
the TGA pattern is substantially similar to that of FIG. 27.

106.-109. (canceled)

110. An amorphous solid dispersion comprising the free base of claim 103.

111.-112. (canceled)

113. The free base of claim 1, wherein the free base is Free Base Form J.

114. The Free Base Form J of claim 113, wherein:
the X-ray powder diffraction pattern exhibits diffraction peaks at about 4.6, 9.9, 14.2, 15.7, and 17.0±0.2° 2θ; or
the differential thermal analysis thermogram exhibits an endotherm, wherein the endotherm has an endotherm maximum between about 192° C. and 214° C.; or
the Free Base Form J exhibits a weight loss in a thermogravimetric analysis (TGA) in the range of about 5% to about 10% when heated from about 54° C. to about 299° C.

115. The Free Base Form J of claim 113, wherein:
the X-ray powder diffraction pattern is substantially similar to that of FIG. 28; or
the differential thermal analysis thermogram is substantially similar to that of FIG. 30; or
the TGA pattern is substantially similar to that of FIG. 30.

116.-121. (canceled)

122. An amorphous solid dispersion comprising the free base of claim 113.

123.-124. (canceled)

125. A salt of a compound of Formula (I):

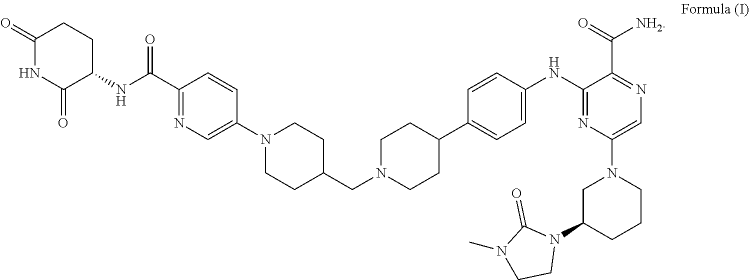 [SMILES: CN1CCN([C@@H]2CCCN(c3cnc(C(N)=O)c(Nc4ccc(C5CCN(CC6CCN(c7ccc(C(=O)N[C@H]8CCC(=O)NC8=O)nc7)CC6)CC5)cc4)n3)C2)C1=O]

126. The salt of claim 125, wherein the salt is a fumarate salt.

127. The salt of claim 125, wherein:
the salt is a fumarate salt, and the salt is of Formula (I-A):

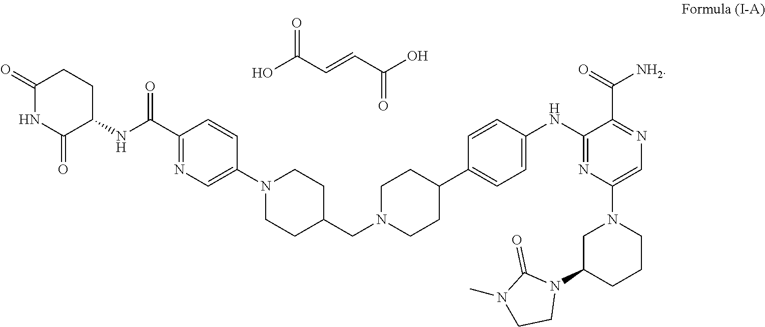 [SMILES: CN1CCN([C@@H]2CCCN(c3cnc(C(N)=O)c(Nc4ccc(C5CCN(CC6CCN(c7ccc(C(=O)N[C@H]8CCC(=O)NC8=O)nc7)CC6)CC5)cc4)n3)C2)C1=O ; O=C(O)/C=C/C(=O)O]

128. The salt of claim 125, wherein the salt is crystalline, semi-crystalline, amorphous, a hydrate, or a solvate.

129. The salt of claim 125, wherein the salt exhibits at least one diffraction peak at about 5.0−40° 2θ in its X-ray powder diffraction pattern.

130.-136. (canceled)

137. The salt of claim 125, wherein the salt is Fumarate Salt Form A.

138. The Fumarate Salt Form A of claim 137, wherein:
the X-ray powder diffraction pattern exhibits diffraction peaks at about 14.18, 16.04, 18.00, 18.48, 19.18, 21.16, and 22.56±0.2° 2θ; or
the differential scanning calorimetry thermogram exhibits one endotherm, wherein the endotherm has an endotherm maximum between about 240° C. and about 250° C.; or
the Fumarate Salt Form A exhibits a weight loss in a thermogravimetric analysis (TGA) in the range of about 0.1% to about 5% when heated from about 25° C. to about 125° C., and in the range of about 10% to about 15% when heated from about 125° C. to about 275° C.

139. The Fumarate Salt Form A of claim 138, wherein:
the X-ray powder diffraction pattern is substantially similar to that of FIG. 32; or
the differential scanning calorimetry thermogram is substantially similar to that of FIG. 37; or
the TGA pattern is substantially similar to that of FIG. 37.

140.-145. (canceled)

146. An amorphous solid dispersion comprising the salt of claim 137.

147.-148. (canceled)

149. The salt of claim 125, wherein the salt is Fumarate Salt Amorphous Form.

150. The Fumarate Salt Amorphous Form of claim 149, wherein:
the Fumarate Salt Amorphous Form is shown to be amorphous by X-ray powder diffraction; or
the differential scanning calorimetry thermogram exhibits one endotherm, where the endotherm has an endotherm maximum between about 30° C. and about 130° C.; or
the Fumarate Salt Amorphous Form exhibits a weight loss in a thermogravimetric analysis (TGA) in the range of about 1% to about 10% when heated from about 35° C. to about 180° C.

151. The Fumarate Salt Amorphous Form of claim 149, wherein:
the X-ray powder diffraction pattern is substantially similar to that of FIG. 39; or
the differential scanning calorimetry thermogram is substantially similar to that of FIG. 42; or
the TGA pattern is substantially similar to that of FIG. 41.

152. An amorphous solid dispersion comprising the salt of claim 149.

153.-154. (canceled)

155. The salt of claim 125, wherein the salt is Fumarate Salt Form B.

156. The Fumarate Salt Form B of claim 155, wherein the X-ray powder diffraction pattern exhibits diffraction peaks at about 6.60, 7.54, 7.67, 8.57, 16.42, 18.18, and 21.56±0.2° 2θ.

157. The Fumarate Salt Form B of claim 155, wherein the X-ray powder diffraction pattern is substantially similar to that of FIG. 45 (2nd from bottom)

158. An amorphous solid dispersion comprising the salt of claim 155.

159.-160. (canceled)

161. The salt of claim 125, wherein the salt is Fumarate Salt Form C.

162. The Fumarate Salt Form C of claim 161, wherein the X-ray powder diffraction pattern is substantially similar to that of FIG. 47 (2nd from bottom).

163. The Fumarate Salt Form C of claim 161, wherein the X-ray powder diffraction pattern exhibits diffraction peaks at about 6.14, 7.69, 8.08, 8.18, 12.14, 15.44, 20.89, 21.13, and 24.03±0.2°2θ.

164. An amorphous solid dispersion comprising the salt of claim 161.

165.-166. (canceled)

167. The salt of claim 125, wherein the salt is Fumarate Salt Form D.

168. The Fumarate Salt Form D of claim 167, wherein:
the differential thermal analysis thermogram exhibits one endotherm, wherein the endotherm has an endotherm maximum between about 220° C. and about 250° C.; or
the Fumarate Salt Form D exhibits a weight loss in a thermogravimetric analysis (TGA) in the range of about 30% to about 40% when heated from about 30° C. to about 300° C.

169. The Fumarate Salt Form D of claim 167, wherein:
the X-ray powder diffraction pattern is substantially similar to that of FIG. 49; or
the differential thermal analysis thermogram is substantially similar to that of FIG. 50; or
the TGA pattern is substantially similar to that of FIG. 50.

170. An amorphous solid dispersion comprising the salt of claim 167.

171.-173. (canceled)

174. The salt of claim 125, wherein the salt is Fumarate Salt Form E.

175. The Fumarate Salt Form E of claim 174, wherein:
the differential thermal analysis thermogram exhibits one endotherm, wherein the endotherm has an endotherm maximum between about 200° C. and about 240° C.; or
the Fumarate Salt Form E exhibits a weight loss in a thermogravimetric analysis (TGA) in the range of about 5% to about 15% when heated from about 40° C. to about 215° C.

176. The Fumarate Salt Form E of claim 175, wherein:
the X-ray powder diffraction pattern is substantially similar to that of FIG. 51; or
the differential thermal analysis thermogram is substantially similar to that of FIG. 52; or
the TGA pattern is substantially similar to that of FIG. 52.

177. An amorphous solid dispersion comprising the salt of claim 174.

178.-184. (canceled)

185. The salt of claim 125, wherein the salt is a maleate salt, a tosylate salt, a besylate salt, a cyclamate salt, a malate salt, a malonate salt, a napsylate salt, or a succinate salt.

186. The salt of claim 125, wherein the salt is of Formula (I-B), Formula (I-C), Formula (I-D), Formula (I-E), Formula (I-F), Formula (I-G), Formula (I-H), or Formula (I-J):

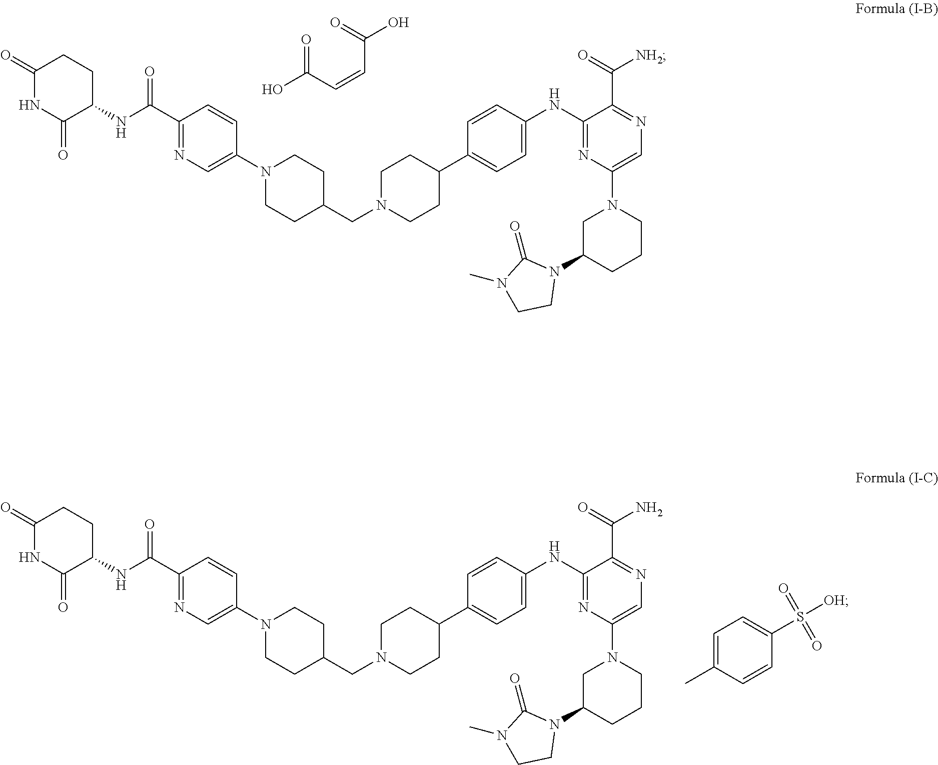

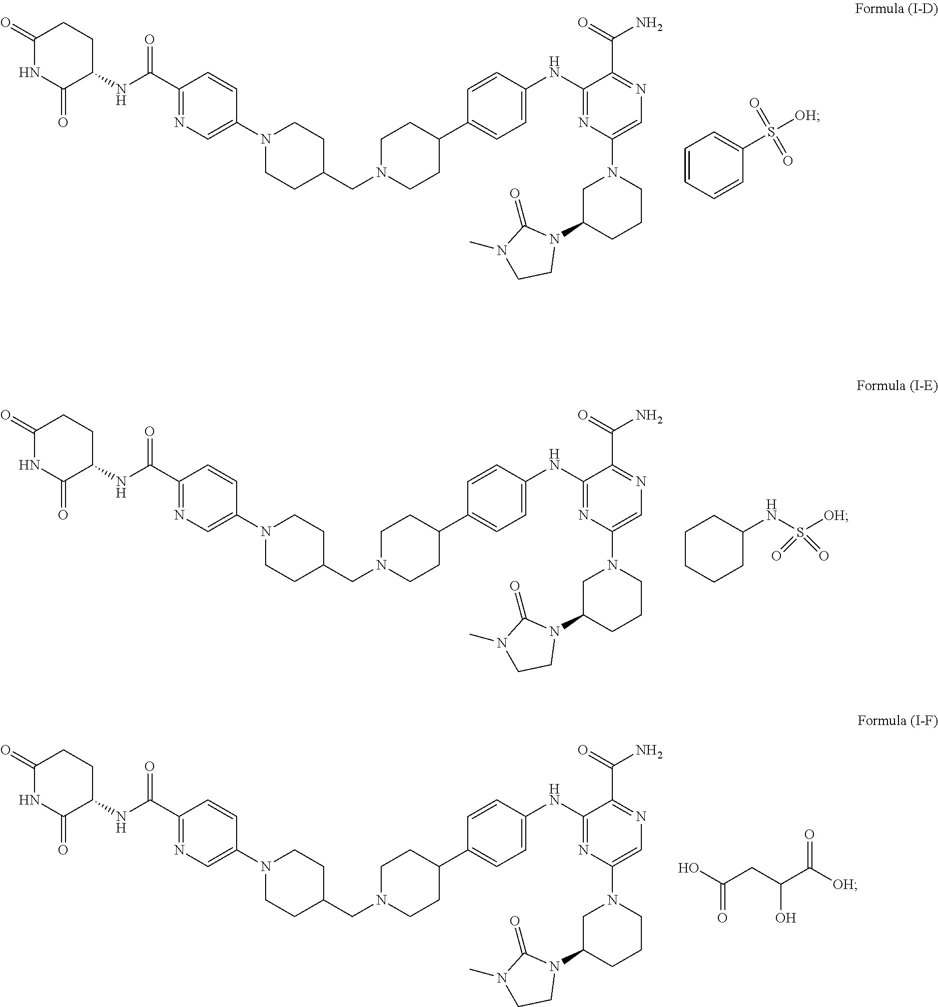

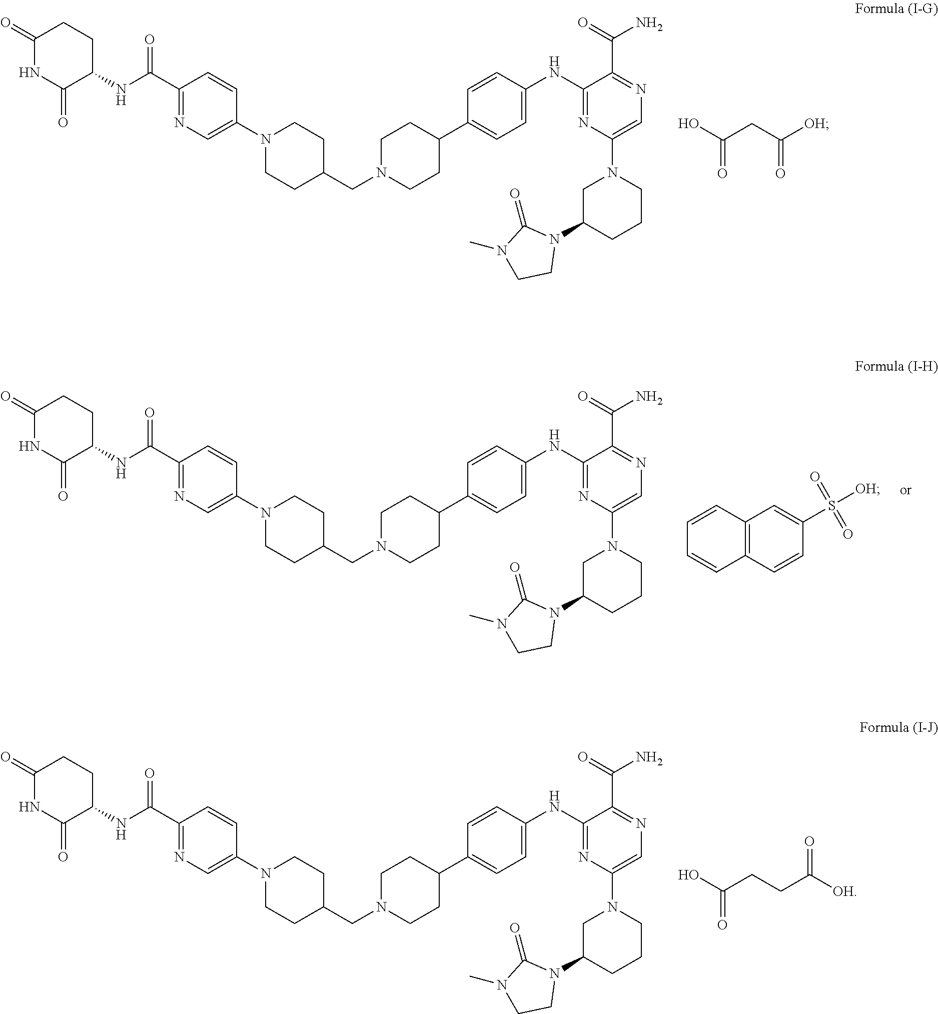 [SMILES: CN1CCN([C@@H]2CCCN(c3cnc(C(N)=O)c(Nc4ccc(C5CCN(CC6CCN(c7ccc(C(=O)N[C@H]8CCC(=O)NC8=O)nc7)CC6)CC5)cc4)n3)C2)C1=O ; O=C(O)CC(=O)O ; CN1CCN([C@@H]2CCCN(c3cnc(C(N)=O)c(Nc4ccc(C5CCN(CC6CCN(c7ccc(C(=O)N[C@H]8CCC(=O)NC8=O)nc7)CC6)CC5)cc4)n3)C2)C1=O ; O=S(=O)(O)c1ccc2ccccc2c1 ; CN1CCN([C@@H]2CCCN(c3cnc(C(N)=O)c(Nc4ccc(C5CCN(CC6CCN(c7ccc(C(=O)N[C@H]8CCC(=O)NC8=O)nc7)CC6)CC5)cc4)n3)C2)C1=O ; O=C(O)CCC(=O)O]

187. The salt of claim 185, wherein the salt is crystalline, semi-crystalline, amorphous, a salt, or a hydrate.

188.-196. (canceled)

197. The salt of claim 185, wherein the salt is Maleate Salt Form A.

198. The Maleate Salt Form A of claim 197, wherein:
the salt is a THF solvate;
the X-ray powder diffraction pattern exhibits diffraction peaks at about 14.20, 16.48, 18.28, 19.46, and 21.74±0.2°2θ; or
the differential scanning calorimetry thermogram exhibits one endotherm, where the endotherm has an endotherm maximum between about 225° C. and about 235° C.; or
the Maleate Salt Form A exhibits a weight loss in a thermogravimetric analysis (TGA) in the range of about 0.1% to about 5% when heated from about 25° C. to about 175° C., and in the range of about 5% to about 10% when heated from about 175° C. to about 250° C.

199. The Maleate Salt Form A of claim 197, wherein:
the X-ray powder diffraction pattern is substantially similar to that of FIG. 55; or
the differential scanning calorimetry thermogram is substantially similar to that of FIG. 56; or
the TGA pattern is substantially similar to that of FIG. 56.

200. An amorphous solid dispersion comprising the salt of claim 197.

201.-202. (canceled)

203. The salt of claim 185, wherein the salt is Tosylate Salt Form A.

204. The Tosylate Salt Form A of claim 203, wherein the X-ray powder diffraction pattern exhibits diffraction peaks at about 15.76, 19.34, 20.96, 22.86, and 24.02±0.2°2θ; or

205. The Tosylate Salt Form A of claim 203, wherein the X-ray powder diffraction pattern is substantially similar to that of FIG. 57.

206. An amorphous solid dispersion comprising the salt of claim 203.

207.-208. (canceled)

209. The salt of claim 185, wherein the salt is Tosylate Salt Form B.

210. The Tosylate Salt Form B of claim 209, wherein the X-ray powder diffraction pattern exhibits diffraction peaks at about 18.40, 19.12, 19.68, and 22.08±0.2°2θ.

211. The Tosylate Salt Form B of claim 209, wherein the X-ray powder diffraction pattern is substantially similar to that of FIG. 58.

212. An amorphous solid dispersion comprising the salt of claim 209.

213.-214. (canceled)

215. The salt of claim 185, wherein the salt is Besylate Salt Form A.

216. The Besylate Salt Form A of claim 215, wherein:
the differential thermal analysis thermogram exhibits two endotherms, wherein the first endotherm has an endotherm maximum between about 85° C. and about 160° C., and the second endotherm maxima between about 205° C. and about 255° C.; or
the Besylate Salt Form A exhibits a weight loss in a thermogravimetric analysis (TGA) in the range of about 0.1% to about 5% when heated from about 30° C. to about 90° C., and a weight loss in the range of about 30% to about 40% when heated from about 85° C. to about 160° C.

217. The Besylate Salt Form A of claim 215, wherein:
the X-ray powder diffraction pattern is substantially similar to that of FIG. 59; or
the differential thermal analysis thermogram is substantially similar to that of FIG. 60; or
the TGA pattern is substantially similar to that of FIG. 60.

218. An amorphous solid dispersion comprising the salt of claim 215.

219.-220. (canceled)

221. The salt of claim 185, wherein the salt is Cyclamate Salt Form A.

222. The Cyclamate Salt Form A of claim 221, wherein:
the differential thermal analysis thermogram exhibits two endotherms, wherein the first endotherm has an endotherm maximum between about 220° C. and about 245° C., and the second endotherm has an endotherm maximum between about 275° C. to about 305° C.; or
the Cyclamate Salt Form A exhibits a weight loss in a thermogravimetric analysis (TGA) in the range of about 1% to about 5% when heated from about 30° C. to about 235° C.

223. The Cyclamate Salt Form A of claim 221, wherein:
the X-ray powder diffraction pattern is substantially similar to that of FIG. 61; or
the differential thermal analysis thermogram is substantially similar to that of FIG. 62; or
the TGA pattern is substantially similar to that of FIG. 62.

224. An amorphous solid dispersion comprising the salt of claim 221.

225.-226. (canceled)

227. The salt of claim 185, wherein the salt is Malate Salt Form A.

228. The Malate Salt Form A of claim 227, wherein:
the differential thermal analysis thermogram exhibits two endotherms, wherein the first endotherm has an endotherm maximum between about 60° C. and about 130° C., and the second endotherm has an endotherm maximum between about 220° C. and about 260° C.; or
the Malate Salt Form A exhibits a weight loss in a thermogravimetric analysis (TGA) in the range of about 1% to about 10% when heated from about 30° C. to about 225° C.

229. The Malate Salt Form A of claim 227, wherein:
the X-ray powder diffraction pattern is substantially similar to that of FIG. 63; or
the differential thermal analysis thermogram is substantially similar to that of FIG. 64; or
the TGA pattern is substantially similar to that of FIG. 64.

230. An amorphous solid dispersion comprising the salt of claim 227.

231.-232. (canceled)

233. The salt of claim 185, wherein the salt is Malonate Salt Form A.

234. The Malonate Salt Form A of claim 233, wherein:
the differential thermal analysis thermogram exhibits one endotherm, wherein the endotherm has an endotherm maximum between about 140° C. and about 190° C.;
or
the Malonate Salt Form A exhibits a weight loss in a thermogravimetric analysis (TGA) in the range of about 1% to about 10% when heated from about 30° C. to about 140° C., about 1% to about 10% when heated from about 135° C. to about 180° C., and about 1% to about 10% when heated from about 180° C. to about 305° C.

235. The Malonate Salt Form A of claim 233, wherein:
the X-ray powder diffraction pattern is substantially similar to that of FIG. 65; or
the differential thermal analysis thermogram is substantially similar to that of FIG. 66; or
the TGA pattern is substantially similar to that of FIG. 66.

236. An amorphous solid dispersion comprising the salt of claim 233.

237.-238. (canceled)

239. The salt of claim 185, wherein the salt is Napsylate Salt Form A.

240. The Napsylate Salt Form A of claim 239, wherein:
the differential thermal analysis thermogram exhibits one endotherm, wherein the endotherm has an endotherm maximum between about 240° C. and about 280° C.; or
the Napsylate Salt Form A exhibits a weight loss in a thermogravimetric analysis (TGA) in the range of about 1% to about 5% when heated from about 30° C. to about 255° C.

241. The Napsylate Salt Form A, wherein:
the X-ray powder diffraction pattern is substantially similar to that of FIG. 67; or
the differential thermal analysis thermogram is substantially similar to that of FIG. 68; or
the TGA pattern is substantially similar to that of FIG. 68.

242. An amorphous solid dispersion comprising the salt of claim 239.

243.-244. (canceled)

245. The salt of claim 185, wherein the salt is Napsylate Salt Form B.

246. The Napsylate Salt Form B of claim 185, wherein the X-ray powder diffraction pattern is substantially similar to that of FIG. 69 (2^{nd} from top).

247. An amorphous solid dispersion comprising the salt of claim 245.

248.-249. (canceled)

250. The salt of claim 185, wherein the salt is Napsylate Salt Form C.

251. The Napsylate Salt Form C of claim 250, wherein the X-ray powder diffraction pattern is substantially similar to that of FIG. 69 (2^{nd} from bottom).

252. An amorphous solid dispersion comprising the salt of claim 250.

253.-254. (canceled)

255. The salt of claim 185, wherein the salt is Napsylate Salt Form D.

256. The Napsylate Salt Form D of claim 255, wherein:
the differential thermal analysis thermogram exhibits one endotherm, wherein the endotherm has an endotherm maximum between about 75° C. and about 140° C.; or
the Napsylate Salt Form D exhibits a weight loss in a thermogravimetric analysis (TGA) in the range of about 1% to about 5% when heated from about 25° C. to about 85° C., a range of about 20% to about 30% when heated from about 85° C. to about 125° C., and a range of about 0.1% to 5% when heated from about 150° C. to about 180° C.

257. The Napsylate Salt Form D of claim 255, wherein:
the X-ray powder diffraction pattern is substantially similar to that of FIG. 69 (bottom) or FIG. 70; or
the differential thermal analysis thermogram is substantially similar to that of FIG. 71; or
the TGA pattern is substantially similar to that of FIG. 71.

258. An amorphous solid dispersion comprising the salt of claim 255.

259.-260. (canceled)

261. The salt of claim 185, wherein the salt is Succinate Salt Form A.

262. The Succinate Salt Form A of claim 261, wherein the X-ray powder diffraction pattern is substantially similar to that of FIG. 72 (2nd from top, and bottom) and FIG. 73.

263. An amorphous solid dispersion comprising the salt of claim 261.

264.-265. (canceled)

266. The salt of claim 185, wherein the salt is Succinate Salt Form B.

267. The Succinate Salt Form B of claim 266, wherein:
the differential thermal analysis thermogram exhibits two endotherms, wherein the first endotherm has an endotherm maximum between about 75° C. and about 125° C., and the second endotherm has an endotherm maximum between about 200° C. and about 230° C.; or
the Succinate Salt Form B exhibits a weight loss in a thermogravimetric analysis (TGA) in the range of about 1% to about 5% when heated from about 30° C. to about 215° C.

268. The Succinate Salt Form B of claim 266, wherein:
the X-ray powder diffraction pattern is substantially similar to that of FIG. 72 (2^{nd} from bottom); or
the differential thermal analysis thermogram is substantially similar to that of FIG. 74; or
the TGA pattern is substantially similar to that of FIG. 74.

269. An amorphous solid dispersion comprising the salt of claim 266.

270.-271. (canceled)

272. The salt of claim 185, wherein the salt is Succinate Salt Form C.

273. The Succinate Salt Form C of claim 272, wherein:
the differential thermal analysis thermogram exhibits two endotherms, wherein the first endotherm has an endotherm maximum between about 80° C. and about 155° C., and the second endotherm has an endotherm maximum between about 200° C. and about 235° C.; or
the Succinate Salt Form C exhibits a weight loss in a thermogravimetric analysis (TGA) in the range of about 20% to about 30% when heated from about 70° C. to about 155° C.

274. The Succinate Salt Form C of claim 272, wherein:
the X-ray powder diffraction pattern is substantially similar to that of FIG. 75; or
the differential thermal analysis thermogram is substantially similar to that of FIG. 76; or
the TGA pattern is substantially similar to that of FIG. 76.

275. An amorphous solid dispersion comprising the salt of claim 272.

276.-284. (canceled)

285. A pharmaceutical composition comprising the free base of the compound of Formula (I) of claim 1 and a pharmaceutically acceptable carrier, vehicle, or adjuvant.

286. A method of treating a disease, disorder, or condition mediated by degrading Bruton's tyrosine kinase, comprising administering to a patient or biological sample the free base of the compound of Formula (I) of claim 1.

287. The method of claim 286, wherein the disease, disorder, or condition is selected from the group consisting of diffuse large B cell lymphoma (DLBCL), chronic lymphocytic leukemia (CLL), small lymphocytic lymphoma (SLL), follicular lymphoma (FL), mantle cell lymphoma (MCL), marginal zone lymphoma (MZL), Waldenström macroglobulinemia (WM), and primary central nervous system lymphoma (PCNSL).

288. The method of claim 287, wherein the DLBCL is non-Germinal center B-cell like subtype (non-GCB) DLBCL, and/or the MCL is non-GCB MCL.

289. The method of claim 287, wherein the disease, disorder, or condition includes secondary central nervous system (CNS) lymphoma.

290. A method of degrading splenocyte Bruton's tyrosine kinase in a subject in need thereof, comprising the step of orally administering to the subject an amount of the free base of the compound of Formula (I) of claim 1, wherein said salt or free base is capable of inducing proteolytic degradation of Bruton's tyrosine kinase, and wherein said amount is effective to degrade splenocyte Bruton's tyrosine kinase in the subject.

291. A pharmaceutical composition comprising the salt of the compound of Formula (I) of claim 125 and a pharmaceutically acceptable carrier, vehicle, or adjuvant.

292. A method of treating a disease, disorder, or condition mediated by degrading Bruton's tyrosine kinase, comprising administering to a patient or biological sample the salt of the compound of Formula (I) of claim 125.

293. The method of claim 292, wherein the disease, disorder, or condition is selected from the group consisting of diffuse large B cell lymphoma (DLBCL), chronic lymphocytic leukemia (CLL), small lymphocytic lymphoma (SLL), follicular lymphoma (FL), mantle cell lymphoma (MCL), marginal zone lymphoma (MZL), Waldenström macroglobulinemia (WM), and primary central nervous system lymphoma (PCNSL).

294. The method of claim 293, wherein the DLBCL is non-Germinal center B-cell like subtype (non-GCB) DLBCL, and/or the MCL is non-GCB MCL.

295. The method of claim 293, wherein the disease, disorder, or condition includes secondary central nervous system (CNS) lymphoma.

296. A method of degrading splenocyte Bruton's tyrosine kinase in a subject in need thereof, comprising the step of orally administering to the subject an amount of the salt of the compound of Formula (I) of claim 125, wherein said salt is capable of inducing proteolytic degradation of Bruton's tyrosine kinase, and wherein said amount is effective to degrade splenocyte Bruton's tyrosine kinase in the subject.

297. An amorphous solid dispersion comprising the free base of claim 1.

298. The amorphous solid dispersion of claim 297, further comprising a dispersion aid selected from the group consisting of a hydroxypropyl methylcellulose acetate succinate (HPMCAS), an anionic copolymer consisting of methacrylic acid/ethyl acrylate, or a random copolymer of N-vinyl pyrrolidone and vinyl acetate (PVP/VA).

299. The amorphous solid dispersion of claim 298, wherein the HPMCAS is HPMCAS-H or HPMCAS-L, the anionic copolymer consisting of methacrylic acid and ethyl acrylate is Eudragit L100, or the PVP/VA is PVP-VA64.

300. An amorphous solid dispersion comprising the salt of claim 125.

301. The amorphous solid dispersion of claim 300, further comprising a dispersion aid selected from the group consisting of a hydroxypropyl methylcellulose acetate succinate (HPMCAS), an anionic copolymer consisting of methacrylic acid/ethyl acrylate, or a random copolymer of N-vinyl pyrrolidone and vinyl acetate (PVP/VA).

302. The amorphous solid dispersion of claim 301, wherein the HPMCAS is HPMCAS-H or HPMCAS-L, the anionic copolymer consisting of methacrylic acid and ethyl acrylate is Eudragit L100, or the PVP/VA is PVP-VA64.
